(19) United States
(12) Patent Application Publication
(10) Pub. No.: US 2021/0324082 A1
(43) Pub. Date: Oct. 21, 2021

(54) PD-L1 TARGETING MOLECULES COMPRISING SHIGA TOXIN A SUBUNIT SCAFFOLDS

(71) Applicant: Molecular Templates, Inc., Austin, TX (US)

(72) Inventors: Eric Poma, New York, NY (US); Hilario Ramos, Austin, TX (US); Erin Willert, Round Rock, TX (US)

(21) Appl. No.: 17/204,486

(22) Filed: Mar. 17, 2021

Related U.S. Application Data
(60) Provisional application No. 63/010,671, filed on Apr. 15, 2020.

(51) Int. Cl. C07K 16/28; C07K 14/25

(52) U.S. Cl. CPC C07K 16/2827; C07K 14/25; C07K 2317/33; C07K 2317/565; C07K 2319/30

(57) ABSTRACT

Provided herein are PD-L1 binding molecules comprising Shiga toxin A Subunit derived polypeptides and methods of using the same. Certain PD-L1 targeting molecules embodiments are cytotoxic. Certain PD-L1 targeting molecules embodiments exhibit reduced immunogenic potential in mammals and/or are capable of delivering an immunogenic epitope to an MHC class I molecule of a PD-L1 positive cell. Certain PD-L1 targeting molecules embodiments are well-tolerated by mammals while retaining one or more of the features mentioned above. The PD-L1 targeting molecules have uses for selectively killing specific cells (e.g., PD-L1 positive tumor and/or immune cells); for selectively delivering cargos to specific cells (e.g., PD-L1 positive tumor and/or immune cells), and as therapeutic and/or diagnostic molecules for treating and diagnosing a variety of conditions, including cancers and tumors involving PD-L1 expressing cells.

### CROSS-REFERENCE TO RELATED APPLICATIONS

This application claims priority to U.S. Provisional Application No. 63/010,671, filed Apr. 15, 2020, the contents of which are incorporated by reference herein in their entirety.

### SEQUENCE LISTING

This application contains a Sequence Listing which has been submitted electronically and is hereby incorporated by reference in its entirety. The Sequence Listing was recorded Mar. 16, 2021, is named MTEM_030_01US_SeqList_ST25.txt, and is about 653 kilobytes in size.

### TECHNICAL FIELD

The present application relates to PD-L1 targeing molecules comprising Shiga toxin effector polypeptides, derived from the A Subunits of naturally occurring Shiga toxins. In some embodiments, the PD-L1 targeing molecules comprise Shiga toxin effector polypeptides that comprise a combination of mutations providing (1) de-immunization, (2) a reduction in protease sensitivity, and/or (3) an embedded, T-cell epitope(s); wherein the Shiga toxin effector polypeptides retain one or more Shiga toxin functions, such as, e.g., potent cytotoxicity. In some embodiments, the Shiga toxin effector polypeptides (1) exhibit reduced immunogenic potential in mammals and/or (2) are capable of delivering a CD8+ T-cell epitope to the MHC class I system of a PD-L1 expressing cell. The PD-L1 targeting molecules are useful for administration to multicellular organisms in order to kill PD-L1 expressing cells, such as, e.g., in situations when it is desirable to (1) eliminate or reduce non-specific toxicities, (2) eliminate or reduce certain immune responses, and/or (3) target a beneficial immune response(s) to a specific epitope delivered to a specific cell-type, such as, e.g., to recruit and activate CD8+ T-cells to a cell and/or tissue locus. The PD-L1 targeting molecules are useful (1) for selectively killing specific PD-L1 positive cell type(s) amongst other cells and (2) as therapeutic molecules for treating a variety of diseases, disorders, and conditions involving PD-L1 expressing cells, including cancers and tumors.

### BACKGROUND

The following includes information that may be useful in understanding the invention(s) described herein. It is not an admission that any of the information provided herein is prior art or relevant to the presently described or claimed invention(s), or that any publication or document that is specifically or implicitly referenced herein is prior art.

PD-L1, programmed cell death ligand 1 (also known as CD274), is expressed on the cell surface of tumors from a variety of malignancies and can bind to PD-1 on T-cells, leading to immune evasion of the tumor. Blockade of the PD-L1/PD-1 axis by therapeutic monoclonal antibodies has clinical efficacy. Novel binding domains targeting human PD-L1 are of interest in developing PD-L1 targeted agents for therapeutic or diagnostic purposes. Oncology indications include but are not limited to lung cancer, melanoma, bladder cancer, Hogkin's lymphoma, breast cancer (including triple negative for HER2, estrogen receptor, and progesterone receptor) as well as other malignancies with PD-L1 expression. PD-L1 is also expressed on the surface of some immune cells and could be a target for delivery of vaccines or other immunomodulatory agents to a subset of immune cells.

There is an urgent need for new therapeutics to supplement present day therapies for PD-L1 bearing neoplasms. Thus, it would be desirable to have cytotoxic PD-L1 targeting molecules which target PD-L1 for use as therapeutic molecules to treat a variety of diseases, such as, e.g., cancers and tumors, that can be treated by selective killing of a PD-L1 positive cell, or selective delivery of a beneficial agent thereto. In particular, it would be desirable to have PD-L1-binding, cytotoxic, PD-L1 targeting molecules exhibiting low antigenicity and/or immunogenicity, low off-target toxicity, and potent cytotoxicity. Furthermore, it would be desirable to have PD-L1 targeting therapeutic and/or diagnostic molecules exhibiting low antigenicity and/or immunogenicity, low off-target toxicity, high stability, and/or the ability to deliver peptide-epitope cargos for presentation by the MHC class I system of a target cell. For example, it would be desirable to have PD-L1 targeting molecules comprising Shiga toxin A Subunit derived components that maintain potent cytotoxicity while 1) reducing the potential for unwanted antigenicities and/or immunogenicities, 2) reducing the potential for non-specific toxicities, and/or 3) having the ability to deliver peptide-epitope cargos for presentation by the MHC class I system of a target cell, and which also exhibit potent Shiga toxin A Subunit-based cytotoxicity to target cells.

### BRIEF SUMMARY

Provided herein are various embodiments of PD-L1 targeting molecules, and compositions thereof, wherein each PD-L1 targeting molecule comprises (1) at least one Shiga toxin A Subunit effector polypeptide derived from the A Subunit of at least one member of the Shiga toxin family and (2) at least one PD-L1 binding region capable of specifically binding an extracellular part of a PD-L1 molecule. For each PD-L1 targeting molecule, the at least one binding region is heterologous to the Shiga toxin A Subunit effector polypeptide, such as, e.g., an immunoglobulin-type binding region.

The direct targeting of PD-L1 expressing immune cells may represent a treatment modality that is quite different compared to approved immune checkpoint inhibitors that reinvigorate antitumor immune responses by sterically blocking inhibitory signaling pathways. However, immune checkpoint inhibitors are not always effective, and checkpoint inhibitor therapy has been associated with severe side effects in patients, especially in combination. Thus, there is a need in the art for cancer therapies that are more effective, deplete immunosuppressive immune cells, and have less side effects compared to approved checkpoint inhibitors.

In some embodiments, the PD-L1 binding region comprises a heavy chain variable region (HVR-H) comprising three CDRs, each having at least 85%, 90%, 91%, 92%, 93%, 94%, 95%, 96%, 97%, 98%, or 99% identity to SEQ ID NO:22, SEQ ID NO:23, SEQ ID NO:24, SEQ ID NO:36, or SEQ ID NO:37; or consisting essentially of an amino acid sequence show in any one of SEQ ID NO:22, SEQ ID NO:23, SEQ ID NO:24, SEQ ID NO:36, and SEQ ID NO:37. In some embodiments, the binding region further comprises: (a) a light chain variable region (HVR-L) comprising three CDRs, having at least 85%, 90%, 91%, 92%, 93%, 94%, 95%, 96%, 97%, 98%, or 99% identity to SEQ ID NO:19, SEQ ID NO:20, and SEQ ID NO:21; or consisting essentially of an amino acid sequence shown in any one of SEQ ID NO:19, SEQ ID NO:20, and SEQ ID NO:21. In some embodiments, the binding region comprises: (a) a light chain variable region (HVR-L) comprising three CDRs, each comprising or consisting essentially of an amino acid sequence shown in any one of SEQ ID NO:19, SEQ ID NO:20, and SEQ ID NO:21; and (b) a heavy chain variable region (HVR-H) comprising three CDRs, each comprising or consisting essentially of an amino acid sequence shown in any one of SEQ ID NO:22, SEQ ID NO:23, SEQ ID NO:24, SEQ ID NO:36, and SEQ ID NO:37.

In some embodiments, the binding region comprises: (a) a light chain region having at least 85%, 90%, 91%, 92%, 93%, 94%, 95%, 96%, 97%, 98%, or 99%, identity to SEQ ID NO:25 or SEQ ID NO:27 or consisting essentially of the amino acid sequence of SEQ ID NO:25 or SEQ ID NO:27; and/or (b) a heavy chain region having at least 85%, 90%, 91%, 92%, 93%, 94%, 95%, 96%, 97%, 98%, or 99% identity to SEQ ID NO:26 or consisting essentially of the amino acid sequence of SEQ ID NO:26. In some embodiments, the binding region comprises a polypeptide having at least 85%, 90%, 91%, 92%, 93%, 94%, 95%, 96%, 97%, 98%, or 99% identity to any one of SEQ ID NOs: 28-35 and 38-40 or consists essentially of the polypeptide shown in any one of SEQ ID NOs: 28-35 and 38-40. In some embodiments, the binding region is a single-chain variable fragment, such as, e.g., consisting of, comprising, or consisting essentially of the polypeptide of any one of SEQ ID NOs: 28-35 and 38-40.

In some embodiments, a PD-L1 binding molecule comprises: A) a Shiga toxin A subunit effector polypeptide; and B) a binding region capable of specifically binding an extracellular part of PD-L1, wherein the binding region comprises (a) a light chain variable region comprising: (i) a CDR1 comprising the amino acid sequence TGTSSDVGSYNRVS (SEQ ID NO:19), (ii) a CDR2 comprising the amino acid sequence EVSNRPS (SEQ ID NO:20), and (iii) a CDR3 comprising the amino acid sequence SSHTTSGTYV (SEQ ID NO:21); and (b) a heavy chain variable region comprising: (i) a CDR1 comprising the amino acid sequence SYAIS (SEQ ID NO:22), (ii) a CDR2 comprising the amino acid sequence GIIPIFGTANYAQKFQG (SEQ ID NO:23), and (iii) a CDR3 comprising the amino acid sequence DQGYAHAFDI (SEQ ID NO:24).

In some embodiments, a method of killing a PD-L1 expressing cell comprises the step of contacting the cell with a PD-L1 binding molecule or a pharmaceutical composition comprising the same. In some embodiments, administration of the PD-L1 targeting molecule to a PD-L1 expressing cell results in (i) the internalization of the PD-L1 targeting molecule by the cell and (ii) the death of the cell. In some embodiments, administration of the PD-L1 targeting molecule to a PD-L1 expressing cell results in (i) the internalization of the PD-L1 targeting molecule by the cell and (ii) the death of the cell due to a catalytically active Shiga toxin A subunit effector polypeptide. In some embodiments, administration of the PD-L1 targeting molecule to a PD-L1 expressing cell results in (i) the internalization of the PD-L1 targeting molecule by the cell and (ii) the death of the cell due to delivery and presentation of T-cell epitope cargo. In some embodiments, administration of the PD-L1 targeting molecule to a PD-L1 expressing cell results in (i) the internalization of the PD-L1 targeting molecule by the cell and (ii) the cell presenting on a cellular surface a heterologous, CD8+ T-cell epitope-peptide cargo delivered by the PD-L1 targeting molecule complexed with a MHC class I molecule. In some embodiments, the PD-L1 targeting molecules are capable, when introduced into to cells, of exhibiting a cytotoxicity with a half-maximal inhibitory concentration (CD₅₀) value of 300 nM or less and/or capable of exhibiting a significant level of Shiga toxin cytotoxicity. In some embodiments, the Shiga toxin A Subunit effector polypeptide is capable of exhibiting a ribosome inhibition activity with a half-maximal inhibitory concentration (IC₅₀) value of less than 10,000, 5,000, 1,000, 500, or 200 picomolar.

In some embodiments, the at least one Shiga toxin A Subunit derived polypeptide comprises a combination of features (e.g., de-immunized sub-region(s), heterologous epitope comprising sub-region(s), a protease-cleavage resistant sub-region, and/or a carboxy-terminal, endoplasmic reticulum retention/retrieval signal motif). In some embodiments, the PD-L1 targeting molecules provide a combination of several properties in a single molecule, such as, e.g., efficient cellular internalization, potent cell-targeted cytotoxicity, selective cytotoxicity, de-immunization, low non-specific toxicity at high dosages, high stability, CD8+ T-cell hyper-immunization, and/or the ability to deliver a heterologous, T-cell epitope(s) to the MHC I class pathway of a target cell.

In some embodiments, the PD-L1 targeting molecules are useful for administration to chordates, such as, e.g., when it is desirable to (1) reduce or eliminate a certain immune response(s) resulting from the administered molecule, (2) reduce or eliminate non-specific toxicities resulting from the administered molecule, (3) specifically kill a PD-L1 expressing target cell(s) in vivo, and/or (4) target a beneficial immune response(s) to a target cell-type, a tumor mass comprising a target cell-type, and/or a tissue locus comprising such a target cell-type, such as via stimulating intercellular engagement of a CD8+ T-cell(s) of the chordate with a specific MHC class I-eptiope complex displaying target cell-type.

In some embodiments, a method of treating a disease, disorder, or condition involving a PD-L1 expressing cell type comprises the step administering to a patient in need thereof an effective amount of a PD-L1 binding molecule or a pharmaceutical composition comprising the same. The disease, disorder, or condition may be selected from, for example, cancer, tumor, immune disorder, and microbial infection. In some embodiments, the cancer is selected from: bone cancer, breast cancer, central/peripheral nervous system cancer, gastrointestinal cancer, germ cell cancer, glandular cancer, head-neck cancer, hematological cancer, kidney-urinary tract cancer, liver cancer, lung/pleura cancer, prostate cancer, sarcoma, skin cancer, and uterine cancer. In some embodiments, the immune disorder is associated with a disease selected from: amyloidosis, ankylosing spondylitis, asthma, Crohn's disease, diabetes, graft rejection, graft versus host disease, Hashimoto's thyroiditis, hemolytic uremic syndrome, HIV-related disease, lupus erythmatosus, multiple sclerosis, polyarteritis, psoriasis, psoriatic arthritis, rheumatoid arthritis, scleroderma, septic shock, Sjögren's syndrome, ulcerative colitis, and vasculitis.

In some embodiments, a method for purifying the PD-L1 binding molecule from a cellular lysate comprising the PD-L1 binding molecule comprises (i) contacting the cellular lysate with a bacterial protein L to create a protein L-PD-L1 binding molecule complex, and (ii) separating the protein L-PD-L1 binding molecule complex from the cellular lysate.

These and other features, aspects and advantages will become better understood with regard to the following description, appended claims, and accompanying figures. The aforementioned elements may be individually combined or removed freely in order to make other embodiments of the invention, without any statement to object to such combination or removal hereinafter.

### BRIEF DESCRIPTION OF THE DRAWINGS

FIG. 1A-1D provide schematic drawings of illustrative PD-L1 targeting molecules. These exemplary PD-L1 binding molecules comprise one or more toxins or toxin subunits, such as Shiga toxin A Subunit effector polypeptides, and one or more PD-L1 binding regions (FIG. 1A, 1B). In some exemplary PD-L1 binding molecules, the PD-L1 binding region is a scFv, and the scFv is shown participating in intermolecular variable domain exchange with a neighboring scFv (FIG. 1B, bottom left). In some embodiments, the PD-L1 binding molecules comprise a heterologous CD8+ T-cell epitope, which may be located, for example, at the N-terminus or C-terminus of the molecule (FIG. 1C, 1B).

FIG. 2 is a graph showing binding of illustrative PD-L1 targeting molecules to PD-L1 expressing target cells, as measured by flow cytometry.

FIG. 3 is a graph showing cytotoxicity of illustrative PD-L1 targeting molecules to PD-L1 expressing cells.

FIG. 4 is a graph showing interferon-gamma (IFN-γ) secretion induced by illustrative PD-L1 targeting molecules in donor cytotoxic T lymphocytes (CTL) and tumor cell co-cultures.

FIG. 5 is a graph showing PD-1/PD-L1 intercellular signaling effects induced by an anti-PD-L1 monoclonal antibody (mAb), a PD-L1 targeting molecule (DI-SLTA-1-non target), a molecule comprising a Shiga toxin A subsunit effector and a binding domain that does not bind to PD-L1 (DI-SLTA-1-nontarget), and 115695. The assay was performed in a cell line that expresses a luciferase (LUC) reporter driven by a Nuclear Factor of Activated T cells (NFAT) response element. Luciferase expression was detected by measuring relative light units (RLU).

FIG. 6 is a graph showing dose-dependent killing of target cells in the presence of donor cytotoxic T lymphocytes (CTLs).

FIG. 7 is a graph showing cytotoxicity of illustrative PD-L1 targeting molecules to PD-L1 positive/HLA:A02 positive cells in the presence of donor cytotoxic T lymphocytes (CTLs). “Ina” refers to catalytically inactivated DI-SLTA subunits.

FIG. 8 is a graph demonstrating that ER-DI-SLTA-1 constructs (“ER” indicating that the constructs comprise an endoplasmic reticulum retention signal) display less cytotoxicity compared to their DI-SLTA-1 counterparts.

FIG. 9 is a graph demonstrating that ER-DI-SLTA-1 constructs display cytotoxicity to PD-L1 positive/HLA:A02 positive cells in the presence of donor cytotoxic T lymphocytes.

FIG. 10 is a graph showing interferon-gamma (IFN-γ) secretion induced by illustrative PD-L1 targeting molecules in donor CTL and tumor cell co-cultures.

FIG. 11A is a schematic that shows the protocol for a cytotoxicity assay in the presence of CMV-antigen specific cytotoxic T lymphocytes (CTLs), after 4-hour acute exposure and 24-hour sustained exposure.

FIG. 11B shows cytotoxicity of illustrative PD-L1 targeting molecules to PD-L1 positive/HLA:A02 positive cells in the presence of cytotoxic T lymphocytes after acute or sustained exposure. “C1” referes to a polypeptide comprising only the CD8+ T-cell epitope.

FIG. 12 shows cytotoxicity of illustrative PD-L1 targeting molecules to PD-L1 positive/HLA:A02 positive cells in the presence of cytotoxic T lymphocytes, after acute (4 hour) or sustained (24 hour) exposure.

FIG. 13A shows a membrane proteome array for molecule DI-SLTA-scFva::C1::C1, also referred to herein as 115765 (SEQ ID NO: 87). The second dot (on the X-axis, near 4000) corresponds to BACE2, which binds to poorly folded molecules.

FIG. 13B shows a membrane proteome array (no antigen control) for molecule DI-SLTA-1::scFva (SEQ ID NO: 86), also referred to herein as 115695.

FIG. 14 provides graphs showing that treatment with a PD-L1 targeting molecule induces cytokine secretion by CTLs, which is specific and correlated with both MEW allele expression and PD-L1 expression. Surface expression of HLA:A2 and PD-L1 was determined using flow cytometry.

FIG. 15 is a graph showing viability of MDA-MB231 cells over time, after treatment with various PD-L1 targeting molecules (right panel). FIG. 15 also shows interferon-gamma (IFN-γ) secretion as determined by ELISA, after treatment with various PD-L1 targeting molecules. FIG. 15 demonstrates that two cytotoxic mechanisms (inhibition of ribosomes (MOA-1) and delivery of a CD8+ T-cell epitope (MOA-2)) function together, resulting in greater target cell cytotoxicity.

FIG. 16 is a graph showing cytotoxicity in MDA-Mb231 cells resulting from acute or sustained PD-L1 targeting molecule exposure in the presence of antigen-specific CTLs.

FIG. 17 shows cytokine secretion (interferon gamma, IFN-γ) resulting from acute or sustained PD-L1 targeting molecule exposure in the presence of antigen-specific CTLs.

FIG. 18 is a schematic showing two mechanisms of action available in a single PD-L1 targeting scaffold. First, ribosomal inactivation requires cytosolic localization and a catalytically active Shiga toxin A Subunit effector polypeptide component. Second, CD8+ T-cell epitope delivery to the MEW class I presentation pathway within the target cell. In some embodiments, it is possible to deliver a CD8+ T-cell epitope without having a sigfnciant impact on ribosomal inactivation.

FIG. 19 is a schematic futher illustrating that there are two mechanisms of action (MOA) available in a single scaffold. Ribosomal inactivation requires cytosolic localization and catalytically active Shiga toxin A Subunit effector polypeptide (MOA1). Second, CD8+ T-cell epitope delivery to the MHC class I presentation pathway within the target cell is possible (MOA2), and this second mechanism of action does not interfere with the first. For example, CD8+ T-cell epitope delivery to the MEW class I presentation pathway possible without the first mechanism of action, using Shiga toxin A Subunit effector polypeptide inactivated scaffolds, such as catalytically inactive forms and/or carboxy-terminus truncations resulting in ER retention.

FIG. 20 is a chart showing choice of antigen and relative advantages thereof, in connection with antigen seeding technology (AST).

FIG. 21 shows functional profiles for illustrative PD-L1 targeting molecules. Atezolizumab is a fully humanized, engineered monoclonal antibody (IgG1) that binds to PD-L1. Underlined values represent results consistent with a favorable AST profile.

FIG. 22 is a summary of the functional profile for DI-SLTA-1::scFva::C1::C1 (115765, SEQ ID NO: 87).

FIG. 23 provides graphs showing cytotoxicity of various illustrative PD-L1 targeting molecules on HCC 1954 (left panel) and MDA-MB-231 (right panel) cells. Molecule 115765 corresponds to SEQ ID NO: 87.

FIG. 24 is a chart showing immune effects and pharmacodynamic response after dosing with either 115765 (SEQ ID NO: 87) or 115695 (SEQ ID NO: 86). irAE stands for immune-related adverse events.

FIGS. 25A and 25B shows comparative in vitro data for different PD-L1 binding molecules (115749, 115765 (SEQ ID NO: 87), and 115695 (SEQ ID NO: 86)). This data is from an assay testing antigen seeding in MDA-MB-231 cells after incubation with a PD-L1 binding molecule for 4 hours or 24 hours.

FIGS. 26A and 26B show the results of an experiment wherein monocytes (IC) isolated from donor patients were treated with 20 μg/mL of various PD-L1 binding molecules (FIG. 26A), and tumor cells (HCC1954) were treated with 2 μg/mL of the PD-L1 targeting molecules (FIG. 25B). Cell kill was measured using a standard Cell Titer Glo assay. Molecule 115765 corresponds to SEQ ID NO: 87 and 115695 corresponds to SEQ ID NO: 86. “DI-SLTA” refers to the Shiga toxin A subunit alone (i.e., without a PD-L1 binding domain).

### DETAILED DESCRIPTION

The present invention is described more fully hereinafter using illustrative, non-limiting embodiments, and references to the accompanying figures. This invention may, however, be embodied in many different forms and should not be construed as to be limited to the embodiments set forth below. Rather, these embodiments are provided so that this disclosure is thorough and conveys the scope of the invention to those skilled in the art.

In order that the present invention may be more readily understood, certain terms are defined below. Additional definitions may be found within the detailed description of the invention.

As used in the specification and the appended claims, the terms “a,” “an” and “the” include both singular and the plural referents unless the context clearly dictates otherwise.

As used in the specification and the appended claims, the term “and/or” when referring to two species, A and B, means at least one of A and B. As used in the specification and the appended claims, the term “and/or” when referring to greater than two species, such as A, B, and C, means at least one of A, B, or C, or at least one of any combination of A, B, or C (with each species in singular or multiple possibility).

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout this specification, the term “including” is used to mean “including but not limited to.” “Including” and “including but not limited to” are used interchangeably.

The term “amino acid residue” or “amino acid” includes reference to an amino acid that is incorporated into a protein, polypeptide, or peptide. The term “polypeptide” includes any polymer of amino acids or amino acid residues. The term “polypeptide sequence” refers to a series of amino acids or amino acid residues which physically comprise a polypeptide. A “protein” is a macromolecule comprising one or more polypeptides or polypeptide “chains.” A “peptide” is a small polypeptide of sizes less than about a total of 15 to 20 amino acid residues. The term “amino acid sequence” refers to a series of amino acids or amino acid residues which physically comprise a peptide or polypeptide depending on the length. Unless otherwise indicated, polypeptide and protein sequences disclosed herein are written from left to right representing their order from an amino-terminus to a carboxy-terminus.

The terms “amino acid,” “amino acid residue,” “amino acid sequence,” or polypeptide sequence include naturally occurring amino acids (including L and D isosteriomers) and, unless otherwise limited, also include known analogs of natural amino acids that can function in a similar manner as naturally occurring amino acids, such as selenocysteine, pyrrolysine, N-formylmethionine, gamma-carboxyglutamate, hydroxyprolinehypusine, pyroglutamic acid, and selenomethionine. The amino acids referred to herein are described by shorthand designations as follows in Table 1:

TABLE 1
Amino Acid Nomenclature

| Name | 3-letter | 1-letter |
|---|---|---|
| Alanine | Ala | A |
| Arginine | Arg | R |
| Asparagine | Asn | N |
| Aspartic Acid or Aspartate | Asp | D |
| Cysteine | Cys | C |
| Glutamic Acid or Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The phrase “conservative substitution” with regard to an amino acid residue of a peptide, peptide region, polypeptide region, protein, or molecule refers to a change in the amino acid composition of the peptide, peptide region, polypeptide region, protein, or molecule that does not substantially alter the function and structure of the overall peptide, peptide region, polypeptide region, protein, or molecule (see Creighton, Proteins: Structures and Molecular Properties (W. H. Freeman and Company, New York (2nd ed., 1992))).

As used herein, the phrase “derived from” when referring to a polypeptide or polypeptide region means that the polypeptide or polypeptide region comprises amino acid sequences originally found in a “parental” protein and which may now comprise certain amino acid residue additions, deletions, truncations, rearrangements, or other alterations relative to the original polypeptide or polypeptide region as long as a certain function(s) and/or a structure(s) of the “parental” molecule are substantially conserved. The skilled worker will be able to identify a parental molecule from which a polypeptide or polypeptide region was derived using techniques known in the art, e.g., protein sequence alignment software.

As used herein and with regard to a Shiga toxin polypeptide sequence or Shiga toxin derived polypeptide, the term “wild-type” generally refers to a naturally occurring, Shiga toxin protein sequence(s) found in a living species, such as, e.g., a pathogenic bacterium, wherein that Shiga toxin protein sequence(s) is one of the most frequently occurring variants. This is in contrast to infrequently occurring Shiga toxin protein sequences that, while still naturally occurring, are found in less than one percent of individual organisms of a given species when sampling a statistically powerful number of naturally occurring individual organisms of that species which comprise at least one Shiga toxin protein variant. A clonal expansion of a natural isolate outside its natural environment (regardless of whether the isolate is an organism or molecule comprising biological sequence information) does not alter the naturally occurring requirement as long as the clonal expansion does not introduce new sequence variety not present in naturally occurring populations of that species and/or does not change the relative proportions of sequence variants to each other.

The terms “associated,” “associating,” “linked,” or “linking” as used herein refer to the state of two or more components of a molecule being joined, attached, connected, or otherwise coupled to form a single molecule or the act of making two molecules associated with each other to form a single molecule by creating an association, linkage, attachment, and/or any other connection between the two molecules. For example, the term “linked” may refer to two or more components associated by one or more atomic interactions such that a single molecule is formed and wherein the atomic interactions may be covalent and/or non-covalent. Non-limiting examples of covalent associations between two components include peptide bonds and cysteine-cysteine disulfide bonds. Non-limiting examples of non-covalent associations between two molecular components include ionic bonds.

As used herein, the term “linked” refers to two or more molecular components associated by one or more atomic interactions such that a single molecule is formed and wherein the atomic interactions includes at least one covalent bond. As used herein, the term “linking” refers to the act of creating a linked molecule as described above.

As used herein, the term “fused” refers to two or more proteinaceous components associated by at least one covalent, peptide bond, regardless of whether the peptide bond involves the participation of a carbon atom of a carboxyl acid group or involves another carbon atom, such as, e.g., the α-carbon, β-carbon, γ-carbon, σ-carbon, etc. Non-limiting examples of two proteinaceous components fused together include, e.g., an amino acid, peptide, or polypeptide fused to a polypeptide via a peptide bond such that the resulting molecule is a single, continuous polypeptide. As used herein, the term “fusing” refers to the act of creating a fused molecule as described above, such as, e.g., a fusion protein generated from the recombinant fusion of genetic regions which when translated produces a single proteinaceous molecule.

The symbol “::” means the polypeptide regions before and after it are physically linked together to form a continuous polypeptide.

As used herein, the terms “expressed,” “expressing,” or “expresses,” and grammatical variants thereof, refer to translation of a polynucleotide or nucleic acid into a protein. The expressed protein may remain intracellular, become a component of the cell surface membrane or be secreted into an extracellular space.

As used herein, cells which express a significant amount of an extracellular target biomolecule at least one cellular surface are “target positive cells” or “target+ cells” and are cells physically coupled to the specified, extracellular target biomolecule.

As used herein, the symbol “α” is shorthand for an immunoglobulin-type binding region capable of binding to the biomolecule following the symbol. The symbol “α” is used to refer to the functional characteristic of an immunoglobulin-type binding region based on its ability to bind to the biomolecule following the symbol with a binding affinity described by a dissociation constant (K_{D}) of 10⁻⁵ or less.

As used herein, the term “heavy chain variable (V_{H}) domain” or “light chain variable (V_{L}) domain” respectively refer to any antibody V_{H} or V_{L} domain (e.g. a human V_{H} or V_{L} domain) as well as any derivative thereof retaining at least qualitative antigen binding ability of the corresponding native antibody (e.g. a humanized V_{H} or V_{L} domain derived from a native murine V_{H} or V_{L} domain). A V_{H} or V_{L} domain consists of a “framework” region interrupted by the three CDRs or ABRs. The framework regions serve to align the CDRs or ABRs for specific binding to an epitope of an antigen. From amino-terminus to carboxy-terminus, both V_{H} and V_{L} domains comprise the following framework (FR) and CDR regions or ABR regions: FR1, CDR1, FR2, CDR2, FR3, CDR3, and FR4; or, similarly, FR1, ABR1, FR2, ABR2, FR3, ABR3, and FR4. As used herein, the terms “HCDR1,” “HCDR2,” or “HCDR3” are used to refer to CDRs 1, 2, or 3, respectively, in a V_{H} domain, and the terms “LCDR1,” “LCDR2,” and “LCDR3” are used to refer to CDRs 1, 2, or 3, respectively, in a V_{L} domain. For camelid V_{H}H fragments, IgNARs of cartilaginous fish, V_{NAR} fragments, certain single domain antibodies, and derivatives thereof, there is a single, heavy chain variable domain comprising the same basic arrangement: FR1, CDR1, FR2, CDR2, FR3, CDR3, and FR4. As used herein, the terms “HCDR1,” “HCDR2,” or “HCDR3” may be used to refer to CDRs 1, 2, or 3, respectively, in a single heavy chain variable domain.

As used herein, the term “effector” means providing a biological activity, such as cytotoxicity, biological signaling, enzymatic catalysis, subcellular routing, and/or intermolecular binding resulting in an allosteric effect(s) and/or the recruitment of one or more factors.

The term “Shiga toxin” herein refers to two families of related toxins: Shiga toxin (Stx)/Shiga-like toxin 1 (SLT-1/Stx1) and Shiga-like toxin 2 (SLT-2/Stx2). Stx is produced by Shigella dysenteriae, while SLT-1 and SLT-2 are derived from Escherichia coli. Members of the Shiga toxin family share the same overall structure and mechanism of action (Engedal N et al., Microbial Biotech 4: 32-46 (2011)). For example, Stx, SLT-1 and SLT-2 display indistinguishable enzymatic activity in cell free systems (Head S et al., J Biol Chem 266: 3617-21 (1991); Tesh V et al., Infect Immun 61: 3392-402 (1993); Brigotti M et al., Toxicon 35:1431-1437 (1997)).

Stx, SLT-1, and SLT-2 are multimeric molecules comprised of two polypeptide subunits, A and B. The B Subunit is a pentamer that binds the toxin to glycolipids on host cell membranes and enters the cell via endocytosis. Once inside the cell, the A Subunit undergoes proteolytic cleavage and the reduction of an internal disulfide bond to generate the A1 Subunit and the A2 Subunit. The Shiga toxin or Shiga-like toxin A1 Subunits (e.g., SLT-1-A1) are N-glycosidases that catalytically inactivate the 28S ribosomal RNA subunit to inhibit protein synthesis.

As described herein, the phrase “Shiga toxin effector region” refers to a polypeptide derived from a Shiga toxin A Subunit or Shiga-like toxin A Subunit of the Shiga toxin family, which exhibits at least one Shiga toxin effector function. For example, SEQ ID NO: 49-61 are derived from StxA and/or SLT-1A. In some embodiments, the Shiga toxin effector region of the PD-L1-binding molecule is a Shiga toxin A Subunit, such as StxA. In some embodiments, the Shiga toxin effector region of the PD-L1-binding molecule is a Shiga-like toxin A Subunit, such as SLT-1A or SLT-2A. In some embodiments, the Shiga toxin effector region of the PD-L1-binding molecule is an A1 Subunit of SLT-1 (e.g., SLT-1-A1). In some embodiments, the Shiga toxin effector region of the PD-L1-binding molecule is an enzymatically active, de-immunized Shiga-like toxin A1 Subunit of SLT-1 (e.g., SLT-1-A1 V1). In some embodiments, the Shiga toxin effector region has a sequence of SEQ ID NO: 41, or a sequence at least 85%, at least 90%, at least 95%, or at least 99% identical thereto. In some embodiments, the Shiga toxin effector region has a sequence of SEQ ID NO: 41 with 1-10, 10-20, 20-30, 30-40, 40-50 or more amino acid substitutions. In some embodiments, the Shiga toxin effector region has a sequence of any one of SEQ ID NO: 259 or 261-284, or a sequence at least 85%, at least 90%, at least 95%, or at least 99% identical thereto. In some embodiments, the Shiga toxin effector region has a sequence of any one of SEQ ID NO: 259 or 264-284 with 1-10, 10-20, 20-30, 30-40, 40-50 or more amino acid substitutions.

As used herein, a Shiga toxin effector function is a biological activity conferred by a polypeptide region derived from a Shiga toxin A Subunit. Non-limiting examples of Shiga toxin effector functions include promoting cell entry; lipid membrane deformation; promoting cellular internalization; stimulating clathrin-mediated endocytosis; directing intracellular routing to various intracellular compartments such as, e.g., the Golgi, endoplasmic reticulum, and cytosol; directing intracellular routing with a cargo; inhibiting a ribosome function(s); catalytic activities, such as, e.g., N-glycosidase activity and catalytically inhibiting ribosomes; reducing protein synthesis, inducing caspase activity, activating effector caspases, effectuating cytostatic effects, and cytotoxicity. Shiga toxin catalytic activities include, for example, ribosome inactivation, protein synthesis inhibition, N-glycosidase activity, polynucleotide:adenosine glycosidase activity, RNAase activity, and DNAase activity. Shiga toxins are ribosome inactivating proteins (RIPs). RIPs can depurinate nucleic acids, polynucleosides, polynucleotides, rRNA, ssDNA, dsDNA, mRNA (and polyA), and viral nucleic acids (see e.g., Barbieri L et al., Biochem J 286: 1-4 (1992); Barbieri L et al., Nature 372: 624 (1994); Ling J et al., FEBS Lett 345: 143-6 (1994); Barbieri L et al., Biochem J 319: 507-13 (1996); Roncuzzi L, Gasperi-Campani A, FEBS Lett 392: 16-20 (1996); Stirpe F et al., FEBS Lett 382: 309-12 (1996); Barbieri L et al., Nucleic Acids Res 25: 518-22 (1997); Wang P, Turner N, Nucleic Acids Res 27: 1900-5 (1999); Barbieri L et al., Biochim Biophys Acta 1480: 258-66 (2000); Barbieri L et al., J Biochem 128: 883-9 (2000); Brigotti M et al., Toxicon 39: 341-8 (2001); Brigotti M et al., FASEB J 16: 365-72 (2002); Bagga S et al., J Biol Chem 278: 4813-20 (2003); Picard D et al., J Biol Chem 280: 20069-75 (2005)). Some RIPs show antiviral activity and superoxide dismutase activity (Erice A et al., Antimicrob Agents Chemother 37: 835-8 (1993); Au T et al., FEBS Lett 471: 169-72 (2000); Parikh B, Turner N, Mini Rev Med Chem 4: 523-43 (2004); Sharma N et al., Plant Physiol 134: 171-81 (2004)). Shiga toxin catalytic activities have been observed both in vitro and in vivo. Non-limiting examples of assays for Shiga toxin effector activity measure various activities, such as, e.g., protein synthesis inhibitory activity, depurination activity, inhibition of cell growth, cytotoxicity, supercoiled DNA relaxation activity, and nuclease activity.

As used herein, the retention of Shiga toxin effector function refers to being capable of exhibiting a level of Shiga toxin functional activity, as measured by an appropriate quantitative assay with reproducibility, comparable to a wild-type, Shiga toxin effector polypeptide control (e.g. a Shiga toxin A1 fragment) or PD-L1 targeting molecule comprising a wild-type Shiga toxin effector polypeptide (e.g. a Shiga toxin A1 fragment) under the same conditions. For the Shiga toxin effector function of ribosome inactivation or ribosome inhibition, retained Shiga toxin effector function is exhibiting an IC₅₀ of 10,000 pM or less in an in vitro setting, such as, e.g., by using an assay known to the skilled worker and/or described herein. For the Shiga toxin effector function of cytotoxicity in a target positive cell-kill assay, retained Shiga toxin effector function is exhibiting a CD₅₀ of 1,000 nM or less, depending on the cell type and its expression of the appropriate extracellular target biomolecule, as shown, e.g., by using an assay known to the skilled worker and/or described herein.

As used herein, the term “equivalent” with regard to ribosome inhibition means an empirically measured level of ribosome inhibitory activity, as measured by an appropriate quantitative assay with reproducibility, which is reproducibly within 10% or less of the activity of the reference molecule (e.g., the second PD-L1 targeting molecule or third PD-L1 targeting molecule) under the same conditions.

As used herein, the term “equivalent” with regard to cytotoxicity means an empirically measured level of cytotoxicity, as measured by an appropriate quantitative assay with reproducibility, which is reproducibly within 10% or less of the activity of the reference molecule (e.g., the second PD-L1 targeting molecule or third PD-L1 targeting molecule) under the same conditions.

As used herein, the term “attenuated” with regard to cytotoxicity means a molecule exhibits or exhibited a CD₅₀ between 10-fold to 100-fold of a CD₅₀ exhibited by a reference molecule under the same conditions.

Inaccurate IC₅₀ and CD₅₀ values should not be considered when determining a level of Shiga toxin effector function activity. For some samples, accurate values for either IC₅₀ or CD₅₀ might be unobtainable due to the inability to collect the required data points for an accurate curve fit. For example, theoretically, neither an IC₅₀ nor CD₅₀ can be determined if greater than 50% ribosome inhibition or cell death, respectively, does not occur in a concentration series for a given sample. Data insufficient to accurately fit a curve as described in the analysis of the data from Shiga toxin effector function assays, such as, e.g., assays described in the Examples below, should not be considered as representative of actual Shiga toxin effector function.

A failure to detect activity in Shiga toxin effector function may be due to improper expression, polypeptide folding, and/or protein stability rather than a lack of cell entry, subcellular routing, and/or enzymatic activity. Assays for Shiga toxin effector functions may not require much polypeptide to measure significant amounts of Shiga toxin effector function activity. To the extent that an underlying cause of low or no effector function is determined empirically to relate to protein expression or stability, one of skill in the art may be able to compensate for such factors using protein chemistry and molecular engineering techniques known in the art, such that a Shiga toxin functional effector activity may be restored and measured. As examples, improper cell-based expression may be compensated for by using different expression control sequences; and improper polypeptide folding and/or stability may benefit from stabilizing terminal sequences, or compensatory mutations in non-effector regions which stabilize the three-dimensional structure of the molecule.

Certain Shiga toxin effector functions are not easily measurable, e.g. subcellular routing functions. For example, there is no routine, quantitative assay to distinguish whether the failure of a Shiga toxin effector polypeptide to be cytotoxic and/or deliver a heterologous epitope is due to improper subcellular routing, but at a time when tests are available, then Shiga toxin effector polypeptides may be analyzed for any significant level of subcellular routing as compared to the appropriate wild-type Shiga toxin effector polypeptide. However, if a Shiga toxin effector polypeptide component of a PD-L1 targeting molecule exhibits cytotoxicity comparable or equivalent to a wild-type Shiga toxin A Subunit construct, then the subcellular routing activity level is inferred to be comparable or equivalent, respectively, to the subcellular routing activity level of a wild-type Shiga toxin A Subunit construct at least under the conditions tested.

When new assays for individual Shiga toxin functions become available, Shiga toxin effector polypeptides and/or PD-L1 targeting molecules comprising Shiga toxin effector polypeptides may be analyzed for any level of those Shiga toxin effector functions, such as being within 1000-fold or 100-fold or less the activity of a wild-type Shiga toxin effector polypeptide or exhibiting 3-fold to 30-fold or greater activity as compared to a functional knockout, Shiga toxin effector polypeptide.

Sufficient subcellular routing may be merely deduced by observing a molecule's cytotoxic activity levels in cytotoxicity assays, such as, e.g., cytotoxicity assays based on T-cell epitope presentation or based on a toxin effector function involving a cytosolic and/or endoplasmic reticulum-localized, target substrate.

As used herein, the retention of “significant” Shiga toxin effector function refers to a level of Shiga toxin functional activity, as measured by an appropriate quantitative assay with reproducibility comparable to a wild-type Shiga toxin effector polypeptide control (e.g. a Shiga toxin A1 fragment). For in vitro ribosome inhibition, significant Shiga toxin effector function is exhibiting an IC₅₀ of 300 pM or less depending on the source of the ribosomes used in the assay (e.g. a bacterial, archaeal, or eukaryotic (algal, fungal, plant, or animal) source). This is significantly greater inhibition as compared to the approximate IC₅₀ of 100,000 pM for the catalytically disrupted SLT-1A 1-251 double mutant (Y77S/E167D, numbering relative to SEQ ID NO: 1). For cytotoxicity in a target-positive cell-kill assay in laboratory cell culture, significant Shiga toxin effector function is exhibiting a CD₅₀ of 100, 50, 30 nM, or less, depending on the target biomolecule(s) of the binding region and the cell type, particularly that cell type's expression and/or cell-surface representation of the appropriate extracellular target biomolecule(s) and/or the extracellular epitope(s) targeted by the molecule being evaluated. This is significantly greater cytotoxicity to the appropriate, target-positive cell population as compared to a Shiga toxin A Subunit alone (or a wild-type Shiga toxin A1 fragment), without a cell targeting binding region, which has a CD₅₀ of 100-10,000 nM, depending on the cell line.

As used herein and with regard to the Shiga toxin effector function of a molecule as described herein, the term “reasonable activity” refers to exhibiting at least a moderate level (e.g. within 11-fold to 1,000-fold) of Shiga toxin effector activity as defined herein in relation to a molecule comprising a naturally occurring Shiga toxin, wherein the Shiga toxin effector activity is selected from the group consisting of: internalization efficiency, subcellular routing efficiency to the cytosol, delivered epitope presentation by a target cell(s), ribosome inhibition, and cytotoxicity. For cytotoxicity, a reasonable level of Shiga toxin effector activity includes being within 1,000-fold of a wild-type, Shiga toxin construct, such as, e.g., exhibiting a CD₅₀ of 500 nM or less when a wild-type Shiga toxin construct exhibits a CD₅₀ of 0.5 nM (e.g. a PD-L1 targeting molecule comprising a wild-type Shiga toxin A1 fragment).

As used herein and with regard to the cytotoxicity of a molecule described herein, the term “optimal” refers to a level of Shiga toxin catalytic domain mediated cytotoxicity that is within 2, 3, 4, 5, 6, 7, 8, 9, or 10-fold of the cytotoxicity of a molecule comprising wild-type Shiga toxin A1 fragment (e.g. a Shiga toxin A Subunit or certain truncated variants thereof) and/or a naturally occurring Shiga toxin.

It should be noted that even if the cytotoxicity of a Shiga toxin effector polypeptide is reduced relative to a wild-type Shiga toxin A Subunit or fragment thereof, in practice, applications using attenuated, Shiga toxin effector polypeptides might be equally or more effective than using wild-type Shiga toxin effector polypeptides because the highest potency variants might exhibit undesirable effects which are minimized or reduced in reduced cytotoxic-potency variants. Wild-type Shiga toxins are very potent, being able to kill an intoxicated cell after only one toxin molecule has reached the cytosol of the intoxicated cell or perhaps after only forty toxin molecules have been internalized into the intoxicated cell. Shiga toxin effector polypeptides with even considerably reduced Shiga toxin effector functions, such as, e.g., subcellular routing or cytotoxicity, as compared to wild-type Shiga toxin effector polypeptides might still be potent enough for practical applications, such as, e.g., applications involving targeted cell-killing, heterologous epitope delivery, and/or detection of specific cells and their subcellular compartments. In addition, certain reduced-activity Shiga toxin effector polypeptides may be particularly useful for delivering cargos (e.g. an additional exogenous material or T-cell epitope) to certain intracellular locations or subcellular compartments of target cells.

The term “selective cytotoxicity” with regard to the cytotoxic activity of a molecule refers to the relative level of cytotoxicity between a biomolecule target positive cell population (e.g. a targeted cell-type) and a non-targeted bystander cell population (e.g. a biomolecule target negative cell-type), which can be expressed as a ratio of the half-maximal cytotoxic concentration (CD₅₀) for a targeted cell type over the CD₅₀ for an untargeted cell type to provide a metric of cytotoxic selectivity or indication of the preferentiality of killing of a targeted cell versus an untargeted cell.

The cell surface representation and/or density of a given extracellular target biomolecule (or extracellular epitope of a given target biomolecule) may influence the applications for which certain PD-L1 targeting molecules may be most suitably used. Differences in cell surface representation and/or density of a given target biomolecule between cells may alter, both quantitatively and qualitatively, the efficiency of cellular internalization and/or cytotoxicity potency of a given PD-L1 targeting molecule. The cell surface representation and/or density of a given target biomolecule can vary greatly among target biomolecule positive cells or even on the same cell at different points in the cell cycle or cell differentiation. The total cell surface representation of a given target biomolecule and/or of certain extracellular epitopes of a given target biomolecule on a particular cell or population of cells may be determined using methods known to the skilled worker, such as methods involving fluorescence-activated cell sorting (FACS) flow cytometry.

As used herein, the terms “disrupted,” “disruption,” or “disrupting,” and grammatical variants thereof, with regard to a polypeptide region or feature within a polypeptide refers to an alteration of at least one amino acid within the region or composing the disrupted feature. Amino acid alterations include various mutations, such as, e.g., a deletion, inversion, insertion, or substitution which alter the amino acid sequence of the polypeptide. Amino acid alterations also include chemical changes, such as, e.g., the alteration one or more atoms in an amino acid functional group or the addition of one or more atoms to an amino acid functional group.

As used herein, “de-immunized” means reduced antigenic and/or immunogenic potential after administration to a chordate as compared to a reference molecule, such as, e.g., a wild-type peptide region, polypeptide region, or polypeptide. This includes a reduction in overall antigenic and/or immunogenic potential despite the introduction of one or more, de novo, antigenic and/or immunogenic epitopes as compared to a reference molecule. For some embodiments, “de-immunized” means a molecule exhibited reduced antigenicity and/or immunogenicity after administration to a mammal as compared to a “parental” molecule from which it was derived, such as, e.g., a wild-type Shiga toxin A1 fragment or PD-L1 targeting molecule comprising the aforementioned. In some embodiments, the de-immunized, Shiga toxin effector polypeptide is capable of exhibiting a relative antigenicity compared to a reference “parental” molecule which is reduced by 10%, 20%, 30%, 40%, 50%, 60%, 70%, 80%, 90%, or greater than the antigenicity of the reference molecule under the same conditions measured by the same assay, such as, e.g., an assay known to the skilled worker and/or described herein like a quantitative ELISA or Western blot analysis. In some embodiments, the de-immunized, Shiga toxin effector polypeptide is capable of exhibiting a relative immunogenicity compared to a reference “parental” molecule which is reduced by 10%, 20%, 30%, 40%, 50%, 60%, 70%, 80%, 90%, 95%, 97%, 99%, or greater than the immunogenicity of the reference molecule under the same conditions measured by the same assay, such as, e.g., an assay known to the skilled worker and/or described herein like a quantitative measurement of anti-molecule antibodies produced in a mammal(s) after receiving parenteral administration of the molecule at a given time-point.

The relative immunogenicities of PD-L1 targeting molecules may be determined using an assay for in vivo antibody responses to the PD-L1 targeting molecules after repeat, parenteral administrations over periods of time.

As used herein, the phrase “B-cell and/or CD4+ T-cell de-immunized” means that the molecule has a reduced antigenic and/or immunogenic potential after administration to a mammal regarding either B-cell antigenicity or immunogenicity and/or CD4+ T-cell antigenicity or immunogenicity. For some embodiments, “B-cell de-immunized” means a molecule exhibited reduced B-cell antigenicity and/or immunogenicity after administration to a mammal as compared to a “parental” molecule from which it was derived, such as, e.g., a wild-type Shiga toxin A1 fragment. For some embodiments, “CD4+ T-cell de-immunized” means a molecule exhibited reduced CD4 T-cell antigenicity and/or immunogenicity after administration to a mammal as compared to a “parental” molecule from which it was derived, such as, e.g., a wild-type Shiga toxin A1 fragment.

The term “endogenous” with regard to a B-cell epitope, CD4+ T-cell epitope, B-cell epitope region, or CD4+ T-cell epitope region in a Shiga toxin effector polypeptide refers to an epitope present in a wild-type Shiga toxin A Subunit.

As used herein, the phrase “CD8+ T-cell hyper-immunized” means that the molecule, when present inside a nucleated, chordate cell within a living chordate, has an increased antigenic and/or immunogenic potential regarding CD8+ T-cell antigenicity or immunogenicity. Commonly, CD8+ T-cell immunized molecules are capable of cellular internalization to an early endosomal compartment of a nucleated, chordate cell due either to an inherent feature(s) or as a component of a PD-L1 targeting molecule.

As used herein, the term “heterologous” means of a different source than an A Subunit of a naturally occurring Shiga toxin, e.g. a heterologous polypeptide is not naturally found as part of any A Subunit of a native Shiga toxin. The term “heterologous” with regard to T-cell epitope or T-cell epitope-peptide component of a PD-L1 targeting molecule refers to an epitope or peptide sequence which did not initially occur in the polypeptide component to be modified, but which has been added to the polypeptide, whether added via the processes of embedding, fusion, insertion, and/or amino acid substitution as described herein, or by any other engineering means. The result is a modified polypeptide comprising a T-cell epitope foreign to the original, unmodified polypeptide, i.e. the T-cell epitope was not present in the original polypeptide.

The term “embedded” and grammatical variants thereof with regard to a T-cell epitope or T-cell epitope-peptide component of a PD-L1 targeting molecule refers to the internal replacement of one or more amino acids within a polypeptide region with different amino acids in order to generate a new polypeptide sequence sharing the same total number of amino acid residues with the starting polypeptide region. Thus, the term “embedded” does not include any external, terminal fusion of any additional amino acid, peptide, or polypeptide component to the starting polypeptide nor any additional internal insertion of any additional amino acid residues, but rather includes only substitutions for existing amino acids. The internal replacement may be accomplished merely by amino acid residue substitution or by a series of substitutions, deletions, insertions, and/or inversions. If an insertion of one or more amino acids is used, then the equivalent number of proximal amino acids must be deleted next to the insertion to result in an embedded T-cell epitope. This is in contrast to use of the term “inserted” with regard to a T-cell epitope contained within a polypeptide component of a PD-L1 targeting molecule to refer to the insertion of one or more amino acids internally within a polypeptide resulting in a new polypeptide having an increased number of amino acids residues compared to the starting polypeptide.

The term “inserted” and grammatical variants thereof with regard to a T-cell epitope contained within a polypeptide component of a PD-L1 targeting molecule refers to the insertion of one or more amino acids within a polypeptide resulting in a new polypeptide sequence having an increased number of amino acids residues compared to the starting polypeptide. The “pure” insertion of a T-cell epitope-peptide is when the resulting polypeptide increased in length by the number of amino acid residues equivalent to the number of amino acid residues in the entire, inserted T-cell epitope-peptide. The phrases “partially inserted,” “embedded and inserted,” and grammatical variants thereof with regard to a T-cell epitope contained within a polypeptide component of a PD-L1 targeting molecule, refers to when the resulting polypeptide increased in length, but by less than the number of amino acid residues equivalent to the length of the entire, inserted T-cell epitope-peptide. Insertions, whether “pure” or “partial,” include any of the previously described insertions even if other regions of the polypeptide not proximal to the insertion site within the polypeptide are deleted thereby resulting in a decrease in the total length of the final polypeptide because the final polypeptide still comprises an internal insertion of one or more amino acids of a T-cell epitope-peptide within a polypeptide region.

As used herein, the term “T-cell epitope delivering” when describing a functional activity of a molecule means that a molecule provides the biological activity of localizing within a cell to a subcellular compartment that is competent to result in the proteasomal cleavage of a proteinaceous part of the molecule which comprises a T-cell epitope-peptide. The “T-cell epitope delivering” function of a molecule can be assayed by observing the MHC presentation of a T-cell epitope-peptide cargo of the molecule on a cell surface of a cell to which the molecule is exogenously administered, or in which the assay was begun with the cell containing the molecule in one or more of its endosomal compartments. Generally, the ability of a molecule to deliver a T-cell epitope to a proteasome can be determined where the initial location of the “T-cell epitope delivering” molecule is an early endosomal compartment of a cell, and then, the molecule is empirically shown to deliver the epitope-peptide to the proteasome of the cell. However, a “T-cell epitope delivering” ability may also be determined where the molecule starts at an extracellular location and is empirically shown, either directly or indirectly, to deliver the epitope into a cell and to proteasomes of the cell. For example, certain “T-cell epitope delivering” molecules pass through an endosomal compartment of the cell, such as, e.g. after endocytotic entry into that cell. Alternatively, “T-cell epitope delivering” activity may be observed for a molecule starting at an extracellular location whereby the molecule does not enter any endosomal compartment of a cell—instead the “T-cell epitope delivering” molecule enters a cell and delivers a T-cell epitope-peptide to proteasomes of the cell, presumably because the “T-cell epitope delivering” molecule directed its own routing to a subcellular compartment competent to result in proteasomal cleavage of its T-cell epitope-peptide component.

As used herein, the phrase “proximal to an amino-terminus” with reference to the position of a Shiga toxin effector polypeptide region of a PD-L1 targeting molecule refers to a distance wherein at least one amino acid residue of the Shiga toxin effector polypeptide region is within 1, 2, 3, 4, 5, 6, 7, 8, 9, 10, 11, 12 or more, e.g., up to 18-20 amino acid residues, of an amino-terminus of the PD-L1 targeting molecule as long as the PD-L1 targeting molecule is capable of exhibiting the appropriate level of Shiga toxin effector functional activity noted herein (e.g., a certain level of cytotoxic potency). Thus for some embodiments, any amino acid residue(s) fused amino-terminal to the Shiga toxin effector polypeptide should not reduce any Shiga toxin effector function (e.g., by sterically hindering a structure(s) near the amino-terminus of the Shiga toxin effector polypeptide region) such that a functional activity of the Shiga toxin effector polypeptide is reduced below the appropriate activity level required herein.

As used herein, the phrase “more proximal to an amino-terminus” with reference to the position of a Shiga toxin effector polypeptide region within a PD-L1 targeting molecule as compared to another component (e.g., a cell-targeting, binding region, molecular moiety, and/or additional exogenous material) refers to a position wherein at least one amino acid residue of the amino-terminus of the Shiga toxin effector polypeptide is closer to the amino-terminus of a linear, polypeptide component of the PD-L1 targeting molecule as compared to the other referenced component.

As used herein, the phrase “active enzymatic domain derived from one A Subunit of a member of the Shiga toxin family” refers to having the ability to inhibit protein synthesis via a catalytic ribosome inactivation mechanism. The enzymatic activities of naturally occurring Shiga toxins may be defined by the ability to inhibit protein translation using assays known to the skilled worker, such as, e.g., in vitro assays involving RNA translation in the absence of living cells or in vivo assays involving RNA translation in a living cell. Using assays known to the skilled worker and/or described herein, the potency of a Shiga toxin enzymatic activity may be assessed directly by observing N-glycosidase activity toward ribosomal RNA (rRNA), such as, e.g., a ribosome nicking assay, and/or indirectly by observing inhibition of ribosome function and/or protein synthesis.

As used herein, the term “Shiga toxin A1 fragment region” refers to a polypeptide region consisting essentially of a Shiga toxin A1 fragment and/or derived from a Shiga toxin A1 fragment of a Shiga toxin.

As used herein, the terms “terminus,” “amino-terminus,” or “carboxy-terminus” with regard to a PD-L1 targeting molecule refers generally to the last amino acid residue of a polypeptide chain of the PD-L1 targeting molecule (e.g., a single, continuous polypeptide chain). A PD-L1 targeting molecule may comprise more than one polypeptide or protein, and, thus, a PD-L1 targeting molecule may comprise multiple amino-terminals and carboxy-terminals. For example, the “amino-terminus” of a PD-L1 targeting molecule may be defined by the first amino acid residue of a polypeptide chain representing the amino-terminal end of the polypeptide, which is generally characterized by a starting, amino acid residue which does not have a peptide bond with any amino acid residue involving the primary amino group of the starting amino acid residue or involving the equivalent nitrogen for starting amino acid residues which are members of the class of N-alkylated alpha amino acid residues. Similarly, the “carboxy-terminus” of a PD-L1 targeting molecule may be defined by the last amino acid residue of a polypeptide chain representing the carboxyl-terminal end of the polypeptide, which is generally characterized by a final, amino acid residue which does not have any amino acid residue linked by a peptide bond to the alpha-carbon of its primary carboxyl group.

As used herein, the terms “terminus,” “amino-terminus,” or “carboxy-terminus” with regard to a polypeptide region refers to the regional boundaries of that region, regardless of whether additional amino acid residues are linked by peptide bonds outside of that region. In other words, the terminals of the polypeptide region regardless of whether that region is fused to other peptides or polypeptides. For example, a fusion protein comprising two proteinaceous regions, e.g., a binding region comprising a peptide or polypeptide and a Shiga toxin effector polypeptide, may have a Shiga toxin effector polypeptide region with a carboxy-terminus ending at amino acid residue 251 of the Shiga toxin effector polypeptide region despite a peptide bond involving residue 251 to an amino acid residue at position 252 representing the beginning of another proteinaceous region, e.g., the binding region. In this example, the carboxy-terminus of the Shiga toxin effector polypeptide region refers to residue 251, which is not a terminus of the fusion protein but rather represents an internal, regional boundary. Thus, for polypeptide regions, the terms “terminus,” “amino-terminus,” and “carboxy-terminus” are used to refer to the boundaries of polypeptide regions, whether the boundary is a physically terminus or an internal, position embedded within a larger polypeptide chain.

As used herein, the phrase “carboxy-terminus region of a Shiga toxin A1 fragment” refers to a polypeptide region derived from a naturally occurring Shiga toxin A1 fragment, the region beginning with a hydrophobic residue (e.g., V236 of StxA-A1 and SLT-1A1, and V235 of SLT-2A1) that is followed by a hydrophobic residue and the region ending with the furin-cleavage site conserved among Shiga toxin A1 fragment polypeptides and ending at the junction between the A1 fragment and the A2 fragment in native, Shiga toxin A Subunits. In some embodiments, the carboxy-terminal region of a Shiga toxin A1 fragment includes a peptidic region derived from the carboxy-terminus of a Shiga toxin A1 fragment polypeptide, such as, e.g., a peptidic region comprising or consisting essentially of the carboxy-terminus of a Shiga toxin A1 fragment. Non-limiting examples of peptidic regions derived from the carboxy-terminus of a Shiga toxin A1 fragment include the amino acid residue sequences natively positioned from position 236 to position 239, 240, 241, 242, 243, 244, 245, 246, 247, 248, 249, 250, or 251 in Stx1A (SEQ ID NO:2) or SLT-1A (SEQ ID NO:1); and from position 235 to position 239, 240, 241, 242, 243, 244, 245, 246, 247, 248, 249, or 250 in SLT-2A (SEQ ID NO:3).

As used herein, the phrase “proximal to the carboxy-terminus of an A1 fragment polypeptide” with regard to a linked molecular moiety and/or binding region refers to being within 1, 2, 3, 4, 5, 6, 7, 8, 9, 10, 11, or 12 amino acid residues from the amino acid residue defining the last residue of the Shiga toxin A1 fragment polypeptide.

As used herein, the phrase “sterically covers the carboxy-terminus of the A1 fragment-derived region” includes any molecular moiety of a size of 4.5 kDa or greater (e.g., an immunoglobulin-type binding region) linked and/or fused to an amino acid residue in the carboxy-terminus of the A1 fragment-derived region, such as, e.g., the amino acid residue derived from the amino acid residue natively positioned at any one of positions 236 to 251 in Stx1A (SEQ ID NO:2) or SLT-1A (SEQ ID NO:1) or from 235 to 250 in SLT-2A (SEQ ID NO:3). As used herein, the phrase “sterically covers the carboxy-terminus of the A1 fragment-derived region” also includes any molecular moiety of a size of 4.5 kDa or greater (e.g., an immunoglobulin-type binding region) linked and/or fused to an amino acid residue in the carboxy-terminus of the A1 fragment-derived region, such as, e.g., the amino acid residue carboxy-terminal to the last amino acid A1 fragment-derived region and/or the Shiga toxin effector polypeptide. As used herein, the phrase “sterically covers the carboxy-terminus of the A1 fragment-derived region” also includes any molecular moiety of a size of 4.5 kDa or greater (e.g., an immunoglobulin-type binding region) physically preventing cellular recognition of the carboxy-terminus of the A1 fragment-derived region, such as, e.g. recognition by the ERAD machinery of a eukaryotic cell.

As described herein, a binding region, such as, e.g., an immunoglobulin-type binding region, that comprises a polypeptide comprising at least forty amino acids and that is linked (e.g., fused) to the carboxy-terminus of the Shiga toxin effector polypeptide region comprising an A1 fragment-derived region is a molecular moiety which is “sterically covering the carboxy-terminus of the A1 fragment-derived region.”

As described herein, a binding region, such as, e.g., an immunoglobulin-type binding region, that comprises a polypeptide comprising at least forty amino acids and that is linked (e.g., fused) to the carboxy-terminus of the Shiga toxin effector polypeptide region comprising an A1 fragment-derived region is a molecular moiety “encumbering the carboxy-terminus of the A1 fragment-derived region.”

As used herein, the term “A1 fragment of a member of the Shiga toxin family” refers to the remaining amino-terminal fragment of a Shiga toxin A Subunit after proteolysis by furin at the furin-cleavage site conserved among Shiga toxin A Subunits and positioned between the A1 fragment and the A2 fragment in wild-type Shiga toxin A Subunits.

As used herein, the phrase “furin-cleavage site at the carboxy-terminus of the A1 fragment region” refers to a specific, furin-cleavage siteconserved among Shiga toxin A Subunits and bridging the junction between the A1 fragment and the A2 fragment in naturally occurring, Shiga toxin A Subunits.

As used herein, the phrase “furin-cleavage site proximal to the carboxy-terminus of the A1 fragment region” refers to any identifiable, furin-cleavage site having an amino acid residue within a distance of less than 1, 2, 3, 4, 5, 6, 7, or more amino acid residues of the amino acid residue defining the last amino acid residue in the A1 fragment region or A1 fragment derived region, including a furin-cleavage site located carboxy-terminal of an A1 fragment region or A1 fragment derived region, such as, e.g., at a position proximal to the linkage of the A1 fragment-derived region to another component of the molecule, such as, e.g., a molecular moiety of a PD-L1 targeting molecule.

As used herein, the phrase “disrupted furin-cleavage site” refers to (i) a specific furin-cleavage site as described herein in Section I-B and (ii) which comprises a mutation and/or truncation that can confer a molecule with a reduction in furin-cleavage as compared to a reference molecule, such as, e.g., a reduction in furin-cleavage reproducibly observed to be 30%, 40%, 50%, 60%, 70%, 80%, 90%, 95%, 97%, 98%, 99%, or less (including 100% for no cleavage) than the furin-cleavage of a reference molecule observed in the same assay under the same conditions. The percentage of furin-cleavage as compared to a reference molecule can be expressed as a ratio of cleaved:uncleaved material of the molecule of interest divided by the cleaved:uncleaved material of the reference molecule (see e.g. WO 2015/191764; WO 2016/196344). Non-limiting examples of suitable reference molecules include certain molecules comprising a wild-type Shiga toxin furin-cleavage site and/or furin-cleavage site as described herein.

As used herein, the phrase “furin-cleavage resistant” means a molecule or specific polypeptide region thereof exhibits reproducibly less furin cleavage than (i) the carboxy-terminus of a Shiga toxin A1 fragment in a wild-type Shiga toxin A Subunit or (ii) the carboxy-terminus of the Shiga toxin A1 fragment derived region of construct wherein the naturally occurring furin-cleavage site natively positioned at the junction between the A1 and A2 fragments is not disrupted; as assayed by any available means to the skilled worker, including by using a method described herein.

As used herein, the phrase “active enzymatic domain derived form an A Subunit of a member of the Shiga toxin family” refers to a polypeptide structure having the ability to inhibit protein synthesis via catalytic inactivation of a ribosome based on a Shiga toxin enzymatic activity. The ability of a molecular structure to exhibit inhibitory activity of protein synthesis and/or catalytic inactivation of a ribosome may be observed using various assays known to the skilled worker, such as, e.g., in vitro assays involving RNA translation assays in the absence of living cells or in vivo assays involving the ribosomes of living cells. For example, using assays known to the skilled worker, the enzymatic activity of a molecule based on a Shiga toxin enzymatic activity may be assessed directly by observing N-glycosidase activity toward ribosomal RNA (rRNA), such as, e.g., a ribosome nicking assay, and/or indirectly by observing inhibition of ribosome function, RNA translation, and/or protein synthesis.

As used herein with respect to a Shiga toxin effector polypeptide, a “combination” describes a Shiga toxin effector polypeptide comprising two or more sub-regions wherein each sub-region comprises at least one of the following: (1) a disruption in an endogenous epitope or epitope region; (2) an embedded, heterologous, T-cell epitope-peptide; (3) an inserted, heterologous, T-cell epitope-peptide; and (4) a disrupted furin-cleavage site at the carboxy-terminus of an A1 fragment region.

As used herein, a “PD-L1+PD-L1 targeting molecule” is used interchangeably with a “PD-L1 targeting molecule” or “PD-L1-binding molecule”. As used herein, the terms “PD-L1 targeting molecule”, “PD-L1-binding molecule”, and “PD-L1 targeting molecule” are used interchangably, and such molecules may also be referred to as “DI-SLT-1A fusion proteins” and “SLT-1A fusion proteins.” All of the aforementioned molecule types include various “PD-L1-binding proteins.”

### INTRODUCTION

Provided herein are various PD-L1 targeting molecules which bind PD-L1 and comprise a Shiga toxin A Subunit effector polypeptide (referred to herein as “PD-L1 targeting molecules”). The PD-L1 targeting molecules described herein are useful, for example, (1) as cytotoxic molecules for killing PD-L1 expressing cells, (2) for selectively killing specific PD-L1 positive cell type(s) amongst other cells, (3) as nontoxic delivery vehicles for delivering an atom or molecule to the interior of a PD-L1 expressing cell, (4) as diagnostic molecules for the diagnosis, prognosis, or characterization of diseases and conditions involving PD-L1 expressing cells, and (5) as therapeutic molecules for treating a variety of diseases, disorders, and conditions involving PD-L1 expressing cells, such as various cancers and tumors.

Shiga toxin A Subunit effector polypeptides provide robust and powerful scaffolds for engineering novel, PD-L1 targeting molecules (see e.g. WO 2014/164680, WO 2014/164693, WO 2015/138435, WO 2015/138452, WO 2015/113005, WO 2015/113007, WO 2015/191764, WO 2016/196344, WO 2017/019623, WO 2018/106895, and WO 2018/140427). The association of PD-L1 binding immunoglobulin-derived fragments as cell-targeting moieties with Shiga toxin A Subunit effector polypeptides enables the engineering of therapeutic and diagnostic molecules that target PD-L1.

### I. The General Structure of the PD-L1 Targeting Molecules

In some embodiments, the PD-L1 targeting molecules may each comprise (1) a PD-L1 binding region for cell-targeting, and (2) a Shiga toxin effector polypeptide. In some embodiments, a PD-L1 targeting molecule may comprise (1) a binding region capable of specifically binding an extracellular part of PD-L1 associated with a cell surface and (2) a Shiga toxin effector polypeptide region comprising a Shiga toxin A Subunit effector polypeptide (referred to herein as a “Shiga toxin effector polypeptide”). In some embodiments, the PD-L1 binding molecule comprises two or more PD-L1 binding regions, whether the same or different, and two or more Shiga toxin effector polypeptide regions, whether the same or different. One non-limiting example of a PD-L1 targeting molecule comprises a Shiga toxin effector polypeptide fused to an immunoglobulin-type binding region comprising a single-chain variable fragment, or a homo- or hetero-dimer of the aforementioned.

The PD-L1 targeting molecules may optionally comprise a T-cell epitope for delivery to the interior of a target cell and subsequent cell-surface presentation.

In some embodiments, the Shiga toxin A Subunit effector polypeptide of the PD-L1 targeting molecule combines structural elements resulting in two or more properties in a single molecule, such as, e.g., the ability to 1) exhibit reduced antigenicity and/or immunogenicity as compared to molecular variants lacking that particular combination of elements, 2) exhibit reduced protease-cleavage as compared to molecular variants lacking that particular combination of elements, 3) exhibit reduced non-specific toxicity to a multicellular organism at certain dosages as compared to molecular variants lacking that particular combination of elements, 4) deliver an embedded or inserted T-cell epitope to the MHC class I system of a cell for cell-surface presentation, and/or 5) exhibit potent cytotoxicity.

### A. PD-L1 Binding Regions

In some embodiments, the PD-L1 targeting molecule comprises a binding region comprising an immunoglobulin-type polypeptide capable of exhibiting specific and high-affinity binding to human PD-L1 and/or PD-L1 present on a cellular surface of a cell, such as, e.g., PD-L1 expressing cell or PD-L1 positive cell.

In some embodiments, a binding region of a PD-L1 targeting molecule is a cell-targeting component, such as, e.g., a domain, molecular moiety, or agent, capable of binding specifically to an extracellular part of a PD-L1 target biomolecule on a cell surface (i.e. an extracellular target biomolecule) with high affinity. As used herein, the term “PD-L1 binding region” refers to a molecular moiety (e.g. a proteinaceous molecule) or agent capable of specifically binding an extracellular part of a PD-L1 molecule with high affinity, such as, e.g., having a dissociation constant with regard to PD-L1 of 10⁻⁵ to 10⁻¹² moles per liter. As used herein, PD-L1 binding refers to the ability to bind to an extracellular part of PD-L1, including an isoform or variant of human PD-L1.

An extracellular part of a target biomolecule refers to a portion of its structure exposed to the extracellular environment when the molecule is physically coupled to a cell, such as, e.g., when the target biomolecule is expressed at a cellular surface by the cell. In this context, exposed to the extracellular environment means that part of the target biomolecule is accessible by, e.g., an antibody or at least a binding moiety smaller than an antibody such as a single-domain antibody domain, a nanobody, a heavy-chain antibody domain derived from camelids or cartilaginous fishes, a single-chain variable fragment, or any number of engineered alternative scaffolds to immunoglobulins (see below). The exposure to the extracellular environment of or accessibility to a part of target biomolecule physically coupled to a cell may be empirically determined by the skilled worker using methods well known in the art.

In some embodiments, a PD-L1 targeting molecule comprises a binding region comprising one or more polypeptides capable of selectively and specifically binding an extracellular part of PD-L1.

In some embodiments, the PD-L1 binding region is an immunoglobulin-type binding region. In some embodiments, the immunoglobulin-type, PD-L1 binding region is derived from an immunoglobulin, PD-L1 binding region, such as an antibody paratope capable of binding an extracellular part of PD-L1. This engineered polypeptide may optionally include polypeptide scaffolds comprising or consisting essentially of complementary determining regions and/or antigen binding regions from immunoglobulins as described herein.

In some embodiments, the binding region comprises a polypeptide comprising an immunoglobulin-type binding region. In some embodiments, the binding region comprises a heavy chain variable region (HVR-H) comprising three CDRs, each having at least 85%, 90%, 91%, 92%, 93%, 94%, 95%, 96%, 97%, 98%, or 99% identity to SEQ ID NO:22, SEQ ID NO:23, SEQ ID NO:24, SEQ ID NO:36, or SEQ ID NO:37; or consisting essentially of an amino acid sequence show in any one of SEQ ID NO:22, SEQ ID NO:23, SEQ ID NO:24, SEQ ID NO:36, and SEQ ID NO:37. In some embodiments, the binding region further comprises: (a) a light chain variable region (HVR-L) comprising three CDRs, having at least 85%, 90%, 91%, 92%, 93%, 94%, 95%, 96%, 97%, 98%, or 99% identity to SEQ ID NO:19, SEQ ID NO:20, and SEQ ID NO:21; or consisting essentially of an amino acid sequence shown in any one of SEQ ID NO:19, SEQ ID NO:20, and SEQ ID NO:21. In some embodiments, the binding region comprises: (a) a light chain variable region (HVR-L) comprising three CDRs, each comprising or consisting essentially of an amino acid sequence shown in any one of SEQ ID NO:19, SEQ ID NO:20, and SEQ ID NO:21; and (b) a heavy chain variable region (HVR-H) comprising three CDRs, each comprising or consisting essentially of an amino acid sequence show in any one of SEQ ID NO:22, SEQ ID NO:23, SEQ ID NO:24, SEQ ID NO:36, and SEQ ID NO:37.

In some embodiments, the binding region comprises: (a) a light chain variable region (HVR-L) comprising three CDRs, each comprising or consisting essentially of an amino acid sequence shown in any one of SEQ ID NO:19, SEQ ID NO:20, and SEQ ID NO:21; and/or (b) a heavy chain variable region (HVR-H) comprising three CDRs, each comprising or consisting essentially of an amino acid sequence show in any one of SEQ ID NO:22, SEQ ID NO:23, SEQ ID NO:24, SEQ ID NO:36, and SEQ ID NO:37.

In some embodiments, the binding region comprises: (a) a light chain variable region comprising: (i) HVR-L1 comprising or consisting essentially of the amino acid sequence TGTSSDVGSYNRVS (SEQ ID NO:19), (ii) HVR-L2 comprising or consisting essentially of the amino acid sequence EVSNRPS (SEQ ID NO:20), and (iii) HVR-L3 comprising or consisting essentially of the amino acid sequence SSHTTSGTYV (SEQ ID NO:21); and/or (b) a heavy chain variable region comprising: (i) HVR-H1 comprising or consisting essentially of the amino acid sequence SYAIS (SEQ ID NO:22), (ii) HVR-H2 comprising or consisting essentially of the amino acid sequence GIIPIFGTANYAQKFQG (SEQ ID NO:23), and (iii) HVR-H3 comprising or consisting essentially of the amino acid sequence DQGYAHAFDI (SEQ ID NO:24).

In some embodiments, the binding region comprises: (a) a light chain variable region (HVR-L) comprising three CDRs: (i) a LCDR1 comprising or consisting essentially of the amino acid sequence TGTSSDVGSYNRVS (SEQ ID NO:19), (ii) a LCDR2 comprising or consisting essentially of the amino acid sequence EVSNRPS (SEQ ID NO:20), and (iii) a LCDR3 comprising or consisting essentially of the amino acid sequence SSHTTSGTYV (SEQ ID NO:21); and/or (b) a heavy chain variable region (HVR-H) comprising: (i) a HCDR1 comprising or consisting essentially of the amino acid sequence GGTFSSY (SEQ ID NO:22), (ii) a HCDR2 comprising or consisting essentially of the amino acid sequence IPIFGT (SEQ ID NO:23), and (iii) a HCDR3 comprising or consisting essentially of the amino acid sequence DQGYAHAFDI (SEQ ID NO:24).

In some embodiments, the binding region comprises: an immunoglobulin region comprising three CDRs comprising or consisting essentially of the amino acid sequences show in SEQ ID NO:22, SEQ ID NO:23, and SEQ ID NO:24, respectively.

In some embodiments, the binding region comprises an immunoglobulin region comprising three CDRs comprising or consisting essentially of the amino acid sequences show in SEQ ID NO:22, SEQ ID NO:37, and SEQ ID NO:24, respectively.

In some embodiments, the binding region comprises three CDRs comprising or consisting essentially of the amino acid sequences show in SEQ ID NO:36, SEQ ID NO:23, and SEQ ID NO:24, respectively.

In some embodiments, the binding region comprises an immunoglobulin region comprising three CDRs comprising or consisting essentially of the amino acid sequences show in SEQ ID NO:36, SEQ ID NO:37, and SEQ ID NO:24, respectively.

In some embodiments, the binding region comprises: (a) a light chain region having at least 85%, 90%, 91%, 92%, 93%, 94%, 95%, 96%, 97%, 98%, or 99%, identity to SEQ ID NO:25 or SEQ ID NO:27 or consisting essentially of the amino acid sequence of SEQ ID NO:25 or SEQ ID NO:27; and/or (b) a heavy chain region having at least 85%, 90%, 91%, 92%, 93%, 94%, 95%, 96%, 97%, 98%, or 99% identity to SEQ ID NO:26 or consisting essentially of the amino acid sequence of SEQ ID NO:26.

In some embodiments, the binding region comprises a polypeptide having at least 85%, 90%, 91%, 92%, 93%, 94%, 95%, 96%, 97%, 98%, or 99% identity to any one of SEQ ID NOs: 28-35 and 38-40 or consists essentially of the polypeptide shown in any one of SEQ ID NOs: 28-35 and 38-40.

In some embodiments, the binding region is a single-chain variable fragment, such as, e.g., consisting of, comprising, or consisting essentially of the polypeptide of any one of SEQ ID NOs: 28-35 and 38-40.

In some embodiments, the binding region comprises a polypeptide(s) selected from the group consisting of: (a) a heavy chain variable (V_{H}) domain comprising (i) a HCDR1 comprising or consisting essentially of the amino acid sequence of SEQ ID NO:36 or SEQ ID NO: 22; (ii) a HCDR2 comprising or consisting essentially of the amino acid sequence of SEQ ID NO: 37 or SEQ ID NO: 23; and/or (iii) a HCDR3 comprising or consisting essentially of the amino acid sequence of SEQ ID NO:24; and/or (b) a light chain variable (V_{L}) domain comprising (i) a LCDR1 comprising or consisting essentially of the amino acid sequence of SEQ ID NO:19; (ii) a LCDR2 comprising or consisting essentially of the amino acid sequence of SEQ ID NO:20; and/or (iii) a LCDR3 comprising or consisting essentially of the amino acid sequence of SEQ ID NO:21.

A binding region of a PD-L1 targeting molecule may be, e.g., a monoclonal antibody or engineered antibody derivative.

In some embodiments, the binding region is an antibody fragment, e.g., a Fv, Fab, Fab′, scFv, diabody, or F(ab′) 2 fragment. In another embodiment, the binding region is a full-length antibody, e.g., an intact IgG1 antibody or other antibody class or isotype as defined herein.

In some embodiments, the binding region is a synthetically engineered antibody derivate, such as, e.g. an autonomous V_{H} domain (such as, e.g., from camelids, murine, or human sources), single-domain antibody domain (sdAb), heavy-chain antibody domains derived from a camelid (V_{H}H fragment or V_{H} domain fragment), heavy-chain antibody domains derived from a camelid V_{H}H fragments or V_{H} domain fragments, heavy-chain antibody domain derived from a cartilaginous fish, immunoglobulin new antigen receptor (IgNAR), V_{NAR} fragment, single-chain variable (scFv) fragment, nanobody, “camelized” scaffolds comprising a V_{H} domain, Fd fragment consisting of the heavy chain and C_{H}1 domains, single chain Fv-C_{H}3 minibody, Fc antigen binding domain (Fcabs), scFv-Fc fusion, multimerizing scFv fragment (diabodies, triabodies, tetrabodies), disulfide-stabilized antibody variable (Fv) fragment (dsFv), disulfide-stabilized antigen-binding (Fab) fragment consisting of the V_{L}, V_{H}, C_{L} and C_{H}1 domains, single-chain variable-region fragments comprising a disulfide-stabilized heavy and light chain (sc-dsFvs), bivalent nanobodies, bivalent minibodies, bivalent F(ab′)₂ fragments (Fab dimers), bispecific tandem V_{H}H fragments, bispecific tandem scFv fragments, bispecific nanobodies, bispecific minibodies, and any genetically manipulated counterparts of the foregoing that retain its paratope and binding function, such as, e.g., wherein the relative orientation or order of the heavy and light chains is reversed or flipped.

According to one specific, but non-limiting aspect, the binding region may comprise an immunoglobulin-type binding region. The term “immunoglobulin-type binding region” as used herein refers to a polypeptide region capable of binding one or more target biomolecules, such as an antigen or epitope. Binding regions may be functionally defined by their ability to bind to target molecules. Immunoglobulin-type binding regions are commonly derived from antibody or antibody-like structures.

Immunoglobulin (Ig) proteins have a structural domain known as an Ig domain. Ig domains range in length from about 70-110 amino acid residues and possess a characteristic Ig-fold, in which typically 7 to 9 antiparallel beta strands arrange into two beta sheets which form a sandwich-like structure. The Ig fold is stabilized by hydrophobic amino acid interactions on inner surfaces of the sandwich and highly conserved disulfide bonds between cysteine residues in the strands. Ig domains may be variable (IgV or V-set), constant (IgC or C-set) or intermediate (IgI or I-set). Some Ig domains may be associated with a complementarity determining region (CDR), also called a “complementary determining region,” which is important for the specificity of antibodies binding to their epitopes. Ig-like domains are also found in non-immunoglobulin proteins and are classified on that basis as members of the Ig superfamily of proteins. The HUGO Gene Nomenclature Committee (HGNC) provides a list of members of the Ig-like domain containing family.

An immunoglobulin-type binding region may be a polypeptide sequence of an antibody or antigen-binding fragment thereof wherein the amino acid sequence has been varied from that of a native antibody or an Ig-like domain of a non-immunoglobulin protein, for example by molecular engineering or selection by library screening. Because of the relevance of recombinant DNA techniques and in vitro library screening in the generation of immunoglobulin-type binding regions, antibodies can be redesigned to obtain desired characteristics, such as smaller size, cell entry, or other improvements for in vivo and/or therapeutic applications. The possible variations are many and may range from the changing of just one amino acid to the complete redesign of, for example, a variable region. Typically, changes in the variable region will be made in order to improve the antigen-binding characteristics, improve variable region stability, or reduce the potential for immunogenic responses.

There are numerous immunoglobulin-type binding regions contemplated as components of the molecules described herein. In some embodiments, the immunoglobulin-type binding region is derived from an immunoglobulin binding region, such as an antibody paratope capable of binding an extracellular part of PD-L1. In certain other embodiments, the immunoglobulin-type binding region comprises an engineered polypeptide not derived from any immunoglobulin domain but which functions like an immunoglobulin binding region by providing high-affinity binding to an extracellular part of PD-L1. This engineered polypeptide may optionally include polypeptide scaffolds comprising or consisting essentially of complementary determining regions from immunoglobulins as described herein.

There are also numerous binding regions in the prior art that are useful for targeting polypeptides to specific cell-types via their high-affinity binding characteristics. In some embodiments, the binding region of the PD-L1 targeting molecule is selected from the group which includes autonomous V_{H} domains, single-domain antibody domains (sdAbs), heavy-chain antibody domains derived from camelids (V_{H}H fragments or V_{H} domain fragments), heavy-chain antibody domains derived from camelid V_{H}H fragments or V_{H} domain fragments, heavy-chain antibody domains derived from cartilaginous fishes, immunoglobulin new antigen receptors (IgNARs), V_{NAR} fragments, single-chain variable (scFv) fragments, nanobodies, Fd fragments consisting of the heavy chain and C_{H}1 domains, single chain Fv-C_{H}3 minibodies, dimeric C_{H}2 domain fragments (C_{H}2D), Fc antigen binding domains (Fcabs), isolated complementary determining region 3 (CDR3) fragments, constrained framework region 3, CDR3, framework region 4 (FR3-CDR3-FR4) polypeptides, small modular immunopharmaceutical (SMIP) domains, scFv-Fc fusions, multimerizing scFv fragments (diabodies, triabodies, tetrabodies), disulfide stabilized antibody variable (Fv) fragments, disulfide stabilized antigen-binding (Fab) fragments consisting of the V_{L}, V_{H}, C_{L} and C_{H}1 domains, bivalent nanobodies, bivalent minibodies, bivalent F(ab′)₂ fragments (Fab dimers), bispecific tandem V_{H}H fragments, bispecific tandem scFv fragments, bispecific nanobodies, bispecific minibodies, and any genetically manipulated counterparts of the foregoing that retain its paratope and binding function, such as, e.g., wherein the relative orientation or order of the heavy and light chains is reversed or flipped (see Ward E et al., Nature 341: 544-6 (1989); Davies J, Riechmann L, Biotechnology (NY) 13: 475-9 (1995); Reiter Y et al., Mol Biol 290: 685-98 (1999); Riechmann L, Muyldermans S, J Immunol Methods 231: 25-38 (1999); Tanha J et al., J Immunol Methods 263: 97-109 (2002); Vranken W et al., Biochemistry 41: 8570-9 (2002); Jespers L et al., J Mol Biol 337: 893-903 (2004); Jespers L et al., Nat Biotechnol 22: 1161-5 (2004); To R et al., J Biol Chem 280: 41395-403 (2005); Saerens D et al., Curr Opin Pharmacol 8: 600-8 (2008); Dimitrov D, MAbs 1:26-8 (2009); Weiner L, Cell 148: 1081-4 (2012); Ahmad Z et al., Clin Dev Immunol 2012: 980250 (2012)).

There are a variety of binding regions comprising polypeptides derived from the constant regions of immunoglobulins, such as, e.g., engineered dimeric Fc domains, monomeric Fcs (mFcs), scFv-Fcs, V_{H}H-Fcs, C_{H}2 domains, monomeric C_{H}3s domains (mC_{H}3s), synthetically reprogrammed immunoglobulin domains, and/or hybrid fusions of immunoglobulin domains with ligands (Hofer T et al., Proc Natl Acad Sci U.S.A 105: 12451-6 (2008); Xiao J et al., J Am Chem Soc 131: 13616-13618 (2009); Xiao X et al., Biochem Biophys Res Commun 387: 387-92 (2009); Wozniak-Knopp G et al., Protein Eng Des Sel 23 289-97 (2010); Gong R et al., PLoS ONE 7: e42288 (2012); Wozniak-Knopp G et al., PLoS ONE 7: e30083 (2012); Ying T et al., J Biol Chem 287: 19399-408 (2012); Ying T et al., J Biol Chem 288: 25154-64 (2013); Chiang M et al., J Am Chem Soc 136: 3370-3 (2014); Rader C, Trends Biotechnol 32: 186-97 (2014); Ying T et al., Biochimica Biophys Acta 1844: 1977-82 (2014)).

In accordance with certain other embodiments, the binding region comprises an engineered, alternative scaffold to immunoglobulin domains. Engineered alternative scaffolds are known in the art which exhibit similar functional characteristics to immunoglobulin-derived structures, such as high-affinity and specific binding of target biomolecules, and might provide improved characteristics to certain immunoglobulin domains, such as, e.g., greater stability or reduced immunogenicity. Generally, alternative scaffolds to immunoglobulins are less than 20 kilodaltons, consist of a single polypeptide chain, lack cysteine residues, and exhibit relatively high thermodynamic stability.

Any of the aforementioned PD-L1 binding molecules may be suitable for use as a PD-L1 binding region or modified to create one or more PD-L1 binding regions for use in a PD-L1 targeting molecule. Any of the above binding region structures may be used as a component of a molecule described herein as long as the binding region component has a dissociation constant of 10⁻⁵ to 10⁻¹² moles per liter, preferably less than 200 nanomolar (nM), towards an extracellular part of a PD-L1 molecule.

### B. Shiga Toxin Effector Polypeptides

The PD-L1 targeting molecules described herein may comprise at least one, Shiga toxin effector polypeptide derived from a Shiga toxin A Subunit. A Shiga toxin effector polypeptide is a polypeptide derived from a Shiga toxin A Subunit member of the Shiga toxin family that is capable of exhibiting one or more Shiga toxin functions (see e.g., Cheung M et al., Mol Cancer 9: 28 (2010); WO 2014/164680, WO 2014/164693, WO 2015/138435, WO 2015/138452, WO 2015/113005, WO 2015/113007, WO 2015/191764, WO 2016/196344, WO 2017/019623, WO 2018/106895, and WO 2018/140427). Shiga toxin functions include, e.g., increasing cellular internalization, directing subcellular routing from an endosomal compartment to the cytosol, avoiding intracellular degradation, catalytically inactivating ribosomes, and effectuating cytostatic and/or cytotoxic effects.

The Shiga toxin family of protein toxins is composed of various naturally occurring toxins which are structurally and functionally related, e.g., Shiga toxin, Shiga-like toxin 1, and Shiga-like toxin 2 (Johannes L, Römer W, Nat Rev Microbiol 8: 105-16 (2010)). Holotoxin members of the Shiga toxin family contain targeting domains that preferentially bind a specific glycosphingolipid present on the surface of some host cells and an enzymatic domain capable of permanently inactivating ribosomes once inside a cell (Johannes L, Römer W, Nat Rev Microbiol 8: 105-16 (2010)). Members of the Shiga toxin family share the same overall structure and mechanism of action (Engedal N et al., Microbial Biotech 4: 32-46 (2011)). For example, Stx, SLT-1 and SLT-2 display indistinguishable enzymatic activity in cell-free systems (Head S et al., J Biol Chem 266: 3617-21 (1991); Tesh V et al., Infect Immun 61: 3392-402 (1993); Brigotti M et al., Toxicon 35:1431-1437 (1997)).

The Shiga toxin family encompasses true Shiga toxin (Stx) isolated from S. dysenteriae serotype 1, Shiga-like toxin 1 variants (SLT1 or Stx1 or SLT-1 or Slt-I) isolated from serotypes of enterohemorrhagic E. coli, and Shiga-like toxin 2 variants (SLT2 or Stx2 or SLT-2) isolated from serotypes of enterohemorrhagic E. coli. (see e.g. Gonzalez-Escalona N, Kase J A, PLoS One 14: e0214620 (2019)). SLT1 differs by only one amino acid residue from Stx, and both have been referred to as Verocytotoxins or Verotoxins (VTs) (O'Brien A, Curr Top Microbiol Immunol 180: 65-94 (1992)). Although SLT1 and SLT2 variants are only about 53-60% similar to each other at the primary amino acid sequence level, they share mechanisms of enzymatic activity and cytotoxicity common to the members of the Shiga toxin family (Johannes L, Römer W, Nat Rev Microbiol 8: 105-16 (2010)). Over 39 different Shiga toxins have been described, such as the defined subtypes Stx1a, Stx1c, Stx1d, Stx1e, and Stx2a-g (Scheutz F et al., J Clin Microbiol 50: 2951-63 (2012); Probert W et al., J Clin Microbiol 52: 2346-51 (2014)). Members of the Shiga toxin family are not naturally restricted to any bacterial species (e.g. E. coli, S. dysenteriae, S. flexneri, S. boydii, E. cloacae, and/or S. enterica) because Shiga-toxin-encoding genes can spread among bacterial species via horizontal gene transfer (Strauch E et al., Infect Immun 69: 7588-95 (2001); Bielaszewska M et al., Appl Environ Micrbiol 73: 3144-50 (2007); Zhaxybayeva O, Doolittle W, Curr Biol 21: R242-6 (2011); Khalil R et al., Pathog Dis 74: ftw037 (2016)). As an example of interspecies transfer, a Shiga toxin was discovered in a strain of A. haemolyticus isolated from a patient (Grotiuz G et al., J Clin Microbiol 44: 3838-41 (2006)). Once a Shiga toxin encoding polynucleotide enters a new subspecies or species, the Shiga toxin amino acid sequence is presumed to be capable of developing slight sequence variations due to genetic drift and/or selective pressure while still maintaining a mechanism of cytotoxicity common to members of the Shiga toxin family (see Scheutz F et al., J Clin Microbiol 50: 2951-63 (2012)).

In some embodiments, a Shiga toxin A Subunit effector polypeptide component of the PD-L1 binding molecules described herein comprises a combination of two or more of the following Shiga toxin effector polypeptide sub-regions: (1) a de-immunized sub-region, (2) a protease-cleavage resistant sub-region near the carboxy-terminus of a Shiga toxin A1 fragment region, and (3) a T-cell epitope-peptide embedded or inserted sub-region.

### 1. De-Immunized, Shiga Toxin a Subunit Effector Polypeptides

In some embodiments, the Shiga toxin effector polypeptide of a PD-L1 targeting molecule is de-immunized, such as, e.g., as compared to a wild-type Shiga toxin, wild-type Shiga toxin polypeptide, and/or Shiga toxin effector polypeptide comprising only wild-type polypeptide sequences. A Shiga toxin effector polypeptide and/or Shiga toxin A Subunit polypeptide, whether naturally occurring or not, can be de-immunized by a method described herein, described in WO 2015/113005, WO 2015/113007, WO 2016/196344, and WO 2018/140427, and/or known to the skilled worker, wherein the resulting molecule retains one or more Shiga toxin A Subunit functions. The de-immunized, Shiga toxin effector polypeptide may comprise a disruption of at least one, putative, endogenous, epitope region in order to reduce the antigenic and/or immunogenic potential of the Shiga toxin effector polypeptide after administration of the polypeptide to a chordate.

In some embodiments, the Shiga toxin effector polypeptide comprises a disruption of an endogenous epitope or epitope region, such as, e.g., a B-cell and/or CD4+ T-cell epitope. In some embodiments, the Shiga toxin effector polypeptide comprises a disruption of at least one, endogenous, epitope region described herein, wherein the disruption reduces the antigenic and/or immunogenic potential of the Shiga toxin effector polypeptide after administration of the polypeptide to a chordate, and wherein the Shiga toxin effector polypeptide is capable of exhibiting one or more Shiga toxin A Subunit functions, such as, e.g., a significant level of Shiga toxin cytotoxicity.

The term “disrupted” or “disruption” as used herein with regard to an epitope region refers to the deletion of at least one amino acid residue in an epitope region, inversion of two or more amino acid residues where at least one of the inverted amino acid residues is in an epitope region, insertion of at least one amino acid into an epitope region, and a substitution of at least one amino acid residue in an epitope region. An epitope region disruption by mutation includes amino acid substitutions with non-standard amino acids and/or non-natural amino acids. Epitope regions may alternatively be disrupted by mutations comprising the modification of an amino acid by the addition of a covalently-linked chemical structure which masks at least one amino acid in an epitope region, see, e.g. PEGylation (see Zhang C et al., BioDrugs 26: 209-15 (2012), small molecule adjuvants (Flower D, Expert Opin Drug Discov 7: 807-17 (2012), and site-specific albumination (Lim S et al., J Control Release 207-93 (2015)).

Certain epitope regions and disruptions are indicated herein by reference to specific amino acid positions of native Shiga toxin A Subunits provided in the Sequence Listing, noting that naturally occurring Shiga toxin A Subunits may comprise precursor forms containing signal sequences of about 22 amino acids at their amino-terminals which are removed to produce mature Shiga toxin A Subunits and are recognizable to the skilled worker. Further, certain epitope region disruptions are indicated herein by reference to specific amino acids (e.g. S for a serine residue) natively present at specific positions within native Shiga toxin A Subunits (e.g. S33 for the serine residue at position 33 from the amino-terminus) followed by the amino acid with which that residue has been substituted in the particular mutation under discussion (e.g. S33I represents the amino acid substitution of isoleucine for serine at amino acid residue 33 from the amino-terminus).

In some embodiments, the de-immunized, Shiga toxin effector polypeptide comprises a disruption of at least one epitope region provided herein. In some embodiments, the de-immunized, Shiga toxin effector polypeptide comprises a disruption of at least one epitope region described in WO 2015/113005 or WO 2015/113007.

In some embodiments, the de-immunized, Shiga toxin effector polypeptide comprises or consists essentially of a full-length Shiga toxin A Subunit (e.g. SLT-1A (SEQ ID NO:1), StxA (SEQ ID NO:2), or SLT-2A (SEQ ID NO:3)) comprising at least one disruption of the amino acid sequence selected from the group of natively positioned amino acids consisting of: 1-15 of SEQ ID NO:1 or SEQ ID NO:2; 3-14 of SEQ ID NO:3; 26-37 of SEQ ID NO:3; 27-37 of SEQ ID NO:1 or SEQ ID NO:2; 39-48 of SEQ ID NO:1 or SEQ ID NO:2; 42-48 of SEQ ID NO:3; 53-66 of SEQ ID NO:1, SEQ ID NO:2, or SEQ ID NO:3; 94-115 of SEQ ID NO:1, SEQ ID NO:2, or SEQ ID NO:3; 141-153 of SEQ ID NO:1 or SEQ ID NO:2; 140-156 of SEQ ID NO:3; 179-190 of SEQ ID NO:1 or SEQ ID NO:2; 179-191 of SEQ ID NO:3; 204 of SEQ ID NO:3; 205 of SEQ ID NO:1 or SEQ ID NO:2; 210-218 of SEQ ID NO:3; 240-258 of SEQ ID NO:3; 243-257 of SEQ ID NO:1 or SEQ ID NO:2; 254-268 of SEQ ID NO:1 or SEQ ID NO:2; 262-278 of SEQ ID NO:3; 281-297 of SEQ ID NO:3; and 285-293 of SEQ ID NO:1 or SEQ ID NO:2, or the equivalent position in a Shiga toxin A Subunit polypeptide, conserved Shiga toxin effector polypeptide sub-region, and/or non-native, Shiga toxin effector polypeptide sequence.

In some embodiments, the Shiga toxin effector polypeptide comprises or consists essentially of a truncated Shiga toxin A Subunit. Truncations of Shiga toxin A Subunits might result in the deletion of an entire epitope region(s) without affecting Shiga toxin effector function(s). The smallest, Shiga toxin A Subunit fragment shown to exhibit significant enzymatic activity was a polypeptide composed of residues 75-247 of StxA (Al-Jaufy A et al., Infect Immun 62: 956-60 (1994)). Truncating the carboxy-terminus of SLT-1A, StxA, or SLT-2A to amino acids 1-251 removes two predicted B-cell epitope regions, two predicted CD4 positive (CD4+) T-cell epitopes, and a predicted, discontinuous, B-cell epitope. Truncating the amino-terminus of SLT-1A, StxA, or SLT-2A to 75-293 removes at least three, predicted, B-cell epitope regions and three predicted CD4+ T-cell epitopes. Truncating both amino- and carboxy-terminals of SLT-1A, StxA, or SLT-2A to 75-251 deletes at least five, predicted, B-cell epitope regions; four, putative, CD4+ T-cell epitopes; and one, predicted, discontinuous, B-cell epitope.

In some embodiments, a Shiga toxin effector polypeptide comprises or consists essentially of a full-length or truncated Shiga toxin A Subunit with at least one mutation, e.g. deletion, insertion, inversion, or substitution, in a provided epitope region. In some embodiments, the polypeptides comprise a disruption which comprises a deletion of at least one amino acid within the epitope region. In some embodiments, the polypeptides comprise a disruption which comprises an insertion of at least one amino acid within the epitope region. In some embodiments, the polypeptides comprise a disruption which comprises an inversion of amino acids, wherein at least one inverted amino acid is within the epitope region. In some embodiments, the polypeptides comprise a disruption which comprises a mutation, such as an amino acid substitution to a non-standard amino acid or an amino acid with a chemically modified side chain.

In some embodiments, the Shiga toxin effector polypeptides comprise or consist essentially of a full-length or truncated Shiga toxin A Subunit with one or more mutations as compared to the native sequence which comprises at least one amino acid substitution selected from the group consisting of: A, G, V, L, I, P, C, M, F, S, D, N, Q, H, and K. In some embodiments, the polypeptide comprises or consists essentially of a full-length or truncated Shiga toxin A Subunit with a single mutation as compared to the native sequence wherein the substitution is selected from the group consisting of: D to A, D to G, D to V, D to L, D to I, D to F, D to S, D to Q, E to A, E to G, E to V, E to L, E to I, E to F, E to S, E to Q, E to N, E to D, E to M, E to R, G to A, H to A, H to G, H to V, H to L, H to I, H to F, H to M, K to A, K to G, K to V, K to L, K to I, K to M, K to H, L to A, L to G, N to A, N to G, N to V, N to L, N to I, N to F, P to A, P to G, P to F, R to A, R to G, R to V, R to L, R to I, R to F, R to M, R to Q, R to S, R to K, R to H, S to A, S to G, S to V, S to L, S to I, S to F, S to M, T to A, T to G, T to V, T to L, T to I, T to F, T to M, T to S, Y to A, Y to G, Y to V, Y to L, Y to I, Y to F, and Y to M.

In some embodiments, the Shiga toxin effector polypeptides comprise or consist essentially of a full-length or truncated Shiga toxin A Subunit with one or more mutations as compared to the native amino acid residue sequence which comprises at least one amino acid substitution of an immunogenic residue and/or within an epitope region, wherein at least one substitution occurs at the natively positioned group of amino acids selected from the group consisting of: 1 of SEQ ID NO:1 or SEQ ID NO:2; 4 of SEQ ID NO:1, SEQ ID NO:2, or SEQ ID NO:3; 8 of SEQ ID NO:1, SEQ ID NO:2, or SEQ ID NO:3; 9 of SEQ ID NO:1, SEQ ID NO:2, or SEQ ID NO:3; 11 of SEQ ID NO:1, SEQ ID NO:2, or SEQ ID NO:3; 33 of SEQ ID NO:1 or SEQ ID NO:2; 43 of SEQ ID NO:1 or SEQ ID NO:2; 44 of SEQ ID NO:1 or SEQ ID NO:2; 45 of SEQ ID NO:1 or SEQ ID NO:2; 46 of SEQ ID NO:1 or SEQ ID NO:2; 47 of SEQ ID NO:1 or SEQ ID NO:2; 48 of SEQ ID NO:1, SEQ ID NO:2, or SEQ ID NO:3; 49 of SEQ ID NO:1 or SEQ ID NO:2; 50 of SEQ ID NO:1 or SEQ ID NO:2; 51 of SEQ ID NO:1 or SEQ ID NO:2; 53 of SEQ ID NO:1 or SEQ ID NO:2; 54 of SEQ ID NO:1 or SEQ ID NO:2; 55 of SEQ ID NO:1 or SEQ ID NO:2; 56 of SEQ ID NO:1 or SEQ ID NO:2; 57 of SEQ ID NO:1 or SEQ ID NO:2; 58 of SEQ ID NO:1, SEQ ID NO:2, or SEQ ID NO:3; 59 of SEQ ID NO:1, SEQ ID NO:2, or SEQ ID NO:3; 60 of SEQ ID NO:1 or SEQ ID NO:2; 61 of SEQ ID NO:1 or SEQ ID NO:2; 62 of SEQ ID NO:1 or SEQ ID NO:2; 84 of SEQ ID NO:1 or SEQ ID NO:2; 88 of SEQ ID NO:1 or SEQ ID NO:2; 94 of SEQ ID NO:1, SEQ ID NO:2, or SEQ ID NO:3; 96 of SEQ ID NO:1, SEQ ID NO:2, or SEQ ID NO:3; 104 of SEQ ID NO:1 or SEQ ID NO:2; 105 of SEQ ID NO:1 or SEQ ID NO:2; 107 of SEQ ID NO:1 or SEQ ID NO:2; 108 of SEQ ID NO:1 or SEQ ID NO:2; 109 of SEQ ID NO:1, SEQ ID NO:2, or SEQ ID NO:3; 110 of SEQ ID NO:1 or SEQ ID NO:2; 111 of SEQ ID NO:1 or SEQ ID NO:2; 112 of SEQ ID NO:1, SEQ ID NO:2, or SEQ ID NO:3; 141 of SEQ ID NO:1 or SEQ ID NO:2; 147 of SEQ ID NO:1, SEQ ID NO:2, or SEQ ID NO:3; 154 of SEQ ID NO:1 or SEQ ID NO:2; 179 of SEQ ID NO:1, SEQ ID NO:2, or SEQ ID NO:3; 180 of SEQ ID NO:1 or SEQ ID NO:2; 181 of SEQ ID NO:1 or SEQ ID NO:2; 183 of SEQ ID NO:1, SEQ ID NO:2, or SEQ ID NO:3; 184 of SEQ ID NO:1, SEQ ID NO:2, or SEQ ID NO:3; 185 of SEQ ID NO:1 or SEQ ID NO:2; 186 of SEQ ID NO:1, SEQ ID NO:2, or SEQ ID NO:3; 187 of SEQ ID NO:1 or SEQ ID NO:2; 188 of SEQ ID NO:1 or SEQ ID NO:2; 189 of SEQ ID NO:1 or SEQ ID NO:2; 198 of SEQ ID NO:1 or SEQ ID NO:2; 204 of SEQ ID NO:3; 205 of SEQ ID NO:1 or SEQ ID NO:2; 241 of SEQ ID NO:3; 242 of SEQ ID NO:1 or SEQ ID NO:2; 247 of SEQ ID NO:1 or SEQ ID NO:2; 247 of SEQ ID NO:3; 248 of SEQ ID NO:1 or SEQ ID NO:2; 250 of SEQ ID NO:3; 251 of SEQ ID NO:1 or SEQ ID NO:2; 264 of SEQ ID NO:1, SEQ ID NO:2, or SEQ ID NO:3; 265 of SEQ ID NO:1 or SEQ ID NO:2; and 286 of SEQ ID NO:1 or SEQ ID NO:2.

In some embodiments, the Shiga toxin effector polypeptides comprise or consist essentially of a full-length or truncated Shiga toxin A Subunit with at least one substitution of an immunogenic residue and/or within an epitope region, wherein at least one amino acid substitution is to a non-conservative amino acid (see, e.g., Table 3, infra) relative to a natively occurring amino acid positioned at one of the following native positions: 1 of SEQ ID NO:1 or SEQ ID NO:2; 4 of SEQ ID NO:1, SEQ ID NO:2, or SEQ ID NO:3; 8 of SEQ ID NO:1, SEQ ID NO:2, or SEQ ID NO:3; 9 of SEQ ID NO:1, SEQ ID NO:2, or SEQ ID NO:3; 11 of SEQ ID NO:1, SEQ ID NO:2, or SEQ ID NO:3; 33 of SEQ ID NO:1 or SEQ ID NO:2; 43 of SEQ ID NO:1 or SEQ ID NO:2; 44 of SEQ ID NO:1 or SEQ ID NO:2; 45 of SEQ ID NO:1 or SEQ ID NO:2; 46 of SEQ ID NO:1 or SEQ ID NO:2; 47 of SEQ ID NO:1 or SEQ ID NO:2; 48 of SEQ ID NO:1, SEQ ID NO:2, or SEQ ID NO:3; 49 of SEQ ID NO:1 or SEQ ID NO:2; 50 of SEQ ID NO:1 or SEQ ID NO:2; 51 of SEQ ID NO:1 or SEQ ID NO:2; 53 of SEQ ID NO:1 or SEQ ID NO:2; 54 of SEQ ID NO:1 or SEQ ID NO:2; 55 of SEQ ID NO:1 or SEQ ID NO:2; 56 of SEQ ID NO:1 or SEQ ID NO:2; 57 of SEQ ID NO:1 or SEQ ID NO:2; 58 of SEQ ID NO:1, SEQ ID NO:2, or SEQ ID NO:3; 59 of SEQ ID NO:1, SEQ ID NO:2, or SEQ ID NO:3; 60 of SEQ ID NO:1 or SEQ ID NO:2; 61 of SEQ ID NO:1 or SEQ ID NO:2; 62 of SEQ ID NO:1 or SEQ ID NO:2; 84 of SEQ ID NO:1 or SEQ ID NO:2; 88 of SEQ ID NO:1 or SEQ ID NO:2; 94 of SEQ ID NO:1, SEQ ID NO:2, or SEQ ID NO:3; 96 of SEQ ID NO:1, SEQ ID NO:2, or SEQ ID NO:3; 104 of SEQ ID NO:1 or SEQ ID NO:2; 105 of SEQ ID NO:1 or SEQ ID NO:2; 107 of SEQ ID NO:1 or SEQ ID NO:2; 108 of SEQ ID NO:1 or SEQ ID NO:2; 109 of SEQ ID NO:1, SEQ ID NO:2, or SEQ ID NO:3; 110 of SEQ ID NO:1 or SEQ ID NO:2; 111 of SEQ ID NO:1 or SEQ ID NO:2; 112 of SEQ ID NO:1, SEQ ID NO:2, or SEQ ID NO:3; 141 of SEQ ID NO:1 or SEQ ID NO:2; 147 of SEQ ID NO:1, SEQ ID NO:2, or SEQ ID NO:3; 154 of SEQ ID NO:1 or SEQ ID NO:2; 179 of SEQ ID NO:1, SEQ ID NO:2, or SEQ ID NO:3; 180 of SEQ ID NO:1 or SEQ ID NO:2; 181 of SEQ ID NO:1 or SEQ ID NO:2; 183 of SEQ ID NO:1, SEQ ID NO:2, or SEQ ID NO:3; 184 of SEQ ID NO:1, SEQ ID NO:2, or SEQ ID NO:3; 185 of SEQ ID NO:1 or SEQ ID NO:2; 186 of SEQ ID NO:1, SEQ ID NO:2, or SEQ ID NO:3; 187 of SEQ ID NO:1 or SEQ ID NO:2; 188 of SEQ ID NO:1 or SEQ ID NO:2; 189 of SEQ ID NO:1 or SEQ ID NO:2; 198 of SEQ ID NO:1 or SEQ ID NO:2; 204 of SEQ ID NO:3; 205 of SEQ ID NO:1 or SEQ ID NO:2; 241 of SEQ ID NO:3; 242 of SEQ ID NO:1 or SEQ ID NO:2; 247 of SEQ ID NO:1 or SEQ ID NO:2; 247 of SEQ ID NO:3; 248 of SEQ ID NO:1 or SEQ ID NO:2; 250 of SEQ ID NO:3; 251 of SEQ ID NO:1 or SEQ ID NO:2; 264 of SEQ ID NO:1, SEQ ID NO:2, or SEQ ID NO:3; 265 of SEQ ID NO:1 or SEQ ID NO:2; and 286 of SEQ ID NO:1 or SEQ ID NO:2.

In some embodiments, the Shiga toxin effector polypeptides comprise or consist essentially of a full-length or truncated Shiga toxin A Subunit with at least one amino acid substitution selected from the group consisting of: K1 to A, G, V, L, I, F, M and H; T4 to A, G, V, L, I, F, M, and S; D6 to A, G, V, L, I, F, S, and Q; S8 to A, G, V, I, L, F, and M; T8 to A, G, V, I, L, F, M, and S; T9 to A, G, V, I, L, F, M, and S; S9 to A, G, V, L, I, F, and M; K11 to A, G, V, L, I, F, M and H; T12 to A, G, V, I, L, F, M, and S; S33 to A, G, V, L, I, F, and M; S43 to A, G, V, L, I, F, and M; G44 to A and L; S45 to A, G, V, L, I, F, and M; T45 to A, G, V, L, I, F, and M; G46 to A and P; D47 to A, G, V, L, I, F, S, and Q; N48 to A, G, V, L, and M; L49 to A or G; F50; A51 to V; D53 to A, G, V, L, I, F, S, and Q; V54 to A, G, and L; R55 to A, G, V, L, I, F, M, Q, S, K, and H; G56 to A and P; 157 to A, G, M, and F; L57 to A, G, M, and F; D58 to A, G, V, L, I, F, S, and Q; P59 to A, G, and F; E60 to A, G, V, L, I, F, S, Q, N, D, M, and R; E61 to A, G, V, L, I, F, S, Q, N, D, M, and R; G62 to A; D94 to A, G, V, L, I, F, S, and Q; R84 to A, G, V, L, I, F, M, Q, S, K, and H; V88 to A and G; I88 to A, G, and V; D94; S96 to A, G, V, I, L, F, and M; T104 to A, G, V, I, L, F, M, and S; A105 to L; T107 to A, G, V, I, L, F, M, and S; S107 to A, G, V, L, I, F, and M; L108 to A, G, and M; S109 to A, G, V, I, L, F, and M; T109 to A, G, V, I, L, F, M, and S; G110 to A; D111 to A, G, V, L, I, F, S, and Q; S112 to A, G, V, L, I, F, and M; D141 to A, G, V, L, I, F, S, and Q; G147 to A; V154 to A and G; R179 to A, G, V, L, I, F, M, Q, S, K, and H; T180 to A, G, V, L, I, F, M, and S; T181 to A, G, V, L, I, F, M, and S; D183 to A, G, V, L, I, F, S, and Q; D184 to A, G, V, L, I, F, S, and Q; L185 to A, G, and V; S186 to A, G, V, I, L, F, and M; G187 to A; R188 to A, G, V, L, I, F, M, Q, S, K, and H; S189 to A, G, V, I, L, F, and M; D197 to A, G, V, L, I, F, S, and Q; D198 to A, G, V, L, I, F, S, and Q; R204 to A, G, V, L, I, F, M, Q, S, K, and H; R205 to A, G, V, L, I, F, M, Q, S, K and H; C242 to A, G, V, and S; S247 to A, G, V, I, L, F, and M; Y247 to A, G, V, L, I, F, and M; R248 to A, G, V, L, I, F, M, Q, S, K, and H; R250 to A, G, V, L, I, F, M, Q, S, K, and H; R251 to A, G, V, L, I, F, M, Q, S, K, and H; C262 to A, G, V, and S; D264 to A, G, V, L, I, F, S, and Q; G264 to A; and T286 to A, G, V, L, I, F, M, and S.

In some embodiments, the Shiga toxin effector polypeptides comprise or consist essentially of a full-length or truncated Shiga toxin A Subunit with at least one of the following amino acid substitutions K1A, K1M, T4I, D6R, S8I, T8V, T9I, S9I, K11A, K11H, T12K, S33I, S33C, S43N, G44L, S45V, S45I, T45V, T45I, G46P, D47M, D47G, N48V, N48F, L49A, F50T, A51V, D53A, D53N, D53G, V54L, V54I, R55A, R55V, R55L, G56P, I57F, I57M, D58A, D58V, D58F, P59A, P59F, E60I, E60T, E60R, E61A, E61V, E61L, G62A, R84A, V88A, D94A, S96I, T104N, A105L, T107P, L108M, S109V, T109V, G110A, D111T, S112V, D141A, G147A, V154A, R179A, T180G, T181I, D183A, D183G, D184A, D184A, D184F, L185V, L185D, S186A, S186F, G187A, G187T, R188A, R188L, S189A, D198A, R204A, R205A, C242S, S247I, Y247A, R248A, R250A, R251A, or D264A, G264A, T286A, and/or T286I, wherein the amino acid numbering is relative to any one of SEQ ID NOs: 1-3. These epitope disrupting substitutions may be combined to form a de-immunized, Shiga toxin effector polypeptide with multiple substitutions per epitope region and/or multiple epitope regions disrupted while still retaining Shiga toxin effector function. For example, substitutions at the natively positioned K1A, K1M, T4I, D6R, S8I, T8V, T9I, S9I, K11A, K11H, T12K, S33I, S33C, S43N, G44L, S45V, S45I, T45V, T45I, G46P, D47M, D47G, N48V, N48F, L49A, F50T, A51V, D53A, D53N, D53G, V54L, V54I, R55A, R55V, R55L, G56P, I57F, I57M, D58A, D58V, D58F, P59A, P59F, E60I, E60T, E60R, E61A, E61V, E61L, G62A, R84A, V88A, D94A, S96I, T104N, A105L, T107P, L108M, S109V, T109V, G110A, D111T, S112V, D141A, G147A, V154A, R179A, T180G, T181I, D183A, D183G, D184A, D184A, D184F, L185V, L185D, S186A, S186F, G187A, G187T, R188A, R188L, S189A, D198A, R204A, R205A, C242S, S247I, Y247A, R248A, R250A, R251A, or D264A, G264A, T286A, and/or T286I may be combined, where possible, with substitutions at the natively positioned residues K1A, K1M, T4I, D6R, S8I, T8V, T9I, S9I, K11A, K11H, T12K, S33I, S33C, S43N, G44L, S45V, S45I, T45V, T45I, G46P, D47M, D47G, N48V, N48F, L49A, F50T, A51V, D53A, D53N, D53G, V54L, V54I, R55A, R55V, R55L, G56P, I57F, I57M, D58A, D58V, D58F, P59A, P59F, E60I, E60T, E60R, E61A, E61V, E61L, G62A, R84A, V88A, D94A, S96I, T104N, A105L, T107P, L108M, S109V, T109V, G110A, D111T, S112V, D141A, G147A, V154A, R179A, T180G, T181I, D183A, D183G, D184A, D184A, D184F, L185V, L185D, S186A, S186F, G187A, G187T, R188A, R188L, S189A, D198A, R204A, R205A, C242S, S247I, Y247A, R248A, R250A, R251A, or D264A, G264A, T286A, and/or T286I to create de-immunized, Shiga toxin effector polypeptides.

Any of the de-immunized, Shiga toxin effector polypeptide sub-regions and/or epitope disrupting mutations described herein may be used alone or in combination with each individual embodiment described herein, including methods.

### 2. Protease-Cleavage Resistant, Shiga Toxin A Subunit Effector Polypeptides

In some embodiments, the Shiga toxin effector polypeptide of the PD-L1 targeting molecule comprises (1) a Shiga toxin A1 fragment derived region having a carboxy-terminus and (2) a disrupted furin-cleavage site at the carboxy-terminus of the Shiga toxin A1 fragment region. Improving the stability of connections between the Shiga toxin component and other components of PD-L1 targeting molecules, e.g., cell-targeting binding regions, can improve their toxicity profiles after administration to organisms by reducing non-specific toxicities caused by the breakdown of the connection and loss of cell-targeting, such as, e.g., as a result of proteolysis.

Shiga toxin A Subunits of members of the Shiga toxin family comprise a conserved, furin-cleavage site at the carboxy-terminal of their A1 fragment regions important for Shiga toxin function. Furin-cleavage site sites and furin-cleavage sites can be identified by the skilled worker using standard techniques and/or by using the information herein.

The model of Shiga toxin cytotoxicity is that intracellular proteolytic processing of Shiga toxin A Subunits by furin in intoxicated cells is essential for 1) liberation of the A1 fragment from the rest of the Shiga holotoxin, 2) escape of the A1 fragment from the endoplasmic reticulum by exposing a hydrophobic domain in the carboxy-terminus of the A1 fragment, and 3) enzymatic activation of the A1 fragment (see Johannes L, Römer W, Nat Rev Microbiol 8: 105-16 (2010)). The efficient liberation of the Shiga toxin A1 fragment from the A2 fragment and the rest of the components of the Shiga holotoxin in the endoplasmic reticulum of intoxicated cells is essential for efficient intracellular routing to the cytosol, maximal enzymatic activity, efficient ribosome inactivation, and achieving optimal cytotoxicity, i.e. comparable to a wild-type Shiga toxin (see e.g. WO 2015/191764 and references therein).

During Shiga toxin intoxication, the A Subunit is proteolytically cleaved by furin at the carboxy bond of a conserved arginine residue (e.g. the arginine residue at position 251 in StxA and SLT-1A and the arginine residue at position 250 in Stx2A and SLT-2A). Furin cleavage of Shiga toxin A Subunits occurs in endosomal and/or Golgi compartments. Furin is a specialized serine endoprotease which is expressed by a wide variety of cell types, in all human tissues examined, and by most animal cells. Furin cleaves polypeptides comprising accessible sites often centered on the minimal, dibasic, consensus site R-x-(R/K/x)-R (SEQ ID NO: 161). The A Subunits of members of the Shiga toxin family comprise a conserved, surface-exposed, extended loop structure (e.g. 242-261 in StxA and SLT-1A, and 241-260 in SLT-2) with a conserved S-R/Y-x-x-R site (SEQ ID NO: 162) which is cleaved by furin. The surface exposed, extended loop structure positioned at amino acid residues 242-261 in StxA is required for furin-induced cleavage of StxA, including features flanking the minimal, furin-cleavage site R-x-x-R (SEQ ID NO: 160).

Furin-cleavage sites and furin-cleavage sites in Shiga toxin A Subunits and Shiga toxin effector polypeptides can be identified by the skilled worker using standard methods and/or by using the information herein. Furin cleaves the minimal, consensus site R-x-x-R (SEQ ID NO: 160) (Schalken J et al., J Clin Invest 80: 1545-9 (1987); Bresnahan P et al., J Cell Biol 111: 2851-9 (1990); Hatsuzawa K et al., J Biol Chem 265: 22075-8 (1990); Wise R et al., Proc Natl Acad Sci USA 87: 9378-82 (1990); Molloy S et al., J Biol Chem 267: 16396-402 (1992)). Consistent with this, many furin inhibitors comprise peptides comprising the site R-x-x-R (SEQ ID NO: 160). An example of a synthetic inhibitor of furin is a molecule comprising the peptide R-V-K-R (SEQ ID NO: 163) (Henrich S et al., Nat Struct Biol 10: 520-6 (2003)). In general, a peptide or protein comprising a surface accessible, dibasic amino acid site with two positively charged, amino acids separated by two amino acid residues can be predicted to be sensitive to furin-cleavage with cleavage occurring at the carboxy bond of the last basic amino acid in the site.

Consensus sites in substrates cleaved by furin have been identified with some degree of specificity. A furin-cleavage site site has been described that comprises a region of twenty, continuous, amino acid residues, which can be labeled P14 through P6′ (Tian S et al., Int J Mol Sci 12: 1060-5 (2011)) using the nomenclature described in Schechter I, Berger, A, Biochem Biophys Res Commun 32: 898-902 (1968). According to this nomenclature, the furin-cleavage site is at the carboxy bond of the amino acid residue designated P1, and the amino acid residues of the furin-cleavage site are numbered P2, P3, P4, etc., in the direction going toward the amino-terminus from this reference P1 residue. The amino acid residues of the site going toward the carboxy-terminus from the P1 reference residue are numbered with the prime notation P2′, P3′, P4′, etc. Using this nomenclature, the P6 to P2′ region delineates the core substrate of the furin cleavage site which is bound by the enzymatic domain of furin. The two flanking regions P14 to P7 and P3′ to P6′ are often rich in polar, amino acid residues to increase the accessibility to the core furin cleavage site located between them.

A general, furin-cleavage site is often described by the consensus sequence R-x-x-R (SEQ ID NO: 160) which corresponds to P4-P3-P2-P1; where “R” represents an arginine residue (see Table 1, supra), a dash “-” represents a peptide bond, and a lowercase “x” represents any amino acid residue. However, other residues and positions may help to further define furin-cleavage sites. A slightly more refined furin-cleavage site, consensus site is often reported as the consensus sequence R-x-[K/R]-R (SEQ ID NO: 164) (where a forward slash “/” means “or” and divides alternative amino acid residues at the same position), which corresponds to P4-P3-P2-P1, because it was observed that furin has a strong preference for cleaving substrates containing this site.

In addition to the minimal, furin-cleavage site R-x-x-R (SEQ ID NO: 160), a larger, furin-cleavage site has been described with certain amino acid residue preferences at certain positions. By comparing various known furin substrates, certain physicochemical properties have been characterized for the amino acid residues in a 20 amino acid residue long, furin-cleavage site site. The P6 to P2′ region of the furin-cleavage site delineates the core furin-cleavage site which physically interacts with the enzymatic domain of furin. The two flanking regions P14 to P7 and P3′ to P6′ are often hydrophilic being rich in polar, amino acid residues to increase the surface accessibility of the core furin-cleavage site located between them.

In general, the furin-cleavage site from position P5 to P1 tends to comprise amino acid residues with a positive charge and/or high isoelectric points. In particular, the P1 position, which marks the position of furin proteolysis, is generally occupied by an arginine but other positively charged, amino acid residues can occur in this position. Positions P2 and P3 tend to be occupied by flexible, amino acid residues, and in particular P2 tends to be occupied by arginine, lysine, or sometimes by very small and flexible amino acid residues like glycine. The P4 position tends to be occupied by positively charged, amino acid residues in furin substrates. However, if the P4 position is occupied by an aliphatic, amino acid residue, then the lack of a positively charged, functional group can be compensated for by a positively charged residue located at position(s) P5 and/or P6. Positions P1′ and P2′ are commonly occupied by aliphatic and/or hydrophobic amino acid residues, with the P1′ position most commonly being occupied by a serine.

The two, hydrophilic, flanking regions tend to be occupied by amino acid residues which are polar, hydrophilic, and have smaller amino acid functional groups; however, in certain verified furin substrates, the flanking regions do not contain any hydrophilic, amino acid residues (see Tian S, Biochem Insights 2: 9-20 (2009)).

The twenty amino acid residue, furin-cleavage site found in native, Shiga toxin A Subunits at the junction between the Shiga toxin A1 fragment and A2 fragment is well characterized in certain Shiga toxins. For example in StxA (SEQ ID NO:2) and SLT-1A (SEQ ID NO:1), this furin-cleavage site is natively positioned from L238 to F257, and in SLT-2A (SEQ ID NO:3), this furin-cleavage site is natively positioned from V237 to Q256. Based on amino acid homology, experiment, and/or furin-cleavage assays described herein, the skilled worker can identify furin-cleavage sites in other native, Shiga toxin A Subunits or Shiga toxin effector polypeptides, where the sites are actual furin-cleavage sites or are predicted to result in the production of A1 and A2 fragments after furin cleavage of those molecules within a eukaryotic cell.

In some embodiments, the Shiga toxin effector polypeptide comprises (1) a Shiga toxin A1 fragment derived polypeptide having a carboxy-terminus and (2) a disrupted furin-cleavage site at the carboxy-terminus of the Shiga toxin A1 fragment derived polypeptide. The carboxy-terminus of a Shiga toxin A1 fragment derived polypeptide may be identified by the skilled worker by using techniques known in the art, such as, e.g., by using protein sequence alignment software to identify (i) a furin-cleavage site conserved with a naturally occurring Shiga toxin, (ii) a surface exposed, extended loop conserved with a naturally occurring Shiga toxin, and/or (iii) a stretch of amino acid residues which are predominantly hydrophobic (i.e. a hydrophobic “patch”) that may be recognized by the ERAD system.

A protease-cleavage resistant, Shiga toxin effector polypeptide of the PD-L1 targeting molecule (1) may be completely lacking any furin-cleavage site at a carboxy-terminus of its Shiga toxin A1 fragment region and/or (2) comprise a disrupted furin-cleavage site at the carboxy-terminus of its Shiga toxin A1 fragment region and/or region derived from the carboxy-terminus of a Shiga toxin A1 fragment. A disruption of a furin-cleavage site include various alterations to an amino acid residue in the furin-cleavage site, such as, e.g., a post-translation modification(s), an alteration of one or more atoms in an amino acid functional group, the addition of one or more atoms to an amino acid functional group, the association to a non-proteinaceous moiety(ies), and/or the linkage to an amino acid residue, peptide, polypeptide such as resulting in a branched proteinaceous structure.

Protease-cleavage resistant, Shiga toxin effector polypeptides may be created from a Shiga toxin effector polypeptide and/or Shiga toxin A Subunit polypeptide, whether naturally occurring or not, using a method described herein, described in WO 2015/191764, and/or known to the skilled worker, wherein the resulting molecule still retains one or more Shiga toxin A Subunit functions.

As used herein with regard to a furin-cleavage site, the term “disruption” or “disrupted” refers to an alteration from the naturally occurring furin-cleavage site, such as, e.g., a mutation, that results in a reduction in furin-cleavage proximal to the carboxy-terminus of a Shiga toxin A1 fragment region, or identifiable region derived thereof, as compared to the furin-cleavage of a wild-type Shiga toxin A Subunit or a polypeptide derived from a wild-type Shiga toxin A Subunit comprising only wild-type polypeptide sequences. An alteration to an amino acid residue in the furin-cleavage site includes a mutation in the furin-cleavage site, such as, e.g., a deletion, insertion, inversion, substitution, and/or carboxy-terminal truncation of the furin-cleavage site, as well as a post-translation modification, such as, e.g., as a result of glycosylation, albumination, and the like which involve conjugating or linking a molecule to the functional group of an amino acid residue. Because the furin-cleavage site is comprised of about twenty, amino acid residues, in theory, alterations, modifications, mutations, deletions, insertions, and/or truncations involving one or more amino acid residues of any one of these twenty positions might result in a reduction of furin-cleavage sensitivity (Tian S et al., Sci Rep 2: 261 (2012)). The disruption of a furin-cleavage site furin-cleavage sitemight or might not increase resistance to cleavage by other proteases, such as, e.g., trypsin and extracellular proteases common in the vascular system of mammals. The effects of a given disruption to cleavage sensitivity of a given protease may be tested by the skilled worker using techniques known in the art.

As used herein, a “disrupted furin-cleavage site” is furin-cleavage site comprising an alteration to one or more amino acid residues derived from the 20 amino acid residue region representing a conserved, furin-cleavage site found in native, Shiga toxin A Subunits at the junction between the Shiga toxin A1 fragment and A2 fragment regions and positioned such that furin cleavage of a Shiga toxin A Subunit results in the production of the A1 and A2 fragments; wherein the disrupted furin-cleavage site exhibits reduced furin cleavage in an experimentally reproducible way as compared to a reference molecule comprising a wild-type, Shiga toxin A1 fragment region fused to a carboxy-terminal polypeptide of a size large enough to monitor furin cleavage using the appropriate assay known to the skilled worker and/or described herein.

Examples of types of mutations which can disrupt a furin-cleavage site furin-cleavage site are amino acid residue deletions, insertions, truncations, inversions, and/or substitutions, including substitutions with non-standard amino acids and/or non-natural amino acids. In addition, furin-cleavage sites furin-cleavage site can be disrupted by mutations comprising the modification of an amino acid by the addition of a covalently-linked structure which masks at least one amino acid in the site or site, such as, e.g., as a result of PEGylation, the coupling of small molecule adjuvants, and/or site-specific albumination.

If a furin-cleavage site has been disrupted by mutation and/or the presence of non-natural amino acid residues, certain disrupted furin-cleavage sites may not be easily recognizable as being related to any furin-cleavage site; however, the carboxy-terminus of the Shiga toxin A1 fragment derived region will be recognizable and will define where the furin-cleavage site would be located were it not disrupted. For example, a disrupted furin-cleavage site may comprise less than the twenty, amino acid residues of the furin-cleavage site due to a carboxy-terminal truncation as compared to a Shiga toxin A Subunit and/or Shiga toxin A1 fragment.

In some embodiments, the Shiga toxin effector polypeptide comprises (1) a Shiga toxin A1 fragment derived polypeptide having a carboxy-terminus and (2) a disrupted furin-cleavage site at the carboxy-terminus of the Shiga toxin A1 fragment polypeptide region; wherein the Shiga toxin effector polypeptide (and any PD-L1 targeting molecule comprising it) is more furin-cleavage resistant as compared to a reference molecule, such as, e.g., a wild-type Shiga toxin polypeptide comprising the carboxy-terminus of an A1 fragment and/or the conserved, furin-cleavage site between A1 and A2 fragments. For example, a reduction in furin cleavage of one molecule compared to a reference molecule may be determined using an in vitro, furin-cleavage assay described in WO 2015/191764, conducted using the same conditions, and then performing a quantitation of the band density of any fragments resulting from cleavage to quantitatively measure in change in furin cleavage.

In some embodiments, the Shiga toxin effector polypeptide is more resistant to furin-cleavage in vitro and/or in vivo as compared to a wild-type, Shiga toxin A Subunit.

In general, the protease-cleavage sensitivity of a PD-L1 targeting molecule is tested by comparing it to the same molecule having its furin-cleavage resistant, Shiga toxin effector polypeptide replaced with a wild-type, Shiga toxin effector polypeptide comprising a Shiga toxin A1 fragment. In some embodiments, the molecules comprising a disrupted furin-cleavage site exhibits a reduction in in vitro furin cleavage of 30%, 40%, 50%, 60%, 70%, 80%, 90%, 95%, 97%, 98% or greater compared to a reference molecule comprising a wild-type, Shiga toxin A1 fragment fused at its carboxy-terminus to a peptide or polypeptide.

Several furin-cleavage site disruptions have been described. For example, mutating the two conserved arginines to alanines in the minimal R-x-x-R site (SEQ ID NO: 160) completely blocked processing by furin and/or furin-like proteases (see e.g Duda A et al., J Virology 78: 13865-70 (2004)). Because the furin-cleavage site site comprises about twenty amino acid residues, in theory, certain mutations involving one or more of any one of these twenty, amino acid residue positions might abolish furin cleavage or reduce furin cleavage efficiency (see e.g. Tian S et al., Sci Rep 2: 261 (2012)).

In some embodiments, the molecules comprise a Shiga toxin effector polypeptide derived from at least one A Subunit of a member of the Shiga toxin family wherein the Shiga toxin effector polypeptide comprises a disruption in one or more amino acids derived from the conserved, highly accessible, protease-cleavage sensitive loop of Shiga toxin A Subunits. For example, in StxA and SLT-1A, this highly accessible, protease-sensitive loop is natively positioned from amino acid residues 242 to 261, and in SLT-2A, this conserved loop is natively positioned from amino acid residues 241 to 260. Based on polypeptide sequence homology, the skilled worker can identify this conserved, highly accessible loop structure in other Shiga toxin A Subunits. Certain mutations to the amino acid residues in this loop can reduce the accessibility of certain amino acid residues within the loop to proteolytic cleavage and this might reduce furin-cleavage sensitivity.

In some embodiments, a molecule described herein comprises a Shiga toxin effector polypeptide comprising a disrupted furin-cleavage site comprising a mutation in the surface-exposed, protease sensitive loop conserved among Shiga toxin A Subunits. In some embodiments, a molecule comprises a Shiga toxin effector polypeptide comprising a disrupted furin-cleavage site comprising a mutation in this protease-sensitive loop of Shiga toxin A Subunits, the mutation which reduce the surface accessibility of certain amino acid residues within the loop such that furin-cleavage sensitivity is reduced.

In some embodiments, the disrupted furin-cleavage site of a Shiga toxin effector polypeptide comprises a disruption in terms of existence, position, or functional group of one or both of the consensus amino acid residues P1 and P4, such as, e.g., the amino acid residues in positions 1 and 4 of the minimal furin-cleavage site R/Y-x-x-R (SEQ ID NO: 159). For example, mutating one or both of the two arginine residues in the minimal, furin consensus site R-x-x-R (SEQ ID NO: 160) to alanine will disrupt a furin-cleavage site and prevent furin-cleavage at that site. Similarly, amino acid residue substitutions of one or both of the arginine residues in the minimal furin-cleavage site R-x-x-R (SEQ ID NO: 160) to any non-conservative amino acid residue known to the skilled worker will reduced the furin-cleavage sensitivity of the site. In particular, amino acid residue substitutions of arginine to any non-basic amino acid residue which lacks a positive charge, such as, e.g., A, G, P, S, T, D, E, Q, N, C, I, L, M, V, F, W, and Y, will result in a disrupted furin-cleavage site.

In some embodiments, the disrupted furin-cleavage site of a Shiga toxin effector polypeptide comprises a disruption in the spacing between the consensus amino acid residues P4 and P1 in terms of the number of intervening amino acid residues being other than two, and, thus, changing either P4 and/or P1 into a different position and eliminating the P4 and/or P1 designations. For example, deletions within the furin-cleavage site of the minimal furin-cleavage site or the core, furin-cleavage site will reduce the furin-cleavage sensitivity of the furin-cleavage site.

In some embodiments, the disrupted furin-cleavage site comprises one or more amino acid residue substitutions, as compared to a wild-type, Shiga toxin A Subunit. In some embodiments, the disrupted furin-cleavage site comprises one or more amino acid residue substitutions within the minimal furin-cleavage site R/Y-x-x-R (SEQ ID NO: 159), such as, e.g., for StxA and SLT-1A derived Shiga toxin effector polypeptides, the natively positioned amino acid residue R248 substituted with any non-positively charged, amino acid residue and/or R251 substituted with any non-positively charged, amino acid residue; and for SLT-2A derived Shiga toxin effector polypeptides, the natively positioned amino acid residue Y247 substituted with any non-positively charged, amino acid residue and/or R250 substituted with any non-positively charged, amino acid residue.

In some embodiments, the disrupted furin-cleavage site comprises an un-disrupted, minimal furin-cleavage site R/Y-x-x-R (SEQ ID NO: 159) but instead comprises a disrupted flanking region, such as, e.g., amino acid residue substitutions in one or more amino acid residues in the furin-cleavage site flanking regions natively position at, e.g., 241-247 and/or 252-259. In some embodiments, the disrupted furin cleavage site comprises a substitution of one or more of the amino acid residues located in the P1-P6 region of the furin-cleavage site; mutating P1′ to a bulky amino acid, such as, e.g., R, W, Y, F, and H; and mutating P2′ to a polar and hydrophilic amino acid residue; and substituting one or more of the amino acid residues located in the P1′-P6′ region of the furin-cleavage site with one or more bulky and hydrophobic amino acid residues

In some embodiments, the disruption of the furin-cleavage site comprises a deletion, insertion, inversion, and/or mutation of at least one amino acid residue within the furin-cleavage site. In some embodiments, a protease-cleavage resistant, Shiga toxin effector polypeptide comprises a disruption of the amino acid sequence natively positioned at 249-251 of the A Subunit of Shiga-like toxin 1 (SEQ ID NO:1) or Shiga toxin (SEQ ID NO:2), or at 247-250 of the A Subunit of Shiga-like toxin 2 (SEQ ID NO:3) or the equivalent position in a conserved Shiga toxin effector polypeptide and/or non-native Shiga toxin effector polypeptide sequence. In some embodiments, protease-cleavage resistant, Shiga toxin effector polypeptides comprise a disruption which comprises a deletion of at least one amino acid within the furin-cleavage site. In some embodiments, protease-cleavage resistant, Shiga toxin effector polypeptides comprise a disruption which comprises an insertion of at least one amino acid within the protease-cleavage site. In some embodiments, the protease-cleavage resistant, Shiga toxin effector polypeptides comprise a disruption which comprises an inversion of amino acids, wherein at least one inverted amino acid is within the protease site. In some embodiments, the protease-cleavage resistant, Shiga toxin effector polypeptides comprise a disruption which comprises a mutation, such as an amino acid substitution to a non-standard amino acid or an amino acid with a chemically modified side chain.

In some embodiments, the disrupted furin-cleavage site comprises the deletion of nine, ten, eleven, or more of the carboxy-terminal amino acid residues within the furin-cleavage site. In these embodiments, the disrupted furin-cleavage site will not comprise a minimal furin-cleavage site. In other words, some embodiments lack a furin-cleavage site at the carboxy-terminus of the A1 fragment region.

In some embodiments, the disrupted furin-cleavage site comprises both an amino acid residue deletion and an amino acid residue substitution as compared to a wild-type, Shiga toxin A Subunit. In some embodiments, the disrupted furin-cleavage site comprises one or more amino acid residue deletions and substitutions within the minimal furin-cleavage site R/Y-x-x-R (SEQ ID NO: 159), such as, e.g., for StxA and SLT-1A derived Shiga toxin effector polypeptides, the natively positioned amino acid residue R248 substituted with any non-positively charged, amino acid residue and/or R251 substituted with any non-positively charged, amino acid residue; and for SLT-2A derived Shiga toxin effector polypeptides, the natively positioned amino acid residue Y247 substituted with any non-positively charged, amino acid residue and/or R250 substituted with any non-positively charged, amino acid residue.

In some embodiments, the disrupted furin-cleavage site comprises an amino acid residue deletion and an amino acid residue substitution as well as a carboxy-terminal truncation as compared to a wild-type, Shiga toxin A Subunit. In some embodiments, the disrupted furin-cleavage site comprises one or more amino acid residue deletions and substitutions within the minimal furin-cleavage site R/Y-x-x-R (SEQ ID NO: 159), such as, e.g., for StxA and SLT-1A derived Shiga toxin effector polypeptides, the natively positioned amino acid residue R248 substituted with any non-positively charged, amino acid residue and/or R251 substituted with any non-positively charged, amino acid residue; and for SLT-2A derived Shiga toxin effector polypeptides, the natively positioned amino acid residue Y247 substituted with any non-positively charged, amino acid residue and/or R250 substituted with any non-positively charged, amino acid residue.

In some embodiments, the disrupted furin-cleavage site comprises both an amino acid substitution within the minimal furin-cleavage site R/Y-x-x-R (SEQ ID NO: 159) and a carboxy-terminal truncation as compared to a wild-type, Shiga toxin A Subunit, such as, e.g., for StxA and SLT-1A derived Shiga toxin effector polypeptides, truncations ending at the natively amino acid position 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and comprising the natively positioned amino acid residue R248 and/or R251 substituted with any non-positively charged, amino acid residue where appropriate; and for SLT-2A derived Shiga toxin effector polypeptides, truncations ending at the natively amino acid position 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and comprising the natively positioned amino acid residue Y247 and/or R250 substituted with any non-positively charged, amino acid residue where appropriate.

In some embodiments, the disrupted furin-cleavage site comprises an insertion of one or more amino acid residues as compared to a wild-type, Shiga toxin A Subunit as long as the inserted amino residue(s) does not create a de novo furin-cleavage site. In some embodiments, the insertion of one or more amino acid residues disrupts the natural spacing between the arginine residues in the minimal, furin-cleavage site R/Y-x-x-R (SEQ ID NO: 159), such as, e.g., StxA and SLT-1A derived polypeptides comprising an insertion of one or more amino acid residues at 249 or 250 and thus between R248 and R251; or SLT-2A derived polypeptides comprising an insertion of one or more amino acid residues at 248 or 249 and thus between Y247 and R250.

In some embodiments, the disrupted furin-cleavage site comprises both an amino acid residue insertion and a carboxy-terminal truncation as compared to a wild-type, Shiga toxin A Subunit. In some embodiments, the disrupted furin-cleavage site comprises both an amino acid residue insertion and an amino acid residue substitution as compared to a wild-type, Shiga toxin A Subunit. In some embodiments, the disrupted furin-cleavage site comprises both an amino acid residue insertion and an amino acid residue deletion as compared to a wild-type, Shiga toxin A Subunit.

In some embodiments, the disrupted furin-cleavage site comprises an amino acid residue deletion, an amino acid residue insertion, and an amino acid residue substitution as compared to a wild-type, Shiga toxin A Subunit.

In some embodiments, the disrupted furin-cleavage site comprises an amino acid residue deletion, insertion, substitution, and carboxy-terminal truncation as compared to a wild-type, Shiga toxin A Subunit.

In some embodiments, the Shiga toxin effector polypeptide comprising a disrupted furin-cleavage site is directly fused by a peptide bond to a molecular moiety comprising an amino acid, peptide, and/or polypeptide wherein the fused structure involves a single, continuous polypeptide. In these fusion embodiments, the amino acid sequence following the disrupted furin-cleavage site should not create a de novo, furin-cleavage site at the fusion junction.

Any of the above protease-cleavage resistant, Shiga toxin effector polypeptide sub-regions and/or disrupted furin-cleavage sites may be used alone or in combination with each individual embodiment described herein, including methods.

### 3. T-Cell Hyper-Immunized, Shiga Toxin a Subunit Effector Polypeptides

In some embodiments, the Shiga toxin effector polypeptide of a PD-L1 targeting molecule described herein comprises an embedded or inserted epitope-peptide. In some embodiments, the epitope-peptide is a heterologous, T-cell epitope-peptide, such as, e.g., an epitope considered heterologous to Shiga toxin A Subunits. In some embodiments, the epitope-peptide is a CD8+ T-cell epitope. In some embodiments, the CD8+ T-cell epitope-peptide has a binding affinity to a MHC class I molecule characterized by a dissociation constant (K_{D}) of 10⁻⁴ molar or less and/or the resulting MHC class I-epitope-peptide complex has a binding affinity to a T-cell receptor (TCR) characterized by a dissociation constant (K_{D}) of 10⁻⁴ molar or less.

In some embodiments, the Shiga toxin effector polypeptide comprises an embedded or inserted, heterologous, T-cell epitope, such as, e.g., a human CD8+ T-cell epitope. In some embodiments, the heterologous, T-cell epitope is embedded or inserted so as to disrupt an endogenous epitope or epitope region (e.g. a B-cell epitope and/or CD4+ T-cell epitope) identifiable in a naturally occurring Shiga toxin polypeptide or parental Shiga toxin effector polypeptide from which the Shiga toxin effector polypeptide is derived.

In some embodiments, the Shiga toxin effector polypeptide (and any PD-L1 targeting molecule comprising it) is CD8+ T-cell hyper-immunized, such as, e.g., as compared to a wild-type Shiga toxin polypeptide. Each CD8+ T-cell hyper-immunized, Shiga toxin effector polypeptide comprises an embedded or inserted T-cell epitope-peptide. Hyper-immunized, Shiga toxin effector polypeptides can be created from Shiga toxin effector polypeptides and/or Shiga toxin A Subunit polypeptides, whether naturally occurring or not, using a method described herein, described in WO 2015/113005, and/or known to the skilled worker, wherein the resulting molecule still retains one or more Shiga toxin A Subunit functions.

For the purposes of the present disclosure, a “T-cell epitope” is a molecular structure which is comprised by an antigenic peptide and can be represented by a linear, amino acid sequence. Commonly, T-cell epitopes are peptides of sizes of eight to eleven amino acid residues (Townsend A, Bodmer H, Annu Rev Immunol 7: 601-24 (1989)); however, certain T-cell epitope-peptides have lengths that are smaller than eight or larger than eleven amino acids long (see e.g. Livingstone A, Fathman C, Annu Rev Immunol 5: 477-501 (1987); Green K et al., Eur J Immunol 34: 2510-9 (2004)). In some embodiments, the embedded or inserted epitope is at least seven amino acid residues in length. In some embodiments, the embedded or inserted epitope is bound by a TCR with a binding affinity characterized by a K_{D} less than 10 mM (e.g. 1-100 μM) as caluclated using the formula in Stone J et al., Immunology 126: 165-76 (2009). However, it should be noted that the binding affinity within a given range between the MHC-epitope and TCR may not correlate with antigenicity and/or immunogenicity (see e.g. Al-Ramadi B et al., J Immunol 155: 662-73 (1995)), such as due to factors like MHC-peptide-TCR complex stability, MHC-peptide density and MHC-independent functions of TCR cofactors such as CD8 (Baker B et al., Immunity 13: 475-84 (2000); Hornell T et al., J Immunol 170: 4506-14 (2003); Woolridge L et al., J Immunol 171: 6650-60 (2003)).

A heterologous, T-cell epitope is an epitope not already present in a wild-type Shiga toxin A Subunit; a naturally occurring Shiga toxin A Subunit; and/or a parental, Shiga toxin effector polypeptide used as a source polypeptide for modification by a method described herein, described in WO 2015/113005, and/or known to the skilled worker.

A heterologous, T-cell epitope-peptide may be incorporated into a source polypeptide via numerous methods known to the skilled worker, including, e.g., the processes of creating one or more amino acid substitutions within the source polypeptide, fusing one or more amino acids to the source polypeptide, inserting one or more amino acids into the source polypeptide, linking a peptide to the source polypeptide, and/or a combination of the aforementioned processes. The result of such a method is the creation of a modified variant of the source polypeptide which comprises one or more embedded or inserted, heterologous, T-cell epitope-peptides.

T-cell epitopes may be chosen or derived from a number of source molecules. T-cell epitopes may be created or derived from various naturally occurring proteins. T-cell epitopes may be created or derived from various naturally occurring proteins foreign to mammals, such as, e.g., proteins of microorganisms. T-cell epitopes may be created or derived from mutated human proteins and/or human proteins aberrantly expressed by malignant human cells. T-cell epitopes may be synthetically created or derived from synthetic molecules (see e.g., Carbone F et al., J Exp Med 167: 1767-9 (1988); Del Val M et al., J Virol 65: 3641-6 (1991); Appella E et al., Biomed Pept Proteins Nucleic Acids 1: 177-84 (1995); Perez S et al., Cancer 116: 2071-80 (2010)).

Although any T-cell epitope-peptide is contemplated as being used as a heterologous, T-cell epitope, certain epitopes may be selected based on desirable properties. One objective is to create CD8+ T-cell hyper-immunized, Shiga toxin effector polypeptides for administration to vertebrates, meaning that the heterologous, T-cell epitope is highly immunogenic and can elicit robust immune responses in vivo when displayed complexed with a MHC class I molecule on the surface of a cell. In some embodiments, the Shiga toxin effector polypeptide comprises one or more, embedded or inserted, heterologous, T-cell epitopes which are CD8+ T-cell epitopes. A Shiga toxin effector polypeptide that comprises a heterologous, CD8+ T-cell epitope is considered a CD8+ T-cell hyper-immunized, Shiga toxin effector polypeptide.

T-cell epitope components may be chosen or derived from a number of source molecules already known to be capable of eliciting a vertebrate immune response. T-cell epitopes may be derived from various naturally occurring proteins foreign to vertebrates, such as, e.g., proteins of pathogenic microorganisms and non-self, cancer antigens. In particular, infectious microorganisms may contain numerous proteins with known antigenic and/or immunogenic properties. Further, infectious microorganisms may contain numerous proteins with known antigenic and/or immunogenic sub-regions or epitopes.

For example, the proteins of intracellular pathogens with mammalian hosts are sources for T-cell epitopes. There are numerous intracellular pathogens, such as viruses, bacteria, fungi, and single-cell eukaryotes, with well-studied antigenic proteins or peptides. T-cell epitopes can be selected or identified from human viruses or other intracellular pathogens, such as, e.g., bacteria like mycobacterium, fungi like toxoplasmae, and protists like trypanosomes.

For example, there are many immunogenic, viral peptide components of viral proteins from viruses that are infectious to humans. Numerous, human T-cell epitopes have been mapped to peptides within proteins from influenza A viruses, such as peptides in the proteins HA glycoproteins FE17, S139/1, CH65, C05, hemagglutin 1 (HA1), hemagglutinin 2 (HA2), nonstructural protein 1 and 2 (NS1 and NS 2), matrix protein 1 and 2 (M1 and M2), nucleoprotein (NP), neuraminidase (NA)), and many of these peptides have been shown to elicit human immune responses, such as by using ex vivo assay. Similarly, numerous, human T-cell epitopes have been mapped to peptide components of proteins from human cytomegaloviruses (HCMV), such as peptides in the proteins pp65 (UL83), UL128-131, immediate-early 1 (IE-1; UL123), glycoprotein B, tegument proteins, and many of these peptides have been shown to elicit human immune responses, such as by using ex vivo assays.

Another example is there are many immunogenic, cancer antigens in humans. The CD8+ T-cell epitopes of cancer and/or tumor cell antigens can be identified by the skilled worker using techniques known in the art, such as, e.g., differential genomics, differential proteomics, immunoproteomics, prediction then validation, and genetic approaches like reverse-genetic transfection (see e.g., Admon A et al., Mol Cell Proteomics 2: 388-98 (2003); Purcell A, Gorman J, Mol Cell Proteomics 3: 193-208 (2004); Comber J, Philip R, Ther Adv Vaccines 2: 77-89 (2014)). There are many antigenic and/or immunogenic T-cell epitopes already identified or predicted to occur in human cancer and/or tumor cells. For example, T-cell epitopes have been predicted in human proteins commonly mutated or overexpressed in neoplastic cells, such as, e.g., ALK, CEA, N-acetylglucosaminyl-transferase V (GnT-V), HCA587, PD-L1/neu, MAGE, Melan-A/MART-1, MUC-1, p53, and TRAG-3 (see e.g., van der Bruggen P et al., Science 254: 1643-7 (1991); Kawakami Y et al., J Exp Med 180: 347-52 (1994); Fisk B et al., J Exp Med 181: 2109-17 (1995); Guilloux Y et al., J Exp Med 183: 1173 (1996); Skipper J et al., J Exp Med 183: 527 (1996); Brossart P et al., 93: 4309-17 (1999); Kawashima I et al., Cancer Res 59: 431-5 (1999); Papadopoulos K et al., Clin Cancer Res 5: 2089-93 (1999); Zhu B et al., Clin Cancer Res 9: 1850-7 (2003); Li B et al., Clin Exp Immunol 140: 310-9 (2005); Ait-Tahar K et al., Int J Cancer 118: 688-95 (2006); Akiyama Y et al., Cancer Immunol Immunother 61: 2311-9 (2012)). In addition, synthetic variants of T-cell epitopes from human cancer cells have been created (see e.g., Lazoura E, Apostolopoulos V, Curr Med Chem 12: 629-39 (2005); Douat-Casassus C et al., J Med Chem 50: 1598-609 (2007)).

While any T-cell epitope may be used in the polypeptides and molecules described herein, certain T-cell epitopes may be preferred based on their known and/or empirically determined characteristics. For example, in many species, the MHC alleles in its genome encode multiple MHC-I molecular variants. Because MHC class I protein polymorphisms can affect antigen-MHC class I complex recognition by CD8+ T-cells, T-cell epitopes may be chosen based on knowledge about certain MHC class I polymorphisms and/or the ability of certain antigen-MHC class I complexes to be recognized by T-cells having different genotypes.

There are well-defined peptide-epitopes that are known to be immunogenic, MHC class I restricted, and/or matched with a specific human leukocyte antigen (HLA) variant(s). For applications in humans or involving human target cells, HLA-class I-restricted epitopes can be selected or identified by the skilled worker using standard techniques known in the art. The ability of peptides to bind to human MHC class I molecules can be used to predict the immunogenic potential of putative T-cell epitopes. The ability of peptides to bind to human MHC class I molecules can be scored using software tools. T-cell epitopes may be chosen for use as a heterologous, T-cell epitope component based on the peptide selectivity of the HLA variants encoded by the alleles more prevalent in certain human populations. For example, the human population is polymorphic for the alpha chain of MHC class I molecules due to the varied alleles of the HLA genes from individual to individual. In certain T-cell epitopes may be more efficiently presented by a specific HLA molecule, such as, e.g., the commonly occurring HLA variants encoded by the HLA-A allele groups HLA-A2 and HLA-A3.

An example of a CD8+ T-cell epitope is SIINFEKYL (SEQ ID NO:79). According to the Immune Epitope Database (IEDB), this peptide-epitope is bound by human HLA-A*02:01. This peptide-epitope is scored for the ability to bind to common human MHC class I human leukocyte antigen (HLA) variants encoded by the more prevalent alleles in human populations using the Immune Epitope Database (IEDB) Analysis Resource MHC-I binding prediction's consensus tool and recommended parameters (Kim Y et al., Nucleic Acids Res 40: W252-30 (2012)). The IEDB MHC-I binding prediction analysis indicated higher-affinity binders with lower percentile rankings. This peptide-epitope percentile rank was predicted to be 0.74 for binding to human HLA-A*02:01.

When choosing T-cell epitopes for use as a heterologous, T-cell epitope component, multiple factors may be considered that can influence epitope generation and transport to receptive MHC class I molecules, such as, e.g., the presence and epitope specificity of the following factors in the target cell: proteasome, ERAAP/ERAP1, tapasin, and TAPs.

When choosing T-cell epitopes for use as a heterologous, T-cell epitope component of the molecules described herein, an epitope may be selected which best match the MHC class I molecules present in the cell-type or cell populations to be targeted. Different MHC class I molecules exhibit preferential binding to particular peptide sequences, and particular peptide-MHC class I variant complexes are specifically recognized by the t-cell receptors (TCRs) of effector T-cells. The skilled worker can use knowledge about MHC class I molecule specificities and TCR specificities to optimize the selection of heterologous, T-cell epitopes.

In addition, multiple, immunogenic, T-cell epitopes for MHC class I presentation may be embedded in the same Shiga toxin effector polypeptide for use, such as, e.g., in the targeted delivery of a plurality of T-cell epitopes simultaneously.

Any of the protease-cleavage resistant, Shiga toxin effector polypeptide sub-regions and/or disrupted furin-cleavage sites described herein may be used alone or in combination with each individual embodiment described herein, including methods.

### C. Additional Exogenous Materials

In some embodiments, the PD-L1 targeting molecules comprise an additional exogenous material. An “additional exogenous material” as used herein refers to one or more atoms or molecules, often not generally present in both Shiga toxins and native target cells, where the PD-L1 targeting molecule can be used to specifically transport such material to the interior of a cell. In one sense, the entire PD-L1 targeting molecule is an exogenous material which will enter the cell; thus, the “additional” exogenous materials are heterologous materials linked to but other than the core PD-L1 targeting molecule itself. Non-limiting examples of additional exogenous materials are radionucleides, peptides, detection promoting agents, proteins, small molecule chemotherapeutic agents, and polynucleotides.

In some embodiments of the PD-L1 targeting molecules, the additional exogenous material is one or more radionucleides, such as, e.g., ²¹¹At, ¹³¹I, ¹²⁵I, ⁹⁰Y, ¹¹¹In, ¹⁸⁶Re, ¹⁸⁸Re, ¹⁵³Sm, ²¹²Bi, ³²P, ⁶⁰C, and/or radioactive isotopes of lutetium.

In some embodiments, the additional exogenous material comprises a proapoptotic peptide, polypeptide, or protein, such as, e.g., BCL-2, caspases (e.g. fragments of caspase-3 or caspase-6), cytochromes, granzyme B, apoptosis-inducing factor (AIF), BAX, tBid (truncated Bid), and proapoptotic fragments or derivatives thereof (see e.g., Ellerby H et al., Nat Med 5: 1032-8 (1999); Mai J et al., Cancer Res 61: 7709-12 (2001); Jia L et al., Cancer Res 63: 3257-62 (2003); Liu Y et al., Mol Cancer Ther 2: 1341-50 (2003); Perea S et al., Cancer Res 64: 7127-9 (2004); Xu Y et al., J Immunol 173: 61-7 (2004), Dälken B et al., Cell Death Differ 13: 576-85 (2006); Wang T et al., Cancer Res 67: 11830-9 (2007); Kwon M et al., Mol Cancer Ther 7: 1514-22 (2008); Qiu X et al., Mol Cancer Ther 7: 1890-9 (2008); Shan L et al., Cancer Biol Ther 11: 1717-22 (2008); Wang F et al., Clin Cancer Res 16: 2284-94 (2010); Kim J et al., J Virol 85: 1507-16 (2011)).

In some embodiments, the additional exogenous material comprises a protein or polypeptide comprising an enzyme. In certain other embodiments, the additional exogenous material is a nucleic acid, such as, e.g. a ribonucleic acid that functions as a small inhibiting RNA (siRNA) or microRNA (miRNA). In some embodiments, the additional exogenous material is an antigen, such as antigens derived from pathogens, bacterial proteins, viral proteins, proteins mutated in cancer, proteins aberrantly expressed in cancer, or T-cell complementary determining regions. For example, exogenous materials include antigens, such as those characteristic of antigen-presenting cells infected by bacteria, and T-cell complementary determining regions capable of functioning as exogenous antigens. Exogenous materials comprising polypeptides or proteins may optionally comprise one or more antigens whether known or unknown to the skilled worker.

In some embodiments of the PD-L1 targeting molecules, all heterologous antigens and/or epitopes associated with the Shiga toxin effector polypeptide are arranged in the PD-L1 targeting molecule amino-terminal to the carboxy-terminus of the Shiga toxin A1 fragment region of the Shiga toxin effector polypeptide. In some embodiments, all heterologous antigens and/or epitopes associated with the Shiga toxin effector polypeptide are associated, either directly or indirectly, with the Shiga toxin effector polypeptide at a position amino-terminal to the carboxy-terminus of the Shiga toxin A1 fragment region of the Shiga toxin effector polypeptide. In some embodiments, all additional exogenous material(s) which is an antigen is arranged amino-terminal to the Shiga toxin effector polypeptide, such as, e.g., fused directly or indirectly to the amino terminus of the Shiga toxin effector polypeptide.

In some embodiments of the PD-L1 targeting molecules, the additional exogenous material is a cytotoxic agent, such as, e.g., a small molecule chemotherapeutic agent, anti-neoplastic agent, cytotoxic antibiotic, alkylating agent, antimetabolite, topoisomerase inhibitor, and/or tubulin inhibitor (e.g., monomethylauristatin F (MMAF), paclitazel, laulimalide, vinblastine, colchicine, ZD6126, CA-4, CA-4P, oxi4503, AVE8062, phenstatin, CC-5079, podophyllotoxin, steganacin, nocodazole, curacin A, 2-ME, ENMD-1198, ABT-751, T138067, BCN-105P, indibulin, crolibulin, MPI-0441138, MPC-6827, CYT997, MN-029, CI-980, CP-248, CP-461, and TN16). Non-limiting examples of cytotoxic agents suitable for use as described herein include aziridines, cisplatins, tetrazines, procarbazine, hexamethylmelamine, vinca alkaloids, taxanes, camptothecins, etoposide, doxorubicin, mitoxantrone, teniposide, novobiocin, aclarubicin, anthracyclines, actinomycin, amanitin, amatoxins, bleomycin, centanamycin (indolecarboxamide), plicamycin, mitomycin, daunorubicin, epirubicin, idarubicins, dolastatins, maytansines, maytansionoids, duromycin, docetaxel, duocarmycins, adriamycin, calicheamicin, auristatins, pyrrolobenzodiazepines, pyrrolobenzodiazepine dimers (PBDs), carboplatin, 5-fluorouracil (5-FU), capecitabine, mitomycin C, paclitaxel, 1,3-Bis(2-chloroethyl)-1-nitrosourea (BCNU), rifampicin, cisplatin, methotrexate, gemcitabine, aceglatone, acetogenins (e.g. bullatacin and bullatacinone), aclacinomysins, AG1478, AG1571, aldophosphamide glycoside, alkyl sulfonates (e.g., busulfan, improsulfan, and piposulfan), alkylating agents (e.g. thiotepa and cyclosphosphamide), aminolevulinic acid, aminopterin, amsacrine, ancitabine, anthramycin, arabinoside, azacitidine, azaserine, aziridines (e.g., benzodopa, carboquone, meturedopa, and uredopa), azauridine, bestrabucil, bisantrene, bisphosphonates (e.g. clodronate), bleomycins, bortezomib, bryostatin, cactinomycin, callystatin, carabicin, carminomycin, carmofur, carmustine, carzinophilin, CC-1065, chlorambucil, chloranbucil, chlornaphazine, chlorozotocin, chromomycinis, chromoprotein enediyne antibiotic chromophores, CPT-11, cryptophycins (e.g. cryptophycin 1 and cryptophycin 8), cyclophosphamide, cytarabine, dacarbazine, dactinomycin, daunomycin, defofamine, demecolcine, detorubicin, diaziquone, 6-diazo-5-oxo-L-norleucine, dideoxyuridine, difluoromethylornithine (DMFO), doxifluridine, doxorubicins (e.g., morpholinodoxorubicin, cyanomorpholino-doxorubicin, 2-pyrrolinodoxorubicin, and deoxydoxorubicin), dynemicins, edatraxate, edatrexate, el eutherobins, elformithine, elliptinium acetate, enediyne antibiotics (e.g. calicheamicins), eniluracil, enocitabine, epirubicins, epothilone, esorubicins, esperamicins, estramustine, ethylenimines, 2-ethylhydrazide, etoglucid, fludarabine, folic acid analogues (e.g., denopterin, methotrexate, pteropterin, and trimetrexate), folic acid replenishers (e.g. frolinic acid), fotemustine, fulvestrant, gacytosine, gallium nitrate, gefitinib, gemcitabine, hydroxyurea, ibandronate, ifosfamide, imatinib mesylate, erlotinib, fulvestrant, letrozole, PTK787/ZK 222584 (Novartis, Basel, CH), oxaliplatin, leucovorin, rapamycin, lapatinib, lonafarnib, sorafenib, methylamelamines (e.g., altretamine, triethylenemelamine, triethy lenephosphoramide, triethylenethiophosphoramide and trimethylomelamine), pancratistatins, sarcodictyins, spongistatins, nitrogen mustards (e.g., chlorambucil, chlornaphazine, cyclophosphamide, mechlorethamine, mechlorethamine oxide hydrochloride, melphalan, novembichin, phenesterine, prednimustine, trofosfamide, and uracil mustard), nitrosureas (e.g., carmustine, fotemustine, lomustine, nimustine, and ranimnustine), dynemicins, neocarzinostatin chromophores, anthramycin, detorubicin, epirubicins, marcellomycins, mitomycins (e.g. mitomycin C), mycophenolic acid, nogalamycins, olivomycins, peplomycins, potfiromycins, puromycins, quelamycins, rodorubicins, ubenimex, zinostatins, zorubicins, purine analogs (e.g., fludarabine, 6-mercaptopurine, thiamiprine, and thioguanine), pyrimidine analogs (e.g., ancitabine, azacitidine, 6-azauridine, dideoxyuridine, doxifluridine, enocitabine, and floxuridine), aceglatone, lentinan, lonidainine, maytansinoids (e.g. maytansins and ansamitocins), mitoguazone, mitoxantrone, mopidanmol, nitraerine, pentostatin, phenamet, pirarubicin, podophyllinic acid, 2-ethylhydrazide, rhizoxin, sizofuran, spirogermanium, tenuazonic acid, triaziquone, 2,2′,2″trichlorotriethylamine, trichothecenes (e.g., T-2 toxin, verracurin A, roridin A, and anguidine), urethan, vindesine, mannomustine, mitobronitol, mitolactol, pipobroman, arabinoside, cyclophosphamide, toxoids (e.g. paclitaxel and doxetaxel), 6-thioguanine, mercaptopurine, platinum, platinum analogs (e.g. cisplatin and carboplatin), etoposide (VP-16), mitoxantrone, vinorelbine, novantrone, daunomycin, xeloda, topoisomerase inhibitor RFS 2000, retinoids (e.g. retinoic acid), capecitabine, lomustine, losoxantrone, mercaptopurines, nimustine, nitraerine, rapamycin, razoxane, roridin A, spongistatins, streptonigrins, streptozocins, sutent, T-2 toxin, thiamiprine, thiotepa, toxoids (e.g. paclitaxel and doxetaxel), tubercidins, verracurin A, vinblastine, vincristine, and structural analogs of any of the aforementioned (e.g. synthetic analogs), and/or derivatives of any of the aforementioned (see e.g., Lindell T et al., Science 170: 447-9 (1970); Remillard S et al., Science 189: 1002-5 (1975); Ravry M et al., Am J Clin Oncol 8: 148-50 (1985); Ravry M et al., Cancer Treat Rep 69: 1457-8 (1985); Sternberg C et al., Cancer 64: 2448-58 (1989); Bai R et al., Biochem Pharmacol 39: 1941-9 (1990); Boger D, Johnson D, Proc Natl Acad Sci USA 92: 3642-9 (1995); Beck J et al., Leuk Lymphoma 41: 117-24 (2001); Cassady J et al., Chem Pharm Bull (Tokyo) 52: 1-26 (2004); Sapra P et al., Clin Cancer Res 11: 5257-64 (2005); Okeley N et al., Clinc Cancer Res 16: 888-97 (2010); Oroudjev E et al., Mol Cancer Ther 9: 2700-13 (2010); Ellestad G, Chirality 23: 660-71 (2011); Kantarjian H et al., Lancet Oncol 13: 403-11 (2012); Moldenhauer G et al., J Natl Cancer Inst 104: 622-34 (2012); Meulendijks D et al., Invest New Drugs 34: 119-28 (2016)).

II. Linkages Connecting Components and/or their Subcomponents

Individual PD-L1 binding regions, Shiga toxin effector polypeptides, and/or components of the PD-L1 targeting molecules described herein may be suitably linked to each other via one or more linkers well known in the art and/or described herein. Individual polypeptide subcomponents of the binding regions, e.g. heavy chain variable regions (V_{H}), light chain variable regions (V_{L}), CDR, and/or ABR regions, may be suitably linked to each other via one or more linkers well known in the art and/or described herein. Proteinaceous components, e.g., multi-chain binding regions, may be suitably linked to each other or other polypeptide components via one or more linkers well known in the art. Peptide components, e.g., KDEL family endoplasmic reticulum retention/retrieval signal motifs, may be suitably linked to another component via one or more linkers, such as a proteinaceous linker, which are well known in the art.

Suitable linkers are generally those which allow each polypeptide component to fold with a three-dimensional structure very similar to the polypeptide components produced individually without any linker or other component. Suitable linkers include single amino acids, peptides, polypeptides, and linkers lacking any of the aforementioned, such as various non-proteinaceous carbon chains, whether branched or cyclic.

Suitable linkers may be proteinaceous and comprise one or more amino acids, peptides, and/or polypeptides. Proteinaceous linkers are suitable for both recombinant fusion proteins and chemically linked conjugates. A proteinaceous linker typically has from about 2 to about 50 amino acid residues, such as, e.g., from about 5 to about 30 or from about 6 to about 25 amino acid residues. The length of the linker selected will depend upon a variety of factors, such as, e.g., the desired property or properties for which the linker is being selected. In some embodiments, the linker is proteinaceous and is linked near the terminus of a protein component, typically within about 20 amino acids of the terminus. In some embodiments, a proteinaceous linker comprises a sequence of any one of SEQ ID NO: 70-76, 289, or 285.

Suitable linkers may be non-proteinaceous, such as, e.g. chemical linkers. Various non-proteinaceous linkers known in the art may be used to link cell-targeting binding regions to the Shiga toxin effector polypeptide components of the PD-L1 targeting molecules, such as linkers commonly used to conjugate immunoglobulin polypeptides to heterologous polypeptides. For example, polypeptide regions may be linked using the functional side chains of their amino acid residues and carbohydrate moieties such as, e.g., a carboxy, amine, sulfhydryl, carboxylic acid, carbonyl, hydroxyl, and/or cyclic ring group. For example, disulfide bonds and thioether bonds may be used to link two or more polypeptides. In addition, non-natural amino acid residues may be used with other functional side chains, such as ketone groups. Examples of non-proteinaceous chemical linkers include but are not limited to N-succinimidyl (4-iodoacetyl)-aminobenzoate, S—(N-succinimidyl) thioacetate (SATA), N-succinimidyl-oxycarbonyl-cu-methyl-α-(2-pyridyldithio) toluene (SMPT), N-succinimidyl 4-(2-pyridyldithio)-pentanoate (SPP), succinimidyl 4-(N-maleimidomethyl) cyclohexane carboxylate (SMCC or MCC), sulfosuccinimidyl (4-iodoacetyl)-aminobenzoate, 4-succinimidyl-oxycarbonyl-α-(2-pyridyldithio) toluene, sulfosuccinimidyl-6-(α-methyl-α-(pyridyldithiol)-toluamido) hexanoate, N-succinimidyl-3-(-2-pyridyldithio)-proprionate (SPDP), succinimidyl 6(3(-(-2-pyridyldithio)-proprionamido) hexanoate, sulfosuccinimidyl 6(3(-(-2-pyridyldithio)-propionamido) hexanoate, maleimidocaproyl (MC), maleimidocaproyl-valine-citrulline-p-aminobenzyloxycarbonyl (MC-vc-PAB), 3-maleimidobenzoic acid N-hydroxysuccinimide ester (MBS), alpha-alkyl derivatives, sulfoNHS-ATMBA (sulfosuccinimidyl N-[3-(acetylthio)-3-methylbutyryl-beta-alanine]), sulfodichlorophenol, 2-iminothiolane, 3-(2-pyridyldithio)-propionyl hydrazide, Ellman's reagent, dichlorotriazinic acid, and S-(2-thiopyridyl)-L-cysteine.

Suitable linkers, whether proteinaceous or non-proteinaceous, may include, e.g., protease sensitive, environmental redox potential sensitive, pH sensitive, acid cleavable, photocleavable, and/or heat sensitive linkers.

Proteinaceous linkers may be chosen for incorporation into recombinant fusion PD-L1 targeting molecules. For recombinant fusion cell-targeting proteins, linkers typically comprise about 2 to 50 amino acid residues, preferably about 5 to 30 amino acid residues. Commonly, proteinaceous linkers comprise a majority of amino acid residues with polar, uncharged, and/or charged residues, such as, e.g., threonine, proline, glutamine, glycine, and alanine. Non-limiting examples of proteinaceous linkers include alanine-serine-glycine-glycine-proline-glutamate (ASGGPE) (SEQ ID NO: 167), valine-methionine (VM), alanine-methionine (AM), AM(G_{2 to 4}S)ₓAM where G is glycine, S is serine, and x is an integer from 1 to 10 (SEQ ID NO: 168).

Proteinaceous linkers may be selected based upon the properties desired. Proteinaceous linkers may be chosen by the skilled worker with specific features in mind, such as to optimize one or more of the fusion molecule's folding, stability, expression, solubility, pharmacokinetic properties, pharmacodynamic properties, and/or the activity of the fused domains in the context of a fusion construct as compared to the activity of the same domain by itself. For example, proteinaceous linkers may be selected based on flexibility, rigidity, and/or cleavability. The skilled worker may use databases and linker design software tools when choosing linkers. In certain linkers may be chosen to optimize expression. In certain linkers may be chosen to promote intermolecular interactions between identical polypeptides or proteins to form homomultimers or different polypeptides or proteins to form heteromultimers. For example, proteinaceous linkers may be selected which allow for desired non-covalent interactions between polypeptide components of the PD-L1 targeting molecules, such as, e.g., interactions related to the formation dimers and other higher order multimers.

Flexible proteinaceous linkers are often greater than 12 amino acid residues long and rich in small, non-polar amino acid residues, polar amino acid residues, and/or hydrophilic amino acid residues, such as, e.g., glycines, serines, and threonines. Flexible proteinaceous linkers may be chosen to increase the spatial separation between components and/or to allow for intramolecular interactions between components. For example, various “GS” linkers are known to the skilled worker and are composed of multiple glycines and/or one or more serines, sometimes in repeating units, such as, e.g., (GₓS)ₙ (SEQ ID NO: 169), (SₓG)ₙ (SEQ ID NO: 170), (GGGGS)ₙ (SEQ ID NO: 171), and (G)ₙ (SEQ ID NO: 172), in which x is 1 to 6 and n is 1 to 30. Non-limiting examples of flexible proteinaceous linkers include GKSSGSGSESKS (SEQ ID NO: 173), EGKSSGSGSESKEF (SEQ ID NO: 174), GSTSGSGKSSEGKG (SEQ ID NO: 175), GSTSGSGKSSEGSGSTKG (SEQ ID NO: 176), GSTSGSGKPGSGEGSTKG (SEQ ID NO: 177), SRSSG (SEQ ID NO: 178), and SGSSC (SEQ ID NO: 179).

Rigid proteinaceous linkers are often stiff alpha-helical structures and rich in proline residues and/or one or more strategically placed prolines. Rigid linkers may be chosen to prevent intramolecular interactions between linked components.

Suitable linkers may be chosen to allow for in vivo separation of components, such as, e.g., due to cleavage and/or environment-specific instability. In vivo cleavable proteinaceous linkers are capable of unlinking by proteolytic processing and/or reducing environments often at a specific site within an organism or inside a certain cell type. In vivo cleavable proteinaceous linkers often comprise protease sensitive motifs and/or disulfide bonds formed by one or more cysteine pairs. In vivo cleavable proteinaceous linkers may be designed to be sensitive to proteases that exist only at certain locations in an organism, compartments within a cell, and/or become active only under certain physiological or pathological conditions (such as, e.g., involving proteases with abnormally high levels, proteases overexpressed at certain disease sites, and proteases specifically expressed by a pathogenic microorganism). For example, there are proteinaceous linkers known in the art which are cleaved by proteases present only intracellularly, proteases present only within specific cell types, and proteases present only under pathological conditions like cancer or inflammation, such as, e.g., R-x-x-R (SEQ ID NO: 160) motif and AMGRSGGGCAGNRVGSSLSCGGLNLQAM (SEQ ID NO: 165). In some embodiments, the linker comprises the sequence HHAA (SEQ ID NO: 187). In some embodiments, the linker comprises the sequence HHHHHHAA (SEQ ID NO: 260).

In some embodiments of the PD-L1 targeting molecules, a linker may be used which comprises one or more protease sensitive sites to provide for cleavage by a protease present within a target cell. In some embodiments of the PD-L1 targeting molecules, a linker may be used which is not cleavable to reduce unwanted toxicity after administration to a vertebrate organism.

Suitable linkers may include, e.g., protease sensitive, environmental redox potential sensitive, pH sensitive, acid cleavable, photocleavable, and/or heat sensitive linkers, whether proteinaceous or non-proteinaceous (see e.g., Doronina S et al., Bioconjug Chem 17: 114-24 (2003); Saito G et al., Adv Drug Deliv Rev 55: 199-215 (2003); Jeffrey S et al., J Med Chem 48: 1344-58 (2005); Sanderson R et al., Clin Cancer Res 11: 843-52 (2005); Erickson H et al., Cancer Res 66: 4426-33 (2006); Chen X et al., Adv Drug Deliv Rev 65: 1357-69 (2013)). Suitable cleavable linkers may include linkers comprising cleavable groups which are known in the art.

Suitable linkers may include pH sensitive linkers. For example, certain suitable linkers may be chosen for their instability in lower pH environments to provide for dissociation inside a subcellular compartment of a target cell (see e.g., van Der Velden V et al., Blood 97: 3197-204 (2001); Ulbrich K, Subr V, Adv Drug Deliv Rev 56: 1023-50 (2004)). For example, linkers that comprise one or more trityl groups, derivatized trityl groups, bismaleimideothoxy propane groups, adipic acid dihydrazide groups, and/or acid labile transferrin groups, may provide for release of components of the PD-L1 targeting molecules, e.g. a polypeptide component, in environments with specific pH ranges. In certain linkers may be chosen which are cleaved in pH ranges corresponding to physiological pH differences between tissues, such as, e.g., the pH of tumor tissue is lower than in healthy tissues.

Photocleavable linkers are linkers that are cleaved upon exposure to electromagnetic radiation of certain wavelength ranges, such as light in the visible range. Photocleavable linkers may be used to release a component of a PD-L1 targeting molecule, e.g. a polypeptide component, upon exposure to light of certain wavelengths. Non-limiting examples of photocleavable linkers include a nitrobenzyl group as a photocleavable protective group for cysteine, nitrobenzyloxycarbonyl chloride cross-linkers, hydroxypropylmethacrylamide copolymer, glycine copolymer, fluorescein copolymer, and methylrhodamine copolymer. Photocleavable linkers may have particular uses in linking components to form PD-L1 targeting molecules designed for treating diseases, disorders, and conditions that can be exposed to light using fiber optics.

In some embodiments of the PD-L1 targeting molecules, a PD-L1 binding region is linked to a Shiga toxin effector polypeptide using any number of means known to the skilled worker, including both covalent and noncovalent linkages.

In some embodiments of the PD-L1 targeting molecules, the molecule comprises a binding region which is a scFv with a linker connecting a heavy chain variable (V_{H}) domain and a light chain variable (V_{L}) domain. There are numerous linkers known in the art suitable for this purpose, such as, e.g., the 15-residue (Gly₄Ser)₃ peptide (SEQ ID NO: 180). Suitable scFv linkers which may be used in forming non-covalent multivalent structures include GGS, GGGS (SEQ ID NO: 181), GGGGS (SEQ ID NO: 182), GGGGSGGG (SEQ ID NO: 183), GGSGGGG (SEQ ID NO: 184), GSTSGGGSGGGSGGGGSS (SEQ ID NO: 185), and GSTSGSGKPGSSEGSTKG (SEQ ID NO: 186).

Suitable methods for linkage of the components of the PD-L1 targeting molecules may be by any method presently known in the art for accomplishing such, so long as the attachment does not substantially impede the binding capability of the cell-targeting binding region, the cellular internalization of the Shiga toxin effector polypeptide component, and/or when appropriate the desired Shiga toxin effector function(s) as measured by an appropriate assay, including assays described herein.

The components of the PD-L1 targeting molecule, e.g. a Shiga toxin A Subunit effector polypeptide and/or immunoglobulin-type PD-L1-binding region, may be engineered to provide a suitable attachment moiety for the linkage of additional components, e.g. an additional exogenous material (see WO 2018/106895).

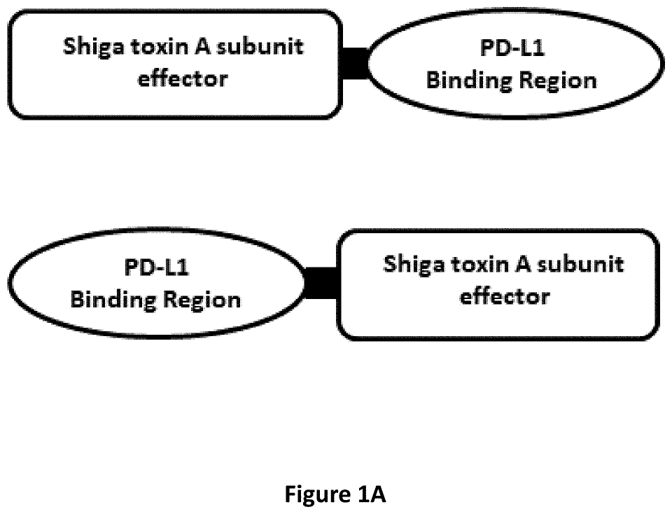

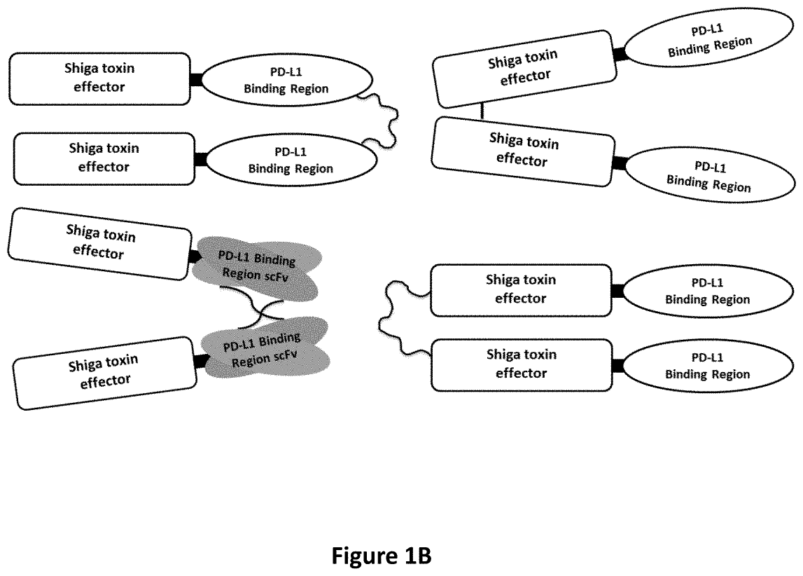

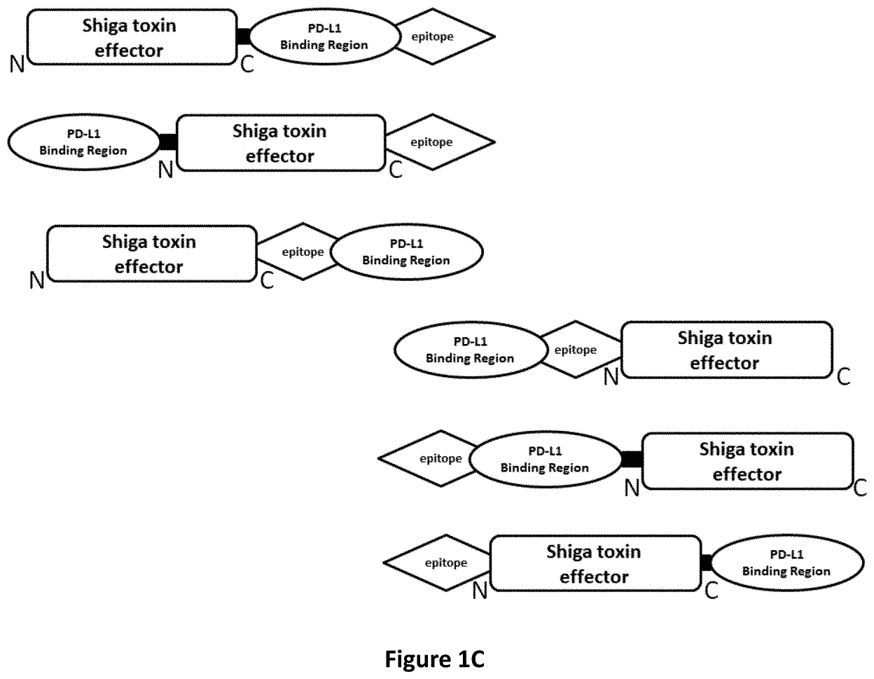

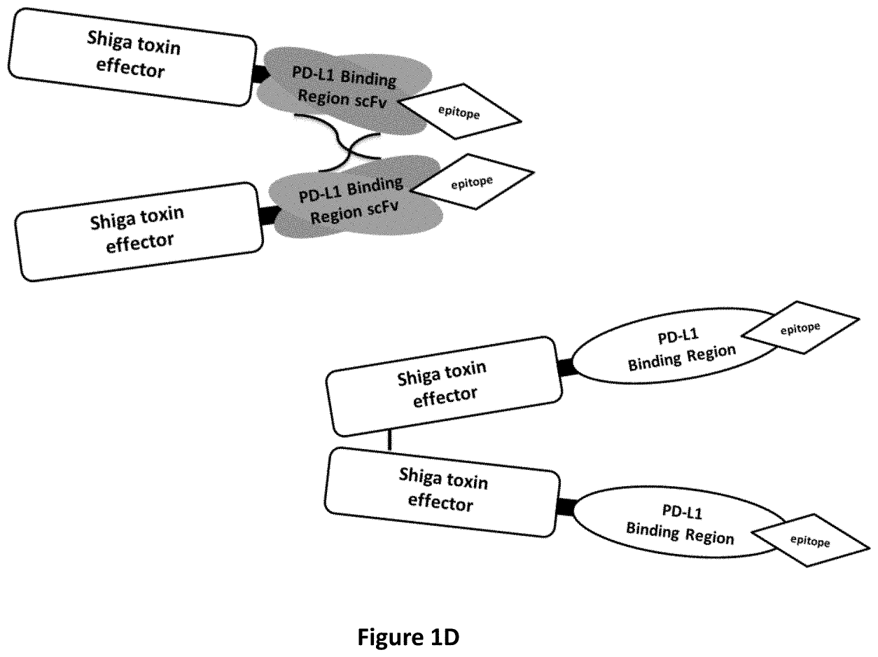

For the purposes of the PD-L1 targeting molecules, the specific order or orientation is not fixed for the components: the Shiga toxin effector polypeptide(s), the binding region(s), and any optional linker(s), in relation to each other or the entire PD-L1 targeting molecule (see e.g. FIG. 1) unless specifically noted. The components of the PD-L1 targeting molecules may be arranged in any order provided that the desired activity(ies) of the binding region and Shiga toxin effector polypeptide are not eliminated.

### III. Examples of Structural Variations of the PD-L1 Targeting Molecules

In some embodiments, a Shiga toxin effector polypeptide of the PD-L1 targeting molecule comprises or consists essentially of a truncated Shiga toxin A Subunit. Truncations of Shiga toxin A Subunits might result in the deletion of an entire epitope(s) and/or epitope region(s), B-cell epitopes, CD4+ T-cell epitopes, and/or furin-cleavage sites without affecting Shiga toxin effector functions, such as, e.g., catalytic activity and cytotoxicity. The smallest Shiga toxin A Subunit fragment shown to exhibit full enzymatic activity was a polypeptide composed of residues 1-239 of Slt1A (LaPointe P et al., J Biol Chem 280: 23310-18 (2005)). The smallest Shiga toxin A Subunit fragment shown to exhibit significant enzymatic activity was a polypeptide composed of residues 75-247 of StxA (Al-Jaufy A et al., Infect Immun 62: 956-60 (1994)).

Although Shiga toxin effector polypeptides may commonly be smaller than the full-length Shiga toxin A Subunit, it is preferred that the Shiga toxin effector polypeptide region of a PD-L1 targeting molecule maintain the polypeptide region from amino acid position 77 to 239 (SLT-1A (SEQ ID NO:1) or StxA (SEQ ID NO:2)) or the equivalent in other A Subunits of members of the Shiga toxin family (e.g. 77 to 238 of (SEQ ID NO:3)). For example, in some embodiments of the molecules, the Shiga toxin effector polypeptide derived from SLT-1A may comprise or consist essentially of amino acids 75 to 251 of SEQ ID NO:1, 1 to 241 of SEQ ID NO:1, 1 to 251 of SEQ ID NO:1, or amino acids 1 to 261 of SEQ ID NO:1, wherein relative to a wild-type Shiga toxin A Subunit at least one amino acid residue is mutated or has been deleted in an endogenous epitope and/or epitope region, and/or wherein there is a disrupted, furin-cleavage site at the carboxy-terminus of a Shiga toxin A1 fragment derived region. Similarly, Shiga toxin effector polypeptide regions derived from StxA may comprise or consist essentially of amino acids 75 to 251 of SEQ ID NO:2, 1 to 241 of SEQ ID NO:2, 1 to 251 of SEQ ID NO:2, or amino acids 1 to 261 of SEQ ID NO:2, wherein relative to a wild-type Shiga toxin A Subunit at least one amino acid residue is mutated or has been deleted in an endogenous epitope and/or epitope region, and/or wherein there is a disrupted, furin-cleavage site at the carboxy-terminus of a Shiga toxin A1 fragment derived region. Additionally, Shiga toxin effector polypeptide regions derived from SLT-2 may comprise or consist essentially of amino acids 75 to 251 of SEQ ID NO:3, 1 to 241 of SEQ ID NO:3, 1 to 251 of SEQ ID NO:3, or amino acids 1 to 261 of SEQ ID NO:3, wherein relative to a wild-type Shiga toxin A Subunit at least one amino acid residue is mutated or has been deleted in an endogenous epitope and/or epitope region, and/or wherein there is a disrupted, furin-cleavage site at the carboxy-terminus of a Shiga toxin A1 fragment derived region.

Also provided herein are variants of Shiga toxin effector polypeptides and PD-L1 targeting molecules, wherein the Shiga toxin effector polypeptide differs from a naturally occurring Shiga toxin A Subunit by only or up to 1, 2, 3, 4, 5, 6, 7, 8, 9, 10, 15, 20, 25, 30, 35, 40 or more amino acid residues (but by no more than that which retains at least 85%, 90%, 95%, 99% or more amino acid sequence identity). Thus, a molecule derived from an A Subunit of a member of the Shiga toxin family may comprise additions, deletions, truncations, or other alterations from the original sequence as long as at least 85%, 90%, 95%, 99% or more amino acid sequence identity is maintained to a naturally occurring Shiga toxin A Subunit and wherein relative to a wild-type Shiga toxin A Subunit at least one amino acid residue is mutated or has been deleted in an endogenous epitope and/or epitope region, and/or wherein there is a disrupted, furin-cleavage site at the carboxy-terminus of a Shiga toxin A1 fragment derived region.

Accordingly, in some embodiments, the Shiga toxin effector polypeptide of a molecule comprises or consists essentially of amino acid sequences having at least 55%, 60%, 65%, 70%, 75%, 80%, 85%, 90%, 95%, 97%, 98%, 99%, 99.5% or 99.7% overall sequence identity to a naturally occurring Shiga toxin A Subunit, such as SLT-1A (SEQ ID NO:1), StxA (SEQ ID NO:2), and/or SLT-2A (SEQ ID NO:3) wherein relative to a wild-type Shiga toxin A Subunit at least one amino acid residue is mutated or has been deleted in an endogenous epitope and/or epitope region, and/or wherein there is a disrupted, furin-cleavage site at the carboxy-terminus of a Shiga toxin A1 fragment derived region.

Optionally, either a full-length or a truncated version of the Shiga toxin A Subunit may comprise the Shiga toxin effector polypeptide region of a molecule of the present, wherein the Shiga toxin derived polypeptide comprises one or more mutations (e.g. substitutions, deletions, insertions, or inversions) as compared to a naturally occurring Shiga toxin. In some embodiments, the Shiga toxin effector polypeptides have sufficient sequence identity to a naturally occurring Shiga toxin A Subunit to retain cytotoxicity after entry into a cell, either by well-known methods of host cell transformation, transfection, infection or induction, or by internalization mediated by a cell-targeting binding region linked with the Shiga toxin effector polypeptide. The most critical residues for enzymatic activity and/or cytotoxicity in the Shiga toxin A Subunits have been mapped to the following residue-positions: asparagine-75, tyrosine-77, glutamate-167, arginine-170, and arginine-176 among others (Di R et al., Toxicon 57: 525-39 (2011)). In any one of the embodiments, the Shiga toxin effector polypeptides may preferably but not necessarily maintain one or more conserved amino acids at positions, such as those found at positions 77, 167, 170, and 176 in StxA, SLT-1A, or the equivalent conserved position in other members of the Shiga toxin family which are typically required for cytotoxic activity. The capacity of a cytotoxic molecule to cause cell death, e.g. its cytotoxicity, may be measured using any one or more of a number of assays well known in the art.

### A. Examples of De-Immunized, Shiga Toxin Effector Polypeptides

In some embodiments, the de-immunized, Shiga toxin effector polypeptide of the PD-L1 targeting molecule may consist essentially of a truncated Shiga toxin A Subunit having two or more mutations. Truncations of Shiga toxin A Subunits might result in the deletion of an entire epitope(s) and/or epitope region(s), B-cell epitopes, CD4+ T-cell epitopes, and/or furin-cleavage sites without affecting Shiga toxin effector functions, such as, e.g., catalytic activity and cytotoxicity. Truncating the carboxy-terminus of SLT-1A, StxA, or SLT-2A to amino acids 1-251 removes two predicted B-cell epitope regions, two predicted CD4 positive (CD4+) T-cell epitopes, and a predicted discontinuous B-cell epitope. Truncating the amino-terminus of SLT-1A, StxA, or SLT-2A to 75-293 removes at least three predicted B-cell epitope regions and three predicted CD4+ T-cell epitopes. Truncating both amino- and carboxy-terminals of SLT-1A, StxA, or SLT-2A to 75-251 deletes at least five predicted B-cell epitope regions, four putative CD4+ T-cell epitopes and one predicted discontinuous B-cell epitope.

In some embodiments, a de-immunized, Shiga toxin effector polypeptide may comprise or consist essentially of a full-length or truncated Shiga toxin A Subunit with at least one mutation (relative to a wild-type Shiga toxin polypeptide), e.g. deletion, insertion, inversion, or substitution, in a provided, endogenous, B-cell and/or CD4+ T-cell epitope region. In some embodiments, the Shiga toxin effector polypeptide comprises a disruption which comprises a mutation (relative to a wild-type Shiga toxin polypeptide) which includes a deletion of at least one amino acid residue within the endogenous, B-cell and/or CD4+ T-cell epitope region. In some embodiments, the Shiga toxin effector polypeptide comprises a disruption which comprises an insertion of at least one amino acid residue within the endogenous, B-cell and/or CD4+ T-cell epitope region. In some embodiments, the Shiga toxin effector polypeptide comprises a disruption which comprises an inversion of amino acid residues, wherein at least one inverted amino acid residue is within the endogenous, B-cell and/or CD4+ T-cell epitope region. In some embodiments, the Shiga toxin effector polypeptide comprises a disruption which comprises a mutation (relative to a wild-type Shiga toxin polypeptide), such as, e.g., an amino acid substitution, an amino acid substitution to a non-standard amino acid, and/or an amino acid residue with a chemically modified side chain. Non-limiting examples of de-immunized, Shiga toxin effector sub-regions are described in WO 2015/113005, WO 2015/113007, WO 2015/191764, WO 2016/196344, and WO 2018/140427.

In other embodiments, the de-immunized, Shiga toxin effector polypeptide comprises a truncated Shiga toxin A Subunit which is shorter than a full-length Shiga toxin A Subunit wherein at least one amino acid residue is disrupted in a natively positioned, B-cell and/or CD4+ T-cell epitope region.

To create a de-immunized, Shiga toxin effector polypeptide, in principle modifying any amino acid residue in a provided epitope region by various means can result in a disruption of an epitope, such as, e.g., a modification which represents a deletion, insertion, inversion, rearrangement, substitution, and chemical modification of a side chain relative to a wild-type Shiga toxin polypeptide. However, modifying certain amino acid residues and using certain amino acid modifications are more likely to successfully reduce antigenicity and/or immunogenicity while maintaining a certain level of a Shiga toxin effector function(s). For example, terminal truncations and internal amino acid substitutions are preferred because these types of modifications maintain the overall spacing of the amino acid residues in a Shiga toxin effector polypeptide and thus are more likely to maintain Shiga toxin effector polypeptide structure and function.

In some embodiments, the de-immunized, Shiga toxin effector polypeptide comprises or consists essentially of amino acids 75 to 251 of SLT-1A (SEQ ID NO:1), StxA (SEQ ID NO:2), and/or SLT-2A (SEQ ID NO:3) wherein at least one amino acid residue is disrupted in a natively positioned, epitope region. In some embodiments, a de-immunized, Shiga toxin effector polypeptide comprises or consists essentially of amino acids 1 to 241 of SLT-1A (SEQ ID NO:1), StxA (SEQ ID NO:2), and/or SLT-2A (SEQ ID NO:3) wherein at least one amino acid residue is disrupted in a natively positioned, epitope region. Some embodiments are de-immunized, Shiga toxin effector polypeptides which comprise or consist essentially of amino acids 1 to 251 of SLT-1A (SEQ ID NO:1), StxA (SEQ ID NO:2), and/or SLT-2A (SEQ ID NO:3) wherein at least one amino acid residue is disrupted in a natively positioned, epitope region provided. Some embodiments are Shiga toxin effector polypeptides comprising amino acids 1 to 261 of SLT-1A (SEQ ID NO:1), StxA (SEQ ID NO:2), and/or SLT-2A (SEQ ID NO:3) wherein at least one amino acid residue is disrupted in a natively positioned, epitope region.

There are numerous, diverse, internal amino acid substitutions that can be used to create de-immunized, Shiga toxin effector polypeptides. Of the possible substitute amino acids to use within an epitope region, the following substitute amino acid residues are predicted to be the most likely to reduce the antigenicity and/or immunogenicity of an epitope—G, D, E, S, T, R, K, and H. Except for glycine, these amino acid residues may all be classified as polar and/or charged residues. Of the possible amino acids to substitute with, the following amino acids A, G, V, L, I, P, C, M, F, S, D, N, Q, H, and K are predicted to be the most likely to reduce antigenicity and/or immunogenicity while providing the retention of a significant level of a Shiga toxin effector function(s), depending on the amino acid substituted for. Generally, the substitution should change a polar and/or charged amino acid residue to a non-polar and uncharged residue (see e.g. WO 2015/113007). In addition, it may be beneficial to epitope disruption to reduce the overall size and/or length of the amino acid residue's R-group functional side chain (see e.g. WO 2015/113007). However despite these generalities of substitutions most likely to confer epitope disruption, because the aim is to preserve significant Shiga toxin effector function(s), the substitute amino acid might be more likely to preserve Shiga toxin effector function(s) if it resembles the amino acid substituted for, such as, e.g., a nonpolar and/or uncharged residue of similar size substituted for a polar and/or charged residue.

In WO 2015/113007, many mutations were empirically tested for effect(s) on the Shiga toxin effector function of various Shiga toxin effector polypeptides and PD-L1 targeting molecules. Table 2 summarizes the results described in WO 2015/113007 and WO 2016/196344 where an amino acid substitution, alone or in combination with one or more other substitutions, did not prevent the exhibition of a potent level of a Shiga toxin effector function(s). Table 2 uses the epitope region numbering scheme described in WO 2016/196344.

TABLE 2
Amino Acid Substitutions in Shiga Toxin Effector Polypeptides

| | natively positioned amino acid positions | | |
|---|---|---|---|
| Epitope Region | | B-Cell Epitope | T-Cell |
| Disrupted | Substitution | Region | Epitope |
| 1 | K1A | 1-15 | |
| 1 | K1M | 1-15 | |
| 1 | T4I | 1-15 | 4-33 |
| 1 | D6R | 1-15 | 4-33 |
| 1 | S8I | 1-15 | 4-33 |
| 1 | T9V | 1-15 | 4-33 |
| 1 | T9I | 1-15 | 4-33 |
| 1 | K11A | 1-15 | 4-33 |
| 1 | K11H | 1-15 | 4-33 |
| 1 | T12K | 1-15 | 4-33 |
| 2 | S33I | 27-37 | 4-33 |
| 2 | S33C | 27-37 | 4-33 |
| 3 | S43N | 39-48 | 34-78 |
| 3 | G44L | 39-48 | 34-78 |
| 3 | T45V | 39-48 | 34-78 |
| 3 | T45I | 39-48 | 34-78 |
| 3 | S45V | 39-48 | 34-78 |
| 3 | S45I | 39-48 | 34-78 |
| 3 | G46P | 39-48 | 34-78 |
| 3 | D47G | 39-48 | 34-78 |
| 3 | D47M | 39-48 | 34-78 |
| 3 | N48V | 39-48 | 34-78 |
| 3 | N48F | 39-48 | 34-78 |
| — | L49A | immunogenic residue | 34-78 |
| — | F50T | | 34-78 |
| — | A51V | | 34-78 |
| 4 | D53A | 53-66 | 34-78 |
| 4 | D53G | 53-66 | 34-78 |
| 4 | D53N | 53-66 | 34-78 |
| 4 | V54L | 53-66 | 34-78 |
| 4 | V54I | 53-66 | 34-78 |
| 4 | R55A | 53-66 | 34-78 |
| 4 | R55V | 53-66 | 34-78 |
| 4 | R55L | 53-66 | 34-78 |
| 4 | G56P | 53-66 | 34-78 |
| 4 | I57M | 53-66 | 34-78 |
| 4 | I57F | 53-66 | 34-78 |
| 4 | D58A | 53-66 | 34-78 |
| 4 | D58V | 53-66 | 34-78 |
| 4 | D58F | 53-66 | 34-78 |
| 4 | P59A | 53-66 | 34-78 |
| 4 | P59F | 53-66 | 34-78 |
| 4 | E60I | 53-66 | 34-78 |
| 4 | E60T | 53-66 | 34-78 |
| 4 | E60R | 53-66 | 34-78 |
| 4 | E61A | 53-66 | 34-78 |
| 4 | E61V | 53-66 | 34-78 |
| 4 | E61L | 53-66 | 34-78 |
| 4 | G62A | 53-66 | 34-78 |
| — | R84A | | 77-103 |
| — | V88A | | 77-103 |
| 5 | D94A | 94-115 | 77-103 |
| 5 | S96I | 94-115 | 77-103 |
| 5 | T104N | 94-115 | |
| 5 | A105L | 94-115 | |
| 5 | T107P | 94-115 | |
| 5 | L108M | 94-115 | |
| 5 | S109V | 94-115 | |
| 5 | G110A | 94-115 | |
| 5 | D111T | 94-115 | |
| 5 | S112V | 94-115 | |
| 6 | D141A | 141-153 | 128-168 |
| 6 | G147A | 141-153 | 128-168 |
| — | V154A | | 128-168 |
| 7 | R179A | 179-190 | 160-183 |
| 7 | T180G | 179-190 | 160-183 |
| 7 | T181I | 179-190 | 160-183 |
| 7 | D183A | 179-190 | 160-183 |
| 7 | D183G | 179-190 | 160-183 |
| 7 | D184A | 179-190 | |
| 7 | D184F | 179-190 | |
| 7 | L185V | 179-190 | |
| 7 | S186A | 179-190 | |
| 7 | S186F | 179-190 | |
| 7 | G187A | 179-190 | |
| 7 | G187T | 179-190 | |
| 7 | R188A | 179-190 | |
| 7 | R188L | 179-190 | |
| 7 | S189A | 179-190 | |
| — | D198A | immunogenic residue | |
| — | R205A | immunogenic residue | |
| — | C242S | | 236-258 |
| 8 | R248A | 243-257 | 236-258 |
| 8 | R251A | 243-257 | 236-258 |

Based on the empirical evidence in WO 2015/113007 and WO 2016/196344, certain amino acid positions in the A Subunits of Shiga toxins are predicted to tolerate epitope disruptions while still retaining significant Shiga toxin effector functions. For example, the following natively occurring positions tolerate amino acid substitutions, either alone or in combination, while retaining a Shiga toxin effector function(s) such as cytotoxicity—1 of SEQ ID NO:1 or SEQ ID NO:2; 4 of SEQ ID NO:1, SEQ ID NO:2, or SEQ ID NO:3; 8 of SEQ ID NO:1, SEQ ID NO:2, or SEQ ID NO:3; 9 of SEQ ID NO:1, SEQ ID NO:2, or SEQ ID NO:3; 11 of SEQ ID NO:1, SEQ ID NO:2, or SEQ ID NO:3; 33 of SEQ ID NO:1 or SEQ ID NO:2; 43 of SEQ ID NO:1 or SEQ ID NO:2; 44 of SEQ ID NO:1 or SEQ ID NO:2; 45 of SEQ ID NO:1 or SEQ ID NO:2; 46 of SEQ ID NO:1 or SEQ ID NO:2; 47 of SEQ ID NO:1 or SEQ ID NO:2; 48 of SEQ ID NO:1, SEQ ID NO:2, or SEQ ID NO:3; 49 of SEQ ID NO:1 or SEQ ID NO:2; 50 of SEQ ID NO:1 or SEQ ID NO:2; 51 of SEQ ID NO:1 or SEQ ID NO:2; 53 of SEQ ID NO:1 or SEQ ID NO:2; 54 of SEQ ID NO:1 or SEQ ID NO:2; 55 of SEQ ID NO:1 or SEQ ID NO:2; 56 of SEQ ID NO:1 or SEQ ID NO:2; 57 of SEQ ID NO:1 or SEQ ID NO:2; 58 of SEQ ID NO:1, SEQ ID NO:2, or SEQ ID NO:3; 59 of SEQ ID NO:1, SEQ ID NO:2, or SEQ ID NO:3; 60 of SEQ ID NO:1 or SEQ ID NO:2; 61 of SEQ ID NO:1 or SEQ ID NO:2; 62 of SEQ ID NO:1 or SEQ ID NO:2; 84 of SEQ ID NO:1 or SEQ ID NO:2; 88 of SEQ ID NO:1 or SEQ ID NO:2; 94 of SEQ ID NO:1, SEQ ID NO:2, or SEQ ID NO:3; 96 of SEQ ID NO:1, SEQ ID NO:2, or SEQ ID NO:3; 104 of SEQ ID NO:1 or SEQ ID NO:2; 105 of SEQ ID NO:1 or SEQ ID NO:2; 107 of SEQ ID NO:1 or SEQ ID NO:2; 108 of SEQ ID NO:1 or SEQ ID NO:2; 109 of SEQ ID NO:1, SEQ ID NO:2, or SEQ ID NO:3; 110 of SEQ ID NO:1 or SEQ ID NO:2; 111 of SEQ ID NO:1 or SEQ ID NO:2; 112 of SEQ ID NO:1, SEQ ID NO:2, or SEQ ID NO:3; 141 of SEQ ID NO:1 or SEQ ID NO:2; 147 of SEQ ID NO:1, SEQ ID NO:2, or SEQ ID NO:3; 154 of SEQ ID NO:1 or SEQ ID NO:2; 179 of SEQ ID NO:1, SEQ ID NO:2, or SEQ ID NO:3; 180 of SEQ ID NO:1 or SEQ ID NO:2; 181 of SEQ ID NO:1 or SEQ ID NO:2; 183 of SEQ ID NO:1, SEQ ID NO:2, or SEQ ID NO:3; 184 of SEQ ID NO:1, SEQ ID NO:2, or SEQ ID NO:3; 185 of SEQ ID NO:1 or SEQ ID NO:2; 186 of SEQ ID NO:1, SEQ ID NO:2, or SEQ ID NO:3; 187 of SEQ ID NO:1 or SEQ ID NO:2; 188 of SEQ ID NO:1 or SEQ ID NO:2; 189 of SEQ ID NO:1 or SEQ ID NO:2; 198 of SEQ ID NO:1 or SEQ ID NO:2; 204 of SEQ ID NO:3; 205 of SEQ ID NO:1 or SEQ ID NO:2; 241 of SEQ ID NO:3; 242 of SEQ ID NO:1 or SEQ ID NO:2; 247 of SEQ ID NO:1 or SEQ ID NO:2; 247 of SEQ ID NO:3; 248 of SEQ ID NO:1 or SEQ ID NO:2; 250 of SEQ ID NO:3; 251 of SEQ ID NO:1 or SEQ ID NO:2; 264 of SEQ ID NO:1, SEQ ID NO:2, or SEQ ID NO:3; 265 of SEQ ID NO:1 or SEQ ID NO:2; and 286 of SEQ ID NO:1 or SEQ ID NO:2.

The empirical data in WO 2015/113007 and WO 2016/196344 point towards other epitope disrupting substitutions and combinations of epitope disrupting substitutions that can reduce antigenicity and/or immunogenicity of a Shiga toxin effector polypeptide while retaining the ability of the Shiga toxin effector polypeptide to exhibit a significant Shiga toxin effector function such as, e.g., new combinations of the aforementioned truncations and positions tolerating substitutions as well as new substitutions at identical positions or conserved positions in related Shiga toxin A Subunits.

It is predictable that other amino acid substitutions to amino acid residues of a conservative functional group of a substitution tested herein may reduce antigenicity and/or immunogenicity while preserving a significant Shiga toxin effector function. For example, other substitutions known to the skilled worker to be similar to any of K1A, K1M, T4I, D6R, S8I, T8V, T9I, S9I, K11A, K11H, T12K, S33I, S33C, S43N, G44L, S45V, S45I, T45V, T45I, G46P, D47M, D47G, N48V, N48F, L49A, F50T, A51V, D53A, D53N, D53G, V54L, V54I, R55A, R55V, R55L, G56P, I57F, I57M, D58A, D58V, D58F, P59A, P59F, E60I, E60T, E60R, E61A, E61V, E61L, G62A, R84A, V88A, D94A, S96I, T104N, A105L, T107P, L108M, S109V, T109V, G110A, D111T, S112V, D141A, G147A, V154A, R179A, T180G, T181I, D183A, D183G, D184A, D184A, D184F, L185V, L185D, S186A, S186F, G187A, G187T, R188A, R188L, S189A, D198A, R204A, R205A, C242S, S247I, Y247A, R248A, R250A, R251A, or D264A, G264A, T286A, and/or T286I may disrupt an endogenous epitope while maintaining at least one Shiga toxin effector function. In particular, amino acid substitutions to conservative amino acid residues similar to K1A, T4I, S8I, T8V, T9I, S9I, K11A, K11H, S33I, S33C, S43N, G44L, S45V, S45I, T45V, T45I, G46P, D47M, N48V, N48F, L49A, A51V, D53A, D53N, V54L, V54I, R55A, R55V, R55L, G56P, I57F, I57M, D58A, D58V, D58F, P59A, E60I, E60T, E61A, E61V, E61L, G62A, R84A, V88A, D94A, S96I, T104N, T107P, L108M, S109V, T109V, G110A, D111T, S112V, D141A, G147A, V154A, R179A, T180G, T181I, D183A, D183G, D184A, D184F, L185V, S186A, S186F, G187A, R188A, R188L, S189A, D198A, R204A, R205A, C242S, S247I, Y247A, R248A, R250A, R251A, D264A, G264A, T286A, and T286I may have the same or similar effects. In some embodiments, a Shiga toxin effector polypeptide may comprise similar conservative amino acid substitutions to empirically tested ones, such as, e.g., K1 to G, V, L, I, F, and H; T4 to A, G, V, L, F, M, and S; S8 to A, G, V, L, F, and M; T9 to A, G, L, F, M, and S; S9 to A, G, L, I, F, and M; K11 to G, V, L, I, F, and M; S33 to A, G, V, L, F, and M; S43 to A, G, V, L, I, F, and M; S45 to A, G, L, F, and M; T45 to A, G, L, F, and M; D47 to A, V, L, I, F, S, and Q; N48 to A, G, L, and M; L49 to G; Y49 to A; D53 to V, L, I, F, S, and Q; R55 to G, I, F, M, Q, S, K, and H; D58 to G, L, I, S, and Q; P59 to G; E60 to A, G, V, L, F, S, Q, N, D, and M; E61 to G, I, F, S, Q, N, D, M, and R; R84 to G, V, L, I, F, M, Q, S, K, and H; V88 to G; I88 to G; D94 to G, V, L, I, F, S, and Q; S96 to A, G, V, L, F, and M; T107 to A, G, V, L, I, F, M, and S; S107 to A, G, V, L, I, F, and M; S109 to A, G, I, L, F, and M; T109 to A, G, I, L, F, M, and S; S112 to A, G, L, I, F, and M; D141 to V, L, I, F, S, and Q; V154 to G; R179 to G, V, L, I, F, M, Q, S, K, and H; T180 to A, V, L, I, F, M, and S; T181 to A, G, V, L, F, M, and S; D183 to V, L, I, F, S, and Q; D184 to G, V, L, I, S, and Q; S186 to G, V, I, L, and M; R188 to G, V, I, F, M, Q, S, K, and H; S189 to G, V, I, L, F, and M; D197 to V, L, I, F, S, and Q; D198 to A, V, L, I, F, S, and Q; R204 to G, V, L, I, F, M, Q, S, K, and H; R205 to G, V, L, I, F, M, Q, S, K and H; S247 to A, G, V, I, L, F, and M; Y247 to A, G, V, L, I, F, and M; R248 to G, V, L, I, F, M, Q, S, K, and H; R250 to G, V, L, I, F, M, Q, S, K, and H; R251 to G, V, L, I, F, M, Q, S, K, and H; D264 to A, G, V, L, I, F, S, and Q; and T286 to A, G, V, L, I, F, M, and S.

Similarly, amino acid substitutions which remove charge, polarity, and/or reduce side chain length can disrupt an epitope while maintaining at least one Shiga toxin effector function. In some embodiments, a Shiga toxin effector polypeptide may comprise one or more epitopes disrupted by substitutions such that side chain charge is removed, polarity is removed, and/or side chain length is reduced such as, e.g., substituting the appropriate amino acid selected from the following group A, G, V, L, I, P, C, M, F, S, D, N, Q, H, or K for the amino acid residue at position 1 of SEQ ID NO:1 or SEQ ID NO:2; 4 of SEQ ID NO:1, SEQ ID NO:2, or SEQ ID NO:3; 6 of SEQ ID NO:1 or SEQ ID NO:2; 8 of SEQ ID NO:1, SEQ ID NO:2, or SEQ ID NO:3; 9 of SEQ ID NO:1, SEQ ID NO:2, or SEQ ID NO:3; 11 of SEQ ID NO:1, SEQ ID NO:2, or SEQ ID NO:3; 12 of SEQ ID NO:1, SEQ ID NO:2, or SEQ ID NO:3; 33 of SEQ ID NO:1 or SEQ ID NO:2; 43 of SEQ ID NO:1 or SEQ ID NO:2; 44 of SEQ ID NO:1 or SEQ ID NO:2; 45 of SEQ ID NO:1 or SEQ ID NO:2; 46 of SEQ ID NO:1 or SEQ ID NO:2; 47 of SEQ ID NO:1 or SEQ ID NO:2; 48 of SEQ ID NO:1, SEQ ID NO:2, or SEQ ID NO:3; 49 of SEQ ID NO:1 or SEQ ID NO:2; 50 of SEQ ID NO:1 or SEQ ID NO:2; 51 of SEQ ID NO:1 or SEQ ID NO:2; 53 of SEQ ID NO:1 or SEQ ID NO:2; 54 of SEQ ID NO:1 or SEQ ID NO:2; 55 of SEQ ID NO:1 or SEQ ID NO:2; 56 of SEQ ID NO:1 or SEQ ID NO:2; 57 of SEQ ID NO:1, SEQ ID NO:2, or SEQ ID NO:3; 58 of SEQ ID NO:1, SEQ ID NO:2, or SEQ ID NO:3; 59 of SEQ ID NO:1, SEQ ID NO:2, or SEQ ID NO:3; 60 of SEQ ID NO:1 or SEQ ID NO:2; 61 of SEQ ID NO:1 or SEQ ID NO:2; 62 of SEQ ID NO:1 or SEQ ID NO:2; 84 of SEQ ID NO:1 or SEQ ID NO:2; 88 of SEQ ID NO:1, SEQ ID NO:2, or SEQ ID NO:3; 94 of SEQ ID NO:1, SEQ ID NO:2, or SEQ ID NO:3; 96 of SEQ ID NO:1, SEQ ID NO:2, or SEQ ID NO:3; 104 of SEQ ID NO:1 or SEQ ID NO:2; 105 of SEQ ID NO:1 or SEQ ID NO:2; 107 of SEQ ID NO:1, SEQ ID NO:2, or SEQ ID NO:3; 108 of SEQ ID NO:1 or SEQ ID NO:2; 109 of SEQ ID NO:1, SEQ ID NO:2, or SEQ ID NO:3; 110 of SEQ ID NO:1 or SEQ ID NO:2; 111 of SEQ ID NO:1 or SEQ ID NO:2; 112 of SEQ ID NO:1, SEQ ID NO:2, or SEQ ID NO:3; 141 of SEQ ID NO:1 or SEQ ID NO:2; 147 of SEQ ID NO:1, SEQ ID NO:2, or SEQ ID NO:3; 154 of SEQ ID NO:1 or SEQ ID NO:2; 179 of SEQ ID NO:1, SEQ ID NO:2, or SEQ ID NO:3; 180 of SEQ ID NO:1 or SEQ ID NO:2; 181 of SEQ ID NO:1 or SEQ ID NO:2; 183 of SEQ ID NO:1, SEQ ID NO:2, or SEQ ID NO:3; 184 of SEQ ID NO:1, SEQ ID NO:2, or SEQ ID NO:3; 185 of SEQ ID NO:1 or SEQ ID NO:2; 186 of SEQ ID NO:1, SEQ ID NO:2, or SEQ ID NO:3; 187 of SEQ ID NO:1 or SEQ ID NO:2; 188 of SEQ ID NO:1 or SEQ ID NO:2; 189 of SEQ ID NO:1 or SEQ ID NO:2; 197 of SEQ ID NO:3; 198 of SEQ ID NO:1 or SEQ ID NO:2; 204 of SEQ ID NO:3; 205 of SEQ ID NO:1 or SEQ ID NO:2; 241 of SEQ ID NO:3; 242 of SEQ ID NO:1 or SEQ ID NO:2; 247 of SEQ ID NO:1 or SEQ ID NO:2; 247 of SEQ ID NO:3; 248 of SEQ ID NO:1 or SEQ ID NO:2; 250 of SEQ ID NO:3; 251 of SEQ ID NO:1 or SEQ ID NO:2; 264 of SEQ ID NO:1, SEQ ID NO:2, or SEQ ID NO:3; 265 of SEQ ID NO:1 or SEQ ID NO:2; and 286 of SEQ ID NO:1 or SEQ ID NO:2. In some embodiments, a Shiga toxin effector polypeptide may comprise one or more of the following amino acid substitutions: K1 to A, G, V, L, I, F, M and H; T4 to A, G, V, L, I, F, M, and S; D6 to A, G, V, L, I, F, S, and Q; S8 to A, G, V, I, L, F, and M; T8 to A, G, V, I, L, F, M, and S; T9 to A, G, V, I, L, F, M, and S; S9 to A, G, V, L, I, F, and M; K11 to A, G, V, L, I, F, M and H; T12 to A, G, V, I, L, F, M, and S; S33 to A, G, V, L, I, F, and M; S43 to A, G, V, L, I, F, and M; G44 to A and L; S45 to A, G, V, L, I, F, and M; T45 to A, G, V, L, I, F, and M; G46 to A and P; D47 to A, G, V, L, I, F, S, and Q; N48 to A, G, V, L, and M; L49 to A or G; F50; A51 to V; D53 to A, G, V, L, I, F, S, and Q; V54 to A, G, and L; R55 to A, G, V, L, I, F, M, Q, S, K, and H; G56 to A and P; 157 to A, G, M, and F; L57 to A, G, M, and F; D58 to A, G, V, L, I, F, S, and Q; P59 to A, G, and F; E60 to A, G, V, L, I, F, S, Q, N, D, M, and R; E61 to A, G, V, L, I, F, S, Q, N, D, M, and R; G62 to A; D94 to A, G, V, L, I, F, S, and Q; R84 to A, G, V, L, I, F, M, Q, S, K, and H; V88 to A and G; I88 to A, G, and V; D94; S96 to A, G, V, I, L, F, and M; T104 to A, G, V, I, L, F, M, and S; A105 to L; T107 to A, G, V, I, L, F, M, and S; S107 to A, G, V, L, I, F, and M; L108 to A, G, and M; S109 to A, G, V, I, L, F, and M; T109 to A, G, V, I, L, F, M, and S; G110 to A; D111 to A, G, V, L, I, F, S, and Q; S112 to A, G, V, L, I, F, and M; D141 to A, G, V, L, I, F, S, and Q; G147 to A; V154 to A and G; R179 to A, G, V, L, I, F, M, Q, S, K, and H; T180 to A, G, V, L, I, F, M, and S; T181 to A, G, V, L, I, F, M, and S; D183 to A, G, V, L, I, F, S, and Q; D184 to A, G, V, L, I, F, S, and Q; L185 to A, G, and V; S186 to A, G, V, I, L, F, and M; G187 to A; R188 to A, G, V, L, I, F, M, Q, S, K, and H; S189 to A, G, V, I, L, F, and M; D197 to A, G, V, L, I, F, S, and Q; D198 to A, G, V, L, I, F, S, and Q; R204 to A, G, V, L, I, F, M, Q, S, K, and H; R205 to A, G, V, L, I, F, M, Q, S, K and H; C242 to A, G, V, and S; S247 to A, G, V, I, L, F, and M; Y247 to A, G, V, L, I, F, and M; R248 to A, G, V, L, I, F, M, Q, S, K, and H; R250 to A, G, V, L, I, F, M, Q, S, K, and H; R251 to A, G, V, L, I, F, M, Q, S, K, and H; C262 to A, G, V, and S; D264 to A, G, V, L, I, F, S, and Q; G264 to A; and T286 to A, G, V, L, I, F, M, and S.

In addition, any amino acid substitution in one epitope region of a Shiga toxin effector polypeptide which disrupts an epitope while retaining significant Shiga toxin effector function is combinable with any other amino acid substitution in the same or a different epitope region which disrupts an epitope while retaining significant Shiga toxin effector function to form a de-immunized, Shiga toxin effector polypeptide with multiple epitope regions disrupted while still retaining a significant level of Shiga toxin effector function. In some embodiments, a Shiga toxin effector polypeptide may comprise a combination of two or more of the aforementioned substitutions and/or the combinations of substitutions described in WO 2015/113007, WO 2016/196344, and/or WO 2018/140427.

Based on work described in WO 2015/113007, WO 2016/196344, and WO 2018/140427, certain amino acid regions in the A Subunits of Shiga toxins are predicted to tolerate epitope disruptions while still retaining significant Shiga toxin effector functions. For example, the epitope regions natively positioned at 1-15, 39-48, 53-66, 55-66, 94-115, 180-190, 179-190, and 243-257 tolerated multiple amino acid substitution combinations simultaneously without compromising Shiga toxin enzymatic activity and cytotoxicity.

### B. Examples of Furin-Cleavage Resistant, Shiga Toxin Effector Polypeptides

In some embodiments, the Shiga toxin effector polypeptide may comprise a disrupted, furin cleavage site at the carboxy-terminus of a Shiga toxin A1 fragment derived region. In some embodiments, the Shiga toxin effector polypeptide does not comprise any known compensatory structure which may provide furin cleavage proximal to the carboxy-terminus of the Shiga toxin A1 fragment derived region. Non-limiting examples of disrupted and furin cleavage sites are described in WO 2015/191764.

Certain furin-cleavage site disruptions are indicated herein by reference to specific amino acid positions of native Shiga toxin A Subunit sequences provided in the Sequence Listing (e.g. SEQ ID NOs: 1-18), noting that naturally occurring Shiga toxin A Subunits includes precursor forms containing signal sequences of about 22 amino acids at their amino-terminals which are removed to produce mature Shiga toxin A Subunits and are recognizable to the skilled worker. Further, certain furin-cleavage site disruptions comprising mutations are indicated herein by reference to specific amino acids (e.g. R for an arginine residue) natively present at specific positions within native Shiga toxin A Subunits (e.g. R251 for the arginine residue at position 251 from the amino-terminus) followed by the amino acid with which that residue has been substituted in the particular mutation under discussion (e.g. R251A represents the amino acid substitution of alanine for arginine at amino acid residue 251 from the amino-terminus).

In some embodiments, the Shiga toxin effector polypeptide comprises a disrupted furin-cleavage site at the carboxy-terminus of a Shiga toxin A1 fragment derived region, and such embodiments are referred to herein as “furin-cleavage resistant” or “protease-cleavage resistant,” Shiga toxin effector polypeptides to describe their property(ies) relative to wild-type, Shiga toxin A Subunits and/or wild-type, Shiga toxin A1 fragment fusion proteins.

In some embodiments, the protease-cleavage resistant, Shiga toxin effector polypeptide consists essentially of a truncated Shiga toxin A Subunit having two or more mutations.

In some embodiments, the protease-cleavage resistant, Shiga toxin effector polypeptide comprises the disrupted furin-cleavage site comprising the amino acid residue substitution (relative to a wild-type Shiga toxin polypeptide) of one or both of the arginine residues in the minimal, furin-cleavage site consensus site with A, G, or H. In some embodiments, the protease-cleavage resistant, Shiga toxin effector polypeptide comprises a disruption which comprises an amino acid substitution within a furin-cleavage site, where in the substitution occurs at the natively positioned amino acid selected from the group consisting of: 247 of SEQ ID NO:3, 248 of SEQ ID NO:1 or SEQ ID NO:2, 250 of SEQ ID NO:3, 251 of SEQ ID NO:1 or SEQ ID NO:2, or the equivalent position in a conserved Shiga toxin effector polypeptide and/or non-native Shiga toxin effector polypeptide sequence. In some embodiments, the substitution is to any non-conservative amino acid and the substitution occurs at the natively positioned amino acid residue position. In some embodiments, the mutation comprises an amino acid substitution selected from the group consisting of: R247A, R248A, R250A R251A, or the equivalent position in a conserved Shiga toxin effector polypeptide and/or non-native Shiga toxin effector polypeptide sequence.

In some embodiments, the protease-cleavage resistant Shiga toxin effector polypeptide comprises the disrupted furin-cleavage site comprising the mutation which is a deletion. In some embodiments, the disrupted furin-cleavage site comprises a mutation which is a deletion of the region natively positioned at 247-252 in StxA (SEQ ID NO:2) and SLT-1A (SEQ ID NO:3), or the region natively positioned at 246-251 in SLT-2A (SEQ ID NO:3); a deletion of the region natively positioned at 244-246 in StxA (SEQ ID NO:2) and SLT-1A (SEQ ID NO:3), or the region natively positioned at 243-245 in SLT-2A (SEQ ID NO:3); or a deletion of the region natively positioned at 253-259 in StxA (SEQ ID NO:2) and SLT-1A (SEQ ID NO:3), or the region natively positioned at 252-258 in SLT-2A (SEQ ID NO:3).

In some embodiments, the protease-cleavage resistant Shiga toxin effector polypeptide comprises the disrupted furin-cleavage site comprising the mutation which is a carboxy-terminal truncation as compared to a wild-type Shiga toxin A Subunit, the truncation which results in the deletion of one or more amino acid residues within the furin-cleavage site. In some embodiments, the disrupted furin-cleavage site comprises the carboxy-terminal truncation which deletes one or more amino acid residues within the minimal cleavage site R/Y-x-x-R (SEQ ID NO: 159), such as, e.g., for StxA and SLT-1A derived Shiga toxin effector polypeptides, truncations ending at the natively amino acid residue position 250, 249, 248, 247, 246, 245, 244, 243, 242, 241, 240, or less; and for SLT-2A derived Shiga toxin effector polypeptides, truncations ending at the natively amino acid residue position 249, 248, 247, 246, 245, 244, 243, 242, 241, or less. Some embodiments comprise the disrupted furin-cleavage site comprising a combination of any of the aforementioned mutations, where possible.

In some embodiments, the disrupted furin-cleavage site comprises the mutation(s) that is a partial, carboxy-terminal truncation of the furin-cleavage site; however, certain molecules do not comprise the disrupted furin-cleavage site which is a complete, carboxy-terminal truncation of the entire 20 amino acid residue, furin-cleavage site. For example, certain Shiga toxin effector polypeptides comprise the disrupted furin-cleavage site comprising a partial, carboxy-terminal truncation of the Shiga toxin A1 fragment region up to native position 240 in StxA (SEQ ID NO:2) or SLT-1A (SEQ ID NO:1) but not a carboxy-terminal truncation at position 239 or less. Similarly, certain Shiga toxin effector polypeptides comprise the disrupted furin-cleavage site comprising a partial, carboxy-terminal truncation of the Shiga toxin A1 fragment region up to native position 239 in SLT-2A (SEQ ID NO:3) but not a carboxy-terminal truncation at position 238 or less. In the largest carboxy-terminal truncation of the furin-cleavage resistant, Shiga toxin effector polypeptide, mutations comprising the disrupted furin-cleavage site, positions P14 and P13 of the furin-cleavage site are still present.

In some embodiments, the disrupted furin-cleavage site comprises both an amino acid residue substitution within the furin-cleavage site and a carboxy-terminal truncation as compared to a wild-type, Shiga toxin A Subunit. In some embodiments, the disrupted furin-cleavage site comprises both an amino acid residue substitution within the minimal furin-cleavage site R/Y-x-x-R (SEQ ID NO: 159) and a carboxy-terminal truncation as compared to a wild-type, Shiga toxin A Subunit, such as, e.g., for StxA and SLT-1A derived Shiga toxin effector polypeptides, truncations ending at the natively amino acid residue position 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and comprising the natively positioned amino acid residue R248 and/or R251 substituted with any non-positively charged, amino acid residue where appropriate; and for SLT-2A derived Shiga toxin effector polypeptides, truncations ending at the natively amino acid residue position 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and comprising the natively positioned amino acid residue Y247 and/or R250 substituted with any non-positively charged, amino acid residue where appropriate. In some embodiments, the truncated Shiga toxin effector polypeptide comprising a disrupted furin-cleavage site also comprises the furin-cleavage site, amino acid residues at positions P9, P8, and/or P7 in order to maintain optimal cytotoxicity.

In some embodiments, the disrupted furin-cleavage site comprises a mutation(s) which is one or more internal, amino acid residue deletions, as compared to a wild-type, Shiga toxin A Subunit. In some embodiments, the disrupted furin-cleavage site comprises a mutation(s) which has one or more amino acid residue deletions within the minimal furin-cleavage site R/Y-x-x-R (SEQ ID NO: 159). For example, StxA and SLT-1A derived Shiga toxin effector polypeptides comprising internal deletions of the natively positioned amino acid residues R248 and/or R251, which may be combined with deletions of surrounding residues such as, e.g., 249, 250, 247, 252, etc.; and SLT-2A derived Shiga toxin effector polypeptides comprising internal deletions of the natively positioned amino acid residues Y247 and/or R250, which may be combined with deletions of surrounding residues such as, e.g., 248, 249, 246, 251, etc. In some embodiments, the disrupted furin-cleavage site comprises a mutation which is a deletion of four, consecutive, amino acid residues which deletes the minimal furin-cleavage site R/Y-x-x-R (SEQ ID NO: 159), such as, e.g., StxA and SLT-1A derived Shiga toxin effector polypeptides lacking R248-R251 and SLT-2A derived Shiga toxin effector polypeptides lacking Y247-R250. In some embodiments, the disrupted furin-cleavage site comprises a mutation(s) having one or more amino acid residue deletions in the amino acid residues flanking the core furin-cleavage site, such as, e.g., a deletion of 244-247 and/or 252-255 in SLT-1A or StxA. In some embodiments, the disrupted furin-cleavage site comprises a mutation which is an internal deletion of the entire surface-exposed, protease-cleavage sensitive loop as compared to a wild-type, Shiga toxin A Subunit, such as, e.g., for StxA and SLT-1A derived Shiga toxin effector polypeptides, a deletion of natively positioned amino acid residues 241-262; and for SLT-2A derived Shiga toxin effector polypeptides, a deletion of natively positioned amino acid residues 240-261.

In some embodiments, the disrupted furin-cleavage site comprises both a mutation which is an internal, amino acid residue deletion within the furin-cleavage site and a mutation which is carboxy-terminal truncation as compared to a wild-type, Shiga toxin A Subunit. In some embodiments, the disrupted furin-cleavage site comprises both a mutation which is an amino acid residue deletion within the minimal furin-cleavage site R/Y-x-x-R (SEQ ID NO: 159) and a mutation which is a carboxy-terminal truncation as compared to a wild-type, Shiga toxin A Subunit. For example, protease-cleavage resistant, Shiga toxin effector polypeptides may comprise a disrupted furin-cleavage site comprising mutation(s) which are deletions of the natively positioned amino acid residues 248-249 and/or 250-251 in a truncated StxA or SLT-1A polypeptide which still has amino acid residue 247 and/or 252, or the amino acid residues 247-248 and/or 249-250 in a truncated SLT-2A which still has amino acid residue 246 and/or 251. In some embodiments, the disrupted furin-cleavage site comprises a mutation having a deletion of four, consecutive, amino acid residues which deletes the minimal furin-cleavage site R/Y-x-x-R (SEQ ID NO: 159) and a carboxy-terminal truncation as compared to a wild-type, Shiga toxin A Subunit, such as, e.g., for StxA and SLT-1A derived Shiga toxin effector polypeptides, truncations ending at the natively amino acid residue position 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and lacking R248-R251; and for SLT-2A derived Shiga toxin effector polypeptides, truncations ending at the natively amino acid residue position 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, or greater and lacking Y247-R250.

### C. Examples of Shiga Toxin Effector Polypeptides Having an Embedded Epitope

In some embodiments, the Shiga toxin effector polypeptide may comprise one or more embedded or inserted, heterologous, T-cell epitopes for purposes of de-immunization and/or delivery to a MHC class I presentation pathway of a target cell. In some embodiments, embedding or partial embedding a T-cell epitope may be preferred over inserting a T-cell epitope because, e.g., embedding-type modifications are more likely to be successful in diverse sub-regions of a Shiga toxin effector polypeptide whereas successful insertions may be more limited to a smaller subset of Shiga toxin effector polypeptide sub-regions. The term “successful” is used here to mean the modification to the Shiga toxin effector polypeptide (e.g. introduction of a heterologous, T-cell epitope) results in a modified Shiga toxin effector polypeptide which retains one or more Shiga toxin effector functions at the requisite level of activity either alone or as a component of a PD-L1 targeting molecule.

Any of the Shiga toxin effector polypeptide sub-regions described in WO 2015/113007 may be suitable for some embodiments, and any of the Shiga toxin effector polypeptides described in WO 2015/113007 may be modified into a Shiga toxin effector polypeptide of a PD-L1 targeting molecule, e.g., by the addition of one or more new epitope region disruptions for de-immunization (such one as described herein) and/or a furin-cleavage site disruption (such as one described herein).

In some embodiments, the Shiga toxin effector polypeptide consists essentially of a truncated Shiga toxin A Subunit comprising an embedded or inserted, heterologous, T-cell epitope and one or more other mutations. In some embodiments, the Shiga toxin effector polypeptide comprises an embedded or inserted, heterologous, T-cell epitope and is smaller than a full-length, Shiga toxin A Subunit, such as, e.g., consisting of the polypeptide represent by amino acids 77 to 239 of SLT-1A (SEQ ID NO:1) or StxA (SEQ ID NO:2) or the equivalent in other A Subunits of members of the Shiga toxin family (e.g. amino acids 77 to 238 of SLT-2A (SEQ ID NO:3)). For example, in some embodiments, the Shiga toxin effector polypeptides is derived from amino acids 75 to 251 of SEQ ID NO:1, 1 to 241 of SEQ ID NO:1, 1 to 251 of SEQ ID NO:1, or amino acids 1 to 261 of SEQ ID NO:1, wherein the Shiga toxin effector polypeptide comprises at least one embedded or inserted, heterologous T-cell epitope and at least one amino acid is disrupted in an endogenous, B-cell and/or CD4+ T-cell epitope region and wherein the disrupted amino acid does not overlap with the embedded or inserted epitope. Similarly in other embodiments, the Shiga toxin effector polypeptide is derived from amino acids 75 to 251 of SEQ ID NO:2, 1 to 241 of SEQ ID NO:2, 1 to 251 of SEQ ID NO:2, or amino acids 1 to 261 of SEQ ID NO:2, wherein the Shiga toxin effector polypeptide comprises at least one embedded or inserted, heterologous T-cell epitope and at least one amino acid is disrupted in an endogenous, B-cell and/or CD4+ T-cell epitope region and wherein the disrupted amino acid does not overlap with the embedded or inserted epitope. Additionally, the Shiga toxin effector polypeptide may be derived from amino acids 75 to 251 of SEQ ID NO:3, 1 to 241 of SEQ ID NO:3, 1 to 251 of SEQ ID NO:3, or amino acids 1 to 261 of SEQ ID NO:3, wherein the Shiga toxin effector polypeptide comprises at least one embedded or inserted, heterologous T-cell epitope and at least one amino acid is disrupted in an endogenous, B-cell and/or CD4+ T-cell epitope region and wherein the disrupted amino acid does not overlap with the embedded or inserted epitope. In some embodiments, the Shiga toxin effector polypeptide comprises an embedded or inserted, heterologous, T-cell epitope and a disrupted furin-cleavage site at the carboxy-terminus of a Shiga toxin A1 fragment derived region. For example in some embodiments, the Shiga toxin effector polypeptide is derived from amino acids 75 to 251 of SEQ ID NO:1, 1 to 241 of SEQ ID NO:1, 1 to 251 of SEQ ID NO:1, or amino acids 1 to 261 of SEQ ID NO:1, wherein the Shiga toxin effector polypeptide comprises at least one embedded or inserted, heterologous T-cell epitope and a disrupted furin-cleavage site at the carboxy-terminus of a Shiga toxin A1 fragment derived region. Similarly in other embodiments, the Shiga toxin effector polypeptide is derived from amino acids 75 to 251 of SEQ ID NO:2, 1 to 241 of SEQ ID NO:2, 1 to 251 of SEQ ID NO:2, or amino acids 1 to 261 of SEQ ID NO:2, wherein the Shiga toxin effector polypeptide comprises at least one embedded or inserted, heterologous T-cell epitope and a disrupted furin-cleavage site at the carboxy-terminus of a Shiga toxin A1 fragment derived region. Additionally, the Shiga toxin effector polypeptide may be derived from amino acids 75 to 251 of SEQ ID NO:3, 1 to 241 of SEQ ID NO:3, 1 to 251 of SEQ ID NO:3, or amino acids 1 to 261 of SEQ ID NO:3, wherein the Shiga toxin effector polypeptide comprises at least one embedded or inserted, heterologous T-cell epitope and a disrupted furin-cleavage site at the carboxy-terminus of a Shiga toxin A1 fragment derived region.

### D. Examples of Combination Shiga Toxin Effector Polypeptides

A combination Shiga toxin effector polypeptide comprises two or more sub-regions (i.e. non-overlapping sub-regions) wherein each sub-region comprises at least one of the following: (1) a disruption in an endogenous epitope or epitope region; (2) an embedded, heterologous, T-cell epitope-peptide; (3) an inserted, heterologous, T-cell epitope-peptide; and (4) a disrupted furin-cleavage site at the carboxy-terminus of an A1 fragment derived region.

Some embodiments of the combination Shiga toxin effector polypeptides comprise both (1) a disruption in an endogenous epitope or epitope region and (2) a disrupted furin-cleavage site at the carboxy-terminus of an A1 fragment derived region. It is predicted that any of the individual, de-immunized, Shiga toxin effector sub-regions described in WO 2015/113007, WO 2016/196344, and WO 2018/140427 (see e.g. Table 2, supra) may generally be combined with any Shiga toxin effector sub-region comprising a disrupted furin-cleavage site described herein, described in WO 2015/191764, and/or known in the art in order to create a Shiga toxin effector polypeptide for use as a component of a PD-L1 targeting molecule.

In some embodiments, the Shiga toxin effector polypeptide consists essentially of the polypeptide shown in any one of SEQ ID NOs: 19-21 which further comprises a disruption of at least one, endogenous, B-cell and/or T-cell epitope region which does not overlap with an embedded or inserted, heterologous, CD8+ T-cell epitope; wherein the disruption comprises one or more amino acid residue substitutions relative to a wild-type Shiga toxin. In some embodiments the substitution is selected from the group consisting of: K1 to A, G, V, L, I, F, M and H; T4 to A, G, V, L, I, F, M, and S; D6 to A, G, V, L, I, F, S, Q and R; S8 to A, G, V, I, L, F, and M; T9 to A, G, V, I, L, F, M, and S; S9 to A, G, V, L, I, F, and M; K11 to A, G, V, L, I, F, M and H; T12 to A, G, V, I, L, F, M, S, and K; S12 to A, G, V, I, L, F, and M; S33 to A, G, V, L, I, F, M, and C; S43 to A, G, V, L, I, F, and M; G44 to A or L; S45 to A, G, V, L, I, F, and M; T45 to A, G, V, L, I, F, and M; G46 to A and P; D47 to A, G, V, L, I, F, S, M, and Q; N48 to A, G, V, L, M and F; L49 to A, V, C, and G; Y49 to A, G, V, L, I, F, M, and T; F50 to A, G, V, L, I, and T; A51; D53 to A, G, V, L, I, F, S, and Q; V54 to A, G, I, and L; R55 to A, G, V, L, I, F, M, Q, S, K, and H; G56 to A and P; 157 to A, G, V, and M; L57 to A, V, C, G, M, and F; D58 to A, G, V, L, I, F, S, and Q; P59 to A, G, and F; E60 to A, G, V, L, I, F, S, Q, N, D, M, T, and R; E61 to A, G, V, L, I, F, S, Q, N, D, M, and R; G62 to A; R84 to A, G, V, L, I, F, M, Q, S, K, and H; V88 to A and G; I88 to A, V, C, and G; D94 to A, G, V, L, I, F, S, and Q; S96 to A, G, V, I, L, F, and M; T104 to A, G, V, L, I, F, M; and N; A105 to L; T107 to A, G, V, L, I, F, M, and P; S107 to A, G, V, L, I, F, M, and P; L108 to A, V, C, and G; S109 to A, G, V, I, L, F, and M; T109 to A, G, V, I, L, F, M, and S; G110 to A; S112 to A, G, V, L, I, F, and M; D111 to A, G, V, L, I, F, S, Q, and T; S112 to A, G, V, L, I, F, and M; D141 to A, G, V, L, I, F, S, and Q; G147 to A; V154 to A and G. R179 to A, G, V, L, I, F, M, Q, S, K, and H; T180 to A, G, V, L, I, F, M, and S; T181 to A, G, V, L, I, F, M, and S; D183 to A, G, V, L, I, F, S, and Q; D184 to A, G, V, L, I, F, S, and Q; L185 to A, G, V and C; S186 to A, G, V, I, L, F, and M; G187 to A; R188 to A, G, V, L, I, F, M, Q, S, K, and H; S189 to A, G, V, I, L, F, and M; D198 to A, G, V, L, I, F, S, and Q; R204 to A, G, V, L, I, F, M, Q, S, K, and H; R205 to A, G, V, L, I, F, M, Q, S, K and H; S247 to A, G, V, I, L, F, and M; Y247 to A, G, V, L, I, F, and M; R248 to A, G, V, L, I, F, M, Q, S, K, and H; R250 to A, G, V, L, I, F, M, Q, S, K, and H; R251 to A, G, V, L, I, F, M, Q, S, K, and H; D264 to A, G, V, L, I, F, S, and Q; G264 to A; and T286 to A, G, V, L, I, F, M, and S. In some embodiments, there are multiple disruptions of multiple, endogenous B-cell and/or CD8+ T-cell epitope regions wherein each disruption involves at least one amino acid residue substitution selected from the group consisting of: K1 to A, G, V, L, I, F, M and H; T4 to A, G, V, L, I, F, M, and S; D6 to A, G, V, L, I, F, S, Q and R; S8 to A, G, V, I, L, F, and M; T9 to A, G, V, I, L, F, M, and S; S9 to A, G, V, L, I, F, and M; K11 to A, G, V, L, I, F, M and H; T12 to A, G, V, I, L, F, M, S, and K; S12 to A, G, V, I, L, F, and M; S33 to A, G, V, L, I, F, M, and C; S43 to A, G, V, L, I, F, and M; G44 to A or L; S45 to A, G, V, L, I, F, and M; T45 to A, G, V, L, I, F, and M; G46 to A and P; D47 to A, G, V, L, I, F, S, M, and Q; N48 to A, G, V, L, M and F; L49 to A, V, C, and G; Y49 to A, G, V, L, I, F, M, and T; F50 to A, G, V, L, I, and T; A51; D53 to A, G, V, L, I, F, S, and Q; V54 to A, G, I, and L; R55 to A, G, V, L, I, F, M, Q, S, K, and H; G56 to A and P; 157 to A, G, V, and M; L57 to A, V, C, G, M, and F; D58 to A, G, V, L, I, F, S, and Q; P59 to A, G, and F; E60 to A, G, V, L, I, F, S, Q, N, D, M, T, and R; E61 to A, G, V, L, I, F, S, Q, N, D, M, and R; G62 to A; R84 to A, G, V, L, I, F, M, Q, S, K, and H; V88 to A and G; I88 to A, V, C, and G; D94 to A, G, V, L, I, F, S, and Q; S96 to A, G, V, I, L, F, and M; T104 to A, G, V, L, I, F, M; and N; A105 to L; T107 to A, G, V, L, I, F, M, and P; S107 to A, G, V, L, I, F, M, and P; L108 to A, V, C, and G; S109 to A, G, V, I, L, F, and M; T109 to A, G, V, I, L, F, M, and S; G110 to A; S112 to A, G, V, L, I, F, and M; D111 to A, G, V, L, I, F, S, Q, and T; S112 to A, G, V, L, I, F, and M; D141 to A, G, V, L, I, F, S, and Q; G147 to A; V154 to A and G. R179 to A, G, V, L, I, F, M, Q, S, K, and H; T180 to A, G, V, L, I, F, M, and S; T181 to A, G, V, L, I, F, M, and S; D183 to A, G, V, L, I, F, S, and Q; D184 to A, G, V, L, I, F, S, and Q; L185 to A, G, V and C; S186 to A, G, V, I, L, F, and M; G187 to A; R188 to A, G, V, L, I, F, M, Q, S, K, and H; S189 to A, G, V, I, L, F, and M; D198 to A, G, V, L, I, F, S, and Q; R204 to A, G, V, L, I, F, M, Q, S, K, and H; R205 to A, G, V, L, I, F, M, Q, S, K and H; S247 to A, G, V, I, L, F, and M; Y247 to A, G, V, L, I, F, and M; R248 to A, G, V, L, I, F, M, Q, S, K, and H; R250 to A, G, V, L, I, F, M, Q, S, K, and H; R251 to A, G, V, L, I, F, M, Q, S, K, and H; D264 to A, G, V, L, I, F, S, and Q; G264 to A; and T286 to A, G, V, L, I, F, M, and S.

In some embodiments, the Shiga toxin effector polypeptide comprises both (1) an embedded or inserted, heterologous, T-cell epitope-peptide and (2) a disrupted furin-cleavage site at the carboxy-terminus of an A1 fragment derived region. Any of the Shiga toxin effector polypeptide sub-regions comprising an embedded or inserted, heterologous, T-cell epitope described in WO 2015/113007 may generally be combined with any protease-cleavage resistant, Shiga toxin effector polypeptide sub-region (e.g., modified, Shiga toxin A Subunit sub-regions described herein, described in WO 2015/191764, and/or known in the art) in order to create a combination, Shiga toxin effector polypeptide which, as a component of a PD-L1 targeting molecule, is both protease-cleavage resistant and capable of delivering a heterologous, T-cell epitope to the MHC class I presentation pathway of a target cell. Non-limiting examples of this type of combination Shiga toxin effector polypeptide are shown in SEQ ID NOs: 19-21.

Some embodiments of the combination Shiga toxin effector polypeptides comprise both (1) a disruption in an endogenous epitope or epitope region and (2) an embedded, heterologous, T-cell epitope-peptide. However, the Shiga toxin effector sub-regions comprising inserted or embedded, heterologous, T-cell epitopes described herein or in WO 2015/191764 are generally not combinable with every de-immunized, Shiga toxin effector sub-regions described herein, except where empirically shown to be successfully combined such that the resulting combination molecule retained a sufficient level of a Shiga toxin effector function(s). The disclosure herein shows how such embodiments may be made and tested to empirically demonstrate success.

The term “successful” is used here to mean two or more amino acid residue substitutions in a Shiga toxin effector polypeptide results in a functional feature, such as, e.g., de-immunization, reduced furin-cleavage, and/or ability to deliver an embedded or inserted epitope, while the modified Shiga toxin effector polypeptide retains one or more Shiga toxin effector functions. The approaches and assays described herein show how to design, make and empirically test embodiments, which represent combination, Shiga toxin effector polypeptides and PD-L1 targeting molecules comprising the same.

The combination, Shiga toxin effector polypeptide may combine the features of their respective sub-regions, such as, e.g., a furin-cleavage site disruption, individual epitope disruptions, and/or a heterologous T-cell epitope cargo, and these combinations sometimes result in Shiga toxin effector polypeptides with synergistic reductions in immunogenicity as compared to the sum of their partially de-immunized sub-regions.

De-immunized, Shiga toxin effector polypeptides which exhibit no cytotoxicity or reduced cytotoxicity at certain concentrations, e.g. Shiga toxin effector polypeptides comprising R179A, may still be useful as de-immunized, Shiga toxin effector polypeptides for delivering exogenous materials into cells. Similarly, CD8+ T-cell hyper-immunized, Shiga toxin effector polypeptides of the which exhibit no cytotoxicity or reduced cytotoxicity at certain concentrations, e.g. a Shiga toxin effector polypeptide comprising an epitope embedded into its catalytic domain (see e.g. WO 2015/113005: Example 1-F), may still be useful for delivering a T-cell epitope(s) to a desired subcellular compartment of a cell in which the Shiga toxin effector polypeptide is present or as a component of a PD-L1 targeting molecule for delivery of a T-cell epitope(s) into a target cell.

### E. Examples of PD-L1 Targeting Molecules

The following embodiments describe in more detail certain structures of illustrative cell-target molecules which target cells physically coupled to PD-L1 at a cellular surface, e.g. cells which express PD-L1.

In some embodiments, the PD-L1 targeting molecule comprises or consists essentially of the polypeptide shown in any one of SEQ ID NOs: 84-166 or 189-256, and optionally the PD-L1 targeting molecule comprises an amino-terminal methionine residue. In some embodiments, the PD-L 1 targeting molecule comprises the sequence of SEQ ID NO: 86, or a sequence at least 80%, at least 85%, at least 90%, at least 95%, or at least 99% identical thereto. In some embodiments, the PD-L1 targeting molecule comprises the sequence of SEQ ID NO: 87, or a sequence at least 80%, at least 85%, at least 90%, at least 95%, or at least 99% identical thereto. In some embodiments, the PD-L1 targeting molecule comprises the sequence of SEQ ID NO: 87 with at least 1, at least 5, at least 10, or at least 20 amino acid mutations. For example, the PD-L1 targeting molecule may comprise the sequence of SEQ ID NO: 87 with 1-5, 1-10, 10-15, 15-20, 20-25, 25-30, or more amino acid mutations. In some embodiments, the PD-L1 targeting molecule comprises the sequence of SEQ ID NO: 86 with at least 1, at least 5, at least 10, or at least 20 amino acid mutations. For example, the PD-L1 targeting molecule may comprise the sequence of SEQ ID NO: 86 with 1-5, 1-10, 10-15, 15-20, 20-25, 25-30, or more amino acid mutations.

In some embodiments, the PD-L1 targeting molecule comprises a binding region comprising the sequence of SEQ ID NO: 38 and a Shiga toxin effector polypeptide. In some embodiments, the PD-L1 targeting molecule comprises a binding region comprising the sequence of SEQ ID NO: 38 and a Shiga toxin effector polypeptide comprising the sequence of SEQ ID NO: 41.

In some embodiments, the PD-L1 targeting molecule comprises a binding region and a Shiga toxin effector polypeptide that are covalently coupled via a linker. In some embodiments, the linker comprises the sequence of SEQ ID NO: 175. In some embodiments, the PD-L1 targeting molecule comprises a Shiga toxin effector polypeptide comprising the sequence of SEQ ID NO: 41, a linker, and a binding region comprising the sequence of SEQ ID NO: 38. In some embodiments, the PD-L1 targeting molecule comprises a Shiga toxin effector polypeptide comprising the sequence of SEQ ID NO: 41, a linker comprising the sequence of SEQ ID NO: 175, and a binding region comprising the sequence of SEQ ID NO: 38.

In some embodiments, the PD-L1 targeting molecule further comprises at least one heterologous CD8+ T-cell epitope. In some embodiments, the at least one heterologous CD8+ T-cell epitope is positioned at or near the C-terminus of the molecule. In some embodiments, the PD-L1 targeting molecule comprises one, two, three, four, five, six, seven, eight, or more copies of the same heterologous CD8+ T-cell epitope. In some embodiments, the PD-L1 targeting molecule coprises one, two, three, four, five, six, seven, eight, or more different heterologous CD8+ T-cell epitopes. In some embodiments, the PD-L1 targeting molecule comprises one copy of a heterologous CD8+ T-cell epitope comprising the sequence of SEQ ID NO: 77. In some embodiments, the PD-L1 targeting molecule comprises two copies of a heterologous CD8+ T-cell epitope comprising the sequence of SEQ ID NO: 77. In some embodiments, the PD-L1 targeting molecule comprises three or more copies of a heterologous CD8+ T-cell epitope comprising the sequence of SEQ ID NO: 77. In some embodiments, the PD-L1 targeting molecule comprises a heterologous CD8+ T-cell epitope having the sequence of SEQ ID NO: 257 or 258. In some embodiments, the PD-L1 targeting molecule comprises one copy of a heterologous CD8+ T-cell epitope comprising the sequence of any noe of SEQ ID NO: 78-83 or 286-288. In some embodiments, the PD-L1 targeting molecule comprises two copies of a heterologous CD8+ T-cell epitope comprising the sequence of any one of SEQ ID NO: 7778-83 or 286-288. In some embodiments, the PD-L1 targeting molecule comprises three or more copies of a heterologous CD8+ T-cell epitope comprising the sequence of any one of SEQ ID NO: 78-83 or 286-288.

In some embodiments, a PD-L1 targeting molecule comprises a Shiga toxin effector polypeptide, a linker, a binding region, and a heterologous CD8+ T-cell epitope. In some embodiments, a PD-L1 targeting molecule comprises a Shiga toxin effector polypeptide, a linker, a binding region, and a heterologous CD8+ T-cell epitope comprising the sequence of SEQ ID NO: 77. In some embodiments, a PD-L1 targeting molecule comprises a Shiga toxin effector polypeptide, a linker, a binding region, and two copies of a heterologous CD8+ T-cell epitope. In some embodiments, a PD-L1 targeting molecule comprises a Shiga toxin effector polypeptide, a linker, a binding region, and two copies of a heterologous CD8+ T-cell epitope comprising the sequence of SEQ ID NO: 77. In some embodiments, a PD-L1 targeting molecule comprises a Shiga tocin effector polypeptide comprising the sequence of SEQ ID NO: 41, a binding region comprising the sequence of SEQ ID NO: 38, and a heterologous CD8+ T-cell epitope comprising the sequence of SEQ ID NO: 77. In some embodiments, a PD-L1 targeting molecule comprises a Shiga tocin effector polypeptide comprising the sequence of SEQ ID NO: 41, a binding region comprising the sequence of SEQ ID NO: 38, and two copies of a heterologous CD8+ T-cell epitope comprising the sequence of SEQ ID NO: 77. In some embodiments, a PD-L1 targeting molecule comprises a Shiga toxin effector polypeptide comprising the sequence of SEQ ID NO: 41, a linker comprising the sequence of SEQ ID NO: 175, a binding region comprising the sequence of SEQ ID NO: 38, and a heterologous CD8+ T-cell epitope comprising the sequence of SEQ ID NO: 77. In some embodiments, a PD-L1 targeting molecule comprises a Shiga toxin effector polypeptide comprising the sequence of SEQ ID NO: 41, a linker comprising the sequence of SEQ ID NO: 175, a binding region comprising the sequence of SEQ ID NO: 38, and two copies of a heterologous CD8+ T-cell epitope comprising the sequence of SEQ ID NO: 77. In some embodiments, the PD-L1 targeting molecule further comprises a cleavable spacer, such as a cleavable spacer having the sequence of SEQ ID NO: 187.

### Other Structural Variations

Also contemplated herein is the use fragments, variants, and/or derivatives of the PD-L1 targeting molecules which contain a functional binding site to any extracellular part of a target biomolecule, and even more preferably capable of binding a target biomolecule with high affinity (e.g. as shown by K_{D}). For example, any binding region which binds an extracellular part of a target biomolecule with a dissociation constant (K_{D}) of 10⁻⁵ to 10⁻¹² moles/liter, preferably less than 200 nM, may be substituted for use in making PD-L1 targeting molecules and methods of use thereof.

The skilled worker will recognize that variations may be made to the Shiga toxin effector polypeptides and PD-L1 targeting molecules, and polynucleotides encoding any of the former, without diminishing their biological activities, e.g., by maintaining the overall structure and function of the Shiga toxin effector polypeptide, such as in conjunction with one or more 1) endogenous epitope disruptions which reduce antigenic and/or immunogenic potential, 2) furin-cleavage site disruptions which reduce proteolytic cleavage, and/or 3) embedded or inserted epitopes which reduce antigenic and/or immunogenic potential or are capable of being delivered to a MHC I molecule for presentation on a cell surface. For example, some modifications may facilitate expression, facilitate purification, improve pharmacokinetic properties, and/or improve immunogenicity. Such modifications are well known to the skilled worker and include, for example, a methionine added at the amino-terminus to provide an initiation site, additional amino acids placed on either terminus to create conveniently located restriction sites or termination codons, and biochemical affinity tags fused to either terminus to provide for convenient detection and/or purification. A common modification to improve the immunogenicity of a polypeptide produced using a non-chordate system (e.g. a prokaryotic cell) is to remove, after the production of the polypeptide, the starting methionine residue, which may be formylated during production, such as, e.g., in a bacterial host system, because, e.g., the presence of N-formylmethionine (fMet) might induce undesirable immune responses in chordates.

Also contemplated herein is the inclusion of additional amino acid residues at the amino and/or carboxy termini of a PD-L1 targeting molecule, or a proteinaceous component of a PD-L1 targeting molecule, such as sequences for epitope tags or other moieties. The additional amino acid residues may be used for various purposes including, e.g., facilitating cloning, facilitating expression, post-translational modification, facilitating synthesis, purification, facilitating detection, and administration. Non-limiting examples of epitope tags and moieties are chitin binding protein domains, enteropeptidase cleavage sites, Factor Xa cleavage sites, FIAsH tags, FLAG tags, green fluorescent proteins (GFP), glutathione-S-transferase moieties, HA tags, maltose binding protein domains, myc tags, polyhistidine tags, ReAsH tags, strep-tags, strep-tag II, TEV protease sites, thioredoxin domains, thrombin cleavage site, and V5 epitope tags.

In certain of the above embodiments, the polypeptide sequence of the Shiga toxin effector polypeptides and/or PD-L1 targeting molecules are varied by one or more conservative amino acid substitutions introduced into the polypeptide region(s) as long as all required structural features are still present and the Shiga toxin effector polypeptide is capable of exhibiting any required function(s), either alone or as a component of a PD-L1 targeting molecule. As used herein, the term “conservative substitution” denotes that one or more amino acids are replaced by another, biologically similar amino acid residue. Examples include substitution of amino acid residues with similar characteristics, e.g. small amino acids, acidic amino acids, polar amino acids, basic amino acids, hydrophobic amino acids and aromatic amino acids (see, for example, Table 3). An example of a conservative substitution with a residue normally not found in endogenous, mammalian peptides and proteins is the conservative substitution of an arginine or lysine residue with, for example, ornithine, canavanine, aminoethylcysteine, or another basic amino acid. For further information concerning phenotypically silent substitutions in peptides and proteins see, e.g., Bowie J et al., Science 247: 1306-10 (1990).

TABLE 3
Examples of Conservative Amino Acid
Substitutions

| I | II | III | IV | V | VI | VII | VIII | IX | X | XI | XII | XIII | XIV |
|---|---|---|---|---|---|---|---|---|---|---|---|---|---|
| A | D | H | C | F | N | A | C | F | A | C | A | A | D |
| G | E | K | I | W | Q | G | M | H | C | D | C | C | E |
| P | Q | R | L | Y | S | I | P | W | F | E | D | D | G |
| S | N | | M | | T | L | | Y | G | H | G | E | K |
| T | | | V | | | V | | | H | K | N | G | P |
| | | | | | | | | | I | N | P | H | Q |
| | | | | | | | | | L | Q | S | K | R |
| | | | | | | | | | M | R | T | N | S |
| | | | | | | | | | R | S | V | Q | T |
| | | | | | | | | | T | T | | R | |
| | | | | | | | | | V | | | S | |
| | | | | | | | | | W | | | P | |
| | | | | | | | | | Y | | | T | |

In the conservative substitution scheme in Table 3, illustrative conservative substitutions of amino acids are grouped by physicochemical properties—I: neutral, hydrophilic; II: acids and amides; III: basic; IV: hydrophobic; V: aromatic, bulky amino acids, VI hydrophilic uncharged, VII aliphatic uncharged, VIII non-polar uncharged, IX cycloalkenyl-associated, X hydrophobic, XI polar, XII small, XIII turn-permitting, and XIV flexible. For example, conservative amino acid substitutions include the following: 1) S may be substituted for C; 2) M or L may be substituted for F; 3) Y may be substituted for M; 4) Q or E may be substituted for K; 5) N or Q may be substituted for H; and 6) H may be substituted for N.

Additional conservative amino acid substitutions include the following: 1) S may be substituted for C; 2) M or L may be substituted for F; 3) Y may be substituted for M; 4) Q or E may be substituted for K; 5) N or Q may be substituted for H; and 6) H may be substituted for N.

In some embodiments, the Shiga toxin effector polypeptides and PD-L1 targeting molecules may comprise functional fragments or variants of a polypeptide region described herein that have, at most, 20, 15, 10, 9, 8, 7, 6, 5, 4, 3, 2, or 1 amino acid substitutions compared to a polypeptide sequence recited herein, as long as it (1) comprises at least one embedded or inserted, heterologous T-cell epitope and at least one amino acid is disrupted in an endogenous, B-cell and/or CD4+ T-cell epitope region, wherein the disrupted amino acid does not overlap with the embedded or inserted epitope; (2) comprises at least one embedded or inserted, heterologous T-cell epitope and a disrupted furin-cleavage site at the carboxy-terminus of a Shiga toxin A1 fragment derived region; or (3) comprises a disrupted furin-cleavage site at the carboxy-terminus of a Shiga toxin A1 fragment derived region and comprises at least one amino acid is disrupted in an endogenous, B-cell and/or CD4+ T-cell epitope region, wherein the disrupted amino acid does not overlap with the disrupted furin-cleavage site. Variants of the Shiga toxin effector polypeptides and PD-L1 targeting molecules may also be prepared by changing a polypeptide described herein by altering one or more amino acid residues or deleting or inserting one or more amino acid residues, such as within the binding region or Shiga toxin effector polypeptide region, in order to achieve desired properties, such as changed cytotoxicity, changed cytostatic effects, changed immunogenicity, and/or changed serum half-life. The Shiga toxin effector polypeptides and PD-L1 targeting molecules may further be prepared with or without a signal sequence.

Accordingly, in some embodiments, the Shiga toxin effector polypeptide comprises or consists essentially of amino acid sequences having at least 55%, at least 60%, at least 65%, at least 70%, at least 75%, at least 80%, at least 85%, at least 90%, at least 95%, at least 97%, at least 98%, or at least 99%, overall sequence identity to a naturally occurring Shiga toxin A Subunit or fragment thereof, such as, e.g., Shiga toxin A Subunit, such as SLT-1A (SEQ ID NO:1), StxA (SEQ ID NO:2), and/or SLT-2A (SEQ ID NO:3), wherein the Shiga toxin effector polypeptide (1) comprises at least one embedded or inserted, heterologous T-cell epitope and at least one amino acid is disrupted in an endogenous, B-cell and/or CD4+ T-cell epitope region, and wherein the disrupted amino acid does not overlap with the embedded or inserted epitope; (2) comprises at least one embedded or inserted, heterologous T-cell epitope and a disrupted furin-cleavage site at the carboxy-terminus of a Shiga toxin A1 fragment derived region; or (3) comprises a disrupted furin-cleavage site at the carboxy-terminus of a Shiga toxin A1 fragment derived region and comprises at least one amino acid is disrupted in an endogenous, B-cell and/or CD4+ T-cell epitope region, and wherein the disrupted amino acid does not overlap with the disrupted furin-cleavage site.

In some embodiments, the Shiga toxin effector polypeptide has one or more amino acid residues may be mutated, inserted, or deleted in order to increase the enzymatic activity of the Shiga toxin effector polypeptide. In some embodiments, the Shiga toxin effector polypeptide has one or more amino acid residues may be mutated or deleted in order to reduce or eliminate catalytic and/or cytotoxic activity of the Shiga toxin effector polypeptide. For example, the catalytic and/or cytotoxic activity of the A Subunits of members of the Shiga toxin family may be diminished or eliminated by mutation or truncation.

The cytotoxicity of the A Subunits of members of the Shiga toxin family may be altered, reduced, or eliminated by mutation and/or truncation. The positions labeled tyrosine-77, glutamate-167, arginine-170, tyrosine-114, and tryptophan-203 have been shown to be important for the catalytic activity of Stx, Stx1, and Stx2 (Hovde C et al., Proc Natl Acad Sci USA 85: 2568-72 (1988); Deresiewicz R et al., Biochemistry 31: 3272-80 (1992); Deresiewicz R et al., Mol Gen Genet 241: 467-73 (1993); Ohmura M et al., Microb Pathog 15: 169-76 (1993); Cao C et al., Microbiol Immunol 38: 441-7 (1994); Suhan M, Hovde C, Infect Immun 66: 5252-9 (1998)). Mutating both glutamate-167 and arginine-170 eliminated the enzymatic activity of Slt-I A1 (i.e., SLT-1A1) in a cell-free ribosome inactivation assay (LaPointe P et al., J Biol Chem 280: 23310-18 (2005)). In another approach using de novo expression of Slt-I A1 in the endoplasmic reticulum, mutating both glutamate-167 and arginine-170 eliminated Slt-I A1 fragment cytotoxicity at that expression level (LaPointe P et al., J Biol Chem 280: 23310-18 (2005)). A truncation analysis demonstrated that a fragment of StxA from residues 75 to 268 still retains significant enzymatic activity in vitro (Haddad J et al., J Bacteriol 175: 4970-8 (1993)). A truncated fragment of Slt-I A1 containing residues 1-239 displayed significant enzymatic activity in vitro and cytotoxicity by de novo expression in the cytosol (LaPointe P et al., J Biol Chem 280: 23310-18 (2005)). Expression of a Slt-I A1 fragment truncated to residues 1-239 in the endoplasmic reticulum was not cytotoxic because it could not retrotranslocate to the cytosol (LaPointe P et al., J Biol Chem 280: 23310-18 (2005)).

The most critical residues for enzymatic activity and/or cytotoxicity in the Shiga toxin A Subunits were mapped to the following residue-positions: asparagine-75, tyrosine-77, tyrosine-114, glutamate-167, arginine-170, arginine-176, and tryptophan-203 among others (Di R et al., Toxicon 57: 525-39 (2011)). In particular, a double-mutant construct of Stx2A containing glutamate-E1 67-to-lysine and arginine-176-to-lysine mutations was completely inactivated; whereas, many single mutations in Stx1 and Stx2 showed a 10-fold reduction in cytotoxicity. Further, truncation of Stx1A to 1-239 or 1-240 reduced its cytotoxicity, and similarly, truncation of Stx2A to a conserved hydrophobic residue reduced its cytotoxicity. The most critical residues for binding eukaryotic ribosomes and/or eukaryotic ribosome inhibition in the Shiga toxin A Subunit have been mapped to the following residue-positions arginine-172, arginine-176, arginine-179, arginine-188, tyrosine-189, valine-191, and leucine-233 among others (McCluskey A et al., PLoS One 7: e31191 (2012). However, certain modification may increase a Shiga toxin functional activity exhibited by a Shiga toxin effector polypeptide. For example, mutating residue-position alanine-231 in Stx1A to glutamate increased Stx1A's enzymatic activity in vitro (Suhan M, Hovde C, Infect Immun 66: 5252-9 (1998)).

In some embodiments, the Shiga toxin effector polypeptide derived from SLT-1A (SEQ ID NO:1) or StxA (SEQ ID NO:2) has one or more amino acid residues mutated include substitution of the asparagine at position 75, tyrosine at position 77, tyrosine at position 114, glutamate at position 167, arginine at position 170, arginine at position 176, and/or substitution of the tryptophan at position 203. Examples of such substitutions will be known to the skilled worker based on the prior art, such as asparagine at position 75 to alanine, tyrosine at position 77 to serine, substitution of the tyrosine at position 114 to serine, substitution of the glutamate position 167 to glutamate, substitution of the arginine at position 170 to alanine, substitution of the arginine at position 176 to lysine, substitution of the tryptophan at position 203 to alanine, and/or substitution of the alanine at 231 with glutamate. Other mutations which either enhance or reduce Shiga toxin enzymatic activity and/or cytotoxicity are contemplated herein and may be determined using well known techniques and assays disclosed herein.

The Shiga toxin effector polypeptides and PD-L1 targeting molecules may optionally be conjugated to one or more additional agents, which may include therapeutic agents, diagnostic agents, and/or other additional exogenous materials known in the art, including such agents as described herein. In some embodiments, the Shiga toxin effector polypeptide or PD-L1 targeting molecule is PEGylated or albuminated, such as, e.g., to provide de-immunization, disrupt furin-cleavage by masking the extended loop and/or the furin-cleavage site at the carboxy-terminus of a Shiga toxin A1 fragment derived region, improve pharmacokinetic properties, and/or improve immunogenicity (see e.g., Wang Q et al., Cancer Res 53: 4588-94 (1993); Tsutsumi Y et al., Proc Natl Acad Sci USA 97: 8548-53 (2000); Buse J, El-Aneed A, Nanomed 5: 1237-60 (2010); Lim S et al., J Control Release 207-93 (2015)).

### IV. General Functions of the PD-L1 Targeting Molecules

The PD-L1 targeting molecules are useful in diverse applications involving, e.g., cell-killing; cell growth inhibition; intracellular, cargo delivery; biological information gathering; immune response stimulation, and/or remediation of a health condition. The PD-L1 targeting molecules are useful as therapeutic and/or diagnostic molecules, such as, e.g., as cell-targeting, cytotoxic, therapeutic molecules; cell-targeting, nontoxic, delivery vehicles; and/or cell-targeting, diagnostic molecules; for examples in applications involving the in vivo targeting of specific cell types for the diagnosis or treatment of a variety of diseases, including cancers, immune disorders, and microbial infections.

In some embodiments, the PD-L1 targeting molecules are capable of binding an extracellular part of PD-L1 molecules associated with cell surfaces of particular cell types and entering those cells. Once internalized within a targeted cell type, the PD-L1 targeting molecules may, in some embodiments, be capable of routing an enzymatically active, cytotoxic, Shiga toxin effector polypeptide fragment into the cytosol of the target cell and eventually killing the cell. Alternatively, nontoxic or reduced-toxicity variants of the PD-L1 targeting molecules may be used to deliver additional exogenous materials into target cells, such as epitopes, peptides, proteins, polynucleotides, and detection-promoting agents. This system is modular, in that any number of diverse Shiga toxin effector polypeptides may be associated with a PD-L1 binding region(s) to produce variants of the PD-L1 targeting molecule with different functional characterstics, such as, e.g. de-immunized effectors for applications involving administration of the PD-L1 targeting molecule to a chordate, reduced protease-cleavage sensitive effectors to improve stability particularly in vivo, and effectors comprising a CD8+ T-cell epitope for immunotherapy applications.

PD-L1 Targeting Molecules Comprising a Wild-Type Shiga Toxin Effector Polypeptide and Optionally a Disrupted, Furin-Cleavage Site and/or a Carboxy-Terminal Endoplasmic Reticulum Retention/Retrieval Signal Motif

In some embodiments, PD-L1 targeting molecules are provided that each comprise (i) a binding region capable of specifically binding an extracellular part of a PD-L1 target molecule, (ii) a Shiga toxin effector polypeptide consisting essentially of a wild-type amino acid sequence (see e.g. WO 2014164693, WO 2015/138435, WO 2015/138452, WO 2017/019623).

In some embodiments, the PD-L1 targeting molecule comprises a Shiga toxin effector polypeptide comprising (iii) a disrupted furin-cleavage site at the carboxy-terminus of its Shiga toxin A1 fragment region (see e.g. WO 2015191764). For some embodiments, the PD-L1 targeting molecule is capable of one or more the following: entering a cell, inhibiting a ribosome function, causing cytostasis, and/or causing cell death. For some embodiments, the PD-L1 targeting molecule is capable when introduced to cells of exhibiting a cytotoxicity comparable or better than a reference molecule, such as, e.g., a molecule similar to the PD-L1 targeting molecule except for all of its Shiga toxin effector polypeptide components comprise a wild-type Shiga toxin furin-cleavage site at the carboxy terminus of its A1 fragment region.

In some embodiments, the disrupted furin-cleavage site comprises one or more mutations, relative to a wild-type Shiga toxin A Subunit, the mutation altering at least one amino acid residue in a region natively positioned at 248-251 of the A Subunit of Shiga toxin (SEQ ID NOs: 1-2 and 4-6), or at 247-250 of the A Subunit of Shiga-like toxin 2 (SEQ ID NOs: 3 and 7-18), or the equivalent region in a Shiga toxin A Subunit or derivative thereof. In some embodiments, the disrupted furin-cleavage site comprises one or more mutations, relative to a wild-type Shiga toxin A Subunit, in a minimal furin cleavage site of the furin-cleavage site. In some embodiments the minimal furin cleavage site is represented by the consensus amino acid sequence R/Y-x-x-R (SEQ ID NO: 159) and/or R-x-x-R (SEQ ID NO: 160).

In some embodiments, the disrupted furin-cleavage site comprises an amino acid residue substitution in the furin-cleavage site relative to a wild-type Shiga toxin A Subunit. In some embodiments, the substitution of the amino acid residue in the furin-cleavage site is of an arginine residue with a non-positively charged, amino acid residue selected from the group consisting of: alanine, glycine, proline, serine, threonine, aspartate, asparagine, glutamate, glutamine, cysteine, isoleucine, leucine, methionine, valine, phenylalanine, tryptophan, and tyrosine. In some embodiments, the substitution of the amino acid residue in the furin-cleavage site is of an arginine residue with a histidine.

In some embodiments, the PD-L1 targeting molecule comprises a molecular moiety located carboxy-terminal to the carboxy-terminus of the Shiga toxin A1 fragment region.

In some embodiments, the binding region sterically covers the carboxy-terminus of the A1 fragment region.

In some embodiments, the molecular moiety sterically covers the carboxy-terminus of the A1 fragment region. In some embodiments, the molecular moiety comprises the binding region.

In some embodiments, the Shiga toxin effector polypeptide is not cytotoxic and the molecular moiety is cytotoxic.

In some embodiments, the PD-L1 targeting molecule comprises a binding region and/or molecular moiety located carboxy-terminal to the carboxy-terminus of the Shiga toxin A1 fragment region. In some embodiments, the mass of the binding region and/or molecular moiety is at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater.

In some embodiments, the PD-L1 targeting molecule comprises a binding region with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the PD-L1 targeting molecule retains the appropriate level of the Shiga toxin biological activity noted herein (e.g., cytotoxicity and/or intracellular routing).

In some embodiments, the binding region is comprised within a relatively large, molecular moiety comprising such as, e.g., a molecular moiety with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the PD-L1 targeting molecule retains the appropriate level of the Shiga toxin biological activity noted herein.

In some embodiments, the PD-L1 targeting molecule is capable when introduced to a chordate of exhibiting improved, in vivo tolerability compared to in vivo tolerability of a reference PD-L1 targeting molecule consisting of the PD-L1 targeting molecule except for all of its Shiga toxin effector polypeptide component(s) each comprise a wild-type Shiga toxin A1 fragment and/or wild-type Shiga toxin furin-cleavage site at the carboxy terminus of its A1 fragment region.

In some embodiments, the PD-L1 targeting molecule is de-immunized due to the furin-cleavage site disruption (see e.g. WO 2015113007; WO 2016196344). For some embodiments, the PD-L1 targeting molecule is capable of exhibiting less relative antigenicity and/or relative immunogenicity as compared to a reference PD-L1 targeting molecule consisting of the PD-L1 targeting molecule except for the furin-cleavage site is wild-type and/or all the Shiga toxin effector polypeptide components consist of a wild-type Shiga toxin A1 fragment.

In some embodiments, the PD-L1 targeting molecule comprises (i) a binding region capable of specifically binding an extracellular part of PD-L1, (ii) a Shiga toxin effector polypeptide consisting essentially of a wild-type amino acid sequence. In some embodiments, the PD-L1 targeting molecule further comprises (iii) a carboxy-terminal, endoplasmic reticulum retention/retrieval signal motif (see e.g. WO 2015138435). In some embodiments, the carboxy-terminal endoplasmic reticulum retention/retrieval signal motif is selected from the group consisting of: KDEL (SEQ ID NO: 111), HDEF (SEQ ID NO: 112), HDEL (SEQ ID NO: 113), RDEF (SEQ ID NO:114), RDEL (SEQ ID NO: 115), WDEL (SEQ ID NO: 116), YDEL (SEQ ID NO: 117), HEEF (SEQ ID NO: 118), HEEL (SEQ ID NO: 119), KEEL (SEQ ID NO: 120), REEL (SEQ ID NO: 121), KAEL (SEQ ID NO: 122), KCEL (SEQ ID NO: 123), KFEL (SEQ ID NO: 124), KGEL (SEQ ID NO: 125), KHEL (SEQ ID NO: 126), KLEL (SEQ ID NO: 127), KNEL (SEQ ID NO: 128), KQEL (SEQ ID NO: 129), KREL (SEQ ID NO: 130), KSEL (SEQ ID NO: 131), KVEL (SEQ ID NO: 132), KWEL (SEQ ID NO: 133), KYEL (SEQ ID NO: 134), KEDL (SEQ ID NO: 135), KIEL (SEQ ID NO: 136), DKEL (SEQ ID NO: 137), FDEL (SEQ ID NO: 138), KDEF (SEQ ID NO: 139), KKEL (SEQ ID NO: 140), HADL (SEQ ID NO: 141), HAEL (SEQ ID NO: 142), HIEL (SEQ ID NO: 143), HNEL (SEQ ID NO: 144), HTEL (SEQ ID NO: 145), KTEL (SEQ ID NO: 146), HVEL (SEQ ID NO: 147), NDEL (SEQ ID NO: 148), QDEL (SEQ ID NO: 149), REDL (SEQ ID NO: 150), RNEL (SEQ ID NO: 151), RTDL (SEQ ID NO: 152), RTEL (SEQ ID NO: 153), SDEL (SEQ ID NO: 154), TDEL (SEQ ID NO: 155), SKEL (SEQ ID NO: 156), STEL (SEQ ID NO: 157), and EDEL (SEQ ID NO: 158).

In some embodiments, the amino-terminus of the Shiga toxin effector polypeptide is at and/or proximal to an amino-terminus of a polypeptide component of the PD-L1 targeting molecule (see e.g. WO 2015138452; US 2016/0177284). In some embodiments, the binding region is not located proximally to the amino-terminus of the PD-L1 targeting molecule relative to the Shiga toxin effector polypeptide. In some embodiments, the binding region and Shiga toxin effector polypeptide are physically arranged or oriented within the PD-L1 targeting molecule such that the binding region is not located proximally to the amino-terminus of the Shiga toxin effector polypeptide. In some embodiments, the binding region is located within the PD-L1 targeting molecule more proximal to the carboxy-terminus of the Shiga toxin effector polypeptide than to the amino-terminus of the Shiga toxin effector polypeptide.

In some embodiments, the binding region and Shiga toxin effector polypeptide are linked together, either directly or indirectly.

PD-L1 Targeting Molecules Comprising a Mutation Relvatice to a Wild-Type Shiga Toxin Providing De-Immunization; an Embedded or Inserted, Heterologous, T-Cell Epitope; a Disrupted, Furin-Cleavage Site; and Optionally Comprising a Carboxy-Terminal Endoplasmic Reticulum Retention/Retrieval Signal Motif

Also provided herein are PD-L1 targeting molecules, each comprising (i) a binding region capable of specifically binding an extracellular part of a PD-L1 target molecule, (ii) a Shiga toxin effector polypeptide comprising one or more mutations relative to a wild-type Shiga toxin amino acid sequence providing de-immunization; an embedded or inserted, heterologous, T-cell epitope; a disrupted, furin-cleavage site; and optionally comprising a carboxy-terminal endoplasmic reticulum retention/retrieval signal motif (see e.g. WO 2016196344, WO 2018/140427).

In some embodiments, the Shiga toxin effector polypeptide comprises one or more mutations relative to a wild-type Shiga toxin amino acid sequence providing de-immunization, such as, e.g., (a) a mutation disrupting at least one, endogenous, B-cell and/or CD4+ T-cell epitope region; (b) a mutation creating at least one inserted or embedded, heterologous, T-cell epitope; and/or a (c) mutations creating both (i) an embedded or inserted, heterologous, T-cell epitope and (ii) a disruption of at least one, endogenous, B-cell and/or T-cell epitope which does not overlap with the embedded or inserted, heterologous, T-cell epitope.

In some embodiments, the PD-L1 targeting molecule comprises a molecular moiety associated with the carboxy-terminus of the Shiga toxin effector polypeptide. In some embodiments, the molecular moiety comprises or consists of the binding region. In some embodiments, the molecular moiety comprises at least one amino acid and the Shiga toxin effector polypeptide is linked to at least one amino acid residue of the molecular moiety. In some embodiments, the molecular moiety and the Shiga toxin effector polypeptide are fused forming a continuous polypeptide.

In some embodiments, the PD-L1 targeting molecule comprises a molecular moiety located carboxy-terminal to the carboxy-terminus of the Shiga toxin A1 fragment region.

In some embodiments, the PD-L1 targeting molecule further comprises a cytotoxic molecular moiety associated with the carboxy-terminus of the Shiga toxin effector polypeptide. For some embodiments, the cytotoxic molecular moiety is a cytotoxic agent, such as, e.g., a small molecule chemotherapeutic agent, anti-neoplastic agent, cytotoxic antibiotic, alkylating agent, antimetabolite, topoisomerase inhibitor, and/or tubulin inhibitor known to the skilled worker and/or described herein. In some embodiments, the cytotoxic molecular moiety is cytotoxic at concentrations of less than 10,000, 5,000, 1,000, 500, or 200 pM.

In some embodiments, the Shiga toxin effector polypeptide is not cytotoxic and the molecular moiety is cytotoxic.

In some embodiments, the PD-L1 targeting molecule and/or its Shiga toxin effector polypeptide is capable of exhibiting subcellular routing efficiency comparable to a reference PD-L1 targeting molecule comprising a wild-type Shiga toxin A1 fragment and lacking a carboxy-terminal, endoplasmic reticulum retention/retrieval signal motif of the KDEL family or wild-type Shiga toxin effector polypeptide.

In some embodiments, the PD-L1 targeting molecule and/or its Shiga toxin effector polypeptide is capable of exhibiting a significant level of intracellular routing activity to the endoplasmic reticulum and/or cytosol from an endosomal starting location of a cell.

In some embodiments, the PD-L1 targeting molecule is capable of delivering an embedded or inserted, heterologous, CD8+ T-cell epitope to a MHC class I presentation pathway of a cell for cell-surface presentation of the epitope bound by a MHC class I molecule.

In some embodiments, the binding region is linked, either directly or indirectly, to the Shiga toxin effector polypeptide by at least one covalent bond which is not a disulfide bond. In some embodiments, the binding region is fused, either directly or indirectly, to the carboxy-terminus of the Shiga toxin effector polypeptide to form a single, continuous polypeptide. In some embodiments, the binding region is an immunoglobulin-type binding region.

In some embodiments, the disrupted furin-cleavage site comprises one or more mutations in the minimal, furin-cleavage site relative to a wild-type Shiga toxin A Subunit. In some embodiments, the disrupted furin-cleavage site is not an amino-terminal truncation of sequences that overlap with part or all of at least one amino acid residue of the minimal furin-cleavage site. In some embodiments, the mutation in the minimal, furin-cleavage site is an amino acid deletion, insertion, and/or substitution of at least one amino acid residue in the R/Y-x-x-R (SEQ ID NO: 159) furin cleavage site. In some embodiments, the disrupted furin-cleavage site comprises at least one mutation relative to a wild-type Shiga toxin A Subunit, the mutation altering at least one amino acid residue in the region natively positioned 1) at 248-251 of the A Subunit of Shiga toxin (SEQ ID NOs: 1-2 and 4-6), or 2) at 247-250 of the A Subunit of Shiga-like toxin 2 (SEQ ID NOs: 3 and 7-18), or the equivalent amino acid sequence position in any Shiga toxin A Subunit. In some embodiments, the mutation is an amino acid residue substitution of an arginine residue with a non-positively charged, amino acid residue.

In some embodiments, the PD-L1 targeting molecule is capable, when introduced to cells, of exhibiting cytotoxicity comparable to a cytotoxicity of a reference molecule, such as, e.g., a molecule similar to the PD-L1 targeting molecule except for all of its Shiga toxin effector polypeptide component(s) each comprise a wild-type Shiga toxin A1 fragment.

In some embodiments, the binding region comprises the peptide or polypeptide shown in any one of SEQ ID NOs: 19-40.

In some embodiments, the binding region sterically covers the carboxy-terminus of the A1 fragment region.

In some embodiments, the molecular moiety sterically covers the carboxy-terminus of the A1 fragment region. In some embodiments, the molecular moiety comprises the binding region.

In some embodiments, the PD-L1 targeting molecule comprises a binding region and/or molecular moiety located carboxy-terminal to the carboxy-terminus of the Shiga toxin A1 fragment region. In some embodiments, the mass of the binding region and/or molecular moiety is at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater.

In some embodiments, the PD-L1 targeting molecule comprises a binding region with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the PD-L1 targeting molecule retains the appropriate level of the Shiga toxin biological activity noted herein (e.g., cytotoxicity and/or intracellular routing).

In some embodiments, the binding region is comprised within a relatively large, molecular moiety comprising such as, e.g., a molecular moiety with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the PD-L1 targeting molecule retains the appropriate level of the Shiga toxin biological activity noted herein.

In some embodiments, the amino-terminus of the Shiga toxin effector polypeptide is at and/or proximal to an amino-terminus of a polypeptide component of the PD-L1 targeting molecule. In some embodiments, the binding region is not located proximally to the amino-terminus of the PD-L1 targeting molecule relative to the Shiga toxin effector polypeptide. In some embodiments, the binding region and Shiga toxin effector polypeptide are physically arranged or oriented within the PD-L1 targeting molecule such that the binding region is not located proximally to the amino-terminus of the Shiga toxin effector polypeptide. In some embodiments, the binding region is located within the PD-L1 targeting molecule more proximal to the carboxy-terminus of the Shiga toxin effector polypeptide than to the amino-terminus of the Shiga toxin effector polypeptide. For some embodiments, the PD-L1 targeting molecule is capable when introduced to cells of exhibiting cytotoxicity that is greater than that of a third PD-L1 targeting molecule having an amino-terminus and comprising the binding region and the Shiga toxin effector polypeptide which is not positioned at or proximal to the amino-terminus of the third PD-L1 targeting molecule. For some embodiments, the PD-L1 targeting molecule exhibits cytotoxicity with better optimized, cytotoxic potency, such as, e.g., 4-fold, 5-fold, 6-fold, 9-fold, or greater cytotoxicity as compared to the cytotoxicity of the third PD-L1 targeting molecule. For some embodiments, the cytotoxicity of the PD-L1 targeting molecule to a population of target positive cells is 3-fold, 4-fold, 5-fold, 6-fold, 7-fold, 8-fold, 9-fold, 10-fold or greater than the cytotoxicity of the third PD-L1 targeting molecule to a second population of target positive cells as assayed by CD₅₀ values. In some embodiments, the third PD-L1 targeting molecule does not comprise any carboxy-terminal, endoplasmic reticulum retention/retrieval signal motif of the KDEL family.

In some embodiments, the amino-terminus of the Shiga toxin effector polypeptide is at and/or proximal to an amino-terminus of a polypeptide component of the PD-L1 targeting molecule. In some embodiments, the binding region is not located proximally to the amino-terminus of the PD-L1 targeting molecule relative to the Shiga toxin effector polypeptide. In some embodiments, the binding region and Shiga toxin effector polypeptide are physically arranged or oriented within the PD-L1 targeting molecule such that the binding region is not located proximally to the amino-terminus of the Shiga toxin effector polypeptide. In some embodiments, the binding region is located within the PD-L1 targeting molecule more proximal to the carboxy-terminus of the Shiga toxin effector polypeptide than to the amino-terminus of the Shiga toxin effector polypeptide. For some embodiments, the PD-L1 targeting molecule is not cytotoxic and is capable, when introduced to cells, of exhibiting a greater subcellular routing efficiency from an extracellular space to a subcellular compartment of an endoplasmic reticulum and/or cytosol as compared to the cytotoxicity of a third PD-L1 targeting molecule having an amino-terminus and comprising the binding region and the Shiga toxin effector polypeptide which is not positioned at or proximal to the amino-terminus of the third PD-L1 targeting molecule. In some embodiments, the third PD-L1 targeting molecule does not comprise any carboxy-terminal, endoplasmic reticulum retention/retrieval signal motif of the KDEL family.

In some embodiments, the amino-terminus of the Shiga toxin effector polypeptide is at and/or proximal to an amino-terminus of a polypeptide component of the PD-L1 targeting molecule. In some embodiments, the binding region is not located proximally to the amino-terminus of the PD-L1 targeting molecule relative to the Shiga toxin effector polypeptide. In some embodiments, the binding region and Shiga toxin effector polypeptide are physically arranged or oriented within the PD-L1 targeting molecule such that the binding region is not located proximally to the amino-terminus of the Shiga toxin effector polypeptide. In some embodiments, the binding region is located within the PD-L1 targeting molecule more proximal to the carboxy-terminus of the Shiga toxin effector polypeptide than to the amino-terminus of the Shiga toxin effector polypeptide. For some embodiments, the PD-L1 targeting molecule exhibits low cytotoxic potency (i.e. is not capable when introduced to certain positive target cell types of exhibiting a cytotoxicity greater than 1% cell death of a cell population at a PD-L1 targeting molecule concentration of 1000 nM, 500 nM, 100 nM, 75 nM, or 50 nM) and is capable when introduced to cells of exhibiting a greater subcellular routing efficiency from an extracellular space to a subcellular compartment of an endoplasmic reticulum and/or cytosol as compared to the cytotoxicity of a third PD-L1 targeting molecule having an amino-terminus and comprising the binding region and the Shiga toxin effector polypeptide which is not positioned at or proximal to the amino-terminus of the third PD-L1 targeting molecule. In some embodiments, the third PD-L1 targeting molecule does not comprise any carboxy-terminal, endoplasmic reticulum retention/retrieval signal motif of the KDEL family.

In some embodiments, a PD-L1 targeting molecule, or a polypeptide component thereof, comprises a carboxy-terminal, endoplasmic reticulum retention/retrieval signal motif of a member of the KDEL family. For some embodiments, the carboxy-terminal endoplasmic reticulum retention/retrieval signal motif is selected from the group consisting of: KDEL (SEQ ID NO: 111), HDEF (SEQ ID NO: 112), HDEL (SEQ ID NO: 113), RDEF (SEQ ID NO:114), RDEL (SEQ ID NO: 115), WDEL (SEQ ID NO: 116), YDEL (SEQ ID NO: 117), HEEF (SEQ ID NO: 118), HEEL (SEQ ID NO: 119), KEEL (SEQ ID NO: 120), REEL (SEQ ID NO: 121), KAEL (SEQ ID NO: 122), KCEL (SEQ ID NO: 123), KFEL (SEQ ID NO: 124), KGEL (SEQ ID NO: 125), KHEL (SEQ ID NO: 126), KLEL (SEQ ID NO: 127), KNEL (SEQ ID NO: 128), KQEL (SEQ ID NO: 129), KREL (SEQ ID NO: 130), KSEL (SEQ ID NO: 131), KVEL (SEQ ID NO: 132), KWEL (SEQ ID NO: 133), KYEL (SEQ ID NO: 134), KEDL (SEQ ID NO: 135), KIEL (SEQ ID NO: 136), DKEL (SEQ ID NO: 137), FDEL (SEQ ID NO: 138), KDEF (SEQ ID NO: 139), KKEL (SEQ ID NO: 140), HADL (SEQ ID NO: 141), HAEL (SEQ ID NO: 142), HIEL (SEQ ID NO: 143), HNEL (SEQ ID NO: 144), HTEL (SEQ ID NO: 145), KTEL (SEQ ID NO: 146), HVEL (SEQ ID NO: 147), NDEL (SEQ ID NO: 148), QDEL (SEQ ID NO: 149), REDL (SEQ ID NO: 150), RNEL (SEQ ID NO: 151), RTDL (SEQ ID NO: 152), RTEL (SEQ ID NO: 153), SDEL (SEQ ID NO: 154), TDEL (SEQ ID NO: 155), SKEL (SEQ ID NO: 156), STEL (SEQ ID NO: 157), and EDEL (SEQ ID NO: 158). In some embodiments, the PD-L1 targeting molecule is capable when introduced to cells of exhibiting cytotoxicity that is greater than that of a reference molecule which is consists of the PD-L1 targeting molecule except for it does not comprise any carboxy-terminal, endoplasmic reticulum retention/retrieval signal motif of the KDEL family.

### PD-L1 Targeting Molecules Comprising a De-Immunized Shiga Toxin Effector Polypeptide Comprising an Embedded or Inserted, Heterologous, T-Cell Epitope and a Non-Overlapping De-Immunized Sub-Region

Also provided herein are PD-L1 targeting molecules, each comprising (i) a binding region capable of specifically binding an extracellular part of a PD-L1 target molecule and (ii) a de-immunized, Shiga toxin effector polypeptide. For example, in some embodiments, the PD-L1 targeting molecule comprises (i) a binding region capable of specifically binding an extracellular part of PD-L1 and (ii) a de-immunized, Shiga toxin effector polypeptide comprising at least one inserted or embedded, heterologous epitope (a) and at least one disrupted, endogenous, B-cell and/or CD4+ T-cell epitope region (b), wherein the heterologous epitope does not overlap with the embedded or inserted, heterologous, T-cell epitope. For some embodiments, the Shiga toxin effector polypeptide is capable of exhibiting at least one Shiga toxin effector function, such as, e.g., directing intracellular routing to the endoplasmic reticulum and/or cytosol of a cell in which the polypeptide is present, inhibiting a ribosome function, enzymatically inactivating a ribosome, causing cytostasis, and/or causing cytotoxicity. In some embodiments, the heterologous, T-cell epitope is a CD8+ T-cell epitope, such as, e.g., with regard to a human immune system. For some embodiments, the heterologous, T-cell epitope is capable of being presented by a MHC class I molecule of a cell. In some embodiments, the PD-L1 targeting molecule is capable of one or more the following: entering a cell, inhibiting a ribosome function, causing cytostasis, causing cell death, and/or delivering the embedded or inserted, heterologous, T-cell epitope to a MHC class I molecule for presentation on a cellular surface. For some embodiments, the PD-L1 targeting molecule is capable when introduced to cells of exhibiting a cytotoxicity comparable or better than a reference molecule, such as, e.g., a PD-L1 targeting molecule consisting of the PD-L1 targeting molecule except for all of its Shiga toxin effector polypeptide component(s) each comprise a wild-type Shiga toxin A1 fragment.

In some embodiments, the PD-L1 targeting molecule comprises a molecular moiety located carboxy-terminal to the carboxy-terminus of the Shiga toxin A1 fragment region.

In some embodiments, the PD-L1 targeting molecule is capable when introduced to a chordate of exhibiting improved in vivo tolerability and/or stability compared to a reference molecule, such as, e.g., a PD-L1 targeting molecule consisting of the PD-L1 targeting molecule except for all of its Shiga toxin effector polypeptide component(s) each comprise a wild-type Shiga toxin A1 fragment and/or wild-type Shiga toxin furin-cleavage site at the carboxy terminus of its A1 fragment region. In some embodiments, the Shiga toxin effector polypeptide is not cytotoxic and the molecular moiety is cytotoxic.

In some embodiments, the binding region and Shiga toxin effector polypeptide are linked together, either directly or indirectly.

In some embodiments, the PD-L1 targeting molecule is capable of exhibiting (i) a catalytic activity level comparable to a wild-type Shiga toxin A1 fragment or wild-type Shiga toxin effector polypeptide, (ii) a ribosome inhibition activity with a half-maximal inhibitory concentration (IC₅₀) value of 10,000 picomolar or less, and/or (iii) a significant level of Shiga toxin catalytic activity.

In some embodiments, the PD-L1 targeting molecule and/or its Shiga toxin effector polypeptide is capable of exhibiting subcellular routing efficiency comparable to a reference PD-L1 targeting molecule comprising a wild-type Shiga toxin A1 fragment or wild-type Shiga toxin effector polypeptide and/or capable of exhibiting a significant level of intracellular routing activity to the endoplasmic reticulum and/or cytosol from an endosomal starting location of a cell.

In some embodiments, whereby administration of the PD-L1 targeting molecule to a cell physically coupled with the extracellular PD-L1 of the PD-L1 targeting molecule's binding region, the PD-L1 targeting molecule is capable of causing death of the cell. In some embodiments, administration of the PD-L1 targeting molecule to two different populations of cell types which differ with respect to the presence or level of the extracellular PD-L1, the PD-L1 targeting molecule is capable of causing cell death to the cell-types physically coupled with an extracellular PD-L1 bound by the cytotoxic PD-L1 targeting molecule's binding region at a CD₅₀ at least three times or less than the CD₅₀ to cell types which are not physically coupled with an extracellular PD-L1 bound by the PD-L1 targeting molecule's binding region. For some embodiments, whereby administration of the PD-L1 targeting molecule to a first populations of cells whose members are physically coupled to extracellular PD-L1 bound by the PD-L1 targeting molecule's binding region, and a second population of cells whose members are not physically coupled to any extracellular PD-L1 bound by the binding region, the cytotoxic effect of the PD-L1 targeting molecule to members of said first population of cells relative to members of said second population of cells is at least 3-fold greater. For some embodiments, whereby administration of the PD-L1 targeting molecule to a first populations of cells whose members are physically coupled to a significant amount of the extracellular PD-L1 bound by the PD-L1 targeting molecule's binding region, and a second population of cells whose members are not physically coupled to a significant amount of any extracellular PD-L1 bound by the binding region, the cytotoxic effect of the PD-L1 targeting molecule to members of said first population of cells relative to members of said second population of cells is at least 3-fold greater. For some embodiments, whereby administration of the PD-L1 targeting molecule to a first population of target biomolecule positive cells, and a second population of cells whose members do not express a significant amount of a target biomolecule of the PD-L1 targeting molecule's binding region at a cellular surface, the cytotoxic effect of the PD-L1 targeting molecule to members of the first population of cells relative to members of the second population of cells is at least 3-fold greater.

In some embodiments, the PD-L1 targeting molecule is capable when introduced to cells of exhibiting a cytotoxicity with a half-maximal inhibitory concentration (CD₅₀) value of 300 nM or less and/or capable of exhibiting a significant level of Shiga toxin cytotoxicity.

In some embodiments, the PD-L1 targeting molecule is capable of delivering an embedded or inserted, heterologous, CD8+ T-cell epitope to a MHC class I presentation pathway of a cell for cell-surface presentation of the epitope bound by a MHC class I molecule.

In some embodiments, the PD-L1 targeting molecule comprises a molecular moiety associated with the carboxy-terminus of the Shiga toxin effector polypeptide. In some embodiments, the molecular moiety comprises or consists of the binding region. In some embodiments, the molecular moiety comprises at least one amino acid and the Shiga toxin effector polypeptide is linked to at least one amino acid residue of the molecular moiety. In some embodiments, the molecular moiety and the Shiga toxin effector polypeptide are fused forming a continuous polypeptide.

In some embodiments, the PD-L1 targeting molecule further comprises a cytotoxic molecular moiety associated with the carboxy-terminus of the Shiga toxin effector polypeptide. For some embodiments, the cytotoxic molecular moiety is a cytotoxic agent, such as, e.g., a small molecule chemotherapeutic agent, anti-neoplastic agent, cytotoxic antibiotic, alkylating agent, antimetabolite, topoisomerase inhibitor, and/or tubulin inhibitor known to the skilled worker and/or described herein. For some embodiments, the cytotoxic molecular moiety is cytotoxic at concentrations of less than 10,000, 5,000, 1,000, 500, or 200 pM.

In some embodiments, the disrupted furin-cleavage site comprises one or more mutations in the minimal, furin-cleavage site relative to a wild-type Shiga toxin A Subunit. In some embodiments, the disrupted furin-cleavage site is not an amino-terminal truncation of sequences that overlap with part or all of at least one amino acid residue of the minimal furin-cleavage site. In some embodiments, the mutation in the minimal, furin-cleavage site is an amino acid deletion, insertion, and/or substitution of at least one amino acid residue in the R/Y-x-x-R furin cleavage site (SEQ ID NO: 159). In some embodiments, the disrupted furin-cleavage site comprises at least one mutation relative to a wild-type Shiga toxin A Subunit, the mutation altering at least one amino acid residue in the region natively positioned 1) at 248-251 of the A Subunit of Shiga toxin (SEQ ID NOs: 1-2 and 4-6), or 2) at 247-250 of the A Subunit of Shiga-like toxin 2 (SEQ ID NOs: 3 and 7-18), or the equivalent amino acid sequence position in any Shiga toxin A Subunit. In some embodiments, the mutation is an amino acid residue substitution of an arginine residue with a non-positively charged, amino acid residue.

In some embodiments, the PD-L1 targeting molecule is capable when introduced to cells of exhibiting cytotoxicity comparable to a cytotoxicity of a reference molecule, such as, e.g., a PD-L1 targeting molecule consisting of the PD-L1 targeting molecule except for all of its Shiga toxin effector polypeptide component(s) each comprise a wild-type Shiga toxin A1 fragment.

In some embodiments, the binding region comprises the peptide or polypeptide shown in any one of SEQ ID NOs: 19-40.

In some embodiments, the binding region sterically covers the carboxy-terminus of the A1 fragment region.

In some embodiments, the molecular moiety sterically covers the carboxy-terminus of the A1 fragment region. In some embodiments, the molecular moiety comprises the binding region.

In some embodiments, the PD-L1 targeting molecule comprises a binding region and/or molecular moiety located carboxy-terminal to the carboxy-terminus of the Shiga toxin A1 fragment region. In some embodiments, the mass of the binding region and/or molecular moiety is at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater.

In some embodiments, the PD-L1 targeting molecule comprises a binding region with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the PD-L1 targeting molecule retains the appropriate level of the Shiga toxin biological activity noted herein (e.g., cytotoxicity and/or intracellular routing).

In some embodiments, the binding region is comprised within a relatively large, molecular moiety comprising such as, e.g., a molecular moiety with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the PD-L1 targeting molecule retains the appropriate level of the Shiga toxin biological activity noted herein.

In some embodiments, the amino-terminus of the Shiga toxin effector polypeptide is at and/or proximal to an amino-terminus of a polypeptide component of the PD-L1 targeting molecule. In some embodiments, the binding region is not located proximally to the amino-terminus of the PD-L1 targeting molecule relative to the Shiga toxin effector polypeptide. In some embodiments, the binding region and Shiga toxin effector polypeptide are physically arranged or oriented within the PD-L1 targeting molecule such that the binding region is not located proximally to the amino-terminus of the Shiga toxin effector polypeptide. In some embodiments, the binding region is located within the PD-L1 targeting molecule more proximal to the carboxy-terminus of the Shiga toxin effector polypeptide than to the amino-terminus of the Shiga toxin effector polypeptide. For some embodiments, the PD-L1 targeting molecule is capable when introduced to cells of exhibiting cytotoxicity that is greater than that of a fourth PD-L1 targeting molecule having an amino-terminus and comprising the binding region and the Shiga toxin effector polypeptide which is not positioned at or proximal to the amino-terminus of the fourth PD-L1 targeting molecule. For some embodiments, the PD-L1 targeting molecule exhibits cytotoxicity with better optimized, cytotoxic potency, such as, e.g., 4-fold, 5-fold, 6-fold, 9-fold, or greater cytotoxicity as compared to the cytotoxicity of the fourth PD-L1 targeting molecule. For some embodiments, the cytotoxicity of the PD-L1 targeting molecule to a population of target positive cells is 3-fold, 4-fold, 5-fold, 6-fold, 7-fold, 8-fold, 9-fold, 10-fold or greater than the cytotoxicity of the fourth PD-L1 targeting molecule to a second population of target positive cells as assayed by CD₅₀ values. In some embodiments, the fourth PD-L1 targeting molecule does not comprise any carboxy-terminal, endoplasmic reticulum retention/retrieval signal motif of the KDEL family.

In some embodiments, the amino-terminus of the Shiga toxin effector polypeptide is at and/or proximal to an amino-terminus of a polypeptide component of the PD-L1 targeting molecule. In some embodiments, the binding region is not located proximally to the amino-terminus of the PD-L1 targeting molecule relative to the Shiga toxin effector polypeptide. In some embodiments, the binding region and Shiga toxin effector polypeptide are physically arranged or oriented within the PD-L1 targeting molecule such that the binding region is not located proximally to the amino-terminus of the Shiga toxin effector polypeptide. In some embodiments, the binding region is located within the PD-L1 targeting molecule more proximal to the carboxy-terminus of the Shiga toxin effector polypeptide than to the amino-terminus of the Shiga toxin effector polypeptide. For some embodiments, the PD-L1 targeting molecule is not cytotoxic and is capable when introduced to cells of exhibiting a greater subcellular routing efficiency from an extracellular space to a subcellular compartment of an endoplasmic reticulum and/or cytosol as compared to the cytotoxicity of a fourth PD-L1 targeting molecule having an amino-terminus and comprising the binding region and the Shiga toxin effector polypeptide which is not positioned at or proximal to the amino-terminus of the fourth PD-L1 targeting molecule. In some embodiments, the fourth PD-L1 targeting molecule does not comprise any carboxy-terminal, endoplasmic reticulum retention/retrieval signal motif of the KDEL family.

In some embodiments, the amino-terminus of the Shiga toxin effector polypeptide is at and/or proximal to an amino-terminus of a polypeptide component of the PD-L1 targeting molecule. In some embodiments, the binding region is not located proximally to the amino-terminus of the PD-L1 targeting molecule relative to the Shiga toxin effector polypeptide. In some embodiments, the binding region and Shiga toxin effector polypeptide are physically arranged or oriented within the PD-L1 targeting molecule such that the binding region is not located proximally to the amino-terminus of the Shiga toxin effector polypeptide. In some embodiments, the binding region is located within the PD-L1 targeting molecule more proximal to the carboxy-terminus of the Shiga toxin effector polypeptide than to the amino-terminus of the Shiga toxin effector polypeptide. In some embodiments, the PD-L1 targeting molecule exhibits low cytotoxic potency (i.e. is not capable when introduced to certain positive target cell types of exhibiting a cytotoxicity greater than 1% cell death of a cell population at a PD-L1 targeting molecule concentration of 1000 nM, 500 nM, 100 nM, 75 nM, or 50 nM) and is capable, when introduced to cells, of exhibiting a greater subcellular routing efficiency from an extracellular space to a subcellular compartment of an endoplasmic reticulum and/or cytosol as compared to the cytotoxicity of a fourth PD-L1 targeting molecule having an amino-terminus and comprising the binding region and the Shiga toxin effector polypeptide which is not positioned at or proximal to the amino-terminus of the fourth PD-L1 targeting molecule. In some embodiments, the fourth PD-L1 targeting molecule does not comprise any carboxy-terminal, endoplasmic reticulum retention/retrieval signal motif of the KDEL family.

In some embodiments, the PD-L1 targeting molecule, or a polypeptide component thereof, comprises a carboxy-terminal, endoplasmic reticulum retention/retrieval signal motif of a member of the KDEL family. For some embodiments, the carboxy-terminal endoplasmic reticulum retention/retrieval signal motif is selected from the group consisting of: KDEL (SEQ ID NO: 111), HDEF (SEQ ID NO: 112), HDEL (SEQ ID NO: 113), RDEF (SEQ ID NO:114), RDEL (SEQ ID NO: 115), WDEL (SEQ ID NO: 116), YDEL (SEQ ID NO: 117), HEEF (SEQ ID NO: 118), HEEL (SEQ ID NO: 119), KEEL (SEQ ID NO: 120), REEL (SEQ ID NO: 121), KAEL (SEQ ID NO: 122), KCEL (SEQ ID NO: 123), KFEL (SEQ ID NO: 124), KGEL (SEQ ID NO: 125), KHEL (SEQ ID NO: 126), KLEL (SEQ ID NO: 127), KNEL (SEQ ID NO: 128), KQEL (SEQ ID NO: 129), KREL (SEQ ID NO: 130), KSEL (SEQ ID NO: 131), KVEL (SEQ ID NO: 132), KWEL (SEQ ID NO: 133), KYEL (SEQ ID NO: 134), KEDL (SEQ ID NO: 135), KIEL (SEQ ID NO: 136), DKEL (SEQ ID NO: 137), FDEL (SEQ ID NO: 138), KDEF (SEQ ID NO: 139), KKEL (SEQ ID NO: 140), HADL (SEQ ID NO: 141), HAEL (SEQ ID NO: 142), HIEL (SEQ ID NO: 143), HNEL (SEQ ID NO: 144), HTEL (SEQ ID NO: 145), KTEL (SEQ ID NO: 146), HVEL (SEQ ID NO: 147), NDEL (SEQ ID NO: 148), QDEL (SEQ ID NO: 149), REDL (SEQ ID NO: 150), RNEL (SEQ ID NO: 151), RTDL (SEQ ID NO: 152), RTEL (SEQ ID NO: 153), SDEL (SEQ ID NO: 154), TDEL (SEQ ID NO: 155), SKEL (SEQ ID NO: 156), STEL (SEQ ID NO: 157), and EDEL (SEQ ID NO: 158). In some embodiments, the PD-L1 targeting molecule is capable when introduced to cells of exhibiting cytotoxicity that is greater than that of a reference PD-L1 targeting molecule consisting of the PD-L1 targeting molecule except for it does not comprise any carboxy-terminal, endoplasmic reticulum retention/retrieval signal motif of the KDEL family. In some embodiments, the PD-L1 targeting molecule is capable of exhibiting a cytotoxicity with better optimized, cytotoxic potency, such as, e.g., 4-fold, 5-fold, 6-fold, 9-fold, or greater cytotoxicity as compared to a reference molecule, such as, e.g. a second PD-L1 targeting molecule comprising the same binding region and Shiga toxin effector polypeptide but wherein the Shiga toxin effector poyleptide is not positioned at or proximal to the amino-terminus of the molecule. In some embodiments, the cytotoxicity of the PD-L1 targeting molecule to a population of target positive cells is 3-fold, 4-fold, 5-fold, 6-fold, 7-fold, 8-fold, 9-fold, 10-fold or greater than the cytotoxicity of the reference PD-L1 targeting molecule to a second population of target positive cells as assayed by CD₅₀ values.

### PD-L1 Targeting Molecules Comprising a Carboxy-Terminal Endoplasmic Reticulum Retention/Retrieval Signal Motif and a Shiga Toxin Effector Polypeptide Comprising an Embedded or Inserted, Heterologous, T-Cell Epitope

Also provided herein are PD-L1 targeting molecules, each comprising (i) a binding region capable of specifically binding an extracellular part of a PD-L1 target molecule; (ii) a Shiga toxin effector polypeptide comprising an inserted or embedded, heterologous, epitope; and (iii) a carboxy-terminal, endoplasmic reticulum retention/retrieval signal motif. In some embodiments, the PD-L1 targeting molecule comprises (a) a binding region capable of specifically binding an extracellular part of a PD-L1 target molecule; (b) a Shiga toxin effector polypeptide comprising an embedded or inserted, heterologous epitope; and (c) a carboxy-terminal, endoplasmic reticulum retention/retrieval signal motif of a member of the KDEL family. In some embodiments, the Shiga toxin effector polypeptide is capable of exhibiting at least one Shiga toxin effector function, such as, e.g., directing intracellular routing to the endoplasmic reticulum and/or cytosol of a cell in which the polypeptide is present, inhibiting a ribosome function, enzymatically inactivating a ribosome, causing cytostasis, and/or causing cytotoxicity. In some embodiments, the heterologous, T-cell epitope is a CD8+ T-cell epitope, such as, e.g., with regard to a human immune system. In some embodiments, the heterologous, T-cell epitope is capable of being presented by a MHC class I molecule of a cell. In some embodiments, the PD-L1 targeting molecule is capable of one or more the following: entering a cell, inhibiting a ribosome function, causing cytostasis, causing cell death, and/or delivering the embedded or inserted, heterologous, T-cell epitope to a MHC class I molecule for presentation on a cellular surface.

In some embodiments, the carboxy-terminal endoplasmic reticulum retention/retrieval signal motif is selected from the group consisting of: KDEL (SEQ ID NO: 111), HDEF (SEQ ID NO: 112), HDEL (SEQ ID NO: 113), RDEF (SEQ ID NO:114), RDEL (SEQ ID NO: 115), WDEL (SEQ ID NO: 116), YDEL (SEQ ID NO: 117), HEEF (SEQ ID NO: 118), HEEL (SEQ ID NO: 119), KEEL (SEQ ID NO: 120), REEL (SEQ ID NO: 121), KAEL (SEQ ID NO: 122), KCEL (SEQ ID NO: 123), KFEL (SEQ ID NO: 124), KGEL (SEQ ID NO: 125), KHEL (SEQ ID NO: 126), KLEL (SEQ ID NO: 127), KNEL (SEQ ID NO: 128), KQEL (SEQ ID NO: 129), KREL (SEQ ID NO: 130), KSEL (SEQ ID NO: 131), KVEL (SEQ ID NO: 132), KWEL (SEQ ID NO: 133), KYEL (SEQ ID NO: 134), KEDL (SEQ ID NO: 135), KIEL (SEQ ID NO: 136), DKEL (SEQ ID NO: 137), FDEL (SEQ ID NO: 138), KDEF (SEQ ID NO: 139), KKEL (SEQ ID NO: 140), HADL (SEQ ID NO: 141), HAEL (SEQ ID NO: 142), HIEL (SEQ ID NO: 143), HNEL (SEQ ID NO: 144), HTEL (SEQ ID NO: 145), KTEL (SEQ ID NO: 146), HVEL (SEQ ID NO: 147), NDEL (SEQ ID NO: 148), QDEL (SEQ ID NO: 149), REDL (SEQ ID NO: 150), RNEL (SEQ ID NO: 151), RTDL (SEQ ID NO: 152), RTEL (SEQ ID NO: 153), SDEL (SEQ ID NO: 154), TDEL (SEQ ID NO: 155), SKEL (SEQ ID NO: 156), STEL (SEQ ID NO: 157), and EDEL (SEQ ID NO: 158).

In some embodiments, the embedded or inserted, heterologous, T-cell epitope disrupts the endogenous, B-cell and/or T-cell epitope region selected from the group of natively positioned Shiga toxin A Subunit regions consisting of: (i) 1-15 of any one of SEQ ID NOs: 1-2 and 4-6; 3-14 of any one of SEQ ID NOs: 3 and 7-18; 26-37 of any one of SEQ ID NOs: 3 and 7-18; 27-37 of any one of SEQ ID NOs: 1-2 and 4-6; 39-48 of any one of SEQ ID NOs: 1-2 and 4-6; 42-48 of any one of SEQ ID NOs: 3 and 7-18; and 53-66 of any one of SEQ ID NOs: 1-18, or the equivalent region in a Shiga toxin A Subunit or derivative thereof; (ii) 94-115 of any one of SEQ ID NOs: 1-18; 141-153 of any one of SEQ ID NOs: 1-2 and 4-6; 140-156 of any one of SEQ ID NOs: 3 and 7-18; 179-190 of any one of SEQ ID NOs: 1-2 and 4-6; 179-191 of any one of SEQ ID NOs: 3 and 7-18; 204 of SEQ ID NO:3; 205 of any one of SEQ ID NOs: 1-2 and 4-6; and 210-218 of any one of SEQ ID NOs: 3 and 7-18, or the equivalent region in a Shiga toxin A Subunit or derivative thereof; and (iii) 240-260 of any one of SEQ ID NOs: 3 and 7-18; 243-257 of any one of SEQ ID NOs: 1-2 and 4-6; 254-268 of any one of SEQ ID NOs: 1-2 and 4-6; 262-278 of any one of SEQ ID NOs: 3 and 7-18; 281-297 of any one of SEQ ID NOs: 3 and 7-18; and 285-293 of any one of SEQ ID NOs: 1-2 and 4-6, or the equivalent region in a Shiga toxin A Subunit or derivative thereof.

In some embodiments, the heterologous epitope is a CD8+ T-cell epitope capable of being presented by a MHC class I molecule of a cell. In some embodiments, the heterologous epitope in is embedded and replaces an equivalent number of amino acid residues in a wild-type Shiga toxin polypeptide region such that the Shiga toxin effector polypeptide has the same total number of amino acid residues as does the wild-type Shiga toxin polypeptide region from which it is derived. In some embodiments of any of the above, the Shiga toxin effector polypeptide is capable of exhibiting at least one Shiga toxin effector function selected from: directing intracellular routing to a cytosol of a cell in which the polypeptide is present, inhibiting a ribosome function, enzymatically inactivating a ribosome, and cytotoxicity.

In some embodiments, the PD-L1 targeting molecule is capable when introduced to cells of exhibiting cytotoxicity that is greater than that of a fifth PD-L1 targeting molecule consisting of the PD-L1 targeting molecule except for it does not comprise any carboxy-terminal, endoplasmic reticulum retention/retrieval signal motif of the KDEL family. In some embodiments, the PD-L1 targeting molecule is capable of exhibiting a cytotoxicity with better optimized, cytotoxic potency, such as, e.g., 4-fold, 5-fold, 6-fold, 9-fold, or greater cytotoxicity as compared to the fifth PD-L1 targeting molecule. In some embodiments, the cytotoxicity of the PD-L1 targeting molecule to a population of target positive cells is 3-fold, 4-fold, 5-fold, 6-fold, 7-fold, 8-fold, 9-fold, 10-fold or greater than the cytotoxicity of the fifth PD-L1 targeting molecule to a second population of target positive cells as assayed by CD₅₀ values.

In some embodiments, the PD-L1 targeting molecule is capable of delivering an embedded or inserted, heterologous, CD8+ T-cell epitope to a MHC class I presentation pathway of a cell for cell-surface presentation of the epitope bound by a MHC class I molecule.

In some embodiments, the PD-L1 targeting molecule is de-immunized due to the embedded or inserted, heterologous, epitope. In some embodiments, the PD-L1 targeting molecule is capable of exhibiting less relative antigenicity and/or relative immunogenicity as compared to a reference molecule, such as, e.g., another PD-L1 targeting molecule consisting of the PD-L1 targeting molecule except for it lacks one or more embedded or inserted epitopes present in the cell targeting molecule.

In some embodiments, the PD-L1 targeting molecule is not cytotoxic and is capable, when introduced to cells, of exhibiting a greater subcellular routing efficiency from an extracellular space to a subcellular compartment of an endoplasmic reticulum and/or cytosol as compared to the cytotoxicity of a reference molecule, such as, e.g., a second molecule similar to the PD-L1 targeting molecule except for that it does not comprise any carboxy-terminal, endoplasmic reticulum retention/retrieval signal motif of the KDEL family.

### PD-L1 Targeting Molecules Comprising a Shiga Toxin Effector Polypeptide Comprising (i) an Embedded or Inserted, Heterologous, T-Cell Epitope and (ii) a Disrupted, Furin-Cleavage Site

Also provided herein are PD-L1 targeting molecules, each comprising (i) a binding region capable of specifically binding an extracellular part of a PD-L1 target molecule; (ii) a Shiga toxin effector polypeptide comprising an inserted or embedded, heterologous, epitope; and (iii) a disrupted furin-cleavage site. In some embodiments, the PD-L1 targeting molecule comprises (i) a binding region capable of specifically binding an extracellular part of PD-L1; (ii) a Shiga toxin effector polypeptide comprising (a) an inserted or embedded, heterologous, epitope; (b) a Shiga toxin A1 fragment derived region having a carboxy terminus; and (c) a disrupted furin-cleavage site at the carboxy-terminus of the A1 fragment region. For some embodiments, the Shiga toxin effector polypeptide is capable of exhibiting at least one Shiga toxin effector function, such as, e.g., directing intracellular routing to the endoplasmic reticulum and/or cytosol of a cell in which the polypeptide is present, inhibiting a ribosome function, enzymatically inactivating a ribosome, causing cytostasis, and/or causing cytotoxicity. In some embodiments, the heterologous, T-cell epitope is a CD8+ T-cell epitope, such as, e.g., with regard to a human immune system. For some embodiments, the heterologous, T-cell epitope is capable of being presented by a MHC class I molecule of a cell. In some embodiments, the PD-L1 targeting molecule is capable of one or more the following: entering a cell, inhibiting a ribosome function, causing cytostasis, causing cell death, and/or delivering the embedded or inserted, heterologous, T-cell epitope to a MHC class I molecule for presentation on a cellular surface. For some embodiments, the PD-L1 targeting molecule is capable when introduced to cells of exhibiting a cytotoxicity comparable or better than a reference molecule, such as, e.g., a PD-L1 targeting molecule consisting of the PD-L1 targeting molecule except for all of its Shiga toxin effector polypeptide components comprise a wild-type Shiga toxin furin-cleavage site at the carboxy terminus of its A1 fragment region.

In some embodiments, the embedded or inserted, heterologous, T-cell epitope disrupts the endogenous, B-cell and/or T-cell epitope region selected from the group of natively positioned Shiga toxin A Subunit regions consisting of: (i) 1-15 of any one of SEQ ID NOs: 1-2 and 4-6; 3-14 of any one of SEQ ID NOs: 3 and 7-18; 26-37 of any one of SEQ ID NOs: 3 and 7-18; 27-37 of any one of SEQ ID NOs: 1-2 and 4-6; 39-48 of any one of SEQ ID NOs: 1-2 and 4-6; 42-48 of any one of SEQ ID NOs: 3 and 7-18; and 53-66 of any one of SEQ ID NOs: 1-18, or the equivalent region in a Shiga toxin A Subunit or derivative thereof; (ii) 94-115 of any one of SEQ ID NOs: 1-18; 141-153 of any one of SEQ ID NOs: 1-2 and 4-6; 140-156 of any one of SEQ ID NOs: 3 and 7-18; 179-190 of any one of SEQ ID NOs: 1-2 and 4-6; 179-191 of any one of SEQ ID NOs: 3 and 7-18; 204 of SEQ ID NO:3; 205 of any one of SEQ ID NOs: 1-2 and 4-6; and 210-218 of any one of SEQ ID NOs: 3 and 7-18, or the equivalent region in a Shiga toxin A Subunit or derivative thereof; and (iii) 240-260 of any one of SEQ ID NOs: 3 and 7-18; 243-257 of any one of SEQ ID NOs: 1-2 and 4-6; 254-268 of any one of SEQ ID NOs: 1-2 and 4-6; 262-278 of any one of SEQ ID NOs: 3 and 7-18; 281-297 of any one of SEQ ID NOs: 3 and 7-18; and 285-293 of any one of SEQ ID NOs: 1-2 and 4-6, or the equivalent region in a Shiga toxin A Subunit or derivative thereof.

In some embodiments, the disrupted furin-cleavage site comprises one or more mutations, relative to a wild-type Shiga toxin A Subunit, the mutation altering at least one amino acid residue in a region natively positioned at 248-251 of the A Subunit of Shiga toxin (SEQ ID NOs: 1-2 and 4-6), or at 247-250 of the A Subunit of Shiga-like toxin 2 (SEQ ID NOs: 3 and 7-18); or the equivalent region in a Shiga toxin A Subunit or derivative thereof. In some embodiments, the disrupted furin-cleavage site comprises one or more mutations, relative to a wild-type Shiga toxin A Subunit, in a minimal furin cleavage site of the furin-cleavage site. In some embodiments the minimal furin cleavage site is represented by the consensus amino acid sequence R/Y-x-x-R (SEQ ID NO: 159) and/or R-x-x-R (SEQ ID NO:160).

In some embodiments, the PD-L1 targeting molecule comprises a molecular moiety located carboxy-terminal to the carboxy-terminus of the Shiga toxin A1 fragment region.

In some embodiments, the binding region sterically covers the carboxy-terminus of the A1 fragment region.

In some embodiments, the molecular moiety sterically covers the carboxy-terminus of the A1 fragment region. In some embodiments, the molecular moiety comprises the binding region.

In some embodiments, the PD-L1 targeting molecule comprises a binding region and/or molecular moiety located carboxy-terminal to the carboxy-terminus of the Shiga toxin A1 fragment region. In some embodiments, the mass of the binding region and/or molecular moiety is at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater.

In some embodiments, the PD-L1 targeting molecule comprises a binding region with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the PD-L1 targeting molecule retains the appropriate level of the Shiga toxin biological activity noted herein (e.g., cytotoxicity and/or intracellular routing).

In some embodiments, the binding region is comprised within a relatively large, molecular moiety comprising such as, e.g., a molecular moiety with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the PD-L1 targeting molecule retains the appropriate level of the Shiga toxin biological activity noted herein.

In some embodiments, the disrupted furin-cleavage site comprises an amino acid residue substitution in the furin-cleavage site relative to a wild-type Shiga toxin A Subunit. In some embodiments, the substitution of the amino acid residue in the furin-cleavage site is of an arginine residue with a non-positively charged, amino acid residue selected from the group consisting of: alanine, glycine, proline, serine, threonine, aspartate, asparagine, glutamate, glutamine, cysteine, isoleucine, leucine, methionine, valine, phenylalanine, tryptophan, and tyrosine. In some embodiments, the substitution of the amino acid residue in the furin-cleavage site is of an arginine residue with a histidine.

In some embodiments, the PD-L1 targeting molecule is capable when introduced to cells of exhibiting cytotoxicity comparable to the cytotoxicity of a reference PD-L1 targeting molecule consisting of the PD-L1 targeting molecule except for all of its Shiga toxin effector polypeptide component(s) each comprise a wild-type Shiga toxin A1 fragment and/or wild-type Shiga toxin furin-cleavage site at the carboxy terminus of its A1 fragment region. In some embodiments, the PD-L1 targeting molecule is capable when introduced to cells of exhibiting cytotoxicity that is in a range of from 0.1-fold, 0.5-fold, or 0.75-fold to 1.2-fold, 1.5-fold, 1.75-fold, 2-fold, 3-fold, 4-fold, or 5-fold of the cytotoxicity exhibited by the reference PD-L1 targeting molecule.

In some embodiments, the PD-L1 targeting molecule is capable when introduced to a chordate of exhibiting improved, in vivo tolerability compared to in vivo tolerability of the reference PD-L1 targeting molecule.

In some embodiments, the PD-L1 targeting molecule is de-immunized due to the embedded or inserted, heterologous, epitope. In some embodiments, the PD-L1 targeting molecule is capable of exhibiting less relative antigenicity and/or relative immunogenicity as compared to a reference molecule, such as, e.g., a PD-L1 targeting molecule consisting of the PD-L1 targeting molecule except for it lacks one or more embedded or inserted epitopes present in the cell targeting molecule.

In some embodiments, the PD-L1 targeting molecule is de-immunized due to the furin-cleavage site disruption. In some embodiments, the PD-L1 targeting molecule is capable of exhibiting less relative antigenicity and/or relative immunogenicity as compared to a reference PD-L1 targeting molecule consisting of the PD-L1 targeting molecule except for the furin-cleavage site is wild-type and/or all the Shiga toxin effector polypeptide components consist of a wild-type Shiga toxin A1 fragment.

### PD-L1 Targeting Molecules Comprising a Shiga Toxin Effector Polypeptide at or Proximal to an Amino-Terminus and Wherein the Shiga Toxin Effector Polypeptide Comprises an Embedded or Inserted, Heterologous, T-Cell Epitope

Also provided herein are PD-L1 targeting molecules, each comprising (i) a binding region capable of specifically binding an extracellular part of a PD-L1 target molecule; (ii) a Shiga toxin effector polypeptide comprising an inserted or embedded, heterologous, epitope; wherein the Shiga toxin effector polypeptide is at or proximal to an amino-terminus of a polypeptide. In some embodiments, the PD-L1 targeting molecule comprises (i) a binding region capable of specifically binding an extracellular part of PD-L1, (ii) a polypeptide component, and (iii) a Shiga toxin effector polypeptide comprising an inserted or embedded, heterologous, epitope; wherein the Shiga toxin effector polypeptide is at or proximal to an amino-terminus of the polypeptide component of the PD-L1 targeting molecule. In some embodiments, the binding region and Shiga toxin effector polypeptide are physically arranged or oriented within the PD-L1 targeting molecule such that the binding region is not located proximally to the amino-terminus of the Shiga toxin effector polypeptide. In some embodiments, the binding region is located within the PD-L1 targeting molecule more proximal to the carboxy-terminus of the Shiga toxin effector polypeptide than to the amino-terminus of the Shiga toxin effector polypeptide. In some embodiments, the binding region is not located proximally to an amino-terminus of the PD-L1 targeting molecule relative to the Shiga toxin effector polypeptide. In some embodiments, the Shiga toxin effector polypeptide is capable of exhibiting at least one Shiga toxin effector function, such as, e.g., directing intracellular routing to the endoplasmic reticulum and/or cytosol of a cell in which the polypeptide is present, inhibiting a ribosome function, enzymatically inactivating a ribosome, causing cytostasis, and/or causing cytotoxicity. In some embodiments, the heterologous, T-cell epitope is a CD8+ T-cell epitope, such as, e.g., with regard to a human immune system. In some embodiments, the heterologous, T-cell epitope is capable of being presented by a MEW class I molecule of a cell. In some embodiments, the PD-L1 targeting molecule is capable of one or more the following: entering a cell, inhibiting a ribosome function, causing cytostasis, causing cell death, and/or delivering the embedded or inserted, heterologous, T-cell epitope to a MEW class I molecule for presentation on a cellular surface.

In some embodiments, the PD-L1 targeting molecule is capable when introduced to cells of exhibiting cytotoxicity that is greater than that of a reference PD-L1 targeting molecule having an amino-terminus and comprising the binding region and the Shiga toxin effector polypeptide region which is not positioned at or proximal to the amino-terminus of the reference PD-L1 targeting molecule. In some embodiments, the PD-L1 targeting molecule is capable of exhibiting a cytotoxicity with better optimized, cytotoxic potency, such as, e.g., 4-fold, 5-fold, 6-fold, 9-fold, or greater cytotoxicity as compared to the reference PD-L1 targeting molecule. In some embodiments, the cytotoxicity of the PD-L1 targeting molecule to a population of target positive cells is 3-fold, 4-fold, 5-fold, 6-fold, 7-fold, 8-fold, 9-fold, 10-fold or greater than the cytotoxicity of the reference PD-L1 targeting molecule to a second population of target positive cells as assayed by CD₅₀ values.

In some embodiments, the PD-L1 targeting molecule is capable of delivering an embedded or inserted, heterologous, CD8+ T-cell epitope to a MEW class I presentation pathway of a cell for cell-surface presentation of the epitope bound by a MEW class I molecule.

In some embodiments, the PD-L1 targeting molecule is de-immunized due to the embedded or inserted, heterologous, epitope. In some embodiments, the PD-L1 targeting molecule is capable of exhibiting less relative antigenicity and/or relative immunogenicity as compared to a reference molecule, such as, e.g., a PD-L1 targeting molecule consisting of the PD-L1 targeting molecule except for it lacks one or more embedded or inserted epitopes present in the cell targeting molecule.

In some embodiments, the PD-L1 targeting molecule is not cytotoxic and is capable when introduced to cells of exhibiting a greater subcellular routing efficiency from an extracellular space to a subcellular compartment of an endoplasmic reticulum and/or cytosol as compared to the cytotoxicity of a reference molecule, such as, e.g. a reference PD-L1 targeting molecule having an amino-terminus and comprising the binding region and the Shiga toxin effector polypeptide region which is not positioned at or proximal to the amino-terminus of the reference PD-L1 targeting molecule.

### PD-L1 Targeting Molecules Comprising a De-Immunized Shiga Toxin Effector Polypeptide Comprising a Disrupted, Furin-Cleavage Site

Also provided herein are PD-L1 targeting molecules, each comprising (i) a binding region capable of specifically binding an extracellular part of a PD-L1 target molecule and (ii) a de-immunized, Shiga toxin effector polypeptide comprising a disrupted furin-cleavage site. In some embodiments, the PD-L1 targeting molecule comprises (i) a binding region capable of specifically binding an extracellular part of PD-L1 and (ii) a de-immunized, Shiga toxin effector polypeptide comprising (a) a Shiga toxin A1 fragment derived region having a carboxy terminus, (b) a disrupted furin-cleavage site at the carboxy-terminus of the A1 fragment region, and (c) at least one disrupted, endogenous, B-cell and/or CD4+ T-cell epitope and/or epitope region. In some embodiments, the Shiga toxin effector polypeptide is capable of exhibiting at least one Shiga toxin effector function, such as, e.g., directing intracellular routing to the endoplasmic reticulum and/or cytosol of a cell in which the polypeptide is present, inhibiting a ribosome function, enzymatically inactivating a ribosome, causing cytostasis, and/or causing cytotoxicity. In some embodiments, the PD-L1 targeting molecule is capable of one or more the following: entering a cell, inhibiting a ribosome function, causing cytostasis, and/or causing cell death. In some embodiments, the PD-L1 targeting molecule is capable when introduced to cells of exhibiting a cytotoxicity comparable or better than a reference molecule, such as, e.g., a PD-L1 targeting molecule consisting of the PD-L1 targeting molecule except for all of its Shiga toxin effector polypeptide components comprise a wild-type Shiga toxin furin-cleavage site at the carboxy terminus of its A1 fragment region.

In some embodiments, the Shiga toxin effector polypeptide comprises a mutation, relative to a wild-type Shiga toxin A Subunit, in the B-cell and/or T-cell epitope region selected from the group of natively positioned Shiga toxin A Subunit regions consisting of: 1-15 of any one of SEQ ID NOs: 1-2 and 4-6; 3-14 of any one of SEQ ID NOs: 3 and 7-18; 26-37 of any one of SEQ ID NOs: 3 and 7-18; 27-37 of any one of SEQ ID NOs: 1-2 and 4-6; 39-48 of any one of SEQ ID NOs: 1-2 and 4-6; 42-48 of any one of SEQ ID NOs: 3 and 7-18; and 53-66 of any one of SEQ ID NOs: 1-18; 94-115 of any one of SEQ ID NOs: 1-18; 141-153 of any one of SEQ ID NOs: 1-2 and 4-6; 140-156 of any one of SEQ ID NOs: 3 and 7-18; 179-190 of any one of SEQ ID NOs: 1-2 and 4-6; 179-191 of any one of SEQ ID NOs: 3 and 7-18; 204 of SEQ ID NO:3; 205 of any one of SEQ ID NOs: 1-2 and 4-6; and 210-218 of any one of SEQ ID NOs: 3 and 7-18; 240-260 of any one of SEQ ID NOs: 3 and 7-18; 243-257 of any one of SEQ ID NOs: 1-2 and 4-6; 254-268 of any one of SEQ ID NOs: 1-2 and 4-6; 262-278 of any one of SEQ ID NOs: 3 and 7-18; 281-297 of any one of SEQ ID NOs: 3 and 7-18; and 285-293 of any one of SEQ ID NOs: 1-2 and 4-6; or the equivalent region in a Shiga toxin A Subunit or derivative thereof. In some embodiments, there is no disruption which is a carboxy-terminal truncation of amino acid residues that overlap with part or all of at least one disrupted, endogenous, B-cell and/or T-cell epitope and/or epitope region.

In some embodiments, the disrupted furin-cleavage site comprises one or more mutations, relative to a wild-type Shiga toxin A Subunit, the mutation altering at least one amino acid residue in a region natively positioned at 248-251 of the A Subunit of Shiga toxin (SEQ ID NOs: 1-2 and 4-6), or at 247-250 of the A Subunit of Shiga-like toxin 2 (SEQ ID NOs: 3 and 7-18), or the equivalent region in a Shiga toxin A Subunit or derivative thereof. In some embodiments, the disrupted furin-cleavage site comprises one or more mutations, relative to a wild-type Shiga toxin A Subunit, in a minimal furin cleavage site of the furin-cleavage site. In some embodiments the minimal furin cleavage site is represented by the consensus amino acid sequence R/Y-x-x-R (SEQ ID NO: 159) and/or R-x-x-R (SEQ ID NO: 160).

In some embodiments, the PD-L1 targeting molecule comprises a molecular moiety located carboxy-terminal to the carboxy-terminus of the Shiga toxin A1 fragment region.

In some embodiments, the binding region sterically covers the carboxy-terminus of the A1 fragment region.

In some embodiments, the molecular moiety sterically covers the carboxy-terminus of the A1 fragment region. In some embodiments, the molecular moiety comprises the binding region.

In some embodiments, the PD-L1 targeting molecule comprises a binding region and/or molecular moiety located carboxy-terminal to the carboxy-terminus of the Shiga toxin A1 fragment region. In some embodiments, the mass of the binding region and/or molecular moiety is at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater.

In some embodiments, the PD-L1 targeting molecule comprises a binding region with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the PD-L1 targeting molecule retains the appropriate level of the Shiga toxin biological activity noted herein (e.g., cytotoxicity and/or intracellular routing).

In some embodiments, the binding region is comprised within a relatively large, molecular moiety comprising such as, e.g., a molecular moiety with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the PD-L1 targeting molecule retains the appropriate level of the Shiga toxin biological activity noted herein.

In some embodiments, the disrupted furin-cleavage site comprises an amino acid residue substitution in the furin-cleavage site relative to a wild-type Shiga toxin A Subunit. In some embodiments, the substitution of the amino acid residue in the furin-cleavage site is of an arginine residue with a non-positively charged, amino acid residue selected from the group consisting of: alanine, glycine, proline, serine, threonine, aspartate, asparagine, glutamate, glutamine, cysteine, isoleucine, leucine, methionine, valine, phenylalanine, tryptophan, and tyrosine. In some embodiments, the substitution of the amino acid residue in the furin-cleavage site is of an arginine residue with a histidine.

In some embodiments, the PD-L1 targeting molecule is capable, when introduced to cells, of exhibiting cytotoxicity comparable to the cytotoxicity of a reference molecule, such as, e.g., a PD-L1 targeting molecule consisting of the PD-L1 targeting molecule except for all of its Shiga toxin effector polypeptide component(s) each comprise a wild-type Shiga toxin A1 fragment and/or wild-type Shiga toxin furin-cleavage site at the carboxy terminus of its A1 fragment region.

In some embodiments, the PD-L1 targeting molecule is de-immunized due to the furin-cleavage site disruption. In some embodiments, the PD-L1 targeting molecule is capable of exhibiting less relative antigenicity and/or relative immunogenicity as compared to a reference molecule similar to the PD-L1 targeting molecule except for the furin-cleavage site is wild-type and/or all the Shiga toxin effector polypeptide components consist of a wild-type Shiga toxin A1 fragment.

### PD-L1 Targeting Molecules Comprising a Carboxy-Terminal Endoplasmic Reticulum Retention/Retrieval Signal Motif and a De-Immunized Shiga Toxin Effector Polypeptide

Also provided herein are PD-L1 targeting molecules, each comprising (i) a binding region capable of specifically binding an extracellular part of a PD-L1 target molecule; (ii) a de-immunized, Shiga toxin effector polypeptide, and (iii) a carboxy-terminal, endoplasmic reticulum retention/retrieval signal motif. In some embodiments, the PD-L1 targeting molecule comprises (i) a binding region capable of specifically binding an extracellular part of PD-L1; (ii) a de-immunized, Shiga toxin effector polypeptide comprising at least one disrupted, endogenous, B-cell and/or CD4+ T-cell epitope and/or epitope region, and (iii) a carboxy-terminal, endoplasmic reticulum retention/retrieval signal motif of a member of the KDEL family. In some embodiments, the Shiga toxin effector polypeptide is capable of exhibiting at least one Shiga toxin effector function, such as, e.g., directing intracellular routing to the endoplasmic reticulum and/or cytosol of a cell in which the polypeptide is present, inhibiting a ribosome function, enzymatically inactivating a ribosome, causing cytostasis, and/or causing cytotoxicity. In some embodiments, the PD-L1 targeting molecule is capable of one or more the following: entering a cell, inhibiting a ribosome function, causing cytostasis, and/or causing cell death.

In some embodiments, the carboxy-terminal endoplasmic reticulum retention/retrieval signal motif is selected from the group consisting of: KDEL (SEQ ID NO: 111), HDEF (SEQ ID NO: 112), HDEL (SEQ ID NO: 113), RDEF (SEQ ID NO:114), RDEL (SEQ ID NO: 115), WDEL (SEQ ID NO: 116), YDEL (SEQ ID NO: 117), HEEF (SEQ ID NO: 118), HEEL (SEQ ID NO: 119), KEEL (SEQ ID NO: 120), REEL (SEQ ID NO: 121), KAEL (SEQ ID NO: 122), KCEL (SEQ ID NO: 123), KFEL (SEQ ID NO: 124), KGEL (SEQ ID NO: 125), KHEL (SEQ ID NO: 126), KLEL (SEQ ID NO: 127), KNEL (SEQ ID NO: 128), KQEL (SEQ ID NO: 129), KREL (SEQ ID NO: 130), KSEL (SEQ ID NO: 131), KVEL (SEQ ID NO: 132), KWEL (SEQ ID NO: 133), KYEL (SEQ ID NO: 134), KEDL (SEQ ID NO: 135), KIEL (SEQ ID NO: 136), DKEL (SEQ ID NO: 137), FDEL (SEQ ID NO: 138), KDEF (SEQ ID NO: 139), KKEL (SEQ ID NO: 140), HADL (SEQ ID NO: 141), HAEL (SEQ ID NO: 142), HIEL (SEQ ID NO: 143), HNEL (SEQ ID NO: 144), HTEL (SEQ ID NO: 145), KTEL (SEQ ID NO: 146), HVEL (SEQ ID NO: 147), NDEL (SEQ ID NO: 148), QDEL (SEQ ID NO: 149), REDL (SEQ ID NO: 150), RNEL (SEQ ID NO: 151), RTDL (SEQ ID NO: 152), RTEL (SEQ ID NO: 153), SDEL (SEQ ID NO: 154), TDEL (SEQ ID NO: 155), SKEL (SEQ ID NO: 156), STEL (SEQ ID NO: 157), and EDEL (SEQ ID NO: 158).

In some embodiments, the Shiga toxin effector polypeptide comprises a mutation, relative to a wild-type Shiga toxin A Subunit, in the B-cell and/or T-cell epitope region selected from the group of natively positioned Shiga toxin A Subunit regions consisting of: 1-15 of any one of SEQ ID NOs: 1-2 and 4-6; 3-14 of any one of SEQ ID NOs: 3 and 7-18; 26-37 of any one of SEQ ID NOs: 3 and 7-18; 27-37 of any one of SEQ ID NOs: 1-2 and 4-6; 39-48 of any one of SEQ ID NOs: 1-2 and 4-6; 42-48 of any one of SEQ ID NOs: 3 and 7-18; and 53-66 of any one of SEQ ID NOs: 1-18; 94-115 of any one of SEQ ID NOs: 1-18; 141-153 of any one of SEQ ID NOs: 1-2 and 4-6; 140-156 of any one of SEQ ID NOs: 3 and 7-18; 179-190 of any one of SEQ ID NOs: 1-2 and 4-6; 179-191 of any one of SEQ ID NOs: 3 and 7-18; 204 of SEQ ID NO:3; 205 of any one of SEQ ID NOs: 1-2 and 4-6; and 210-218 of any one of SEQ ID NOs: 3 and 7-18; 240-260 of any one of SEQ ID NOs: 3 and 7-18; 243-257 of any one of SEQ ID NOs: 1-2 and 4-6; 254-268 of any one of SEQ ID NOs: 1-2 and 4-6; 262-278 of any one of SEQ ID NOs: 3 and 7-18; 281-297 of any one of SEQ ID NOs: 3 and 7-18; and 285-293 of any one of SEQ ID NOs: 1-2 and 4-6; or the equivalent region in a Shiga toxin A Subunit or derivative thereof. In some embodiments, there is no disruption which is a carboxy-terminal truncation of amino acid residues that overlap with part or all of at least one disrupted, endogenous, B-cell and/or T-cell epitope and/or epitope region.

In some embodiments, the PD-L1 targeting molecule is capable when introduced to cells of exhibiting cytotoxicity that is greater than that of a reference PD-L1 targeting molecule consisting of the PD-L1 targeting molecule except for it does not comprise any carboxy-terminal, endoplasmic reticulum retention/retrieval signal motif of the KDEL family. In some embodiments, the PD-L1 targeting molecule is capable of exhibiting a cytotoxicity with better optimized, cytotoxic potency, such as, e.g., 4-fold, 5-fold, 6-fold, 9-fold, or greater cytotoxicity as compared to the reference PD-L1 targeting molecule. In some embodiments, the cytotoxicity of the PD-L1 targeting molecule to a population of target positive cells is 3-fold, 4-fold, 5-fold, 6-fold, 7-fold, 8-fold, 9-fold, 10-fold or greater than the cytotoxicity of the thirteenth PD-L1 targeting molecule to a second population of target positive cells as assayed by CD₅₀ values.

In some embodiments, the PD-L1 targeting molecule is not cytotoxic and is capable when introduced to cells of exhibiting a greater subcellular routing efficiency from an extracellular space to a subcellular compartment of an endoplasmic reticulum and/or cytosol as compared to the cytotoxicity of a reference molecule, such as, e.g. a reference PD-L1 targeting molecule having an amino-terminus and comprising the binding region and the Shiga toxin effector polypeptide region which is not positioned at or proximal to the amino-terminus of the reference PD-L1 targeting molecule and lacks a carboxy-terminal, endoplasmic reticulum retention/retrieval signal motif of the KDEL family.

### PD-L1 Targeting Molecules Comprising a De-Immunized Shiga Toxin Effector Polypeptide at or Proximal to an Amino-Terminus of the Cell Targeting Molecule

Also provided herein are PD-L1 targeting molecules, each comprising (i) a binding region capable of specifically binding an extracellular part of a PD-L1 target molecule, (ii) a de-immunized, Shiga toxin effector polypeptide; wherein the Shiga toxin effector polypeptide is at or proximal to an amino-terminus. In some embodiments, the PD-L1 targeting molecule comprises (i) a binding region capable of specifically binding an extracellular part of PD-L1; (ii) polypeptide component; and (iii) a de-immunized, Shiga toxin effector polypeptide comprising at least one disrupted, endogenous, B-cell and/or CD4+ T-cell epitope and/or epitope region; wherein the Shiga toxin effector polypeptide is at or proximal to an amino-terminus of the polypeptide component of the PD-L1 targeting molecule. In some embodiments, the binding region and Shiga toxin effector polypeptide are physically arranged or oriented within the PD-L1 targeting molecule such that the binding region is not located proximally to the amino-terminus of the Shiga toxin effector polypeptide. In some embodiments, the binding region is located within the PD-L1 targeting molecule more proximal to the carboxy-terminus of the Shiga toxin effector polypeptide than to the amino-terminus of the Shiga toxin effector polypeptide. In some embodiments, the binding region is not located proximally to an amino-terminus of the PD-L1 targeting molecule relative to the Shiga toxin effector polypeptide. For some embodiments, the Shiga toxin effector polypeptide is capable of exhibiting at least one Shiga toxin effector function, such as, e.g., directing intracellular routing to the endoplasmic reticulum and/or cytosol of a cell in which the polypeptide is present, inhibiting a ribosome function, enzymatically inactivating a ribosome, causing cytostasis, and/or causing cytotoxicity. In some embodiments, the PD-L1 targeting molecule is capable of one or more the following: entering a cell, inhibiting a ribosome function, causing cytostasis, and/or causing cell death.

In some embodiments, the Shiga toxin effector polypeptide comprises a mutation, relative to a wild-type Shiga toxin A Subunit, in the B-cell and/or T-cell epitope region selected from the group of natively positioned Shiga toxin A Subunit regions consisting of: 11-15 of any one of SEQ ID NOs: 1-2 and 4-6; 3-14 of any one of SEQ ID NOs: 3 and 7-18; 26-37 of any one of SEQ ID NOs: 3 and 7-18; 27-37 of any one of SEQ ID NOs: 1-2 and 4-6; 39-48 of any one of SEQ ID NOs: 1-2 and 4-6; 42-48 of any one of SEQ ID NOs: 3 and 7-18; and 53-66 of any one of SEQ ID NOs: 1-18; 94-115 of any one of SEQ ID NOs: 1-18; 141-153 of any one of SEQ ID NOs: 1-2 and 4-6; 140-156 of any one of SEQ ID NOs: 3 and 7-18; 179-190 of any one of SEQ ID NOs: 1-2 and 4-6; 179-191 of any one of SEQ ID NOs: 3 and 7-18; 204 of SEQ ID NO:3; 205 of any one of SEQ ID NOs: 1-2 and 4-6; and 210-218 of any one of SEQ ID NOs: 3 and 7-18; 240-260 of any one of SEQ ID NOs: 3 and 7-18; 243-257 of any one of SEQ ID NOs: 1-2 and 4-6; 254-268 of any one of SEQ ID NOs: 1-2 and 4-6; 262-278 of any one of SEQ ID NOs: 3 and 7-18; 281-297 of any one of SEQ ID NOs: 3 and 7-18; and 285-293 of any one of SEQ ID NOs: 1-2 and 4-6; or the equivalent region in a Shiga toxin A Subunit or derivative thereof. In some embodiments, there is no disruption which is a carboxy-terminal truncation of amino acid residues that overlap with part or all of at least one disrupted, endogenous, B-cell and/or T-cell epitope and/or epitope region.

In some embodiments, the PD-L1 targeting molecule is capable, when introduced to cells, of exhibiting cytotoxicity that is greater than that of a reference PD-L1 targeting molecule having an amino-terminus and comprising the binding region and the Shiga toxin effector polypeptide region which is not positioned at or proximal to the amino-terminus of the reference PD-L1 targeting molecule. In some embodiments, the PD-L1 targeting molecule is capable of exhibiting a cytotoxicity with better optimized, cytotoxic potency, such as, e.g., 4-fold, 5-fold, 6-fold, 9-fold, or greater cytotoxicity as compared to the reference PD-L1 targeting molecule. In some embodiments, the cytotoxicity of the PD-L1 targeting molecule to a population of target positive cells is 3-fold, 4-fold, 5-fold, 6-fold, 7-fold, 8-fold, 9-fold, 10-fold or greater than the cytotoxicity of the reference PD-L1 targeting molecule to a second population of target positive cells as assayed by CD₅₀ values.

In some embodiments, the PD-L1 targeting molecule is not cytotoxic and is capable when introduced to cells of exhibiting a greater subcellular routing efficiency from an extracellular space to a subcellular compartment of an endoplasmic reticulum and/or cytosol as compared to the cytotoxicity of a reference molecule, such as, e.g. a reference PD-L1 targeting molecule having an amino-terminus and comprising the binding region and the Shiga toxin effector polypeptide region which is not positioned at or proximal to the amino-terminus of the reference PD-L1 targeting molecule.

### PD-L1 Targeting Molecules Comprising a Carboxy-Terminal Endoplasmic Reticulum Retention/Retrieval Signal Motif and a Shiga Toxin Effector Polypeptide Comprising a Disrupted, Furin-Cleavage Site

Also provided herein are PD-L1 targeting molecules, each comprising (i) a binding region capable of specifically binding an extracellular part of a PD-L1 target molecule; (ii) a Shiga toxin effector polypeptide comprising a disrupted furin-cleavage site; and (iii) a carboxy-terminal endoplasmic reticulum retention/retrieval signal motif. In some embodiments, PD-L1 targeting molecules comprise (i) a binding region capable of specifically binding an extracellular part of PD-L1; (ii) a Shiga toxin effector polypeptide comprising a disrupted furin-cleavage site; and (iii) a carboxy-terminal, endoplasmic reticulum retention/retrieval signal motif of a member of the KDEL family. In some embodiments, the Shiga toxin effector polypeptide is capable of exhibiting at least one Shiga toxin effector function, such as, e.g., directing intracellular routing to the endoplasmic reticulum and/or cytosol of a cell in which the polypeptide is present, inhibiting a ribosome function, enzymatically inactivating a ribosome, causing cytostasis, and/or causing cytotoxicity. In some embodiments, the PD-L1 targeting molecule is capable of one or more the following: entering a cell, inhibiting a ribosome function, causing cytostasis, and/or causing cell death. For some embodiments, the PD-L1 targeting molecule is capable when introduced to cells of exhibiting a cytotoxicity comparable or better than a reference molecule, such as, e.g., a molecule similar to the PD-L1 targeting molecule except for all of its Shiga toxin effector polypeptide components comprise a wild-type Shiga toxin furin-cleavage site at the carboxy terminus of its A1 fragment region.

In some embodiments, the disrupted furin-cleavage site comprises one or more mutations, relative to a wild-type Shiga toxin A Subunit, the mutation altering at least one amino acid residue in a region natively positioned at 248-251 of the A Subunit of Shiga toxin (SEQ ID NOs: 1-2 and 4-6), or at 247-250 of the A Subunit of Shiga-like toxin 2 (SEQ ID NOs: 3 and 7-18), or the equivalent region in a Shiga toxin A Subunit or derivative thereof. In some embodiments, the disrupted furin-cleavage site comprises one or more mutations, relative to a wild-type Shiga toxin A Subunit, in a minimal furin cleavage site of the furin-cleavage site. In some embodiments the minimal furin cleavage site is represented by the consensus amino acid sequence R/Y-x-x-R (SEQ ID NO: 159) and/or R-x-x-R (SEQ ID NO: 160).

In some embodiments, the PD-L1 targeting molecule comprises a molecular moiety located carboxy-terminal to the carboxy-terminus of the Shiga toxin A1 fragment region.

In some embodiments, the binding region sterically covers the carboxy-terminus of the A1 fragment region.

In some embodiments, the molecular moiety sterically covers the carboxy-terminus of the A1 fragment region. In some embodiments, the molecular moiety comprises the binding region.

In some embodiments, the PD-L1 targeting molecule comprises a binding region and/or molecular moiety located carboxy-terminal to the carboxy-terminus of the Shiga toxin A1 fragment region. In some embodiments, the mass of the binding region and/or molecular moiety is at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater.

In some embodiments, the PD-L1 targeting molecule comprises a binding region with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the PD-L1 targeting molecule retains the appropriate level of the Shiga toxin biological activity noted herein (e.g., cytotoxicity and/or intracellular routing).

In some embodiments, the binding region is comprised within a relatively large, molecular moiety comprising such as, e.g., a molecular moiety with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the PD-L1 targeting molecule retains the appropriate level of the Shiga toxin biological activity noted herein.

In some embodiments, the disrupted furin-cleavage site comprises an amino acid residue substitution in the furin-cleavage site relative to a wild-type Shiga toxin A Subunit. In some embodiments, the substitution of the amino acid residue in the furin-cleavage site is of an arginine residue with a non-positively charged, amino acid residue selected from the group consisting of: alanine, glycine, proline, serine, threonine, aspartate, asparagine, glutamate, glutamine, cysteine, isoleucine, leucine, methionine, valine, phenylalanine, tryptophan, and tyrosine. In some embodiments, the substitution of the amino acid residue in the furin-cleavage site is of an arginine residue with a histidine.

In some embodiments, the PD-L1 targeting molecule is capable when introduced to cells of exhibiting cytotoxicity that is greater than that of a reference PD-L1 targeting molecule consisting of the PD-L1 targeting molecule except for it does not comprise any carboxy-terminal, endoplasmic reticulum retention/retrieval signal motif of the KDEL family. In some embodiments, the PD-L1 targeting molecule is capable of exhibiting a cytotoxicity with better optimized, cytotoxic potency, such as, e.g., 4-fold, 5-fold, 6-fold, 9-fold, or greater cytotoxicity as compared to the reference PD-L1 targeting molecule. In some embodiments, the cytotoxicity of the PD-L1 targeting molecule to a population of target positive cells is 3-fold, 4-fold, 5-fold, 6-fold, 7-fold, 8-fold, 9-fold, 10-fold or greater than the cytotoxicity of the reference PD-L1 targeting molecule to a second population of target positive cells as assayed by CD₅₀ values.

In some embodiments, the PD-L1 targeting molecule is capable when introduced to a chordate of exhibiting improved, in vivo tolerability compared to in vivo tolerability of a reference PD-L1 targeting molecule consisting of the PD-L1 targeting molecule except for all of its Shiga toxin effector polypeptide component(s) each comprise a wild-type Shiga toxin A1 fragment and/or wild-type Shiga toxin furin-cleavage site at the carboxy terminus of its A1 fragment region.

In some embodiments, the PD-L1 targeting molecule is de-immunized due to the furin-cleavage site disruption. In some embodiments, the PD-L1 targeting molecule is capable of exhibiting less relative antigenicity and/or relative immunogenicity as compared to a reference PD-L1 targeting molecule consisting of the PD-L1 targeting molecule except for the furin-cleavage site is wild-type and/or all the Shiga toxin effector polypeptide components consist of a wild-type Shiga toxin A1 fragment, such as, e.g., a reference PD-L1 targeting molecule consisting of the PD-L1 targeting molecule except for all of its Shiga toxin effector polypeptide component(s) each comprise a wild-type Shiga toxin A1 fragment and/or wild-type Shiga toxin furin-cleavage site at the carboxy terminus of its A1 fragment region.

In some embodiments, the PD-L1 targeting molecule is not cytotoxic and is capable when introduced to cells of exhibiting a greater subcellular routing efficiency from an extracellular space to a subcellular compartment of an endoplasmic reticulum and/or cytosol as compared to the cytotoxicity of the reference PD-L1 targeting molecule.

### PD-L1 Targeting Molecules Comprising a Furin-Cleavage Resistant Shiga Toxin Effector Polypeptide at or Proximal to an Amino-Terminus of the Cell Targeting Molecule

Also provided herein are PD-L1 targeting molecules, each comprising (i) a binding region capable of specifically binding an extracellular part of a PD-L1 target molecule and (ii) a Shiga toxin effector polypeptide comprising a disrupted furin-cleavage site at the carboxy-terminus of its Shiga toxin A1 fragment region; wherein the amino-terminus of the Shiga toxin effector polypeptide is at and/or proximal to an amino-terminus of a polypeptide component of the PD-L1 targeting molecule. In some embodiments, the PD-L1 targeting molecule comprises (i) a binding region capable of specifically binding an extracellular part of PD-L1, (ii) a Shiga toxin effector polypeptide having an amino-terminus and a Shiga toxin A1 fragment derived region having a carboxy terminus, and (iii) a disrupted furin-cleavage site at the carboxy-terminus of the A1 fragment region; wherein the binding region is not located proximally to the amino-terminus of the PD-L1 targeting molecule relative to the Shiga toxin effector polypeptide. In some embodiments, the binding region and Shiga toxin effector polypeptide are physically arranged or oriented within the PD-L1 targeting molecule such that the binding region is not located proximally to the amino-terminus of the Shiga toxin effector polypeptide. In some embodiments, the binding region is located within the PD-L1 targeting molecule more proximal to the carboxy-terminus of the Shiga toxin effector polypeptide than to the amino-terminus of the Shiga toxin effector polypeptide. In some embodiments, the binding region is not located proximally to an amino-terminus of the PD-L1 targeting molecule relative to the Shiga toxin effector polypeptide. For some embodiments, the Shiga toxin effector polypeptide is capable of exhibiting at least one Shiga toxin effector function, such as, e.g., directing intracellular routing to the endoplasmic reticulum and/or cytosol of a cell in which the polypeptide is present, inhibiting a ribosome function, enzymatically inactivating a ribosome, causing cytostasis, and/or causing cytotoxicity. In some embodiments, the PD-L1 targeting molecule is capable of one or more the following: entering a cell, inhibiting a ribosome function, causing cytostasis, and/or causing cell death. In some embodiments, the PD-L1 targeting molecule is capable, when introduced to cells, of exhibiting a cytotoxicity comparable or better than a reference molecule, such as, e.g., a PD-L1 targeting molecule consisting of the PD-L1 targeting molecule except for all of its Shiga toxin effector polypeptide components comprise a wild-type Shiga toxin furin-cleavage site at the carboxy terminus of its A1 fragment region.

In some embodiments, the disrupted furin-cleavage site comprises one or more mutations, relative to a wild-type Shiga toxin A Subunit, the mutation altering at least one amino acid residue in a region natively positioned at 248-251 of the A Subunit of Shiga toxin (SEQ ID NOs: 1-2 and 4-6), or at 247-250 of the A Subunit of Shiga-like toxin 2 (SEQ ID NOs: 3 and 7-18), or the equivalent region in a Shiga toxin A Subunit or derivative thereof. In some embodiments, the disrupted furin-cleavage site comprises one or more mutations, relative to a wild-type Shiga toxin A Subunit, in a minimal furin cleavage site of the furin-cleavage site. In some embodiments the minimal furin cleavage site is represented by the consensus amino acid sequence R/Y-x-x-R (SEQ ID NO: 159) and/or R-x-x-R (SEQ ID NO: 160).

In some embodiments, the PD-L1 targeting molecule comprises a molecular moiety located carboxy-terminal to the carboxy-terminus of the Shiga toxin A1 fragment region.

In some embodiments, the binding region sterically covers the carboxy-terminus of the A1 fragment region.

In some embodiments, the molecular moiety sterically covers the carboxy-terminus of the A1 fragment region. In some embodiments, the molecular moiety comprises the binding region.

In some embodiments, the PD-L1 targeting molecule comprises a binding region and/or molecular moiety located carboxy-terminal to the carboxy-terminus of the Shiga toxin A1 fragment region. In some embodiments, the mass of the binding region and/or molecular moiety is at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater.

In some embodiments, the PD-L1 targeting molecule comprises a binding region with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the PD-L1 targeting molecule retains the appropriate level of the Shiga toxin biological activity noted herein (e.g., cytotoxicity and/or intracellular routing).

In some embodiments, the binding region is comprised within a relatively large, molecular moiety comprising such as, e.g., a molecular moiety with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the PD-L1 targeting molecule retains the appropriate level of the Shiga toxin biological activity noted herein.

In some embodiments, the disrupted furin-cleavage site comprises an amino acid residue substitution in the furin-cleavage site relative to a wild-type Shiga toxin A Subunit. In some embodiments, the substitution of the amino acid residue in the furin-cleavage site is of an arginine residue with a non-positively charged, amino acid residue selected from the group consisting of: alanine, glycine, proline, serine, threonine, aspartate, asparagine, glutamate, glutamine, cysteine, isoleucine, leucine, methionine, valine, phenylalanine, tryptophan, and tyrosine. In some embodiments, the substitution of the amino acid residue in the furin-cleavage site is of an arginine residue with a histidine.

In some embodiments, the PD-L1 targeting molecule is capable when introduced to cells of exhibiting cytotoxicity that is greater than that of a reference PD-L1 targeting molecule having an amino-terminus and comprising the binding region and the Shiga toxin effector polypeptide region which is not positioned at or proximal to the amino-terminus of the reference PD-L1 targeting molecule. In some embodiments, the PD-L1 targeting molecule is capable of exhibiting a cytotoxicity with better optimized, cytotoxic potency, such as, e.g., 4-fold, 5-fold, 6-fold, 9-fold, or greater cytotoxicity as compared to the reference PD-L1 targeting molecule. In some embodiments, the cytotoxicity of the PD-L1 targeting molecule to a population of target positive cells is 3-fold, 4-fold, 5-fold, 6-fold, 7-fold, 8-fold, 9-fold, 10-fold or greater than the cytotoxicity of the reference PD-L1 targeting molecule to a second population of target positive cells as assayed by CD₅₀ values.

In some embodiments, the PD-L1 targeting molecule is capable when introduced to a chordate of exhibiting improved, in vivo tolerability compared to in vivo tolerability of a reference PD-L1 targeting molecule consisting of the PD-L1 targeting molecule except for all of its Shiga toxin effector polypeptide component(s) each comprise a wild-type Shiga toxin A1 fragment and/or wild-type Shiga toxin furin-cleavage site at the carboxy terminus of its A1 fragment region.

In some embodiments, the PD-L1 targeting molecule is de-immunized due to the furin-cleavage site disruption. In some embodiments, the PD-L1 targeting molecule is capable of exhibiting less relative antigenicity and/or relative immunogenicity as compared to a reference PD-L1 targeting molecule consisting of the PD-L1 targeting molecule except for the furin-cleavage site is wild-type and/or all the Shiga toxin effector polypeptide components consist of a wild-type Shiga toxin A1 fragment.

In some embodiments, the PD-L1 targeting molecule is not cytotoxic and is capable when introduced to cells of exhibiting a greater subcellular routing efficiency from an extracellular space to a subcellular compartment of an endoplasmic reticulum and/or cytosol as compared to the cytotoxicity of a reference molecule, such as, e.g. a reference PD-L1 targeting molecule having an amino-terminus and comprising the binding region and the Shiga toxin effector polypeptide region which is not positioned at or proximal to the amino-terminus of the reference PD-L1 targeting molecule.

### PD-L1 Targeting Molecules Comprising a Carboxy-Terminal Endoplasmic Reticulum Retention/Retrieval Signal Motif and Shiga Toxin Effector Polypeptide at or Proximal to an Amino-Terminus of the Cell Targeting Molecule

Also provided herein are PD-L1 targeting molecules, each comprising (i) a binding region capable of specifically binding an extracellular part of a PD-L1 target molecule, (ii) a carboxy-terminal, endoplasmic reticulum retention/retrieval signal motif, and (iii) a Shiga toxin effector polypeptide; wherein the amino-terminus of the Shiga toxin effector polypeptide is at and/or proximal to an amino-terminus of a polypeptide component of the PD-L1 targeting molecule. In some embodiments, the PD-L1 targeting molecule comprises a (i) binding region capable of specifically binding an extracellular part of PD-L1, (ii) a carboxy-terminal, endoplasmic reticulum retention/retrieval signal motif of a member of the KDEL family, (iii) a polypeptide component, and (iv) a Shiga toxin effector polypeptide; wherein the amino-terminus of the Shiga toxin effector polypeptide is at and/or proximal to an amino-terminus of a polypeptide component of the PD-L1 targeting molecule. In some embodiments, the binding region and Shiga toxin effector polypeptide are physically arranged or oriented within the PD-L1 targeting molecule such that the binding region is not located proximally to the amino-terminus of the Shiga toxin effector polypeptide. In some embodiments, the binding region is located within the PD-L1 targeting molecule more proximal to the carboxy-terminus of the Shiga toxin effector polypeptide than to the amino-terminus of the Shiga toxin effector polypeptide. In some embodiments, the binding region is not located proximally to an amino-terminus of the PD-L1 targeting molecule relative to the Shiga toxin effector polypeptide.

In some embodiments, the Shiga toxin effector polypeptide is capable of exhibiting at least one Shiga toxin effector function, such as, e.g., directing intracellular routing to the endoplasmic reticulum and/or cytosol of a cell in which the polypeptide is present, inhibiting a ribosome function, enzymatically inactivating a ribosome, causing cytostasis, and/or causing cytotoxicity. In some embodiments, the PD-L1 targeting molecule is capable of one or more the following: entering a cell, inhibiting a ribosome function, causing cytostasis, and/or causing cell death.

In some embodiments, the carboxy-terminal endoplasmic reticulum retention/retrieval signal motif is selected from the group consisting of: KDEL (SEQ ID NO: 111), HDEF (SEQ ID NO: 112), HDEL (SEQ ID NO: 113), RDEF (SEQ ID NO:114), RDEL (SEQ ID NO: 115), WDEL (SEQ ID NO: 116), YDEL (SEQ ID NO: 117), HEEF (SEQ ID NO: 118), HEEL (SEQ ID NO: 119), KEEL (SEQ ID NO: 120), REEL (SEQ ID NO: 121), KAEL (SEQ ID NO: 122), KCEL (SEQ ID NO: 123), KFEL (SEQ ID NO: 124), KGEL (SEQ ID NO: 125), KHEL (SEQ ID NO: 126), KLEL (SEQ ID NO: 127), KNEL (SEQ ID NO: 128), KQEL (SEQ ID NO: 129), KREL (SEQ ID NO: 130), KSEL (SEQ ID NO: 131), KVEL (SEQ ID NO: 132), KWEL (SEQ ID NO: 133), KYEL (SEQ ID NO: 134), KEDL (SEQ ID NO: 135), KIEL (SEQ ID NO: 136), DKEL (SEQ ID NO: 137), FDEL (SEQ ID NO: 138), KDEF (SEQ ID NO: 139), KKEL (SEQ ID NO: 140), HADL (SEQ ID NO: 141), HAEL (SEQ ID NO: 142), HIEL (SEQ ID NO: 143), HNEL (SEQ ID NO: 144), HTEL (SEQ ID NO: 145), KTEL (SEQ ID NO: 146), HVEL (SEQ ID NO: 147), NDEL (SEQ ID NO: 148), QDEL (SEQ ID NO: 149), REDL (SEQ ID NO: 150), RNEL (SEQ ID NO: 151), RTDL (SEQ ID NO: 152), RTEL (SEQ ID NO: 153), SDEL (SEQ ID NO: 154), TDEL (SEQ ID NO: 155), SKEL (SEQ ID NO: 156), STEL (SEQ ID NO: 157), and EDEL (SEQ ID NO: 158).

In some embodiments, the PD-L1 targeting molecule is capable, when introduced to cells, of exhibiting cytotoxicity that is greater than that of a reference PD-L1 targeting molecule having an amino-terminus and comprising the binding region and the Shiga toxin effector polypeptide region which is not positioned at or proximal to the amino-terminus of the reference PD-L1 targeting molecule and/or greater than that of a second reference PD-L1 targeting molecule consisting of the PD-L1 targeting molecule except for it does not comprise any carboxy-terminal, endoplasmic reticulum retention/retrieval signal motif of the KDEL family. In some embodiments, the first reference PD-L1 targeting molecule does not comprise any carboxy-terminal, endoplasmic reticulum retention/retrieval signal motif of the KDEL family. In some embodiments, the PD-L1 targeting molecule is capable of exhibiting a cytotoxicity with better optimized, cytotoxic potency, such as, e.g., 4-fold, 5-fold, 6-fold, 9-fold, or greater cytotoxicity as compared to the first or second reference molecules. In some embodiments, the cytotoxicity of the PD-L1 targeting molecule to a population of target positive cells is 3-fold, 4-fold, 5-fold, 6-fold, 7-fold, 8-fold, 9-fold, 10-fold or greater than the cytotoxicity of the first and/or second reference PD-L1 targeting molecules to a second population of target positive cells as assayed by CD₅₀ values.

In some embodiments, the PD-L1 targeting molecule is not cytotoxic and is capable when introduced to cells of exhibiting a greater subcellular routing efficiency from an extracellular space to a subcellular compartment of an endoplasmic reticulum and/or cytosol as compared to the cytotoxicity of the first or second reference molecules.

### Multivalent PD-L1 Targeting Molecules

In some embodiments, the PD-L1 targeting molecule is multivalent. In some embodiments, the multivalent PD-L1 targeting molecule comprises two or more binding regions, wherein each binding region is capable of specifically binding an extracellular part of the same PD-L1 molecule. For some embodiments, upon administration of the multivalent PD-L1 targeting molecule to a population of cells physically coupled with target biomolecule, which have the extracellular part bound by two or more binding regions of the multivalent PD-L1 targeting molecule, results in a cytotoxic effect which is greater than a cytotoxic effect resulting from administration of an equivalent amount, mass, or molarity of a monovalent target-binding molecule component of the multivalent PD-L1 targeting molecule to a population of the same target-positive cells under same conditions by a factor of 2, 2.5, 3, 4, 5, 7.5, 10, or greater than the fold-change in target-binding between the monovalent target-binding molecule component and the multivalent PD-L1 targeting molecule as measured by dissociation constant (K_{D}). In some embodiments of the multivalent PD-L1 targeting molecule, at least one of the binding regions comprises a polypeptide comprising an immunoglobulin-type binding region which comprises a heavy chain variable region (HVR-H) comprising three CDRs, each having at least 85%, 90%, 91%, 92%, 93%, 94%, 95%, 96%, 97%, 98%, or 99% identity to SEQ ID NO:22, SEQ ID NO:23, SEQ ID NO:24, SEQ ID NO:36, or SEQ ID NO:37; or consisting essentially of an amino acid sequence show in any one of SEQ ID NO:22, SEQ ID NO:23, SEQ ID NO:24, SEQ ID NO:36, and SEQ ID NO:37. In some embodiments, the binding region further comprises: (a) a light chain variable region (HVR-L) comprising three CDRs, having at least 85%, 90%, 91%, 92%, 93%, 94%, 95%, 96%, 97%, 98%, or 99% identity to SEQ ID NO:19, SEQ ID NO:20, and SEQ ID NO:21; or consisting essentially of an amino acid sequence shown in any one of SEQ ID NO:19, SEQ ID NO:20, and SEQ ID NO:21. In some embodiments, the binding region comprises: (a) a light chain variable region (HVR-L) comprising three CDRs, each comprising or consisting essentially of an amino acid sequence shown in any one of SEQ ID NO:19, SEQ ID NO:20, and SEQ ID NO:21; and (b) a heavy chain variable region (HVR-H) comprising three CDRs, each comprising or consisting essentially of an amino acid sequence show in any one of SEQ ID NO:22, SEQ ID NO:23, SEQ ID NO:24, SEQ ID NO:36, and SEQ ID NO:37.

In some embodiments, at least one of the binding region comprises: (a) a light chain region having at least 85%, 90%, 91%, 92%, 93%, 94%, 95%, 96%, 97%, 98%, or 99%, identity to SEQ ID NO:25 or SEQ ID NO:27 or consisting essentially of the amino acid sequence of SEQ ID NO:25 or SEQ ID NO:27; and/or (b) a heavy chain region having at least 85%, 90%, 91%, 92%, 93%, 94%, 95%, 96%, 97%, 98%, or 99% identity to SEQ ID NO:26 or consisting essentially of the amino acid sequence of SEQ ID NO:26. In some embodiments, the binding region comprises a polypeptide having at least 85%, 90%, 91%, 92%, 93%, 94%, 95%, 96%, 97%, 98%, or 99% identity to any one of SEQ ID NOs: 28-35 and 38-40 or consists essentially of the polypeptide shown in any one of SEQ ID NOs: 28-35 and 38-40. In some embodiments, the binding region is a single-chain variable fragment, such as, e.g., consisting of, comprising, or consisting essentially of the polypeptide of any one of SEQ ID NOs: 28-35 and 38-40.

### Additional Embodiments

In some embodiments, the Shiga toxin effector polypeptide is fused to the binding region, either directly or indirectly, such as, e.g., via a linker known to the skilled worker.

In some embodiments, the binding region comprises a polypeptide comprising an immunoglobulin-type binding region. In some embodiments, the binding region is an immunoglobulin-type binding region capable of high-affinity and specific binding to PD-L1, such as, e.g., by binding to an extracellular part of a PD-L1 molecule. In some embodiments, the PD-L1 molecule is of human origin. In some embodiments, the binding region comprises a heavy chain variable region (HVR-H) comprising three CDRs, each having at least 85%, 90%, 91%, 92%, 93%, 94%, 95%, 96%, 97%, 98%, or 99% identity to SEQ ID NO:22, SEQ ID NO:23, SEQ ID NO:24, SEQ ID NO:36, or SEQ ID NO:37; or consisting essentially of an amino acid sequence show in any one of SEQ ID NO:22, SEQ ID NO:23, SEQ ID NO:24, SEQ ID NO:36, and SEQ ID NO:37. In some embodiments, the binding region further comprises: (a) a light chain variable region (HVR-L) comprising three CDRs, having at least 85%, 90%, 91%, 92%, 93%, 94%, 95%, 96%, 97%, 98%, or 99% identity to SEQ ID NO:19, SEQ ID NO:20, and SEQ ID NO:21; or consisting essentially of an amino acid sequence shown in any one of SEQ ID NO:19, SEQ ID NO:20, and SEQ ID NO:21. In some embodiments, the binding region comprises: (a) a light chain variable region (HVR-L) comprising three CDRs, each comprising or consisting essentially of an amino acid sequence shown in any one of SEQ ID NO:19, SEQ ID NO:20, and SEQ ID NO:21; and (b) a heavy chain variable region (HVR-H) comprising three CDRs, each comprising or consisting essentially of an amino acid sequence show in any one of SEQ ID NO:22, SEQ ID NO:23, SEQ ID NO:24, SEQ ID NO:36, and SEQ ID NO:37.

In some embodiments, the binding region comprises: (a) a light chain variable region (HVR-L) comprising three CDRs, each comprising or consisting essentially of an amino acid sequence shown in any one of SEQ ID NO:19, SEQ ID NO:20, and SEQ ID NO:21; and/or (b) a heavy chain variable region (HVR-H) comprising three CDRs, each comprising or consisting essentially of an amino acid sequence show in any one of SEQ ID NO:22, SEQ ID NO:23, SEQ ID NO:24, SEQ ID NO:36, and SEQ ID NO:37.

In some embodiments, the binding region comprises: (a) a light chain variable region comprising: (i) HVR-L1 comprising or consisting essentially of the amino acid sequence TGTSSDVGSYNRVS (SEQ ID NO:19), (ii) HVR-L2 comprising or consisting essentially of the amino acid sequence EVSNRPS (SEQ ID NO:20), and (iii) HVR-L3 comprising or consisting essentially of the amino acid sequence SSHTTSGTYV (SEQ ID NO:21); and/or (b) a heavy chain variable region comprising: (i) HVR-H1 comprising or consisting essentially of the amino acid sequence SYAIS (SEQ ID NO:22), (ii) HVR-H2 comprising or consisting essentially of the amino acid sequence GIIPIFGTANYAQKFQG (SEQ ID NO:23), and (iii) HVR-H3 comprising or consisting essentially of the amino acid sequence DQGYAHAFDI (SEQ ID NO:24).

In some embodiments, the binding region comprises: (a) a light chain variable region (HVR-L) comprising three CDRs: (i) a LCDR1 comprising or consisting essentially of the amino acid sequence TGTSSDVGSYNRVS as shown in SEQ ID NO:19, (ii) a LCDR2 comprising or consisting essentially of the amino acid sequence EVSNRPS as shown in SEQ ID NO:20, and (iii) a LCDR3 comprising or consisting essentially of the amino acid sequence SSHTTSGTYV as shown in SEQ ID NO:21; and/or (b) a heavy chain variable region (HVR-H) comprising: (i) a HCDR1 comprising or consisting essentially of the amino acid sequence GGTFSSY as shown in SEQ ID NO:22, (ii) a HCDR2 comprising or consisting essentially of the amino acid sequence IPIFGT as shown in SEQ ID NO:23, and (iii) a HCDR3 comprising or consisting essentially of the amino acid sequence DQGYAHAFDI as shown in SEQ ID NO:24.

In some embodiments, the binding region comprises: an immunoglobulin region comprising three CDRs comprising or consisting essentially of the amino acid sequences show in SEQ ID NO:22, SEQ ID NO:23, and SEQ ID NO:24, respectively.

In some embodiments, the binding region comprises an immunoglobulin region comprising three CDRs comprising or consisting essentially of the amino acid sequences show in SEQ ID NO:22, SEQ ID NO:37, and SEQ ID NO:24, respectively.

In some embodiments, the binding region comprises three CDRs comprising or consisting essentially of the amino acid sequences show in SEQ ID NO:36, SEQ ID NO:23, and SEQ ID NO:24, respectively.

In some embodiments, the binding region comprises an immunoglobulin region comprising three CDRs comprising or consisting essentially of the amino acid sequences show in SEQ ID NO:36, SEQ ID NO:37, and SEQ ID NO:24, respectively.

In some embodiments, the binding region comprises: (a) a light chain region having at least 85%, 90%, 91%, 92%, 93%, 94%, 95%, 96%, 97%, 98%, or 99%, identity to SEQ ID NO:25 or SEQ ID NO:27 or consisting essentially of the amino acid sequence of SEQ ID NO:25 or SEQ ID NO:27; and/or (b) a heavy chain region having at least 85%, 90%, 91%, 92%, 93%, 94%, 95%, 96%, 97%, 98%, or 99% identity to SEQ ID NO:26 or consisting essentially of the amino acid sequence of SEQ ID NO:26.

In some embodiments, the binding region comprises a polypeptide having at least 85%, 90%, 91%, 92%, 93%, 94%, 95%, 96%, 97%, 98%, or 99% identity to any one of SEQ ID NOs: 28-35 and 38-40 or consists essentially of the polypeptide shown in any one of SEQ ID NOs: 28-35 and 38-40.

In some embodiments, the binding region is a single-chain variable fragment, such as, e.g., consisting of, comprising, or consisting essentially of the polypeptide of any one of SEQ ID NOs: 28-35 and 38-40.

In some embodiments, the binding region comprises a polypeptide(s) selected from the group consisting of: (a) a heavy chain variable (V_{H}) domain comprising (i) a HCDR1 comprising or consisting essentially of the amino acid sequence as shown in SEQ ID NO:3 or SEQ ID NO:36; (ii) a HCDR2 comprising or consisting essentially of the amino acid sequence as shown in SEQ ID NO:20, 23, or 37; and/or (iii) a HCDR3 comprising or consisting essentially of the amino acid sequence as shown in SEQ ID NO:24; and/or (b) a light chain variable (V_{L}) domain comprising (i) a LCDR1 comprising or consisting essentially of the amino acid sequence as shown in SEQ ID NO:19; (ii) a LCDR2 comprising or consisting essentially of the amino acid sequence as shown in SEQ ID NO:20; and/or (iii) a LCDR3 comprising or consisting essentially of the amino acid sequence as shown in SEQ ID NO:21.

In some embodiments, a heterologous, CD8+ T-cell epitope-peptide cargo is fused, either directly or indirectly, to the Shiga toxin effector polypeptide and/or the binding region. In some embodiments of the PD-L1 targeting molecule, the CD8+ T-cell epitope-peptide cargo is fused via a peptide bond, either directly or indirectly, to the Shiga toxin A Subunit effector polypeptide and/or the binding region. In some embodiments, the CD8+ T-cell epitope-peptide cargo is fused via a peptide bond, either directly or indirectly, to the Shiga toxin A Subunit effector polypeptide and/or the binding region as a genetic fusion. In some embodiments, the PD-L1 targeting molecule comprises a single-chain polypeptide comprising the binding region, the Shiga toxin effector polypeptide, and the heterologous, CD8+ T-cell epitope-peptide cargo.

In some embodiments, the PD-L1 targeting molecule comprises a polypeptide comprising the binding region, the Shiga toxin effector polypeptide, and the CD8+ T-cell epitope-peptide cargo.

In some embodiments, the binding region comprises two or more polypeptide chains and a heterologous, CD8+ T-cell epitope-peptide cargo is fused either directly or indirectly, to a polypeptide comprising the Shiga toxin effector polypeptide and one of the two or more polypeptide chains of the binding region.

In some embodiments, the PD-L1 targeting molecule comprises a heterologous, CD8+ T-cell epitope-peptide cargo which is positioned within the PD-L1 targeting molecule carboxy-terminal to the Shiga toxin effector polypeptide and/or binding region. In some embodiments, the PD-L1 targeting molecule comprises two, three, four, five, or more heterologous, CD8+ T-cell epitope-peptide cargos positioned within the PD-L1 targeting molecule carboxy-terminal to the Shiga toxin effector polypeptide and/or binding region.

In some embodiments, the PD-L1 targeting molecule comprises a carboxy-terminal, heterologous, CD8+ T-cell epitope-peptide cargo.

In some embodiments, upon administration of the PD-L1 targeting molecule to a target cell physically coupled with an extracellular part of PD-L1 of the binding region, the PD-L1 targeting molecule is capable of causing intercellular engagement of the target cell by a CD8+ immune cell.

In some embodiments, upon administration of the PD-L1 targeting molecule to a target cell physically coupled with an extracellular part of PD-L1 bound by the binding region, the PD-L1 targeting molecule is capable of causing intercellular engagement of the target cell by a CD8+ immune cell. For some embodiments, upon administration of the PD-L1 targeting molecule to a target cell physically coupled with extracellular PD-L1 bound by the binding region, the PD-L1 targeting molecule is capable of causing death of the target cell. For some embodiments, upon administration of the PD-L1 targeting molecule to a first population of cells whose members are physically coupled to extracellular PD-L1 bound by the binding region, and a second population of cells whose members are not physically coupled to any extracellular target biomolecule bound by the binding region, the cytotoxic effect of the PD-L1 targeting molecule to members of said first population of cells relative to members of said second population of cells is at least 3-fold greater. In some embodiments, the Shiga toxin effector polypeptide comprises a mutation relative to a naturally occurring A Subunit of a member of the Shiga toxin family which changes the enzymatic activity of the Shiga toxin effector polypeptide, the mutation selected from at least one amino acid residue deletion, insertion, or substitution. In some embodiments, the mutation is selected from at least one amino acid residue deletion, insertion, or substitution that reduces or eliminates cytotoxicity of the toxin effector polypeptide. In some embodiments, the binding region comprises a heterologous, CD8+ T-cell epitope cargo, whether the CD8+ epitope-peptide is autogenous or heterologous with respect to the binding region.

In some embodiments, the PD-L1 targeting molecule may be used for targeted delivery of a CD8+ T-cell epitope to a nucleated, target cell within a chordate in order to cause the delivered CD8+ T-cell epitope-peptide cargo to be presented on the target cell surface complexed with a MHC class I molecule. The target cells can be of various types, such as, e.g., neoplastic cells, infected cells, cells harboring intracellular pathogens, and other undesirable cells, and the target cell can be targeted by PD-L1 targeting molecules either in vitro or in vivo.

In some embodiments, the PD-L1 targeting molecule comprises a heterologous, CD8+ T-cell epitope-peptide cargo for delivery to a target cell. For some embodiments, upon administration of the PD-L1 targeting molecule to a cell, which is physically coupled with extracellular PD-L1 bound by the binding region of the PD-L1 targeting molecule, results in the cell presenting on a cellular surface the CD8+ T-cell epitope-peptide cargo complexed with a MHC class I molecule. In some embodiments, having or placing the cell in the presence of an immune cell(s) further results in an immune cell response in trans, an inter-cellular engagement of the cell by an immune cell (e.g. a cytotoxic T lymphocyte), and/or death of the cell induced via an inter-cellular action(s) of an immune cell.

In some embodiments, the PD-L1 targeting molecule comprises a heterologous, CD8+ T-cell epitope-peptide cargo for delivery to a target cell. For some embodiments of the PD-L1 targeting molecule, upon administration of the PD-L1 targeting molecule to a chordate, which comprises cells physically coupled with extracellular PD-L1 bound by the binding region of the PD-L1 targeting molecule, results in at least some of said cells presenting on a cellular surface the CD8+ T-cell epitope-peptide cargo complexed with a MHC class I molecule. In some embodiments, the results further include an immune cell response in trans, such as, e.g., the inter-cellular engagement of at least some of said cells by an immune cell and/or death of the cell induced via an inter-cellular action(s) of an immune cell (e.g. a cytotoxic T lymphocyte).

In some embodiments, the Shiga toxin effector polypeptide has a Shiga toxin A1 fragment derived region having a carboxy terminus and further comprises a disrupted furin-cleavage site at the carboxy-terminus of the A1 fragment region.

In some embodiments, the PD-L1 targeting molecule comprises a molecular moiety located carboxy-terminal to the carboxy-terminus of the Shiga toxin A1 fragment region.

In some embodiments, the Shiga toxin effector polypeptide is capable of exhibiting at least one Shiga toxin effector function, such as, e.g., directing intracellular routing to the endoplasmic reticulum and/or cytosol of a cell in which the polypeptide is present, inhibiting a ribosome function, enzymatically inactivating a ribosome, causing cytostasis, and/or causing cytotoxicity. In some embodiments, the PD-L1 targeting molecule is capable of one or more the following: entering a cell, inhibiting a ribosome function, causing cytostasis, and/or causing cell death.

In some embodiments, the PD-L1 targeting molecule is capable of exhibiting (i) a catalytic activity level comparable to a wild-type Shiga toxin A1 fragment or wild-type Shiga toxin effector polypeptide, (ii) a ribosome inhibition activity with a half-maximal inhibitory concentration (IC₅₀) value of 10,000 picomolar or less, and/or (iii) a significant level of Shiga toxin catalytic activity.

In some embodiments, the PD-L1 targeting molecule is capable when introduced to cells of exhibiting a cytotoxicity with a half-maximal inhibitory concentration (CD₅₀) value of 300 nM or less and/or capable of exhibiting a significant level of Shiga toxin cytotoxicity.

In some embodiments, the PD-L1 targeting molecule exhibits low cytotoxic potency (i.e. is not capable when introduced to certain positive target cell types of exhibiting a cytotoxicity greater than 1% cell death of a cell population at a PD-L1 targeting molecule concentration of 1000 nM, 500 nM, 100 nM, 75 nM, or 50 nM).

In some embodiments, the Shiga toxin A Subunit effector polypeptide comprises one or more mutations relative to a naturally occurring A Subunit of a member of the Shiga toxin family which changes an enzymatic activity of the Shiga toxin A Subunit effector polypeptide, the mutation selected from at least one amino acid residue deletion, insertion, or substitution. For some embodiments, the mutation(s), relative to the naturally occurring A Subunit which changes an enzymatic activity of the Shiga toxin A Subunit effector polypeptide, reduces or eliminates a cytotoxicity exhibited by the Shiga toxin A Subunit effector polypeptide without the mutation(s).

In some embodiments, the PD-L1 targeting molecule, or a polypeptide component thereof, comprises a carboxy-terminal, endoplasmic reticulum retention/retrieval signal motif of a member of the KDEL family. For some embodiments, the carboxy-terminal endoplasmic reticulum retention/retrieval signal motif is selected from the group consisting of: KDEL (SEQ ID NO: 111), HDEF (SEQ ID NO: 112), HDEL (SEQ ID NO: 113), RDEF (SEQ ID NO:114), RDEL (SEQ ID NO: 115), WDEL (SEQ ID NO: 116), YDEL (SEQ ID NO: 117), HEEF (SEQ ID NO: 118), HEEL (SEQ ID NO: 119), KEEL (SEQ ID NO: 120), REEL (SEQ ID NO: 121), KAEL (SEQ ID NO: 122), KCEL (SEQ ID NO: 123), KFEL (SEQ ID NO: 124), KGEL (SEQ ID NO: 125), KHEL (SEQ ID NO: 126), KLEL (SEQ ID NO: 127), KNEL (SEQ ID NO: 128), KQEL (SEQ ID NO: 129), KREL (SEQ ID NO: 130), KSEL (SEQ ID NO: 131), KVEL (SEQ ID NO: 132), KWEL (SEQ ID NO: 133), KYEL (SEQ ID NO: 134), KEDL (SEQ ID NO: 135), KIEL (SEQ ID NO: 136), DKEL (SEQ ID NO: 137), FDEL (SEQ ID NO: 138), KDEF (SEQ ID NO: 139), KKEL (SEQ ID NO: 140), HADL (SEQ ID NO: 141), HAEL (SEQ ID NO: 142), HIEL (SEQ ID NO: 143), HNEL (SEQ ID NO: 144), HTEL (SEQ ID NO: 145), KTEL (SEQ ID NO: 146), HVEL (SEQ ID NO: 147), NDEL (SEQ ID NO: 148), QDEL (SEQ ID NO: 149), REDL (SEQ ID NO: 150), RNEL (SEQ ID NO: 151), RTDL (SEQ ID NO: 152), RTEL (SEQ ID NO: 153), SDEL (SEQ ID NO: 154), TDEL (SEQ ID NO: 155), SKEL (SEQ ID NO: 156), STEL (SEQ ID NO: 157), and EDEL (SEQ ID NO: 158). In some embodiments, the PD-L1 targeting molecule is capable, when introduced to cells, of exhibiting cytotoxicity that is greater than that of a reference molecule, such as, e.g., a second PD-L1 targeting molecule consisting of the PD-L1 targeting molecule except for it does not comprise any carboxy-terminal, endoplasmic reticulum retention/retrieval signal motif of the KDEL family.

In some embodiments, the Shiga toxin effector polypeptide further comprises at least one, two, or three disrupted, endogenous, B-cell and/or CD4+ T-cell epitope regions. In some embodiments, the Shiga toxin effector polypeptide comprises a disruption of at least one, two, or three endogenous, B-cell and/or T-cell epitopes and/or epitope regions. In some embodiments, the Shiga toxin effector polypeptide further comprises at least one disrupted, endogenous, B-cell and/or CD4+ T-cell epitope region which does not overlap with at least one inserted or embedded, heterologous epitope.

In some embodiments, the Shiga toxin effector polypeptide further comprises at least one inserted or embedded, heterologous epitope, such as, e.g., a CD8+ T-cell epitope.

In some embodiments, the amino-terminus of the Shiga toxin effector polypeptide is at and/or proximal to an amino-terminus of a polypeptide component of the PD-L1 targeting molecule. In some embodiments, the binding region is not located proximally to the amino-terminus of the PD-L1 targeting molecule relative to the Shiga toxin effector polypeptide. In some embodiments, the binding region and Shiga toxin effector polypeptide are physically arranged or oriented within the PD-L1 targeting molecule such that the binding region is not located proximally to the amino-terminus of the Shiga toxin effector polypeptide. In some embodiments, the binding region is located within the PD-L1 targeting molecule more proximal to the carboxy-terminus of the Shiga toxin effector polypeptide than to the amino-terminus of the Shiga toxin effector polypeptide. In some embodiments, the PD-L1 targeting molecule is not cytotoxic and is capable when introduced to cells of exhibiting a greater subcellular routing efficiency from an extracellular space to a subcellular compartment of an endoplasmic reticulum and/or cytosol as compared to the cytotoxicity of a reference molecule, such as, e.g., a second PD-L1 targeting molecule having an amino-terminus and comprising the binding region and the Shiga toxin effector polypeptide which is not positioned at or proximal to the amino-terminus of the reference PD-L1 targeting molecule. In some embodiments, the PD-L1 targeting molecule exhibits cytotoxicity with better optimized, cytotoxic potency, such as, e.g., 4-fold, 5-fold, 6-fold, 9-fold, or greater cytotoxicity as compared to the cytotoxicity of the second PD-L1 targeting molecule. In some embodiments, the reference PD-L1 targeting molecule does not comprise any carboxy-terminal, endoplasmic reticulum retention/retrieval signal motif of the KDEL family.

In some embodiments, the Shiga toxin effector polypeptide further comprises a disruption in the B-cell and/or T-cell epitope region selected from the group of natively positioned Shiga toxin A Subunit regions consisting of: 1-15 of any one of SEQ ID NOs: 1-2 and 4-6; 3-14 of any one of SEQ ID NOs: 3 and 7-18; 26-37 of any one of SEQ ID NOs: 3 and 7-18; 27-37 of any one of SEQ ID NOs: 1-2 and 4-6; 39-48 of any one of SEQ ID NOs: 1-2 and 4-6; 42-48 of any one of SEQ ID NOs: 3 and 7-18; and 53-66 of any one of SEQ ID NOs: 1-18; 94-115 of any one of SEQ ID NOs: 1-18; 141-153 of any one of SEQ ID NOs: 1-2 and 4-6; 140-156 of any one of SEQ ID NOs: 3 and 7-18; 179-190 of any one of SEQ ID NOs: 1-2 and 4-6; 179-191 of any one of SEQ ID NOs: 3 and 7-18; 204 of SEQ ID NO:3; 205 of any one of SEQ ID NOs: 1-2 and 4-6; and 210-218 of any one of SEQ ID NOs: 3 and 7-18; 240-260 of any one of SEQ ID NOs: 3 and 7-18; 243-257 of any one of SEQ ID NOs: 1-2 and 4-6; 254-268 of any one of SEQ ID NOs: 1-2 and 4-6; 262-278 of any one of SEQ ID NOs: 3 and 7-18; 281-297 of any one of SEQ ID NOs: 3 and 7-18; and 285-293 of any one of SEQ ID NOs: 1-2 and 4-6; or the equivalent region in a Shiga toxin A Subunit or derivative thereof. In some embodiments, there is no disruption which is a carboxy-terminal truncation of amino acid residues that overlap with part or all of at least one disrupted, endogenous, B-cell and/or T-cell epitope and/or epitope region.

In some embodiments, the Shiga toxin effector polypeptide further comprises a mutation, relative to a wild-type Shiga toxin A Subunit, in the B-cell immunogenic, amino acid residue selected from the group of natively positioned Shiga toxin A Subunit amino acid residues: L49, D197, D198, R204, and R205.

In some embodiments, the embedded or inserted, heterologous, T-cell epitope disrupts the endogenous, B-cell and/or T-cell epitope region is selected from the group of natively positioned Shiga toxin A Subunit regions consisting of: (i) 1-15 of any one of SEQ ID NOs: 1-2 and 4-6; 3-14 of any one of SEQ ID NOs: 3 and 7-18; 26-37 of any one of SEQ ID NOs: 3 and 7-18; 27-37 of any one of SEQ ID NOs: 1-2 and 4-6; 39-48 of any one of SEQ ID NOs: 1-2 and 4-6; 42-48 of any one of SEQ ID NOs: 3 and 7-18; and 53-66 of any one of SEQ ID NOs: 1-18, or the equivalent region in a Shiga toxin A Subunit or derivative thereof; (ii) 94-115 of any one of SEQ ID NOs: 1-18; 141-153 of any one of SEQ ID NOs: 1-2 and 4-6; 140-156 of any one of SEQ ID NOs: 3 and 7-18; 179-190 of any one of SEQ ID NOs: 1-2 and 4-6; 179-191 of any one of SEQ ID NOs: 3 and 7-18; 204 of SEQ ID NO:3; 205 of any one of SEQ ID NOs: 1-2 and 4-6; and 210-218 of any one of SEQ ID NOs: 3 and 7-18, or the equivalent region in a Shiga toxin A Subunit or derivative thereof; and (iii) 240-260 of any one of SEQ ID NOs: 3 and 7-18; 243-257 of any one of SEQ ID NOs: 1-2 and 4-6; 254-268 of any one of SEQ ID NOs: 1-2 and 4-6; 262-278 of any one of SEQ ID NOs: 3 and 7-18; 281-297 of any one of SEQ ID NOs: 3 and 7-18; and 285-293 of any one of SEQ ID NOs: 1-2 and 4-6, or the equivalent region in a Shiga toxin A Subunit or derivative thereof.

In some embodiments, the Shiga toxin effector polypeptide comprises a mutation, relative to a wild-type Shiga toxin A Subunit, in the B-cell and/or T-cell epitope region selected from the group of natively positioned Shiga toxin A Subunit regions consisting of: (i) 1-15 of any one of SEQ ID NOs: 1-2 and 4-6; 3-14 of any one of SEQ ID NOs: 3 and 7-18; 26-37 of any one of SEQ ID NOs: 3 and 7-18; 27-37 of any one of SEQ ID NOs: 1-2 and 4-6; 39-48 of any one of SEQ ID NOs: 1-2 and 4-6; 42-48 of any one of SEQ ID NOs: 3 and 7-18; and 53-66 of any one of SEQ ID NOs: 1-18, or the equivalent region in a Shiga toxin A Subunit or derivative thereof, wherein there is no disruption which is an amino-terminal truncation of sequences that overlap with part or all of at least one disrupted epitope region; (ii) 94-115 of any one of SEQ ID NOs: 1-18; 141-153 of any one of SEQ ID NOs: 1-2 and 4-6; 140-156 of any one of SEQ ID NOs: 3 and 7-18; 179-190 of any one of SEQ ID NOs: 1-2 and 4-6; 179-191 of any one of SEQ ID NOs: 3 and 7-18; 204 of SEQ ID NO:3; 205 of any one of SEQ ID NOs: 1-2 and 4-6; and 210-218 of any one of SEQ ID NOs: 3 and 7-18, or the equivalent region in a Shiga toxin A Subunit or derivative thereof, wherein there is no disruption which is an amino-terminal truncation of sequences that overlap with part or all of at least one disrupted epitope region.

In some embodiments, the Shiga toxin effector polypeptide comprises a disruption of at least one endogenous epitope region selected from the group of natively positioned Shiga toxin A Subunits consisting of: (ii) 94-115 of any one of SEQ ID NOs: 1-18; 141-153 of any one of SEQ ID NOs: 1-2 and 4-6; 140-156 of any one of SEQ ID NOs: 3 and 7-18; 179-190 of any one of SEQ ID NOs: 1-2 and 4-6; 179-191 of any one of SEQ ID NOs: 3 and 7-18; 204 of SEQ ID NO:3; 205 of any one of SEQ ID NOs: 1-2 and 4-6; and 210-218 of any one of SEQ ID NOs: 3 and 7-18, or the equivalent region in a Shiga toxin A Subunit or derivative thereof.

In some embodiments, the Shiga toxin effector polypeptide comprises disruptions of at least four, five, six, seven, eight, or more endogenous, B-cell and/or T-cell epitope regions.

In some embodiments, one or more disruptions comprises an amino acid residue substitution relative to a wild-type Shiga toxin A Subunit.

In some embodiments, one or more endogenous, B-cell and/or T-cell epitope regions comprises a plurality of amino acid residue substitutions relative to a wild-type Shiga toxin A Subunit.

In some embodiments, at least one, two, three, or four disruptions comprise a plurality of amino acid residue substitutions in the endogenous, B-cell and/or T-cell epitope region relative to a wild-type Shiga toxin A Subunit.

In some embodiments, at least one disruption comprises at least one, two, three, four, five, six, seven, eight, or more amino acid residue substitutions relative to a wild-type Shiga toxin A Subunit, and optionally wherein at least one substitution occurs at the natively positioned Shiga toxin A Subunit amino acid residue selected form the group consisting of: 1 of SEQ ID NO:1 or SEQ ID NO:2; 4 of SEQ ID NO:1, SEQ ID NO:2, or SEQ ID NO:3; 6 of SEQ ID NO:1, SEQ ID NO:2, or SEQ ID NO:3; 8 of SEQ ID NO:1, SEQ ID NO:2, or SEQ ID NO:3; 9 of SEQ ID NO:1, SEQ ID NO:2, or SEQ ID NO:3; 11 of SEQ ID NO:1, SEQ ID NO:2, or SEQ ID NO:3; 12 of SEQ ID NO:1, SEQ ID NO:2, or SEQ ID NO:3; 33 of SEQ ID NO:1 or SEQ ID NO:2; 43 of SEQ ID NO:1 or SEQ ID NO:2; 44 of SEQ ID NO:1 or SEQ ID NO:2; 45 of SEQ ID NO:1 or SEQ ID NO:2; 46 of SEQ ID NO:1, SEQ ID NO:2, or SEQ ID NO:3; 47 of SEQ ID NO:1 or SEQ ID NO:2; 48 of SEQ ID NO:1, SEQ ID NO:2, or SEQ ID NO:3; 49 of SEQ ID NO:1, SEQ ID NO:2, or SEQ ID NO:3; 50 of SEQ ID NO:1 or SEQ ID NO:2; 51 of SEQ ID NO:1 or SEQ ID NO:2; 53 of SEQ ID NO:1 or SEQ ID NO:2; 54 of SEQ ID NO:1, SEQ ID NO:2, or SEQ ID NO:3; 55 of SEQ ID NO:1 or SEQ ID NO:2; 56 of SEQ ID NO:1, SEQ ID NO:2, or SEQ ID NO:3; 57 of SEQ ID NO:1, SEQ ID NO:2, or SEQ ID NO:3; 58 of SEQ ID NO:1, SEQ ID NO:2, or SEQ ID NO:3; 59 of SEQ ID NO:1, SEQ ID NO:2, or SEQ ID NO:3; 60 of SEQ ID NO:1 or SEQ ID NO:2; 61 of SEQ ID NO:1 or SEQ ID NO:2; 62 of SEQ ID NO:1 or SEQ ID NO:2; 84 of SEQ ID NO:1 or SEQ ID NO:2; 88 of SEQ ID NO:1, SEQ ID NO:2, or SEQ ID NO:3; 94 of SEQ ID NO:1, SEQ ID NO:2, or SEQ ID NO:3; 96 of SEQ ID NO:1, SEQ ID NO:2, or SEQ ID NO:3; 104 of SEQ ID NO:1 or SEQ ID NO:2; 105 of SEQ ID NO:1 or SEQ ID NO:2; 107 of SEQ ID NO:1, SEQ ID NO:2, or SEQ ID NO:3; 108 of SEQ ID NO:1 or SEQ ID NO:2; 109 of SEQ ID NO:1, SEQ ID NO:2, or SEQ ID NO:3; 110 of SEQ ID NO:1 or SEQ ID NO:2; 111 of SEQ ID NO:1, SEQ ID NO:2, or SEQ ID NO:3; 112 of SEQ ID NO:1, SEQ ID NO:2, or SEQ ID NO:3; 141 of SEQ ID NO:1, SEQ ID NO:2, or SEQ ID NO:3; 147 of SEQ ID NO:1, SEQ ID NO:2, or SEQ ID NO:3; 154 of SEQ ID NO:1 or SEQ ID NO:2; 179 of SEQ ID NO:1, SEQ ID NO:2, or SEQ ID NO:3; 180 of SEQ ID NO:1 or SEQ ID NO:2; 181 of SEQ ID NO:1 or SEQ ID NO:2; 183 of SEQ ID NO:1, SEQ ID NO:2, or SEQ ID NO:3; 184 of SEQ ID NO:1, SEQ ID NO:2, or SEQ ID NO:3; 185 of SEQ ID NO:1 or SEQ ID NO:2; 186 of SEQ ID NO:1, SEQ ID NO:2, or SEQ ID NO:3; 187 of SEQ ID NO:1 or SEQ ID NO:2; 188 of SEQ ID NO:1 or SEQ ID NO:2; 189 of SEQ ID NO:1 or SEQ ID NO:2; 197 of SEQ ID NO:3; 198 of SEQ ID NO:1 or SEQ ID NO:2; 204 of SEQ ID NO:3; 205 of SEQ ID NO:1 or SEQ ID NO:2; 247 of SEQ ID NO:1 or SEQ ID NO:2; 247 of SEQ ID NO:3; 248 of SEQ ID NO:1 or SEQ ID NO:2; 250 of SEQ ID NO:3; 251 of SEQ ID NO:1 or SEQ ID NO:2; 264 of SEQ ID NO:1, SEQ ID NO:2, or SEQ ID NO:3; 265 of SEQ ID NO:1 or SEQ ID NO:2; and 286 of SEQ ID NO:1 or SEQ ID NO:2; or the equivalent amino acid residue in a Shiga toxin A Subunit or derivative thereof. In some embodiments, at least two disruptions each comprise at least one amino acid residue substitutions relative to a wild-type Shiga toxin A Subunit selected form the group consisting of: 1 of SEQ ID NO:1 or SEQ ID NO:2; 4 of SEQ ID NO:1, SEQ ID NO:2, or SEQ ID NO:3; 8 of SEQ ID NO:1, SEQ ID NO:2, or SEQ ID NO:3; 9 of SEQ ID NO:1, SEQ ID NO:2, or SEQ ID NO:3; 11 of SEQ ID NO:1, SEQ ID NO:2, or SEQ ID NO:3; 33 of SEQ ID NO:1 or SEQ ID NO:2; 43 of SEQ ID NO:1 or SEQ ID NO:2; 45 of SEQ ID NO:1 or SEQ ID NO:2; 47 of SEQ ID NO:1 or SEQ ID NO:2; 48 of SEQ ID NO:1, SEQ ID NO:2, or SEQ ID NO:3; 49 of SEQ ID NO:1 or SEQ ID NO:2; 53 of SEQ ID NO:1 or SEQ ID NO:2; 55 of SEQ ID NO:1 or SEQ ID NO:2; 58 of SEQ ID NO:1, SEQ ID NO:2, or SEQ ID NO:3; 59 of SEQ ID NO:1, SEQ ID NO:2, or SEQ ID NO:3; 60 of SEQ ID NO:1 or SEQ ID NO:2; 61 of SEQ ID NO:1 or SEQ ID NO:2; 62 of SEQ ID NO:1 or SEQ ID NO:2; 94 of SEQ ID NO:1, SEQ ID NO:2, or SEQ ID NO:3; 96 of SEQ ID NO:1, SEQ ID NO:2, or SEQ ID NO:3; 109 of SEQ ID NO:1, SEQ ID NO:2, or SEQ ID NO:3; 110 of SEQ ID NO:1 or SEQ ID NO:2; 112 of SEQ ID NO:1, SEQ ID NO:2, or SEQ ID NO:3; 147 of SEQ ID NO:1, SEQ ID NO:2, or SEQ ID NO:3; 179 of SEQ ID NO:1, SEQ ID NO:2, or SEQ ID NO:3; 180 of SEQ ID NO:1 or SEQ ID NO:2; 181 of SEQ ID NO:1 or SEQ ID NO:2; 183 of SEQ ID NO:1, SEQ ID SEQ ID NO:2, or SEQ ID NO:3; 184 of SEQ ID NO:1, SEQ ID NO:2, or SEQ ID NO:3; 185 of SEQ ID NO:1 or SEQ ID NO:2; 186 of SEQ ID NO:1, SEQ ID NO:2, or SEQ ID NO:3; 187 of SEQ ID NO:1 or SEQ ID NO:2; 188 of SEQ ID NO:1 or SEQ ID NO:2; 189 of SEQ ID NO:1 or SEQ ID NO:2; 204 of SEQ ID NO:3; 205 of SEQ ID NO:1 or SEQ ID NO:2; 247 of SEQ ID NO:1 or SEQ ID NO:2; 247 of SEQ ID NO:3; 250 of SEQ ID NO:3; 264 of SEQ ID NO:1, SEQ ID NO:2, or SEQ ID NO:3; 265 of SEQ ID NO:1 or SEQ ID NO:2; and 286 of SEQ ID NO:1 or SEQ ID NO:2; or the equivalent amino acid residue in a Shiga toxin A Subunit or derivative thereof.

In some embodiments, the Shiga toxin effector polypeptide comprises disruption of at least three, endogenous, B-cell and/or T-cell epitope regions selected from the group of consisting of: (i) 1-15 of any one of SEQ ID NOs: 1-2 and 4-6; 3-14 of any one of SEQ ID NOs: 3 and 7-18; 26-37 of any one of SEQ ID NOs: 3 and 7-18; 27-37 of any one of SEQ ID NOs: 1-2 and 4-6; 39-48 of any one of SEQ ID NOs: 1-2 and 4-6; 42-48 of any one of SEQ ID NOs: 3 and 7-18; and 53-66 of any one of SEQ ID NOs: 1-18, or the equivalent region in a Shiga toxin A Subunit or derivative thereof, wherein there is no disruption which is an amino-terminal truncation of amino acid residues that overlap with part or all of at least one disrupted, endogenous, B-cell and/or T-cell epitope region; (ii) 94-115 of any one of SEQ ID NOs: 1-18; 141-153 of any one of SEQ ID NOs: 1-2 and 4-6; 140-156 of any one of SEQ ID NOs: 3 and 7-18; 179-190 of any one of SEQ ID NOs: 1-2 and 4-6; 179-191 of any one of SEQ ID NOs: 3 and 7-18; 204 of SEQ ID NO:3; 205 of any one of SEQ ID NOs: 1-2 and 4-6; and 210-218 of any one of SEQ ID NOs: 3 and 7-18, or the equivalent region in a Shiga toxin A Subunit or derivative thereof, wherein there is no disruption which is a carboxy-terminal truncation of amino acid residues that overlap with part or all of at least one disrupted, endogenous, B-cell and/or T-cell epitope and/or epitope region.

In some embodiments, the Shiga toxin effector polypeptide comprises disruptions of at least two, endogenous, B-cell and/or T-cell epitope regions, wherein each disruption comprises one or more amino acid residue substitutions, and wherein the endogenous, B-cell and/or T-cell epitope regions are selected from the group of natively positioned Shiga toxin A Subunit regions consisting of: 3-14 of any one of SEQ ID NOs: 3 and 7-18; 26-37 of any one of SEQ ID NOs: 3 and 7-18; 27-37 of any one of SEQ ID NOs: 1-2 and 4-6; 39-48 of any one of SEQ ID NOs: 1-2 and 4-6; 42-48 of any one of SEQ ID NOs: 3 and 7-18; 53-66 of any one of SEQ ID NOs: 1-18; or the equivalent region in a Shiga toxin A Subunit or derivative thereof.

In some embodiments, the embedded or inserted, heterologous, T-cell epitope does not disrupt any endogenous, B-cell and/or CD4+ T-cell epitope region described herein.

In some embodiments, at least one disruption comprises one or more amino acid residue substitutions relative to a wild-type Shiga toxin A Subunit is selected from the group consisting of: D to A, D to G, D to V, D to L, D to I, D to F, D to S, D to Q, D to M, D to R, E to A, E to G, E to V, E to L, E to I, E to F, E to S, E to Q, E to N, E to D, E to M, E to R, F to A, F to G, F to V, F to L, F to I, G to A, G to P, H to A, H to G, H to V, H to L, H to I, H to F, H to M, I to A, I to V, I to G, I to C, K to A, K to G, K to V, K to L, K to I, K to M, K to H, L to A, L to V, L to G, L to C, N to A, N to G, N to V, N to L, N to I, N to F, P to A, P to G, P to F, R to A, R to G, R to V, R to L, R to I, R to F, R to M, R to Q, R to S, R to K, R to H, S to A, S to G, S to V, S to L, S to I, S to F, S to M, T to A, T to G, T to V, T to L, T to I, T to F, T to M, T to S, V to A, V to G, Y to A, Y to G, Y to V, Y to L, Y to I, Y to F, Y to M, and Y to T. In some embodiments, the one or more amino acid residue substitutions relative to a wild-type Shiga toxin A Subunit is selected from the group consisting of: D to A, D to G, D to V, D to L, D to I, D to F, D to S, D to Q, E to A, E to G, E to V, E to L, E to I, E to F, E to S, E to Q, E to N, E to D, E to M, E to R, G to A, H to A, H to G, H to V, H to L, H to I, H to F, H to M, K to A, K to G, K to V, K to L, K to I, K to M, K to H, L to A, L to G, N to A, N to G, N to V, N to L, N to I, N to F, P to A, P to G, P to F, R to A, R to G, R to V, R to L, R to I, R to F, R to M, R to Q, R to S, R to K, R to H, S to A, S to G, S to V, S to L, S to I, S to F, S to M, T to A, T to G, T to V, T to L, T to I, T to F, T to M, T to S, Y to A, Y to G, Y to V, Y to L, Y to I, Y to F, and Y to M.

In some embodiments, at least one of the disruption(s) comprises one or more amino acid residue substitutions relative to a wild-type Shiga toxin A Subunit selected from the group consisting of: K1 to A, G, V, L, I, F, M and H; T4 to A, G, V, L, I, F, M, and S; D6 to A, G, V, L, I, F, S, Q and R; S8 to A, G, V, I, L, F, and M; T9 to A, G, V, I, L, F, M, and S; S9 to A, G, V, L, I, F, and M; K11 to A, G, V, L, I, F, M and H; T12 to A, G, V, I, L, F, M, S, and K; S12 to A, G, V, I, L, F, and M; S33 to A, G, V, L, I, F, M, and C; S43 to A, G, V, L, I, F, and M; G44 to A or L; S45 to A, G, V, L, I, F, and M; T45 to A, G, V, L, I, F, and M; G46 to A and P; D47 to A, G, V, L, I, F, S, M, and Q; N48 to A, G, V, L, M and F; L49 to A, V, C, and G; Y49 to A, G, V, L, I, F, M, and T; F50 to A, G, V, L, I, and T; A51; D53 to A, G, V, L, I, F, S, and Q; V54 to A, G, I, and L; R55 to A, G, V, L, I, F, M, Q, S, K, and H; G56 to A and P; 157 to A, G, V, and M; L57 to A, V, C, G, M, and F; D58 to A, G, V, L, I, F, S, and Q; P59 to A, G, and F; E60 to A, G, V, L, I, F, S, Q, N, D, M, T, and R; E61 to A, G, V, L, I, F, S, Q, N, D, M, and R; G62 to A; R84 to A, G, V, L, I, F, M, Q, S, K, and H; V88 to A and G; I88 to A, V, C, and G; D94 to A, G, V, L, I, F, S, and Q; S96 to A, G, V, I, L, F, and M; T104 to A, G, V, L, I, F, M; and N; A105 to L; T107 to A, G, V, L, I, F, M, and P; S107 to A, G, V, L, I, F, M, and P; L108 to A, V, C, and G; S109 to A, G, V, I, L, F, and M; T109 to A, G, V, I, L, F, M, and S; G110 to A; S112 to A, G, V, L, I, F, and M; D111 to A, G, V, L, I, F, S, Q, and T; S112 to A, G, V, L, I, F, and M; D141 to A, G, V, L, I, F, S, and Q; G147 to A; V154 to A and G. R179 to A, G, V, L, I, F, M, Q, S, K, and H; T180 to A, G, V, L, I, F, M, and S; T181 to A, G, V, L, I, F, M, and S; D183 to A, G, V, L, I, F, S, and Q; D184 to A, G, V, L, I, F, S, and Q; L185 to A, G, V and C; S186 to A, G, V, I, L, F, and M; G187 to A; R188 to A, G, V, L, I, F, M, Q, S, K, and H; S189 to A, G, V, I, L, F, and M; D197 to A, G, V, L, I, F, S, and Q; D198 to A, G, V, L, I, F, S, and Q; R204 to A, G, V, L, I, F, M, Q, S, K, and H; R205 to A, G, V, L, I, F, M, Q, S, K and H; S247 to A, G, V, I, L, F, and M; Y247 to A, G, V, L, I, F, and M; R248 to A, G, V, L, I, F, M, Q, S, K, and H; R250 to A, G, V, L, I, F, M, Q, S, K, and H; R251 to A, G, V, L, I, F, M, Q, S, K, and H; D264 to A, G, V, L, I, F, S, and Q; G264 to A; and T286 to A, G, V, L, I, F, M, and S.

In some embodiments, the PD-L1 targeting molecule is capable when introduced to a chordate of exhibiting improved in vivo tolerability and/or stability compared to a reference molecule, such as, e.g., a second PD-L1 targeting molecule consisting of the PD-L1 targeting molecule except for all of its Shiga toxin effector polypeptide component(s) each comprise a wild-type Shiga toxin A1 fragment and/or wild-type Shiga toxin furin-cleavage site at the carboxy terminus of its A1 fragment region. In some embodiments, the Shiga toxin effector polypeptide is not cytotoxic and the molecular moiety is cytotoxic.

In some embodiments, the binding region comprises a polypeptide selected from the group consisting of: autonomous V_{H} domains, single-domain antibody domains (sdAbs), heavy-chain antibody domains derived from camelids (V_{H}H fragments or V_{H} domain fragments), heavy-chain antibody domains derived from camelid V_{H}H fragments or V_{H} domain fragments, heavy-chain antibody domains derived from cartilaginous fishes, immunoglobulin new antigen receptors (IgNARs), V_{NAR} fragments, single-chain variable (scFv) fragments, nanobodies, Fd fragments consisting of the heavy chain and C_{H}1 domains, single chain Fv-C_{H}3 minibodies, dimeric C_{H}2 domain fragments (C_{H}2D), Fc antigen binding domains (Fcabs), isolated complementary determining region 3 (CDR3) fragments, constrained framework region 3, CDR3, framework region 4 (FR3-CDR3-FR4) polypeptides, small modular immunopharmaceutical (SMIP) domains, scFv-Fc fusions, multimerizing scFv fragments (diabodies, triabodies, tetrabodies), disulfide stabilized antibody variable (Fv) fragments, disulfide stabilized antigen-binding (Fab) fragments consisting of the V_{L}, V_{H}, C_{L} and C_{H}1 domains, bivalent nanobodies, bivalent minibodies, bivalent F(ab′)₂ fragments (Fab dimers), bispecific tandem V_{H}H fragments, bispecific tandem scFv fragments, bispecific nanobodies, bispecific minibodies, and any genetically manipulated counterparts of the foregoing that retain its paratope and binding functionality.

In some embodiments, the PD-L1 targeting molecule and/or its Shiga toxin effector polypeptide is capable of exhibiting subcellular routing efficiency comparable to a reference PD-L1 targeting molecule comprising a wild-type Shiga toxin A1 fragment or wild-type Shiga toxin effector polypeptide and/or capable of exhibiting a significant level of intracellular routing activity to the endoplasmic reticulum and/or cytosol from an endosomal starting location of a cell.

In some embodiments, whereby administration of the PD-L1 targeting molecule to a cell physically coupled with the extracellular target biomolecule of the PD-L1 targeting molecule's binding region, the PD-L1 targeting molecule is capable of causing death of the cell. In some embodiments, administration of the PD-L1 targeting molecule to two different populations of cell types which differ with respect to the presence or level of the extracellular target biomolecule, the PD-L1 targeting molecule is capable of causing cell death to the cell-types physically coupled with extracellular PD-L1 bound by the cytotoxic PD-L1 targeting molecule's binding region at a CD₅₀ at least three times or less than the CD₅₀ to cell types which are not physically coupled with an extracellular PD-L1 bound by the PD-L1 targeting molecule's binding region. In some embodiments, whereby administration of the PD-L1 targeting molecule to a first populations of cells whose members are physically coupled to PD-L1 bound by the PD-L1 targeting molecule's binding region, and a second population of cells whose members are not physically coupled to any extracellular PD-L1 bound by the binding region, the cytotoxic effect of the PD-L1 targeting molecule to members of said first population of cells relative to members of said second population of cells is at least 3-fold greater. For some embodiments, whereby administration of the PD-L1 targeting molecule to a first populations of cells whose members are physically coupled to a significant amount of the PD-L1 bound by the PD-L1 targeting molecule's binding region, and a second population of cells whose members are not physically coupled to a significant amount of any PD-L1 bound by the binding region, the cytotoxic effect of the PD-L1 targeting molecule to members of said first population of cells relative to members of said second population of cells is at least 3-fold greater. In some embodiments, whereby administration of the PD-L1 targeting molecule to a first population of target biomolecule positive cells, and a second population of cells whose members do not express a significant amount of a target biomolecule of the PD-L1 targeting molecule's binding region at a cellular surface, the cytotoxic effect of the PD-L1 targeting molecule to members of the first population of cells relative to members of the second population of cells is at least 3-fold greater.

In some embodiments, the PD-L1 targeting molecule is capable of delivering an embedded or inserted, heterologous, CD8+ T-cell epitope to a MHC class I presentation pathway of a cell for cell-surface presentation of the epitope bound by a MHC class I molecule.

In some embodiments, the PD-L1 targeting molecule is capable of delivering heterologous, CD8+ T-cell epitope (which is neither embedded nor inserted in the Shiga toxin effector polypeptide) to a MHC class I presentation pathway of a cell for cell-surface presentation of the epitope bound by a MHC class I molecule. In some embodiments, the heterologous, T-cell epitope is a CD8+ T-cell epitope, such as, e.g., with regard to a human immune system. In some embodiments, the heterologous, T-cell epitope is capable of being presented by a MHC class I molecule of a cell. In some embodiments, the PD-L1 targeting molecule is capable of one or more the following: entering a cell, inhibiting a ribosome function, causing cytostasis, causing cell death, and/or delivering the embedded or inserted, heterologous, T-cell epitope to a MHC class I molecule for presentation on a cellular surface.

In some embodiments, the PD-L1 targeting molecule comprises a molecular moiety associated with the carboxy-terminus of the Shiga toxin effector polypeptide. In some embodiments, the molecular moiety comprises or consists of the binding region. In some embodiments, the molecular moiety comprises at least one amino acid and the Shiga toxin effector polypeptide is linked to at least one amino acid residue of the molecular moiety. In some embodiments, the molecular moiety and the Shiga toxin effector polypeptide are fused forming a continuous polypeptide.

In some embodiments, the PD-L1 targeting molecule further comprises a cytotoxic molecular moiety associated with the carboxy-terminus of the Shiga toxin effector polypeptide. For some embodiments, the cytotoxic molecular moiety is a cytotoxic agent, such as, e.g., a small molecule chemotherapeutic agent, anti-neoplastic agent, cytotoxic antibiotic, alkylating agent, antimetabolite, topoisomerase inhibitor, and/or tubulin inhibitor known to the skilled worker and/or described herein. For some embodiments, the cytotoxic molecular moiety is cytotoxic at concentrations of less than 10,000, 5,000, 1,000, 500, or 200 pM.

In some embodiments, the binding region is linked, either directly or indirectly, to the Shiga toxin effector polypeptide by at least one covalent bond which is not a disulfide bond. In some embodiments, the binding region is fused, either directly or indirectly, to the carboxy-terminus of the Shiga toxin effector polypeptide to form a single, continuous polypeptide. In some embodiments, the binding region is an immunoglobulin-type binding region.

In some embodiments, the disrupted furin-cleavage site comprises one or more mutations in the minimal, furin-cleavage site relative to a wild-type Shiga toxin A Subunit. In some embodiments, the disrupted furin-cleavage site is not an amino-terminal truncation of sequences that overlap with part or all of at least one amino acid residue of the minimal furin-cleavage site. In some embodiments, the mutation in the minimal, furin-cleavage site is an amino acid deletion, insertion, and/or substitution of at least one amino acid residue in the R/Y-x-x-R furin cleavage site (SEQ ID NO: 159). In some embodiments, the disrupted furin-cleavage site comprises at least one mutation relative to a wild-type Shiga toxin A Subunit, the mutation altering at least one amino acid residue in the region natively positioned at 248-251 of the A Subunit of Shiga toxin (SEQ ID NOs: 1-2 and 4-6), or at 247-250 of the A Subunit of Shiga-like toxin 2 (SEQ ID NOs: 3 and 7-18), or the equivalent amino acid sequence position in any Shiga toxin A Subunit. In some embodiments, the mutation is an amino acid residue substitution of an arginine residue with a non-positively charged, amino acid residue.

In some embodiments, the PD-L1 targeting molecule is capable when introduced to cells of exhibiting cytotoxicity comparable to a cytotoxicity of a reference molecule, such as, e.g., a second PD-L1 targeting molecule consisting of the PD-L1 targeting molecule except for all of its Shiga toxin effector polypeptide component(s) each comprise a wild-type Shiga toxin A1 fragment.

In some embodiments, the PD-L1 targeting molecule comprises any one of SEQ ID NOs: 1-83.

In some embodiments, one or more binding region(s) comprises the peptide or polypeptide shown in any one of SEQ ID NOs: 19-40.

In some embodiments, two or more binding regions comprise the peptide or polypeptide shown in any one of SEQ ID NOs: 19-40. In some embodiments, two or more binding regions each comprise the same peptide or polypeptide shown in any one of SEQ ID NOs: 19-40.

Some embodiments of the PD-L1 targeting molecule comprise or consist essentially of the polypeptide represented by the amino acid sequence shown in any one of SEQ ID NOs: 84-166 or 188-256.

In some embodiments, at least one binding region sterically covers the carboxy-terminus of the A1 fragment region.

In some embodiments, the molecular moiety sterically covers the carboxy-terminus of the A1 fragment region. In some embodiments, the molecular moiety comprises the binding region.

In some embodiments, the PD-L1 targeting molecule comprises a binding region and/or molecular moiety located carboxy-terminal to the carboxy-terminus of the Shiga toxin A1 fragment region. In some embodiments, the mass of the binding region and/or molecular moiety is at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater.

In some embodiments, the PD-L1 targeting molecule comprises a binding region with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the PD-L1 targeting molecule retains the appropriate level of the Shiga toxin biological activity noted herein (e.g., cytotoxicity and/or intracellular routing).

In some embodiments, the binding region is comprised within a relatively large, molecular moiety comprising such as, e.g., a molecular moiety with a mass of at least 4.5 kDa, 6, kDa, 9 kDa, 12 kDa, 15 kDa, 20 kDa, 25 kDa, 28 kDa, 30 kDa, 41 kDa, 50 kDa, 100 kDa, or greater, as long as the PD-L1 targeting molecule retains the appropriate level of the Shiga toxin biological activity noted herein.

In some embodiments, the PD-L1 targeting molecule exhibits low cytotoxic potency (i.e. is not capable when introduced to certain positive target cell types of exhibiting a cytotoxicity greater than 1% cell death of a cell population at a PD-L1 targeting molecule concentration of 1000 nM, 500 nM, 100 nM, 75 nM, or 50 nM) and is capable when introduced to cells of exhibiting a greater subcellular routing efficiency from an extracellular space to a subcellular compartment of an endoplasmic reticulum and/or cytosol as compared to the cytotoxicity of a reference molecule, such as, e.g., a second PD-L1 targeting molecule having an amino-terminus and comprising the binding region and the Shiga toxin effector polypeptide which is not positioned at or proximal to the amino-terminus of the third PD-L1 targeting molecule. In some embodiments, the reference PD-L1 targeting molecule does not comprise any carboxy-terminal, endoplasmic reticulum retention/retrieval signal motif of the KDEL family.

In some embodiments, the molecular moiety comprises a peptide and/or polypeptide derived from the Shiga toxin A2 fragment of a naturally occurring Shiga toxin.

The embodiments are not intended to cover any naturally occurring Shiga holotoxin. In some embodiments, the PD-L1 targeting molecule does not comprise a naturally occurring Shiga toxin B Subunit. In some embodiments, the PD-L1 targeting molecule does not comprise any polypeptide comprising or consisting essentially of a functional binding domain of a native Shiga toxin B subunit. Rather, in some embodiments, the Shiga toxin A Subunit derived regions are functionally associated with heterologous binding regions to effectuate PD-L1 targeting.

In some embodiments, the target cell is not a professional antigen presenting cell, such as a dendritic cell type.

In some embodiments, the Shiga toxin effector polypeptide comprises at least two, embedded or inserted, heterologous epitopes.

In some embodiments, the Shiga toxin effector polypeptide comprises one or more mutations relative to a naturally occurring A Subunit of a member of the Shiga toxin family which changes an enzymatic activity of the Shiga toxin effector polypeptide, the mutation selected from at least one amino acid residue deletion, insertion, or substitution. In some embodiments, the mutation relative to the naturally occurring A Subunit reduces of eliminates a cytotoxic activity of the Shiga toxin effector polypeptide but the Shiga toxin effector polypeptide retains at least one other Shiga toxin effector function, such as, e.g., promoting cellular internalization and/or directing intracellular routing to a certain subcellular compartment(s). In some embodiments, the mutation relative to the naturally occurring A Subunit is selected from at least one amino acid residue substitution, such as, e.g., A231E, N75A, Y77S, Y114S, E167D, R170A, R176K, W202A, and/or W203A in any one of SEQ ID NOs: 1-18.

In some embodiments, the Shiga toxin effector polypeptide is capable of: (i) routing to a subcellular compartment of a cell in which the Shiga toxin effector polypeptide is present selected from the following: cytosol, endoplasmic reticulum, and lysosome; (ii) intracellular delivery of the epitope-cargo from an early endosomal compartment to a proteasome of a cell in which the Shiga toxin effector polypeptide is present; and/or (iii) intracellular delivery of the epitope to a MHC class I molecule from an early endosomal compartment of a cell in which the Shiga toxin effector polypeptide is present. In some embodiments, the Shiga toxin effector polypeptide is capable of intracellular delivery of the CD8+ T-cell epitope for presentation by a MHC class I molecule on the surface of a cell in which the Shiga toxin effector polypeptide is present.

Also provided herein are pharmaceutical compositions comprising a PD-L1 targeting molecule and at least one pharmaceutically acceptable excipient or carrier; and the use of such a PD-L1 targeting molecule, or a composition comprising it, in methods described herein. Some embodiments are pharmaceutical compositions comprising any PD-L1 targeting molecule; and at least one pharmaceutically acceptable excipient or carrier.

Also provided herein is a diagnostic composition comprising any one of the above PD-L1 targeting molecules and a detection promoting agent for the collection of information, such as diagnostically useful information about a cell-type, tissue, organ, disease, disorder, condition, and/or patient. In some embodiments, the detection promoting agent is a heterologous epitope-peptide cargo and the PD-L1 targeting molecule comprises the heterologous epitope-peptide cargo.

Beyond the PD-L1 targeting molecules and compositions, polynucleotides capable of encoding a PD-L1 targeting molecule, or a protein component thereof, are also contemplated herein, as well as expression vectors which comprise a polynucleotide and host cells comprising an expression vector described herein. Host cells comprising an expression vector may be used, e.g., in methods for producing a PD-L1 targeting molecule, or a protein component or fragment thereof, by recombinant expression.

In some embodiments, any composition of matter described herein may be immobilized on a solid substrate. Such arrangements of the compositions of matter may be utilized, e.g., in methods of screening molecules as described herein.

In some embodiments, a method of delivering into a cell a CD8+ T-cell epitope-peptide cargo capable of being presented by a MHC class I molecule of the cell comprises the step of contacting the cell with the PD-L1 targeting molecule and/or a composition thereof (e.g., a pharmaceutical or diagnostic composition).

Among some embodiments is a method of inducing a cell to present an exogenously administered CD8+ T-cell epitope-peptide cargo complexed to a MHC class I molecule, the method comprising the step of contacting the cell, either in vitro or in vivo, with the PD-L1 targeting molecule, which comprises the CD8+ T-cell epitope, and/or a composition thereof (e.g., a pharmaceutical or diagnostic composition comprising such a PD-L1 targeting molecule).

Among some embodiments is a method of inducing an immune cell-mediated response to target cell via a CD8+ T-cell epitope MHC class I molecule complex, the method comprising the step of contacting the target cell either in vitro or in vivo, with the PD-L1 targeting molecule, which comprises the CD8+ T-cell epitope as a cargo, and/or a composition thereof (e.g., a pharmaceutical or diagnostic composition comprising such a PD-L1 targeting molecule). For some embodiments, the immune response is selected from the group consisting: CD8+ immune cell secretion of a cytokine(s), cytotoxic T lymphocyte-(CTL) induced growth arrest in the target cell, CTL-induced necrosis of the target cell, CTL-induced apoptosis of the target cell, immune cell-mediated cell killing of a cell other than the target cell.

In some embodiments, a method of causing intercellular engagement of a CD8+ immune cell with a target cell comprises the step of contacting the target cell with the PD-L1 targeting molecule in the presence of a CD8+ immune cell or with the subsequent step of contacting the target cell with one or more CD8+ immune cells. For some embodiments, the contacting step occurs in vitro. For certain other embodiments, the contacting step occurs in vivo, such as, e.g., by administering the PD-L1 targeting molecule to a chordate, vertebrate, and/or mammal. For some embodiments, the intercellular engagement occurs in vitro. For some embodiments, the intercellular engagement occurs in vivo.

In some embodiments, a composition comprises a PD-L1 targeting molecule for “seeding” a tissue locus within a chordate.

In some embodiments, a method for “seeding” a tissue locus within a chordate comprises the step of: administering to the chordate a PD-L1 targeting molecule, or a pharmaceutical composition comprising the same. For some embodiments, the method is for “seeding” a tissue locus within a chordate which comprises a malignant, diseased, and/or inflamed tissue. For some embodiments, the method is for “seeding” a tissue locus within a chordate which comprises the tissue selected from the group consisting of: diseased tissue, tumor mass, cancerous growth, tumor, infected tissue, or abnormal cellular mass. For some embodiments, the method for “seeding” a tissue locus within a chordate comprises the step of: administering to the chordate a PD-L1 targeting molecule comprising a heterologous, CD8+ T-cell epitope-peptide cargo selected from the group consisting of: peptides not natively presented by the target cells of the PD-L1 targeting molecule in MHC class I complexes, peptides not natively present within any protein expressed by the target cell, peptides not natively present within the transcriptome and/or proteome of the target cell, peptides not natively present in the extracellular microenvironment of the site to be seeded, and peptides not natively present in the tumor mass or infected tissue site to be targeted.

Additionally, provided herein are methods of killing a cell(s) comprising the step of contacting a cell(s) with a PD-L1 targeting molecule or a pharmaceutical composition comprising a PD-L1 targeting molecule. In some embodiments, the step of contacting the cell(s) occurs in vitro. In some embodiments, the step of contacting the cell(s) occurs in vivo. In some embodiments, the method is capable of selectively killing cell(s) and/or cell-types preferentially over other cell(s) and/or cell-types when contacting a mixture of cells which differ with respect to the extracellular presence and/or expression level of extracellular PD-L1 bound by the binding region of the PD-L1 targeting molecule.

Also provided herein are methods of treating diseases, disorders, and/or conditions in patients in need thereof comprising the step of administering to a patient in need thereof a therapeutically effective amount of a composition comprising a PD-L1 targeting molecule or pharmaceutical composition comprising the same. In some embodiments, the disease, disorder, or condition to be treated is selected from: a cancer, tumor, growth abnormality, immune disorder, or microbial infection. In some embodiments, the cancer to be treated is selected from the group consisting of: bone cancer, breast cancer, central/peripheral nervous system cancer, gastrointestinal cancer, germ cell cancer, glandular cancer, head-neck cancer, hematological cancer, kidney-urinary tract cancer, liver cancer, lung/pleura cancer, prostate cancer, sarcoma, skin cancer, and uterine cancer. In some embodiments, the immune disorder to be treated is an immune disorder associated with a disease selected from the group consisting of: amyloidosis, ankylosing spondylitis, asthma, Crohn's disease, diabetes, graft rejection, graft-versus-host disease, Hashimoto's thyroiditis, hemolytic uremic syndrome, HIV-related diseases, lupus erythematosus, multiple sclerosis, polyarteritis nodosa, polyarthritis, psoriasis, psoriatic arthritis, rheumatoid arthritis, scleroderma, septic shock, Sjorgren's syndrome, ulcerative colitis, and vasculitis.

Also provided herein is a composition comprising a PD-L1 targeting molecule for the treatment or prevention of a cancer, tumor, growth abnormality, immune disorder, or microbial infection. In some embodiments, provided herein is use of a composition of matter as described herein in the manufacture of a medicament for the treatment or prevention of a cancer, tumor, growth abnormality, immune disorder, or microbial infection.

The use of any composition described for the treatment or prevention of a cancer, tumor, growth abnormality, and/or immune disorder is contemplated by the disclosure.

In some embodiments, a method of treating cancer in a patient using immunotherapy comprises the step of administering to the patient in need thereof the PD-L1 targeting molecule and/or a pharmaceutical composition comprising the same.

Any composition of matter described herein may be used for the treatment or prevention of a cancer, tumor, growth abnormality, and/or immune disorder. In some embodiments, a PD-L1 targeting molecule and/or a pharmaceutical composition comprising the same is used for the treatment or prevention of a cancer, tumor, growth abnormality, immune disorder, and/or microbial infection. In some embodiments, provided herein is the use of a PD-L1 targeting molecule and/or pharmaceutical composition thereof in the manufacture of a medicament for the treatment or prevention of a cancer, tumor, growth abnormality, immune disorder, or microbial infection.

In some embodiments, a composition comprising a PD-L1 targeting molecule is for the delivery of one or more additional exogenous materials into a cell physically coupled with PD-L1 bound by the binding region of the PD-L1 targeting molecule. Some embodiments of the PD-L1 targeting molecules may be used to deliver one or more additional exogenous materials into a cell physically coupled with PD-L1 bound by the binding region of the PD-L1 targeting molecule. Additionally, in some embodiments, a method for delivering exogenous material to the inside of a cell(s) comprises contacting the cell(s), either in vitro or in vivo, with a PD-L1 targeting molecule, pharmaceutical composition, and/or diagnostic composition. Also provided herein is a method for delivering exogenous material to the inside of a cell(s) in a patient in need thereof, the method comprising the step of administering to the patient a PD-L1 targeting molecule, wherein the target cell(s) is physically coupled with PD-L1 bound by the binding region of the PD-L1 targeting molecule.

The use of any composition described herein (e.g. a PD-L1 targeting molecule, a pharmaceutical composition, or diagnostic composition) for the diagnosis, prognosis, and/or characterization of a disease, disorder, and/or condition is also contemplated.

In some embodiments, a method of detecting a cell using a PD-L1 targeting molecule and/or diagnostic composition comprises the steps of contacting a cell with said PD-L1 targeting molecule and/or diagnostic composition and detecting the presence of said PD-L1 targeting molecule and/or diagnostic composition. In some embodiments, the step of contacting the cell(s) occurs in vitro. In some embodiments, the step of contacting the cell(s) occurs in vivo. In some embodiments, the step of detecting the cell(s) occurs in vitro. In some embodiments, the step of detecting the cell(s) occurs in vivo.

For example, a diagnostic composition may be used to detect a cell in vivo by administering to a chordate subject a composition comprising PD-L1 targeting molecule which comprises a detection promoting agent and then detecting the presence of the PD-L1 targeting molecule and/or a heterologous, CD8+ T-cell epitope-peptide cargo (of the PD-L1 targeting molecule) either in vitro or in vivo.

Some embodiments of the PD-L1 targeting molecules may be utilized for the delivery of additional exogenous material into a cell physically coupled with PD-L1 bound by the PD-L1 targeting molecule. Additionally, provided herein is a method for delivering exogenous material to the inside of a cell(s) comprising contacting the cell(s), either in vitro or in vivo, with a PD-L1 targeting molecule, pharmaceutical composition, and/or diagnostic composition. Also provided herein is a method for delivering exogenous material to the inside of a cell(s) in a patient, the method comprising the step of administering to the patient a PD-L1 targeting molecule (with or without cytotoxic activity), wherein the target cell(s) is physically coupled with PD-L1 bound by the PD-L1 targeting molecule.

In some embodiments, a method of delivering into a cell a T-cell epitope-peptide cargo capable of being presented by a MHC class I molecule of the cell comprises the step of contacting the cell with the PD-L1 targeting molecule which is associated with a heterologous, T-cell epitope-peptide cargo and/or a composition thereof (e.g., a pharmaceutical or diagnostic composition).

Also provided herein is a method for “seeding” a tissue locus within a chordate, the method comprising the step of: administering to the chordate a PD-L1 targeting molecule, or a pharmaceutical or a diagnostic composition thereof. In some embodiments, the methods for “seeding” a tissue locus are for “seeding” a tissue locus which comprises a malignant, diseased, or inflamed tissue. In some embodiments, the methods for “seeding” a tissue locus are for “seeding” a tissue locus which comprises the tissue selected from the group consisting of: diseased tissue, tumor mass, cancerous growth, tumor, infected tissue, or abnormal cellular mass. In some embodiments, the methods for “seeding” a tissue locus comprises administering to the chordate the PD-L1 targeting molecule, the pharmaceutical composition, or the diagnostic composition comprising a heterologous, T-cell epitope-peptide cargo selected from the group consisting of: peptides not natively presented by the target cells of the PD-L1 targeting molecule in MEW class I complexes, peptides not natively present within any protein expressed by the target cell, peptides not natively present within the proteome of the target cell, peptides not natively present in the extracellular microenvironment of the site to be seeded, and peptides not natively present in the tumor mass or infected tissue site to be targeted.

The use of any composition of matter described herein for the diagnosis, prognosis, and/or characterization of a disease, disorder, and/or condition is contemplated. In some embodiments, a method of using a PD-L1 targeting molecule comprising a detection promoting agent and/or composition (e.g. a diagnostic composition) for the collection of information useful in the diagnosis, prognosis, or characterization of a disease, disorder, or condition. Among some embodiments is the method of detecting a cell (or subcellular compartment thereof) using a PD-L1 targeting molecule and/or diagnostic composition, the method comprising the steps of contacting a cell with the PD-L1 targeting molecule and/or diagnostic composition and detecting the presence of said PD-L1 targeting molecule and/or diagnostic composition. In some embodiments, the step of contacting the cell(s) occurs in vitro. In some embodiments, the step of contacting the cell(s) occurs in vivo. In some embodiments, the step of detecting the cell(s) occurs in vitro. In some embodiments, the step of detecting the cell(s) occurs in vivo. In some embodiments, the method involves the detection of the location of the PD-L1 targeting molecule in an organism using one or more imaging procedures after the administration of the PD-L1 targeting molecule to said organism. For example, PD-L1 targeting molecules which incorporate detection promoting agents as described herein may be used to characterize diseases as potentially treatable by a related pharmaceutical composition. For example, certain PD-L1 targeting molecules and compositions thereof (e.g. pharmaceutical compositions and diagnostic compositions), and methods may be used to determine if a patient belongs to a group that responds to a pharmaceutical composition described herein. For example, certain PD-L1 targeting molecules and compositions thereof may be used to identify cells which present a delivered heterologous epitope-peptide cargo on a cellular surface and/or to identify subjects containing cells which present a heterologous epitope-peptide cargo delivered by a PD-L1 targeting.

Also provided is a method of producing a molecule described herein, the method comprising the step of purifying the molecule or a polypeptide component of thereof using a bacterial cell-wall protein domain interaction, such as, e.g., protein L from P. magnus or derivatives and binding domain fragments thereof. In some embodiments, the purifying step of the method involves the Shiga toxin effector polypeptide comprising or consisting essentially of any one of the polypeptides shown in SEQ ID NOs: 1-18, 41-69 and 261-284.

Also provided herein are kits comprising a composition of matter described herein, and optionally, instructions for use, additional reagent(s), and/or pharmaceutical delivery device(s). The kit may further comprise reagents and other tools for detecting a cell type (e.g. a tumor cell) in a sample or in a subject.

### A. Cell-Kill Via Shiga Toxin a Subunit Cytotoxicity

In some embodiments, the Shiga toxin effector polypeptides and/or PD-L1 targeting molecules are cytotoxic. Some embodiments of the PD-L1 targeting molecules are cytotoxic only due to the presence of one or more Shiga toxin effector polypeptide components. The A Subunits of members of the Shiga toxin family each comprise an enzymatically active polypeptide region capable of killing a eukaryotic cell once in the cell's cytosol. Because members of the Shiga toxin family are adapted to killing eukaryotic cells, molecules derived from Shiga toxins, such as, e.g., PD-L1 targeting molecules comprising some embodiments of the Shiga toxin effector polypeptides can exhibit potent cell-kill activities.

In some embodiments, upon contacting a cell physically coupled with PD-L1 bound by the binding region of the PD-L1 targeting molecule (e.g. a PD-L1 positive cell), the PD-L1 targeting molecule is capable of causing death of the cell. In some embodiments, the CD₅₀ value of the PD-L1 targeting molecule is less than 5, 2.5, 1, 0.5, or 0.25 nM, which is vastly more potent than an untargeted, wild-type, Shiga toxin effector polypeptide (e.g., SEQ ID NOs: 1-18).

Cell-kill may be accomplished using a molecule under varied conditions of target cells, such as, e.g., an ex vivo manipulated target cell, a target cell cultured in vitro, a target cell within a tissue sample cultured in vitro, or a target cell in an in vivo setting like within a multicellular organism.

In some embodiments, the Shiga toxin effector polypeptides and PD-L1 targeting molecules comprise (1) a de-immunized, Shiga toxin effector sub-region, (2) a protease-cleavage resistant region near the carboxy-terminus of a Shiga toxin A1 fragment derived region, (3) a carboxy-terminal, endoplasmic reticulum retention/retrieval signal motif; and/or (4) a heterologous, T-cell epitope embedded or inserted region; however, for some embodiments, these structural modifications do not significantly alter the potency of Shiga toxin cytotoxicity as compared to reference molecules comprising a wild-type Shiga toxin A Subunit polypeptide, such as, e.g., a wild-type Shiga toxin A1 fragment. Thus, Shiga toxin effector polypeptides and PD-L1 targeting molecules which are de-immunized, protease cleavage resistant, and/or carrying embedded or inserted, heterologous, epitopes can maintain potent cytotoxicity while providing one or more various other functionalities or properties.

Already cytotoxic PD-L1 targeting molecules comprising Shiga toxin effector polypeptides may be engineered by the skilled worker using the information and methods provided herein to be more cytotoxic and/or to have redundant, backup cytotoxicities operating via completely different mechanisms. These multiple cytotoxic mechanisms may complement each other by their diversity of functions (such as by providing potent killing via two mechanisms of cell-killing, direct and indirect, as well as mechanisms of immuno-stimulation to the local area), redundantly backup each other (such as by providing one cell-killing mechanism in the absence of the other mechanisms—like if a target cell is resistant to or acquires some immunity to a subset of previously active mechanisms), and/or protect against developed resistance (by limiting resistance to the less probable situation of the malignant or infected cell blocking multiple, different cell-killing mechanisms simultaneously).

In some embodiments, cytotoxic PD-L1 targeting molecules comprising cytotoxic Shiga toxin effector polypeptides are effective at in vivo target cell killing independent of a subject's immune function status because these molecules function via the mechanism of action involving the inhibition of protein synthesis.

### B. Delivery of a T-Cell Epitope for MEW Class I Presentation on a Cell Surface

In some embodiments, the Shiga toxin effector polypeptides and PD-L1 targeting molecules comprise a T-cell epitope, which enables the engineering of “T-cell epitope delivering” molecules with virtually unlimited choices of epitope-peptide cargos for delivery and cell-surface presentation by a nucleated, chordate cell. In some embodiments, the Shiga toxin effector polypeptides and PD-L1 targeting molecules are each capable of delivering one or more T-cell epitopes, associated with the Shiga toxin effector polypeptides and/or PD-L1 targeting molecules, to the proteasome of a cell. The delivered T-cell epitope are then proteolytic processed and presented by the MHC class I pathway on the surface of the cell. By engineering MHC class I epitopes into PD-L1 targeting molecules, the targeted delivery and presentation of immuno-stimulatory antigens may be accomplished in order to harness and direct a beneficial function(s) of a chordate immune system.

In some embodiments, the Shiga toxin effector polypeptide or PD-L1 targeting molecule is capable of delivering a T-cell epitope to a MHC class I molecule of a cell for cell-surface presentation. In some embodiments, the Shiga toxin effector polypeptide or PD-L1 targeting molecule comprises a heterologous, T-cell epitope, whether as an additional exogenous material or embedded or inserted within a Shiga toxin effector polypeptide. In some embodiments, the Shiga toxin effector polypeptide or PD-L1 targeting molecule is capable of delivering an embedded or inserted T-cell epitope to a MHC class I molecule for cell-surface presentation.

In some embodiments, the Shiga toxin effector polypeptide is capable of delivering a T-cell epitope, which is embedded or inserted in the Shiga toxin effector polypeptide, to a MHC class I molecule of a cell in which the Shiga toxin effector polypeptide is present for presentation of the T-cell epitope by the MHC class I molecule on a surface of the cell. In some embodiments, the T-cell epitope is a heterologous, T-cell epitope. In some embodiments, the T-cell epitope functions as CD8+ T-cell epitope, whether already known or identified in the future using methods which are currently routine to the skilled worker.

In some embodiments, the PD-L1 targeting molecule is capable of delivering a T-cell epitope, which is associated with the PD-L1 targeting molecule, to a MHC class I molecule of a cell for presentation of the T-cell epitope by the MHC class I molecule on a surface of the cell. In some embodiments, the T-cell epitope is a heterologous, T-cell epitope which is embedded or inserted in the Shiga toxin effector polypeptide. In some embodiments, the T-cell epitope functions as CD8+ T-cell epitope, whether already known or identified in the future using methods which are currently routine to the skilled worker.

In some embodiments, upon contacting a cell with the PD-L1 targeting molecule, the PD-L1 targeting molecule is capable of delivering a T-cell epitope-peptide, which is associated with the PD-L1 targeting molecule, to a MHC class I molecule of the cell for presentation of the T-cell epitope-peptide by the MHC class I molecule on a surface of the cell. In some embodiments, the T-cell epitope-peptide is a heterologous epitope which is embedded or inserted in a Shiga toxin effector polypeptide. In some embodiments, the T-cell epitope-peptide functions as CD8+ T-cell epitope, whether already known or identified in the future using methods which are currently routine to the skilled worker.

The addition of a heterologous epitope into or presence of a heterologous epitope in a PD-L1 targeting molecule, whether as an additional exogenous material or embedded or inserted within a Shiga toxin effector polypeptide, enables methods of using such PD-L1 targeting molecules for the cell-targeted delivery of a chosen epitope for cell-surface presentation by a nucleated, target cell within a chordate.

One function of certain, CD8+ T-cell hyper-immunized, Shiga toxin effector polypeptides and PD-L1 targeting molecules is the delivery of one or more T-cell epitope-peptides to a MHC class I molecule for MHC class I presentation by a cell. Delivery of exogenous, T-cell epitope-peptides to the MHC class I system of a target cell can be used to induce the target cell to present the T-cell epitope-peptide in association with MHC class I molecules on the cell surface, which subsequently leads to the activation of CD8+ effector T-cells to attack the target cell.

The skilled worker, using techniques known in the art, can associate, couple, and/or link certain, Shiga toxin effector polypeptides to various other PD-L1 targeting binding regions to create PD-L1 targeting molecules which target specific, extracellular, target biomolecules physically coupled to cells and promote target-cell internalization of these PD-L1 targeting molecules. All nucleated vertebrate cells are believed to be capable of presenting intracellular epitopes using the MHC class I system. Thus, extracellular target biomolecules of the PD-L1 targeting molecules may in principle target any nucleated vertebrate cell for T-cell epitope delivery to a MHC class I presentation pathway of such a cell.

The epitope-delivering functions of the Shiga toxin effector polypeptides and PD-L1 targeting molecules can be detected and monitored by a variety of standard methods known in the art to the skilled worker and/or described herein. For example, the ability of PD-L1 targeting molecules to deliver a T-cell epitope-peptide and drive presentation of the epitope-peptide by the MHC class I system of target cells may be investigated using various in vitro and in vivo assays, including, e.g., the direct detection/visualization of MHC class I/peptide complexes, measurement of binding affinities for the heterologous, T-cell epitope-peptide to MHC class I molecules, and/or measurement of functional consequences of MHC class I-peptide complex presentation on target cells by monitoring cytotoxic T-lymphocyte (CTL) responses (see e.g. Examples, infra).

Certain assays to monitor this function of the polypeptides and molecules of involve the direct detection of a specific MHC class I/peptide antigen complex in vitro or ex vivo. Common methods for direct visualization and quantitation of peptide-MHC class I complexes involve various immuno-detection reagents known to the skilled worker. For example, specific monoclonal antibodies can be developed to recognize a particular MHC/class I/peptide antigen complex. Similarly, soluble, multimeric T cell receptors, such as the TCR-STAR reagents (Altor Bioscience Corp., Mirmar, Fla., U.S.) can be used to directly visualize or quantitate specific MHC I/antigen complexes (Zhu X et al., J Immunol 176: 3223-32 (2006)). These specific mAbs or soluble, multimeric T-cell receptors may be used with various detection methods, including, e.g. immunohistochemistry, flow cytometry, and enzyme-linked immuno assay (ELISA).

An alternative method for direct identification and quantification of MHC I/peptide complexes involves mass spectrometry analyses, such as, e.g., the ProPresent Antigen Presentation Assay (ProImmune, Inc., Sarasota, Fla., U.S.) in which peptide-MCH class I complexes are extracted from the surfaces of cells, then the peptides are purified and identified by sequencing mass spectrometry (Falk K et al., Nature 351: 290-6 (1991)).

In certain assays to monitor the T-cell epitope delivery and MHC class I presentation function of the polypeptides and molecules involve computational and/or experimental methods to monitor MHC class I and peptide binding and stability. Several software programs are available for use by the skilled worker for predicting the binding responses of peptides to MHC class I alleles, such as, e.g., The Immune Epitope Database and Analysis Resource (IEDB) Analysis Resource MHC-I binding prediction Consensus tool (Kim Y et al., Nucleic Acid Res 40: W525-30 (2012). Several experimental assays have been routinely applied, such as, e.g., cell surface binding assays and/or surface plasmon resonance assays to quantify and/or compare binding kinetics (Miles K et al., Mol Immunol 48: 728-32 (2011)). Additionally, other MHC-peptide binding assays based on a measure of the ability of a peptide to stabilize the ternary MHC-peptide complex for a given MHC class I allele, as a comparison to known controls, have been developed (e.g., MHC-peptide binding assay from ProImmmune, Inc.).

Alternatively, measurements of the consequence of MHC class I/peptide antigen complex presentation on the cell surface can be performed by monitoring the cytotoxic T-cell (CTL) response to the specific complex. These measurements by include direct labeling of the CTLs with MHC class I tetramer or pentamer reagents. Tetramers or pentamers bind directly to T cell receptors of a particular specificity, determined by the Major Histocompatibility Complex (MHC) allele and peptide complex. Additionally, the quantification of released cytokines, such as interferon gamma or interleukins by ELISA or enzyme-linked immunospot (ELIspot) is commonly assayed to identify specific CTL responses. The cytotoxic capacity of CTL can be measured using a number of assays, including the classical 51 Chromium (Cr) release assay or alternative non-radioactive cytotoxicity assays (e.g., CytoTox96® non-radioactive kits and CellTox™ CellTiter-GLO® kits available from Promega Corp., Madison, Wis., U.S.), Granzyme B ELISpot, Caspase Activity Assays or LAMP-1 translocation flow cytometric assays. To specifically monitor the killing of target cells, carboxyfluorescein diacetate succinimidyl ester (CFSE) can be used to easily and quickly label a cell population of interest for in vitro or in vivo investigation to monitor killing of epitope specific CSFE labeled target cells (Durward M et al., J Vis Exp 45 pii 2250 (2010)).

In vivo responses to MHC class I presentation can be followed by administering a MHC class I/antigen promoting agent (e.g., a peptide, protein or inactivated/attenuated virus vaccine) followed by challenge with an active agent (e.g. a virus) and monitoring responses to that agent, typically in comparison with unvaccinated controls. Ex vivo samples can be monitored for CTL activity with methods similar to those described previously (e.g. CTL cytotoxicity assays and quantification of cytokine release).

HLA-A, HLA-B, and/or HLA-C molecules are isolated from the intoxicated cells after lysis using immune affinity (e.g., an anti-MHC antibody “pulldown” purification) and the associated peptides (i.e., the peptides presented by the isolated MHC molecules) are recovered from the purified complexes. The recovered peptides are analyzed by sequencing mass spectrometry. The mass spectrometry data is compared against a protein database library consisting of the sequence of the exogenous (non-self) peptide (T-cell epitope X) and the international protein index for humans (representing “self” or non-immunogenic peptides). The peptides are ranked by significance according to a probability database. All detected antigenic (non-self) peptide sequences are listed. The data is verified by searching against a scrambled decoy database to reduce false hits (see e.g. Ma B, Johnson R, Mol Cell Proteomics 11: 0111.014902 (2012)). The results will demonstrate that peptides from the T-cell epitope X are presented in MHC complexes on the surface of intoxicated target cells.

The set of presented peptide-antigen-MHC complexes can vary between cells due to the antigen-specific HLA molecules expressed. T-cells can then recognize specific peptide-antigen-MHC complexes displayed on a cell surface using different TCR molecules with different antigen-specificities.

Because multiple T-cell epitopes may be delivered by a PD-L1 targeting molecule, such as, e.g., by embedding two or more different T-cell epitopes in a single proteasome delivering effector polypeptide, a single PD-L1 targeting molecule may be effective chordates of the same species with different MHC class variants, such as, e.g., in humans with different HLA alleles. This may allow for the combining within a single molecule of different T-cell epitopes with different effectiveness in different sub-populations of subjects based on MHC complex protein diversity and polymorphisms. For example, human MHC complex proteins, HLA proteins, vary among humans based on genetic ancestry, e.g. African (sub-Saharan), Amerindian, Caucasiod, Mongoloid, New Guinean and Australian, or Pacific islander.

The applications involving the T-cell epitope delivering polypeptides and molecules described herein are vast. Every nucleated cell in a mammalian organism may be capable of WIC class I pathway presentation of immunogenic, T-cell epitope-peptides on their cell outer surfaces complexed to WIC class I molecules. In addition, the sensitivity of T-cell epitope recognition is so exquisite that only a few MHC-I peptide complexes are required to be presented to result in an immune response, e.g., even presentation of a single complex can be sufficient for recognition by an effector T-cell (Sykulev Y et al., Immunity 4: 565-71 (1996)).

The activation of T-cell responses are desired characteristics of certain anti-cancer, anti-neoplastic, anti-tumor, and/or anti-microbial biologic drugs to stimulate the patient's own immune system toward targeted cells. Activation of a robust and strong T-cell response is also a desired characteristic of many vaccines. The presentation of a T-cell epitope by a target cell within an organism can lead to the activation of robust immune responses to a target cell and/or its general locale within an organism. Thus, the targeted delivery of a T-cell epitope for presentation may be utilized for as a mechanism for activating T-cell responses during a therapeutic regime.

The presentation of a T-cell immunogenic epitope-peptide by the WIC class I system targets the presenting cell for killing by CTL-mediated lysis and also triggers immune stimulation in the local microenvironment. By engineering immunogenic epitope sequences within Shiga toxin effector polypeptide components of target-cell-internalizing therapeutic molecules, the targeted delivery and presentation of immuno-stimulatory antigens may be accomplished. The presentation of immuno-stimulatory non-self antigens, such as e.g. known viral antigens with high immunogenicity, by target cells signals to other immune cells to destroy the target cells as well as to recruit more immune cells to the area.

The presentation of an immunogenic, T-cell epitope-peptide by the MHC class I complex targets the presenting cell for killing by CTL-mediated cytolysis. The presentation by targeted cells of immuno-stimulatory non-self antigens, such as, e.g., known viral epitope-peptides with high immunogenicity, can signal to other immune cells to destroy the target cells and recruit more immune cells to the target cell site within a chordate.

Thus, already cytotoxic molecules, such as e.g. therapeutic or potentially therapeutic molecules comprising Shiga toxin effector polypeptides, may be engineered using methods into more cytotoxic molecules and/or to have an additional cytotoxic mechanism operating via delivery of a T-cell epitope, presentation, and stimulation of effector T-cells. These multiple cytotoxic mechanisms may complement each other (such as by providing both direct target-cell-killing and indirect (CTL-mediated) cell-killing, redundantly backup each other (such as by providing one mechanism of cell-killing in the absence of the other), and/or protect against the development of therapeutic resistance (by limiting resistance to the less probable situation of the malignant or infected cell evolving to block two different cell-killing mechanisms simultaneously).

In addition, a cytotoxic molecule comprising a Shiga toxin effector polypeptide region that exhibits catalytic-based cytotoxicity may be engineered by the skilled worker using routine methods into enzymatically inactive variants. For example, the cytotoxic Shiga toxin effector polypeptide component of a cytotoxic molecule may be conferred with reduced activity and/or rendered inactive by the introduction of one or mutations and/or truncations such that the resulting molecule can still be cytotoxic via its ability to deliver a T-cell epitope to the MHC class I system of a target cell and subsequent presentation to the surface of the target cell. In another example, a T-cell epitope may be inserted or embedded into a Shiga toxin effector polypeptide such that the Shiga toxin effector polypeptide is inactivated by the added epitope (see e.g. WO 2015/113005: Example 1-F). This approach removes one cytotoxic mechanism while retaining or adding another and may also provide a molecule capable of exhibiting immuno-stimulation to the local area of a target cell(s) within an organism via delivered T-cell epitope presentation or “antigen seeding.” Furthermore, non-cytotoxic variants of the PD-L1 targeting molecules which comprise embedded or inserted, heterologous, T-cell epitopes may be useful in applications involving immune-stimulation within a chordate and/or labeling of target cells within a chordate with MEW class I molecule displayed epitopes.

The ability to deliver a T-cell epitope of certain Shiga toxin effector polypeptides and PD-L1 targeting molecules may be accomplished under varied conditions and in the presence of non-targeted bystander cells, such as, e.g., an ex vivo manipulated target cell, a target cell cultured in vitro, a target cell within a tissue sample cultured in vitro, or a target cell in an in vivo setting like within a multicellular organism.

C. Cell-Kill Via Targeted Cytotoxicity and/or Engagement of Cytotoxic T-Cells

For some embodiments, the PD-L1 targeting molecule can provide 1) delivery of a T-cell epitope for MEW class I presentation by a target cell and/or 2) potent cytotoxicity. For some embodiments of the PD-L1 targeting molecules, upon contacting a cell physically coupled with an extracellular PD-L1 bound by the cell-targeting binding region, the PD-L1 targeting molecule is capable of causing death of the cell. The mechanism of cell-kill may be direct, e.g. via the enzymatic activity of a toxin effector polypeptide region, or indirect via CTL-mediated cytolysis.

### 1. Indirect Cell-Kill Via T-Cell Epitope Delivery and MEW Class I Presentation

Some embodiments of the PD-L1 targeting molecules are cytotoxic because they comprise a CD8+ T-cell epitope capable of being delivered to the MEW class I presentation pathway of a target cell and presented on a cellular surface of the target cell. For example, T-cell epitope delivering, CD8+ T-cell hyper-immunized, Shiga toxin effector polypeptides, with or without endogenous epitope de-immunization, may be used as components of PD-L1 targeting molecules for applications involving indirect cell-killing.

In some embodiments of the PD-L1 targeting molecules, upon contacting a cell physically coupled with extracellular PD-L1 bound by the cell-targeting binding region, the PD-L1 targeting molecule is capable of indirectly causing the death of the cell, such as, e.g., via the presentation of one or more T-cell epitopes by the target cell and the subsequent recruitment of CTLs which kill the target cell.

The presentation of an antigenic peptide complexed with a MHC class I molecule by a cell sensitizes the presenting cell to targeted killing by cytotoxic T-cells (CTLs) via the induction of apoptosis, lysis, and/or necrosis. In addition, the CTLs which recognize the target cell may release immuno-stimulatory cytokines, such as, e.g., interferon gamma (IFN-gamma), tumor necrosis factor alpha (TNF), macrophage inflammatory protein-1 beta (MIP-1beta), and interleukins such as IL-17, IL-4, and IL-22. Furthermore, CTLs activated by recognition of a presented epitope may indiscriminately kill other cells proximal to the presenting cell regardless of the peptide-MHC class I complex repertoire presented by those proximal cells (Wiedemann A et al., Proc Natl Acad Sci USA 103: 10985-90 (2006)).

Because of MHC allele diversity within different species, a PD-L1 targeting molecule comprising only a single epitope may exhibit varied effectiveness to different patients or subjects of the same species. However, some embodiments of the PD-L1 targeting molecules may each comprise multiple, T-cell epitopes that are capable of being delivered to the MHC class I system of a target cell simultaneously. Thus, for some embodiments of the PD-L1 targeting molecules, a PD-L1 targeting molecule is used to treat different subjects with considerable differences in their MHC molecules' epitope-peptide binding affinities (i.e. considerable differences in their MHC alleles and/or WIC genotypes). In addition, some embodiments of the PD-L1 targeting molecules reduce or prevent target cell adaptations to escape killing (e.g. a target cancer cell mutating to escape therapeutic effectiveness or “mutant escape”) by using multiple cell-killing mechanisms simultaneously (e.g. direct killing and indirect killing via multiple different T-cell epitopes simultaneously). Some embodiments of the PD-L1 targeting molecules may deliver an antigen to the WIC class I presentation pathway of a target cell and the antigen is presented on a cellular surface of the target cell resulting in the in vivo stimulation of the immune system, such as the promotion of the polyclonal expansion of a T-cell population(s) in the periphery.

Some embodiments of the PD-L1 targeting molecules may deliver an antigen to the WIC class I presentation pathway of a target cell and which is presented on a cellular surface of the target cell resulting in the in vivo recruitment of endogenous polyfunctional memory CTL responses against target cells, e.g. tumor cells. This may also result in a redirection of an endogenous CTL response (e.g. an anti-viral response) against target tumor cells.

### 2. Direct Cell-Kill Via Cell-Targeted, Shiga Toxin Cytotoxicity

Some embodiments of the PD-L1 targeting molecules are cytotoxic because they comprise a catalytically active, Shiga toxin effector polypeptide and regardless of the presence of an immunogenic, CD8+ T-cell epitope in the molecule. For example, CD8+ T-cell hyper-immunized, Shiga toxin effector polypeptides, with or without endogenous epitope de-immunization, may be used as components of PD-L1 targeting molecules for applications involving direct cell-killing, such as, e.g., via the ribotoxic, enzymatic activity of a Shiga toxin effector polypeptide or ribosome binding and interference with ribosome function due to a non-catalytic mechanism(s).

In some embodiments of the CD8+ T-cell hyper-immunized, PD-L1 targeting molecules, upon contacting a cell physically coupled with extracellular PD-L1 bound by the cell-targeting binding region, the PD-L1 targeting molecule is capable of directly causing the death of the cell, such as, e.g., without the involvement of a untargeted, cytotoxic T-cell (see Section V-D, supra). This mechanism of action is independent of a subject's immune function status.

Some embodiments of the PD-L1 targeting molecules are cytotoxic to tumor cells in vivo in the absence of tumor infiltrating lymphocytes and regardless of the immune modulatory status of the tumor microenvironment, e.g. regardless if the tumor is characterized as “hot” or “cold”, non-inflamed, or immune-excluded. Tumors are described as “hot” if they show signs of inflammation, meaning that the tumor has already be infiltrated by T-cells rushing to fight the cancer cells. In some “hot” tumors, the tumor cells may have undergone many mutations that create neoantigens recognized by the T-cells. In contrast, nonimmunogenic “cold” tumors have not yet been infiltrated with T cells. The lack of T cells may make it difficult to provoke an immune response with standard immunotherapy treatments.

For example, certain cytotoxic PD-L1 targeting molecules comprising cytotoxic Shiga toxin effector polypeptides are effective at in vivo target cell killing independent of a subject's immune function status because these molecules function via the mechanism of action involving the inhibition of protein synthesis, which is not significantly affected by the tumor microenvironment. As another example, certain cytotoxic PD-L1 targeting molecules comprising a CD8+ T-cell epitope but not a catalytically active Shiga toxin effector polypeptide are cytotoxic to tumor cells in vivo in the absence of tumor infiltrating lymphocytes and regardless of the immune modulatory status of the tumor microenvironment, e.g. regardless if the tumor is characterized as “hot” or “cold”, non-inflamed, or immune-excluded. These cytotoxic molecules target the tumor directly, which is agnostric of the “hot” or “cold” status of the tumor. This activity is not reliant on enzymatic ribosome inhibition, so either active or inactive Shiga toxin variants can have this activity.

### 3. Alteration of Tumor Immunophenotpye

A PD-L1 targeting molecule treatment(s) may induce anti-tumor effects, such as, e.g., by directly killing PD-L1 expressing tumor cells and PD-L1 positive immune cells resulting in alterations of tumor immunophenotypes. For example, certain PD-L1 targeting molecules can deliver a viral antigen cargo for presentation in complex with MHC class I molecules on the surfaces of HLA*A02+/PD-L1+ target cells, which may lead to alterations in the immunophenotype of the tissue site, tumor, and/or tumor microenvironment thereby allowing for beneficial anti-tumor surveillance by effector T-cells.

Briefly, the mechanism of action involves targeting potent cytotoxicity to PD-L1 expressing target cells. Once the PD-L1 targeting molecule is in the endoplasmic reticulum or cytosol, the antigenic peptide cargo may be cleaved away from the rest of the PD-L1 targeting molecule allowing for transport of the antigenic peptide to the lumen of the endoplasmic reticulum and/or binding of the antigenic peptide by a MHC class I molecule. This allows for an unloaded MHC class I molecule to become loaded with the antigenic peptide cargo, for example, to form a “pp65 loaded MHC-I”. The antigen loaded MHC class I molecule can then be transported from the endoplasmic reticulum through the Golgi to the cell surface to present the epitope for recognition by the adaptive immune system. Once on the cell-surface, the antigen-loaded MHC class I molecule complex may be recognized by T-cells, e.g., leading to cytotoxic T-cell engagement and killing of the antigen-presenting target cell. The presentation of delivered antigens by tumor cells may result in an “altered immune phenotype” and result in antigen seeding as described herein. The re-direction of endogenous cytotoxic T-cells to the tumor target cells via delivery and presentation of an antigen may represent an altered immune phenotype for the tumor or specific locus within the treated subject.

The alteration of the immune phenotype may result in a redirection of an endogenous CTL response (e.g. an anti-viral response) against target tumor cells. For example, the deliver and presentation of a class I CMV antigenic peptide may re-direct endogenous CMV-specific cytotoxic T-cells (CTLs) to the tumor cells. There may exist an endogenous population of CTLs which are CMV antigen specific that are present in the subject in a pool available for tissue homing to a tumor cell site. Once a CMV antigen is delivered and display by the MEW system, then these CTLs may move into the PD-L1 targeting molecule's targeted tissue and infiltrate tumor(s). Thus, a hot tumor environment with suppressed immune cell function (e.g. anergic) may have new CTLs arrive which initially would not be suppressed. Similarly, a cold or tumor-excluded microenvironment may also have new CTLs arrive which initially would not be suppressed and capable of clearing CMV antigen displaying cells.

In addition, the alteration of the immune phenotype may result in the activation or hyperactivation of naïve T-cells undergoing antigen presentation, such as with a tumor cell or at a tumor site, the in vivo stimulation of the immune system, such as the promotion of the polyclonal expansion of a T-cell population(s) in the periphery, and the in vivo recruitment of endogenous polyfunctional memory CTL responses against target cells, e.g. tumor cells. This may also result in a redirection of an endogenous CTL response against target tumor cells. In addition, this may result in the restoration of healthy memory T-cell function specific to the target tumor cell, such as via stimulation of the immune system tipping the balance to active immunosurveillance or relieving immunosuppresion. The stimulation of the immune system could be due to several factors, not limited to but including, responses to antigenicity of the PD-L1 targeting molecule, responses to the delivered and displayed antigen cargo, and cross-presentation of viral and cancer antigens during the CTL mediated killing of target cells initiated by the PD-L1 targeting molecule coming in contact with a target cell in vivo.

The alteration of the immune phenotype may occur in subjects having a specific HLA allele(s) regardless of whether the subject or the target cell is homozygous or heterozygous for that particular allele(s).

### C. Selective Cytotoxicity Among Cell Types

Certain PD-L1 targeting molecules have uses in the selective killing of specific target cells in the presence of untargeted, bystander cells. By targeting the delivery of Shiga toxin effector polypeptides to specific cells via a cell-targeting binding region(s), the PD-L1 targeting molecules can exhibit cell-type specific, restricted cell-kill activities resulting in the exclusive or preferential killing selected cell types in the presence of untargeted cells. Similarly, by targeting the delivery of immunogenic T-cell epitopes to the MEW class I pathway of target cells, the subsequent presentation of T-cell epitopes and CTL-mediated cytolysis of target cells induced by the PD-L1 targeting molecules can be restricted to exclusively or preferentially killing selected cell types in the presence of untargeted cells. In addition, both the cell-targeted delivery of a cytotoxic, Shiga toxin effector polypeptide region and an immunogenic, T-cell epitope can be accomplished by a single PD-L1 targeting molecule such that deliver of both potentially cytotoxic components is restricted exclusively or preferentially to target cells in the presence of untargeted cells.

For some embodiments, the PD-L1 targeting molecule is cytotoxic at certain concentrations. In some embodiments, upon administration of the PD-L1 targeting molecule to a mixture of cell types, the cytotoxic PD-L1 targeting molecule is capable of selectively killing those cells which are physically coupled with extracellular PD-L1 bound by the binding region compared to cell types not physically coupled with any extracellular PD-L1. In some embodiments, the cytotoxic PD-L1 targeting molecule is capable of selectively or preferentially causing the death of a specific cell type within a mixture of two or more different cell types. This enables targeting cytotoxic activity to specific cell types with a high preferentiality, such as a 3-fold cytotoxic effect, over “bystander” cell types that do not express the target biomolecule. Alternatively, the expression of the target biomolecule of the binding region may be non-exclusive to one cell type if the target biomolecule is expressed in low enough amounts and/or physically coupled in low amounts with cell types that are not to be targeted. This enables the targeted cell-killing of specific cell types with a high preferentiality, such as a 3-fold cytotoxic effect, over “bystander” cell types that do not express significant amounts of the target biomolecule or are not physically coupled to significant amounts of the target biomolecule.

For some embodiments, upon administration of the cytotoxic PD-L1 targeting molecule to two different populations of cell types, the cytotoxic PD-L1 targeting molecule is capable of causing cell death as defined by the half-maximal cytotoxic concentration (CD₅₀) on a population of target cells, whose members express an extracellular target biomolecule of the binding region of the cytotoxic PD-L1 targeting molecule, at a dose at least three-times lower than the CD₅₀ dose of the same cytotoxic PD-L1 targeting molecule to a population of cells whose members do not express an extracellular target biomolecule of the binding region of the cytotoxic PD-L1 targeting molecule.

In some embodiments, the cytotoxic activity of a PD-L1 targeting molecule toward populations of cell types physically coupled with an extracellular PD-L1 bound by the binding region is at least 3-fold higher than the cytotoxic activity toward populations of cell types not physically coupled with any extracellular PD-L1 bound by the binding region. In some embodiments, selective cytotoxicity may be quantified in terms of the ratio (a/b) of (a) cytotoxicity towards a population of cells of a specific cell type physically coupled with extracellular PD-L1 bound by the binding region to (b) cytotoxicity towards a population of cells of a cell type not physically coupled with any extracellular PD-L1 bound by binding region. In some embodiments, the cytotoxicity ratio is indicative of selective cytotoxicity which is at least 3-fold, 5-fold, 10-fold, 15-fold, 20-fold, 25-fold, 30-fold, 40-fold, 50-fold, 75-fold, 100-fold, 250-fold, 500-fold, 750-fold, or 1000-fold higher for populations of cells or cell types physically coupled with a target biomolecule of the binding region compared to populations of cells or cell types not physically coupled with a target biomolecule of the binding region.

For some embodiments, the preferential cell-killing function or selective cytotoxicity of a PD-L1 targeting molecule is due to an additional exogenous material (e.g. a cytotoxic material) and/or heterologous, T-cell epitope present in a Shiga toxin effector polypeptide and not necessarily a result of the catalytic activity of a Shiga toxin effector polypeptide region.

This preferential cell-killing function allows a targeted cell to be killed by certain cytotoxic, PD-L1 targeting molecules under varied conditions and in the presence of non-targeted bystander cells, such as ex vivo manipulated mixtures of cell types, in vitro cultured tissues with mixtures of cell types, or in vivo in the presence of multiple cell types (e.g. in situ or in a native location within a multicellular organism).

### D. PD-L1 Modulation Reducing Immunosuppression

In some embodiments, the PD-L1 targeting molecules modulate expression of the PD-L1 target to which the PD-L1 targeting molecules' binding region binds. In some embodiments, the PD-L1 targeting molecules reduce or downregulate expression of the PD-L1 target. In some embodiments, the PD-L1 targeting molecules reduce cell-surface density of the PD-L1 target. In some embodiments in which PD-L1 functions as an immunosuppressive signal, modulation of expression of the PD-L1 target reduces immunosuppression. In some embodiments in which PD-L1 is involved in, e.g., promotes, cell-survival, modulation of expression of the PD-L1 target promotes cell death or weakens the promotion of cell-survival.

Certain PD-L1 targeting molecules are designed to deplete in vivo both PD-L1 expressing tumor cells and PD-L1 expressing immune cells. The targeted killing of PD-L1 immune cells can reduce the inhibition, both systemically and locally, of immune cells that might participate in immunosurveillance of tumor cells. For example, the inhibitory immune state of the tumor microenvironment may be relieved by the actions of a PD-L1 targeting molecule, such as via direct cell kill or PD-L1 modulation. This may lead to the release of effector T cell repression toward target tumor cells. At certain concentrations, the PD-L1 targeting molecule may preferentially deplete those PD-L1 immune cells which express only very high levels of PD-L1 or that are present within a tumor. Further, the MHC display of a viral antigen by PD-L1 expressing immune cells may target them for cytolysis by CTLs.

E. Delivery of Additional Exogenous Material into the Interior of Targeted Cells

In addition to cytotoxic, cytostatic, and immune stimulation applications, PD-L1 targeting molecules optionally may be used for targeted intracellular delivery functions, such as, e.g., in applications involving information gathering and diagnostic functions.

Because the PD-L1 targeting molecules (including reduced cytotoxicity and/or nontoxic forms thereof) are capable of entering cells physically coupled with an extracellular PD-L1 molecule recognized by the PD-L1 targeting molecule's binding region, some embodiments of the PD-L1 targeting molecules may be used to deliver additional exogenous materials into the interior of targeted cell types. For example, non-toxic variants of the cytotoxic, PD-L1 targeting molecules, or optionally cytotoxic variants, may be used to deliver additional exogenous materials to and/or label the interiors of cells physically coupled with an extracellular PD-L1 bound by the binding region of the PD-L1 targeting molecule. Various types of cells and/or cell populations which express target biomolecules to at least one cellular surface may be targeted by the PD-L1 targeting molecules for receiving exogenous materials. The functional components are modular, in that various Shiga toxin effector polypeptides, additional exogenous materials, and binding regions may be associated with each other to provide PD-L1 targeting molecules suitable for diverse applications involving cargo delivery, such as, e.g., non-invasive, in vivo imaging of tumor cells.

This delivery of exogenous material function of certain PD-L1 targeting molecules may be accomplished under varied conditions and in the presence of non-targeted bystander cells, such as, e.g., an ex vivo manipulated target cell, a target cell cultured in vitro, a target cell within a tissue sample cultured in vitro, or a target cell in an in vivo setting like within a multicellular organism. Furthermore, the selective delivery of exogenous material to certain cells by certain PD-L1 targeting molecules may be accomplished under varied conditions and in the presence of non-targeted bystander cells, such as ex vivo manipulated mixtures of cell types, in vitro cultured tissues with mixtures of cell types, or in vivo in the presence of multiple cell types (e.g., in situ or in a native location within a multicellular organism).

Shiga toxin effector polypeptides and PD-L1 targeting molecules which are not capable, such as a certain concentration ranges, of killing a target cell and/or delivering an embedded or inserted epitope for cell-surface presentation by a MHC molecule of a target cell may still be useful for delivering exogenous materials into cells, such as, e.g., detection promoting agents.

For some embodiments, the Shiga toxin effector exhibits low to zero cytotoxicity and thus are referred to herein as “noncytotoxic and/or reduced cytotoxic.” For some embodiments, the PD-L1 targeting molecule exhibits low to zero cytotoxicity and may be referred to as “noncytotoxic” and/or “reduced cytotoxic variants.” For example, some embodiments of the molecules do not exhibit a significant level of Shiga toxin based cytotoxicity wherein at doses of less than 1000 nM, 500 nM, 100 nM, 75 nM, 50 nM, there is no significant amount of cell death as compared to the appropriate reference molecule, such as, e.g., as measured by an assay known to the skilled worker and/or described herein. For some embodiments, the molecules do not exhibit any toxicity at dosages of 1-100 μg per kg of a mammalian recipient. Reduced-cytotoxic variants may still be cytotoxic at certain concentrations or dosages but exhibit reduced cytotoxicity, such as, e.g., are not capable of exhibiting a significant level of Shiga toxin cytotoxicity in certain situations.

Certain PD-L1 targeting molecules comprising the same, can be rendered non-cytotoxic, such as, e.g., via the addition of one or more amino acid substitutions known to the skilled worker to inactivate a Shiga toxin A Subunit and/or Shiga toxin effector polypeptide, including illustrative substitutions described herein. The non-cytotoxic and reduced cytotoxic variants of the PD-L1 targeting molecules may be in certain situations more suitable for delivery of additional exogenous materials than more cytotoxic variants.

### Information Gathering for Diagnostic Functions

Some of the PD-L1 targeting molecules described herein have uses in the in vitro and/or in vivo detection of specific cells, cell types, and/or cell populations, as well as specific subcellular compartments of any of the aforementioned. Reduced-cytotoxicity and/or nontoxic forms of the cytotoxic, PD-L1 targeting molecules that are conjugated to detection promoting agents optionally may be used for diagnostic functions, such as for companion diagnostics used in conjunction with a therapeutic regimen comprising the same or a related binding region, such as, e.g., a binding region with high-affinity binding to the same target biomolecule, an overlapping epitope, and/or the same epitope.

In some embodiments, the PD-L1 targeting molecules described herein are used for both diagnosis and treatment, or for diagnosis alone. When the same cytotoxic PD-L1 targeting molecule is used for both diagnosis and treatment, the PD-L1 targeting molecule variant which incorporates a detection promoting agent for diagnosis may have its cytotoxicity reduced or may be rendered nontoxic by catalytic inactivation of its Shiga toxin effector polypeptide region(s) via one or more amino acid substitutions, including illustrative substitutions described herein. For example, certain nontoxic variants of the PD-L1 targeting molecules exhibit less than 5%, 4%, 3%, 2%, or 1% death of target cells after administration of a dose less than 1 mg/kg. Reduced-cytotoxicity variants may still be cytotoxic at certain concentrations or dosages but exhibit reduced cytotoxicity, such as, e.g., are not capable of exhibiting a significant level of Shiga toxin cytotoxicity as described herein.

The ability to conjugate detection promoting agents known in the art to various PD-L1 targeting molecules provides useful compositions for the detection of certain cells, such as, e.g., cancer, tumor, immune, and/or infected cells. These diagnostic embodiments of the PD-L1 targeting molecules may be used for information gathering via various imaging techniques and assays known in the art. For example, diagnostic embodiments of the PD-L1 targeting molecules may be used for information gathering via imaging of intracellular organelles (e.g. endocytotic, Golgi, endoplasmic reticulum, and cytosolic compartments) of individual cancer cells, immune cells, and/or infected cells in a patient or biopsy sample.

Various types of information may be gathered using the diagnostic embodiments of the PD-L1 targeting molecules whether for diagnostic uses or other uses. This information may be useful, for example, in diagnosing neoplastic cell types, determining therapeutic susceptibilities of a patient's disease, assaying the progression of anti-neoplastic therapies over time, assaying the progression of immunomodulatory therapies over time, assaying the progression of antimicrobial therapies over time, evaluating the presence of infected cells in transplantation materials, evaluating the presence of unwanted cell types in transplantation materials, and/or evaluating the presence of residual tumor cells after surgical excision of a tumor mass.

For example, subpopulations of patients might be ascertained using information gathered using the diagnostic variants of the PD-L1 targeting molecules, and then individual patients could be further categorized into subpopulations based on their unique characteristic(s) revealed using those diagnostic embodiments. For example, the effectiveness of specific pharmaceuticals or therapies might be a criterion used to define a patient subpopulation. For example, a nontoxic diagnostic variant of a particular, cytotoxic PD-L1 targeting molecule may be used to differentiate which patients are in a class or subpopulation of patients predicted to respond positively to a cytotoxic variant of that PD-L1 targeting molecule. Accordingly, associated methods for patient identification, patient stratification, and diagnosis using PD-L1 targeting molecules, including non-toxic variants of cytotoxic, PD-L1 targeting molecules, are contemplated herein.

The expression of the target biomolecule by a cell need not be native in order for cell-targeting by a PD-L1 targeting molecule, such as, e.g., for direct cell-kill, indirect cell-kill, delivery of exogenous materials like T-cell epitopes, and/or information gathering. Cell surface expression of the target biomolecule could be the result of an infection, the presence of a pathogen, and/or the presence of an intracellular microbial pathogen. Expression of a target biomolecule could be artificial such as, for example, by forced or induced expression after infection with a viral expression vector, see e.g. adenoviral, adeno-associated viral, and retroviral systems. Expression of PD-L1 can be induced by exposing a cell to ionizing radiation (Wattenberg M et al., Br J Cancer 110: 1472-80 (2014)).

### V. Production, Manufacture, and Purification of Shiga Toxin Effector Polypeptides and PD-L1 Targeting Molecules

The Shiga toxin effector polypeptides and certain PD-L1 targeting molecules may be produced using techniques well known to those of skill in the art. For example, Shiga toxin effector polypeptides and PD-L1 targeting molecules may be manufactured by standard synthetic methods, by use of recombinant expression systems, or by any other suitable method. Thus, Shiga toxin effector polypeptides and PD-L1 targeting molecules may be synthesized in a number of ways, including, e.g. methods comprising: (1) synthesizing a polypeptide or polypeptide component of a PD-L1 targeting molecule using standard solid-phase or liquid-phase methodology, either stepwise or by fragment assembly, and isolating and purifying the final polypeptide compound product; (2) expressing a polynucleotide that encodes a protein or protein component of a PD-L1 targeting molecule in a host cell and recovering the expression product from the host cell or host cell culture; or (3) cell-free, in vitro expression of a polynucleotide encoding a polypeptide or polypeptide component of a PD-L1 targeting molecule, and recovering the expression product; or by any combination of the methods of (1), (2) or (3) to obtain fragments of the protein component, subsequently joining (e.g. ligating) the peptide or polypeptide fragments to obtain a polypeptide component, and recovering the polypeptide component.

It may be preferable to synthesize a PD-L1 targeting molecule, or a protein component of a PD-L1 targeting molecule, by means of solid-phase or liquid-phase peptide synthesis. Polypeptides and PD-L1 targeting molecules may suitably be manufactured by standard synthetic methods. Thus, peptides may be synthesized by, e.g. methods comprising synthesizing the peptide by standard solid-phase or liquid-phase methodology, either stepwise or by fragment assembly, and isolating and purifying the final peptide product. In this context, reference may be made to WO 1998/011125 or, inter alia, Fields G et al., Principles and Practice of Solid-Phase Peptide Synthesis (Synthetic Peptides, Grant G, ed., Oxford University Press, U.K., 2nd ed., 2002) and the synthesis examples therein.

Shiga toxin effector polypeptides and PD-L1 targeting molecules may be prepared (produced and purified) using recombinant techniques well known in the art. In general, methods for preparing proteins by culturing host cells transformed or transfected with a vector comprising the encoding polynucleotide and purifying or recovering the protein from cell culture are described in, e.g., Sambrook J et al., Molecular Cloning: A Laboratory Manual (Cold Spring Harbor Laboratory Press, NY, U.S., 1989); Dieffenbach C et al., PCR Primer: A Laboratory Manual (Cold Spring Harbor Laboratory Press, N.Y., U.S., 1995). Any suitable host cell may be used to produce a polypeptide and/or cell-targeting protein. Host cells may be cells stably or transiently transfected, transformed, transduced or infected with one or more expression vectors which drive expression of a polypeptide. In addition, a Shiga toxin effector polypeptide and/or PD-L1 targeting molecule may be produced by modifying the polynucleotide encoding a polypeptide or cell-targeting protein that result in altering one or more amino acids or deleting or inserting one or more amino acids in order to achieve desired properties, such as changed cytotoxicity, changed cytostatic effects, and/or changed serum half-life.

There are a wide variety of expression systems which may be chosen to produce a polypeptide or cell-targeting protein. For example, host organisms for expression of cell-targeting proteins include prokaryotes, such as E. coli and B. subtilis, eukaryotic cells, such as yeast and filamentous fungi (like S. cerevisiae, P. pastoris, A. awamori, and K. lactis), algae (like C. reinhardtii), insect cell lines, mammalian cells (like CHO cells), plant cell lines, and eukaryotic organisms such as transgenic plants (like A. thaliana and N. benthamiana).

Accordingly, in some embodiments, the instant disclosure provides methods for producing a Shiga toxin effector polypeptide and/or PD-L1 targeting molecule according to above recited methods and using a polynucleotide encoding part or all of a polypeptide or a protein component of a cell-targeting protein, an expression vector comprising at least one polynucleotide capable of encoding part or all of a polypeptide or cell-targeting protein when introduced into a host cell, and/or a host cell comprising a polynucleotide or expression vector.

When a protein is expressed using recombinant techniques in a host cell or cell-free system, it is advantageous to separate (or purify) the desired protein away from other components, such as host cell factors, in order to obtain preparations that are of high purity or are substantially homogeneous. Purification can be accomplished by methods well known in the art, such as centrifugation techniques, extraction techniques, chromatographic and fractionation techniques (e.g. size separation by gel filtration, charge separation by ion-exchange column, hydrophobic interaction chromatography, reverse phase chromatography, chromatography on silica or cation-exchange resins such as DEAE and the like, chromatofocusing, and Protein A Sepharose chromatography to remove contaminants), and precipitation techniques (e.g. ethanol precipitation or ammonium sulfate precipitation). Any number of biochemical purification techniques may be used to increase the purity of a polypeptide and/or PD-L1 targeting molecule. In some embodiments, the polypeptides and PD-L1 targeting molecules may optionally be purified in homo-multimeric forms (e.g. a molecular complex comprising two or more polypeptides or PD-L1 targeting molecules).

In the Examples below are descriptions of non-limiting examples of methods for producing illustrative, Shiga toxin effector polypeptides and PD-L1 targeting molecules, as well as specific but non-limiting aspects of production methods.

### VI. Pharmaceutical and Diagnostic Compositions Comprising PD-L1 Targeting Molecules

Also provided herein are Shiga toxin effector polypeptides and PD-L1 targeting molecules for use, alone or in combination with one or more additional therapeutic agents, in a pharmaceutical composition, for treatment or prophylaxis of conditions, diseases, disorders, or symptoms described in further detail below (e.g. cancers, malignant tumors, non-malignant tumors, growth abnormalities, immune disorders, and microbial infections). Also provided herein are pharmaceutical compositions comprising a PD-L1 targeting molecule, or a pharmaceutically acceptable salt or solvate thereof, together with at least one pharmaceutically acceptable carrier, excipient, or vehicle. In some embodiments, the pharmaceutical composition may comprise homo-multimeric and/or hetero-multimeric forms of a PD-L1 targeting molecule. The pharmaceutical compositions are useful in methods of treating, ameliorating, or preventing a disease, condition, disorder, or symptom described in further detail below. Each such disease, condition, disorder, or symptom is envisioned to be a separate embodiment with respect to uses of a pharmaceutical composition described herein. Also provided herein are pharmaceutical compositions for use in at least one method of treatment, as described in more detail below.

As used herein, the terms “patient” and “subject” are used interchangeably to refer to any organism, commonly vertebrates such as humans and animals, which presents symptoms, signs, and/or indications of at least one disease, disorder, or condition. These terms include mammals such as the non-limiting examples of primates, livestock animals (e.g. cattle, horses, pigs, sheep, goats, etc.), companion animals (e.g. cats, dogs, etc.) and laboratory animals (e.g. mice, rabbits, rats, etc.).

As used herein, “treat,” “treating,” or “treatment” and grammatical variants thereof refer to an approach for obtaining beneficial or desired clinical results. The terms may refer to slowing the onset or rate of development of a condition, disorder or disease, reducing or alleviating symptoms associated with it, generating a complete or partial regression of the condition, or some combination of any of the above. For the purposes of the instant disclosure, beneficial or desired clinical results include, but are not limited to, reduction or alleviation of symptoms, diminishment of extent of disease, stabilization (e.g. not worsening) of state of disease, delay or slowing of disease progression, amelioration or palliation of the disease state, and remission (whether partial or total), whether detectable or undetectable. “Treat,” “treating,” or “treatment” can also mean prolonging survival relative to expected survival time if not receiving treatment. A subject (e.g. a human) in need of treatment may thus be a subject already afflicted with the disease or disorder in question. The terms “treat,” “treating,” or “treatment” includes inhibition or reduction of an increase in severity of a pathological state or symptoms relative to the absence of treatment, and is not necessarily meant to imply complete cessation of the relevant disease, disorder, or condition. With regard to tumors and/or cancers, treatment includes reduction in overall tumor burden and/or individual tumor size.

As used herein, the terms “prevent,” “preventing,” “prevention” and grammatical variants thereof refer to an approach for preventing the development of, or altering the pathology of, a condition, disease, or disorder. Accordingly, “prevention” may refer to prophylactic or preventive measures. For the purposes of the instant disclosure, beneficial or desired clinical results include, but are not limited to, prevention or slowing of symptoms, progression or development of a disease, whether detectable or undetectable. A subject (e.g. a human) in need of prevention may thus be a subject not yet afflicted with the disease or disorder in question. The term “prevention” includes slowing the onset of disease relative to the absence of treatment and is not necessarily meant to imply permanent prevention of the relevant disease, disorder or condition. Thus “preventing” or “prevention” of a condition may in certain contexts refer to reducing the risk of developing the condition, or preventing or delaying the development of symptoms associated with the condition.

As used herein, an “effective amount” or “therapeutically effective amount” is an amount or dose of a composition (e.g. a therapeutic composition, compound, or agent) that produces at least one desired therapeutic effect in a subject, such as preventing or treating a target condition or beneficially alleviating a symptom associated with the condition. The most desirable therapeutically effective amount is an amount that will produce a desired efficacy of a particular treatment selected by one of skill in the art for a given subject in need thereof. This amount will vary depending upon a variety of factors understood by the skilled worker, including but not limited to the characteristics of the therapeutic composition (including activity, pharmacokinetics, pharmacodynamics, and bioavailability), the physiological condition of the subject (including age, sex, disease type, disease stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a composition and adjusting the dosage accordingly (see e.g. Remington: The Science and Practice of Pharmacy (Gennaro A, ed., Mack Publishing Co., Easton, Pa., U.S., 19th ed., 1995)).

Diagnostic compositions comprise a PD-L1 targeting molecule and one or more detection promoting agents. When producing or manufacturing a diagnostic composition, a PD-L1 targeting molecule may be directly or indirectly linked to one or more detection promoting agents. There are numerous standard techniques known to the skilled worker for incorporating, affixing, and/or conjugating various detection promoting agents to proteins or proteinaceous components of molecules, especially to immunoglobulins and immunoglobulin-derived domains.

There are numerous detection promoting agents known to the skilled worker, such as isotopes, dyes, colorimetric agents, contrast enhancing agents, fluorescent agents, bioluminescent agents, and magnetic agents, which can be operably linked to the polypeptides or PD-L1 targeting molecules for information gathering methods, such as for diagnostic and/or prognostic applications to diseases, disorders, or conditions of an organism (see e.g. Cai W et al., J Nucl Med 48: 304-10 (2007); Nayak T, Brechbiel M, Bioconjug Chem 20: 825-41 (2009); Paudyal P et al., Oncol Rep 22: 115-9 (2009); Qiao J et al., PLoS ONE 6: e18103 (2011); Sano K et al., Breast Cancer Res 14: R61 (2012)). These agents may be associated with, linked to, and/or incorporated within the polypeptide or PD-L1 targeting molecule at any suitable position. For example, the linkage or incorporation of the detection promoting agent may be via an amino acid residue(s) of a molecule or via some type of linkage known in the art, including via linkers and/or chelators. The incorporation of the agent is in such a way to enable the detection of the presence of the diagnostic composition in a screen, assay, diagnostic procedure, and/or imaging technique.

Similarly, there are numerous imaging approaches known to the skilled worker, such as non-invasive in vivo imaging techniques commonly used in the medical arena, for example: computed tomography imaging (CT scanning), optical imaging (including direct, fluorescent, and bioluminescent imaging), magnetic resonance imaging (MM), positron emission tomography (PET), single-photon emission computed tomography (SPECT), ultrasound, and x-ray computed tomography imaging.

VII. Production or Manufacture of Pharmaceutical and/or Diagnostic Compositions Comprising PD-L1 Targeting Molecules

Pharmaceutically acceptable salts or solvates of any of the Shiga toxin effector polypeptides and PD-L1 targeting molecules are also described herein.

The term “solvate” refers to a complex of defined stoichiometry formed between a solute (in casu, a proteinaceous compound or pharmaceutically acceptable salt thereof) and a solvent. The solvent in this connection may, for example, be water, ethanol or another pharmaceutically acceptable, typically small-molecular organic species, such as, but not limited to, acetic acid or lactic acid. When the solvent in question is water, such a solvate is normally referred to as a hydrate.

Polypeptides and proteins, or salts thereof, may be formulated as pharmaceutical compositions prepared for storage or administration, which typically comprise a therapeutically effective amount of a molecule as described herein, or a salt thereof, in a pharmaceutically acceptable carrier. The term “pharmaceutically acceptable carrier” includes any of the standard pharmaceutical carriers. Pharmaceutically acceptable carriers for therapeutic molecule use are well known in the pharmaceutical art, and are described, for example, in Remington's Pharmaceutical Sciences (Mack Publishing Co. (A. Gennaro, ed., 1985). As used herein, “pharmaceutically acceptable carrier” includes any and all physiologically acceptable, i.e. compatible, solvents, dispersion media, coatings, antimicrobial agents, isotonic, and absorption delaying agents, and the like. Pharmaceutically acceptable carriers or diluents include those used in formulations suitable for oral, rectal, nasal or parenteral (including subcutaneous, intramuscular, intravenous, intradermal, and transdermal) administration. Illustrative pharmaceutically acceptable carriers include sterile aqueous solutions or dispersions and sterile powders for the extemporaneous preparation of sterile injectable solutions or dispersions. Examples of suitable aqueous and nonaqueous carriers that may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyloleate. Proper fluidity can be maintained, for example, by the use of coating materials, such as lecithin, by the maintenance of the required particle size in the case of dispersions, and by the use of surfactants. In some embodiments, the carrier is suitable for intravenous, intramuscular, subcutaneous, parenteral, spinal or epidermal administration (e.g. by injection or infusion). Depending on selected route of administration, the protein or other pharmaceutical component may be coated in a material intended to protect the compound from the action of low pH and other natural inactivating conditions to which the active protein may encounter when administered to a patient by a particular route of administration.

The formulations of the pharmaceutical compositions may conveniently be presented in unit dosage form and may be prepared by any of the methods well known in the art of pharmacy. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. It may be provided in single dose injectable form, for example in the form of a pen. Compositions may be formulated for any suitable route and means of administration. Subcutaneous or transdermal modes of administration may be particularly suitable for therapeutic proteins described herein.

The pharmaceutical compositions may also contain adjuvants such as preservatives, wetting agents, emulsifying agents and dispersing agents. Preventing the presence of microorganisms may be ensured both by sterilization procedures, and by the inclusion of various antibacterial and antifungal agents, for example, paraben, chlorobutanol, phenol sorbic acid, and the like. Isotonic agents, such as sugars, sodium chloride, and the like into the compositions, may also be desirable. In addition, prolonged absorption of the injectable pharmaceutical form may be brought about by the inclusion of agents which delay absorption such as, aluminum monostearate and gelatin.

A pharmaceutical composition also optionally includes a pharmaceutically acceptable antioxidant. Illustrative pharmaceutically acceptable antioxidants are water soluble antioxidants such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propylgallate, alpha-tocopherol, and the like; and metal chelating agents, such as citric acid, ethylenediamine tetraacetic acid (EDTA), sorbitol, tartaric acid, phosphoric acid, and the like.

In some embodiments pharmaceutical compositions comprise one or a combination of different PD-L1 targeting molecules, or an ester, salt or amide of any of the foregoing, and at least one pharmaceutically acceptable carrier.

Therapeutic compositions are typically sterile and stable under the conditions of manufacture and storage. The composition may be formulated as a solution, microemulsion, liposome, or other ordered structure suitable to high drug concentration. The carrier may be a solvent or dispersion medium containing, for example, water, alcohol such as ethanol, polyol (e.g., glycerol, propylene glycol, and liquid polyethylene glycol), or any suitable mixtures. The proper fluidity may be maintained, for example, by the use of a coating such as lecithin, by the maintenance of the required particle size in the case of dispersion and by use of surfactants according to formulation chemistry well known in the art. In some embodiments, isotonic agents, e.g., sugars and polyalcohols such as mannitol, sorbitol, or sodium chloride, may be desirable in the composition. Prolonged absorption of injectable compositions may be brought about by including in the composition an agent that delays absorption for example, monostearate salts and gelatin.

Solutions or suspensions used for intradermal or subcutaneous application typically include one or more of: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and tonicity adjusting agents such as, e.g., sodium chloride or dextrose. The pH can be adjusted with acids or bases, such as hydrochloric acid or sodium hydroxide, or buffers with citrate, phosphate, acetate and the like. Such preparations may be enclosed in ampoules, disposable syringes or multiple dose vials made of glass or plastic.

Sterile injectable solutions may be prepared by incorporating a polypeptide or PD-L1 targeting molecule in the required amount in an appropriate solvent with one or a combination of ingredients described above, as required, followed by sterilization microfiltration. Dispersions may be prepared by incorporating the active compound into a sterile vehicle that contains adispersion medium and other ingredients, such as those described above. In the case of sterile powders for the preparation of sterile injectable solutions, the methods of preparation are vacuum drying and freeze-drying (lyophilization) that yield a powder of the active ingredient in addition to any additional desired ingredient from a sterile-filtered solution thereof.

When a therapeutically effective amount of a polypeptide and/or PD-L1 targeting molecule is designed to be administered by, e.g. intravenous, cutaneous or subcutaneous injection, the binding agent will be in the form of a pyrogen-free, parenterally acceptable aqueous solution. Methods for preparing parenterally acceptable protein solutions, taking into consideration appropriate pH, isotonicity, stability, and the like, are within the skill in the art. A preferred pharmaceutical composition for intravenous, cutaneous, or subcutaneous injection will contain, in addition to binding agents, an isotonic vehicle such as sodium chloride injection, Ringer's injection, dextrose injection, dextrose and sodium chloride injection, lactated Ringer's injection, or other vehicle as known in the art. A pharmaceutical composition may also contain stabilizers, preservatives, buffers, antioxidants, or other additives well known to those of skill in the art.

As described elsewhere herein, a polypeptide and/or PD-L1 targeting molecule may be prepared with carriers that will protect the active therapeutic agent against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art (see e.g. Sustained and Controlled Release Drug Delivery Systems (Robinson J, ed., Marcel Dekker, Inc., NY, U.S., 1978)).

In some embodiments, the composition (e.g. a pharmaceutical and/or diagnostic composition) may be formulated to ensure a desired in vivo distribution of a PD-L1 targeting molecule. For example, the blood-brain barrier excludes many large and/or hydrophilic compounds. To target a therapeutic molecule or composition to a particular in vivo location, they can be formulated, for example, in liposomes which may comprise one or more moieties that are selectively transported into specific cells or organs, thus enhancing targeted drug delivery. Illustrative targeting moieties include folate or biotin; mannosides; antibodies; surfactant protein A receptor; p120 catenin and the like.

Pharmaceutical compositions include parenteral formulations designed to be used as implants or particulate systems. Examples of implants are depot formulations composed of polymeric or hydrophobic components such as emulsions, ion exchange resins, and soluble salt solutions. Examples of particulate systems are microspheres, microparticles, nanocapsules, nanospheres, and nanoparticles (see e.g. Honda M et al., Int J Nanomedicine 8: 495-503 (2013); Sharma A et al., Biomed Res Int 2013: 960821 (2013); Ramishetti S, Huang L, Ther Deliv 3: 1429-45 (2012)). Controlled release formulations may be prepared using polymers sensitive to ions, such as, e.g. liposomes, polaxamer 407, and hydroxyapatite.

### VIII. Polynucleotides, Expression Vectors, and Host Cells

Beyond the polypeptides and PD-L1 targeting molecules described herein, the polynucleotides that encode the polypeptide components and PD-L1 targeting molecules, or functional portions thereof, are also described herein. The term “polynucleotide” is equivalent to the term “nucleic acid,” each of which includes one or more of: polymers of deoxyribonucleic acids (DNAs), polymers of ribonucleic acids (RNAs), analogs of these DNAs or RNAs generated using nucleotide analogs, and derivatives, fragments and homologs thereof. The polynucleotide may be single-, double-, or triple-stranded. Such polynucleotides are specifically disclosed to include all polynucleotides capable of encoding an illustrative protein, for example, taking into account the wobble known to be tolerated in the third position of RNA codons, yet encoding for the same amino acid as a different RNA codon (see Stothard P, Biotechniques 28: 1102-4 (2000)).

In some embodiments, a polynucleotide encodes a Shiga toxin effector polypeptide and/or PD-L1 targeting molecule, or a fragment or derivative thereof. The polynucleotides may include, e.g., a nucleic acid sequence encoding a polypeptide at least 50%, 55%, 60%, 65%, 70%, 75%, 80%, 85%, 90%, 95%, 99% or more, identical to a polypeptide comprising one of the amino acid sequences of a polypeptide or PD-L1 targeting molecule. In some embodiments, polynucleotides comprise nucleotide sequences that hybridize under stringent conditions to a polynucleotide which encodes a Shiga toxin effector polypeptide component and/or PD-L1 targeting molecule, or a fragment or derivative thereof, or the antisense or complement of any such sequence.

Derivatives or analogs of the PD-L1 targeting molecules (e.g., PD-L1 targeting molecules) include, inter alia, polynucleotide (or polypeptide) molecules having regions that are substantially homologous to the polynucleotides (or PD-L1 targeting molecules), e.g. by at least about 45%, 50%, 70%, 80%, 95%, 98%, or even 99% identity (with a preferred identity of 80-99%) over a polynucleotide (or polypeptide) sequence of the same size or when compared to an aligned sequence in which the alignment is done by a computer homology program known in the art. An example program is the GAP program (Wisconsin Sequence Analysis Package, Version 8 for UNIX, Genetics Computer Group, University Research Park, Madison, Wis., U.S.) using the default settings, which uses the algorithm of Smith T, Waterman M, Adv Appl Math 2: 482-9 (1981). Also included are polynucleotides capable of hybridizing to the complement of a sequence encoding the cell-targeting proteins under stringent conditions (see e.g. Ausubel F et al., Current Protocols in Molecular Biology (John Wiley & Sons, New York, N.Y., U.S., 1993)), and below. Stringent conditions are known to those skilled in the art and may be found, e.g., in Current Protocols in Molecular Biology (John Wiley & Sons, NY, U.S., Ch. Sec. 6.3.1-6.3.6 (1989)).

Also provided are expression vectors that comprise the polynucleotides described herein. The polynucleotides capable of encoding the Shiga toxin effector polypeptide components and/or PD-L1 targeting molecules may be inserted into known vectors, including bacterial plasmids, viral vectors and phage vectors, using material and methods well known in the art to produce expression vectors. Such expression vectors will include the polynucleotides necessary to support production of contemplated Shiga toxin effector polypeptides and/or PD-L1 targeting molecules within any host cell of choice or cell-free expression systems (e.g. pTxb1 and pIVEX2.3). The specific polynucleotides comprising expression vectors for use with specific types of host cells or cell-free expression systems are well known to one of ordinary skill in the art, can be determined using routine experimentation, and/or may be purchased.

The term “expression vector,” as used herein, refers to a polynucleotide, linear or circular, comprising one or more expression units. The term “expression unit” denotes a polynucleotide segment encoding a polypeptide of interest and capable of providing expression of the nucleic acid segment in a host cell. An expression unit typically comprises a transcription promoter, an open reading frame encoding the polypeptide of interest, and a transcription terminator, all in operable configuration. An expression vector contains one or more expression units. Thus, in some embodiments, an expression vector encoding a Shiga toxin effector polypeptide and/or PD-L1 targeting molecule comprising a single polypeptide chain includes at least an expression unit for the single polypeptide chain, whereas a protein comprising, e.g. two or more polypeptide chains (e.g. one chain comprising a V_{L} domain and a second chain comprising a V_{H} domain linked to a toxin effector polypeptide) includes at least two expression units, one for each of the two polypeptide chains of the protein. For expression of multi-chain cell-targeting proteins, an expression unit for each polypeptide chain may also be separately contained on different expression vectors (e.g. expression may be achieved with a single host cell into which expression vectors for each polypeptide chain has been introduced).

Expression vectors capable of directing transient or stable expression of polypeptides and proteins are well known in the art. The expression vectors generally include, but are not limited to, one or more of the following: a heterologous signal sequence or peptide, an origin of replication, one or more marker genes, an enhancer element, a promoter, and a transcription termination sequence, each of which is well known in the art. Optional regulatory control sequences, integration sequences, and useful markers that can be employed are known in the art.

The term “host cell” refers to a cell which can support the replication or expression of the expression vector. Host cells may be prokaryotic cells, such as E. coli or eukaryotic cells (e.g. yeast, insect, amphibian, bird, or mammalian cells). Creation and isolation of host cell lines comprising a polynucleotide or capable of producing a polypeptide and/or PD-L1 targeting molecule can be accomplished using standard techniques known in the art.

Shiga toxin effector polypeptides and/or proteins may be variants or derivatives of the polypeptides and molecules described herein that are produced by modifying the polynucleotide encoding a polypeptide and/or proteinaceous component of a PD-L1 targeting molecule by altering one or more amino acids or deleting or inserting one or more amino acids that may render it more suitable to achieve desired properties, such as more optimal expression by a host cell.

### IX. Molecules Immobilized on Solid Substrates

Some embodiments include a molecule described herein (e.g. a PD-L1 targeting molecule, fusion protein, or polynucleotide), or any effector fragment thereof, immobilized on a solid substrate. Solid substrates contemplated herein include, but are not limited to, microbeads, nanoparticles, polymers, matrix materials, microarrays, microtiter plates, or any solid surface known in the art (see e.g. U.S. Pat. No. 7,771,955). In accordance with these embodiments, a molecule may be covalently or non-covalently linked to a solid substrate, such as, e.g., a bead, particle, or plate, using techniques known to the skilled worker (see e.g. Jung Y et al., Analyst 133: 697-701 (2008)). Immobilized molecules may be used for screening applications using techniques known in the art (see e.g. Bradbury A et al., Nat Biotechnol 29: 245-54 (2011); Sutton C, Br J Pharmacol 166: 457-75 (2012); Diamante L et al., Protein Eng Des Sel 26: 713-24 (2013); Houlihan G et al., J Immunol Methods 405: 47-56 (2014)).

Non-limiting examples of solid substrates to which a molecule may be immobilized on include: microbeads, nanoparticles, polymers, nanopolymers, nanotubes, magnetic beads, paramagnetic beads, superparamagnetic beads, streptavidin coated beads, reverse-phase magnetic beads, carboxy terminated beads, hydrazine terminated beads, silica (sodium silica) beads and iminodiacetic acid (IDA)-modified beads, aldehyde-modified beads, epoxy-activated beads, diaminodipropylamine (DADPA)-modified beads (beads with primary amine surface group), biodegradable polymeric beads, polystyrene substrates, amino-polystyrene particles, carboxyl-polystyrene particles, epoxy-polystyrene particles, dimethylamino-polystyrene particles, hydroxy-polystyrene particles, colored particles, flow cytometry particles, sulfonate-polystyrene particles, nitrocellulose surfaces, reinforced nitrocellulose membranes, nylon membranes, glass surfaces, activated glass surfaces, activated quartz surfaces, polyvinylidene difluoride (PVDF) membranes, polyacrylamide-based substrates, poly-vinyl chloride substrates, poly-methyl methacrylate substrates, poly(dimethyl siloxane) substrates, and photopolymers which contain photoreactive species (such as nitrenes, carbenes, and ketyl radicals) capable of forming covalent linkages. Other examples of solid substrates to which a molecule may be immobilized on are commonly used in molecular display systems, such as, e.g., cellular surfaces, phages, and virus particles.

### X. Delivery Devices and Kits

In some embodiments, a device comprises one or more compositions of matter described herein, such as a pharmaceutical composition or diagnostic composition, for delivery to a subject in need thereof. Thus, a delivery device comprising one or more compositions can be used to administer to a patient a composition of matter by various delivery methods, including: intravenous, subcutaneous, intramuscular or intraperitoneal injection; oral administration; transdermal administration; pulmonary or transmucosal administration; administration by implant, osmotic pump, cartridge or micro pump; or by other means recognized by a person of skill in the art.

Also provided herein are kits comprising at least one composition of matter, and optionally, packaging and instructions for use. Kits may be useful for drug administration and/or diagnostic information gathering. A kit may optionally comprise at least one additional reagent (e.g., standards, markers and the like). Kits typically include a label indicating the intended use of the contents of the kit. The kit may further comprise reagents and other tools for detecting a cell type (e.g. a tumor cell) in a sample or in a subject, or for diagnosing whether a patient belongs to a group that responds to a therapeutic strategy which makes use of a compound, composition, or related method, e.g., such as a method described herein.

XI. Methods for Using PD-L1 Targeting Molecules and/or Pharmaceutical and/or Diagnostic Compositions Thereof

Generally, it is an object of the instant disclosure to provide pharmacologically active agents, as well as compositions comprising the same, that can be used in the prevention and/or treatment of diseases, disorders, and conditions, such as certain cancers, tumors, growth abnormalities, immune disorders, or further pathological conditions mentioned herein. Accordingly, also provided herein are methods of using the polypeptides, PD-L1 targeting molecules, and pharmaceutical compositions for the targeted killing of cells, for delivering additional exogenous materials into targeted cells, for labeling of the interiors of targeted cells, for collecting diagnostic information, for the delivering of T-cell epitopes to the MHC class I presentation pathway of target cells, and for treating diseases, disorders, and conditions as described herein. For example, the disclosed methods may be used to prevent or treat cancers, cancer initiation, tumor initiation, metastasis, and/or disease reoccurrence.

In particular, it is an object to provide such pharmacologically active agents, compositions, and/or methods that have certain advantages compared to the agents, compositions, and/or methods that are currently known in the art. Accordingly, in some embodiments, methods of using Shiga toxin effector polypeptides and PD-L1 targeting molecules with specified protein sequences and pharmaceutical compositions thereof are provided. For example, any of the amino acid sequences described herein may be specifically utilized as a component of the PD-L1 targeting molecule used in the following methods or any method for using a PD-L1 targeting molecule known to the skilled worker, such as, e.g., various methods described in WO 2014/164680, WO 2014/164693, WO 2015/138435, WO 2015/138452, WO 2015/113005, WO 2015/113007, WO 2015/191764, US20150259428, WO 2016/196344, WO 2017/019623, WO 2018/106895, and WO 2018/140427.

Also provided are methods of killing a cell comprising the step of contacting the cell, either in vitro or in vivo, with a Shiga toxin effector polypeptide, PD-L1 targeting molecule, or pharmaceutical composition. The Shiga toxin effector polypeptides, PD-L1 targeting molecules, and pharmaceutical compositions can be used to kill a specific cell type upon contacting a cell or cells with one of the claimed compositions of matter. In some embodiments, a PD-L1 targeting molecule or pharmaceutical composition can be used to kill specific cell types in a mixture of different cell types, such as mixtures comprising cancer cells, infected cells, and/or hematological cells. In some embodiments, a PD-L1 targeting molecule, or pharmaceutical composition can be used to kill cancer cells in a mixture of different cell types. In some embodiments, a cytotoxic Shiga PD-L1 targeting molecule, or pharmaceutical composition can be used to kill specific cell types in a mixture of different cell types, such as pre-transplantation tissues. In some embodiments, a Shiga toxin effector polypeptide, PD-L1 targeting molecule, or pharmaceutical composition can be used to kill specific cell types in a mixture of cell types, such as pre-administration tissue material for therapeutic purposes. In some embodiments, a PD-L1 targeting molecule or pharmaceutical composition can be used to selectively kill cells infected by viruses or microorganisms, or otherwise selectively kill cells expressing a particular extracellular target biomolecule, such as a cell surface biomolecule. The Shiga toxin effector polypeptides, PD-L1 targeting molecules, and pharmaceutical compositions have varied applications, including, e.g., uses in depleting unwanted cell types from tissues either in vitro or in vivo, uses in modulating immune responses to treat graft versus host, uses as antiviral agents, uses as anti-parasitic agents, and uses in purging transplantation tissues of unwanted cell types.

In some embodiments, certain Shiga toxin effector polypeptides, PD-L1 targeting molecules, and pharmaceutical compositions, alone or in combination with other compounds or pharmaceutical compositions, can show potent cell-kill activity when administered to a population of cells, in vitro or in vivo in a subject such as in a patient in need of treatment. By targeting the delivery of enzymatically active Shiga toxin A Subunit effector polypeptides and/or T-cell epitopes using high-affinity binding regions to specific cell types, cell-kill activities can be restricted to specifically and selectively killing certain cell types within an organism, such as certain cancer cells, neoplastic cells, malignant cells, non-malignant tumor cells, and/or infected cells.

Also provided is a method of killing a cell in a patient in need thereof, the method comprising the step of administering to the patient at least one PD-L1 targeting molecule or a pharmaceutical composition thereof.

In some embodiments, the PD-L1 targeting molecule or pharmaceutical compositions thereof can be used to kill a cancer cell in a patient by targeting an extracellular PD-L1 found physically coupled with a cancer or tumor cell. The terms “cancer cell” or “cancerous cell” refers to various neoplastic cells which grow and divide in an abnormally accelerated and/or unregulated fashion and will be clear to the skilled person. The term “tumor cell” includes both malignant and non-malignant cells. Generally, cancers and/or tumors can be defined as diseases, disorders, or conditions that are amenable to treatment and/or prevention. The cancers and tumors (either malignant or non-malignant) which are comprised of cancer cells and/or tumor cells which may benefit from methods and compositions will be clear to the skilled person. Neoplastic cells are often associated with one or more of the following: unregulated growth, lack of differentiation, local tissue invasion, angiogenesis, and metastasis. The diseases, disorders, and conditions resulting from cancers and/or tumors (either malignant or non-malignant) which may benefit from the methods and compositions targeting certain cancer cells and/or tumor cells will be clear to the skilled person.

Some embodiments of the PD-L1 targeting molecules and compositions may be used to kill cancer stem cells, tumor stem cells, pre-malignant cancer-initiating cells, and tumor-initiating cells, which commonly are slow dividing and resistant to cancer therapies like chemotherapy and radiation. For example, acute myeloid leukemias (AMLs) may be treated by killing AML stem cells and/or dormant AML progenitor cells (see e.g. Shlush L et al., Blood 120: 603-12 (2012)).

Because of the Shiga toxin A Subunit based mechanism of action, compositions of matter may be more effectively used in methods involving their combination with, or in complementary fashion with other therapies, such as, e.g., chemotherapies, immunotherapies, radiation, stem cell transplantation, and immune checkpoint inhibitors, and/or effective against chemoresistant/radiation-resistant and/or resting tumor cells/tumor initiating cells/stem cells. Similarly, compositions of matter may be more effectively used in methods involving in combination with other cell-targeted therapies targeting other than the same epitope on, non-overlapping, or different targets for the same disease disorder or condition.

Some embodiments of the PD-L1 targeting molecules, or pharmaceutical compositions thereof, can be used to kill an immune cell (whether healthy or malignant) in a patient by targeting an extracellular PD-L1 found physically coupled with an immune cell.

Ins ome embodiments, a PD-L1 targeting molecule, or pharmaceutical composition thereof, may be uiltized for the purposes of purging patient cell populations (e.g. bone marrow) of malignant, neoplastic, or otherwise unwanted T-cells and/or B-cells and then reinfusing the T-cell and/or B-cells depleted material into the patient (see e.g. van Heeckeren W et al., Br J Haematol 132: 42-55 (2006); (see e.g. Alpdogan O, van den Brink M, Semin Oncol 39: 629-42 (2012)).

In some embodiments, the PD-L1 targeting molecule, or pharmaceutical composition thereof, is utilized for the purposes of ex vivo depletion of T cells and/or B-cells from isolated cell populations removed from a patient. In one non-limiting example, the PD-L1 targeting molecule can be used in a method for prophylaxis of organ and/or tissue transplant rejection wherein the donor organ or tissue is perfused prior to transplant with a cytotoxic, PD-L1 targeting molecule or a pharmaceutical composition thereof in order to purge the organ of donor T-cells and/or B-cells (see e.g. Alpdogan O, van den Brink M, Semin Oncol 39: 629-42 (2012)).

In some embodiments, the PD-L1 targeting molecule, or pharmaceutical composition thereof, is utilized for the purposes of depleting T-cells and/or B-cells from a donor cell population as a prophylaxis against graft-versus-host disease, and induction of tolerance, in a patient to undergo a bone marrow and or stem cell transplant (see e.g. van Heeckeren W et al., Br J Haematol 132: 42-55 (2006); (see e.g. Alpdogan O, van den Brink M, Semin Oncol 39: 629-42 (2012)).

In some embodiments of the Shiga toxin effector polypeptide or PD-L1 targeting molecule, or pharmaceutical compositions thereof, can be used to kill an infected cell in a patient by targeting an extracellular PD-L1 found physically coupled with an infected cell.

In some embodiments of the PD-L1 targeting molecules, or pharmaceutical compositions thereof, can be used to “seed” a locus within a chordate with non-self, T-cell epitope-peptide presenting cells in order to activate the immune system to enhance policing of the locus. In some embodiments of this “seeding” method, the locus is a tumor mass or infected tissue site. In preferred embodiments of this “seeding” method, the non-self, T-cell epitope-peptide is selected from the group consisting of: peptides not already presented by the target cells of the PD-L1 targeting molecule, peptides not present within any protein expressed by the target cell, peptides not present within the proteome or transcriptome of the target cell, peptides not present in the extracellular microenvironment of the site to be seeded, and peptides not present in the tumor mass or infect tissue site to be targeting.

This “seeding” method functions to label one or more target cells within a chordate with one or more MHC class I presented T-cell epitopes for recognition by effector T-cells and activation of downstream immune responses. By exploiting the cell internalizing, intracellularly routing, and T-cell epitope delivering functions of the PD-L1 targeting molecules, the target cells which display the delivered T-cell epitope are harnessed to induce recognition of the presenting target cell by host T-cells and induction of further immune responses including target-cell-killing by CTLs. This “seeding” method of using a PD-L1 targeting molecule can provide a temporary vaccination-effect by inducing adaptive immune responses to attack the cells within the seeded microenvironment, such as, e.g. a tumor mass or infected tissue site, whether presenting a PD-L1 targeting molecule-delivered T-cell epitope(s) or not. This “seeding” method may also induce the breaking of immuno-tolerance to a target cell population, a tumor mass, and/or infected tissue site within a chordate.

Certain methods involving the seeding of a locus within a chordate with one or more antigenic and/or immunogenic epitopes may be combined with the administration of immunologic adjuvants, whether administered locally or systemically, to stimulate the immune response to certain antigens, such as, e.g., the co-administration of a composition with one or more immunologic adjuvants like a cytokine, bacterial product, or plant saponin. Other examples of immunologic adjuvants which may be suitable for use in the methods include aluminum salts and oils, such as, e.g., alums, aluminum hydroxide, mineral oils, squalene, paraffin oils, peanut oils, and thimerosal.

Additionally, provided herein is a method of treating a disease, disorder, or condition in a patient comprising the step of administering to a patient in need thereof a therapeutically effective amount of at least one of the PD-L1 targeting molecules, or a pharmaceutical composition thereof. Contemplated diseases, disorders, and conditions that can be treated using this method include cancers, malignant tumors, non-malignant tumors, growth abnormalities, immune disorders, and microbial infections. Administration of a “therapeutically effective dosage” of a composition can result in a decrease in severity of disease symptoms, an increase in frequency and duration of disease symptom-free periods, or a prevention of impairment or disability due to the disease affliction.

The therapeutically effective amount of a composition will depend on the route of administration, the type of organism being treated, and the physical characteristics of the specific patient under consideration. These factors and their relationship to determining this amount are well known to skilled practitioners in the medical arts. This amount and the method of administration can be tailored to achieve optimal efficacy, and may depend on such factors as weight, diet, concurrent medication and other factors, well known to those skilled in the medical arts. The dosage sizes and dosing regimen most appropriate for human use may be guided by the results obtained, and may be confirmed in properly designed clinical trials. An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person.

An acceptable route of administration may refer to any administration pathway known in the art, including but not limited to aerosol, enteral, nasal, ophthalmic, oral, parenteral, rectal, vaginal, or transdermal (e.g. topical administration of a cream, gel or ointment, or by means of a transdermal patch). “Parenteral administration” is typically associated with injection at or in communication with the intended site of action, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal administration.

For administration of a pharmaceutical composition, the dosage range will generally be from about 0.001 to 10 milligrams per kilogram (mg/kg), and more, usually 0.001 to 0.5 mg/kg, of the subject's body weight. Suitable dosages may be 0.01 mg/kg body weight, 0.03 mg/kg body weight, 0.07 mg/kg body weight, 0.9 mg/kg body weight or 0.1 mg/kg body weight or within the range of 0.01 to 0.1 mg/kg. A suitable treatment regime is a once or twice daily administration, or a once or twice weekly administration, once every two weeks, once every three weeks, once every four weeks, once a month, once every two or three months or once every three to 6 months. Dosages may be selected and readjusted by the skilled health care professional as required to maximize therapeutic benefit for a particular patient.

Pharmaceutical compositions will typically be administered to the same patient on multiple occasions. Intervals between single dosages can be, for example, two to five days, weekly, monthly, every two or three months, every six months, or yearly. Intervals between administrations can also be irregular, based on regulating blood levels or other markers in the subject or patient. Dosage regimens for a composition include intravenous administration of 1 mg/kg body weight or 3 mg/kg body weight with the composition administered every two to four weeks for six dosages, then every three months at 3 mg/kg body weight or 1 mg/kg body weight.

A pharmaceutical composition may be administered via one or more routes of administration, using one or more of a variety of methods known in the art. As will be appreciated by the skilled worker, the route and/or mode of administration will vary depending upon the desired results. Routes of administration for PD-L1 targeting molecules and pharmaceutical compositions include, e.g. intravenous, intramuscular, intradermal, intraperitoneal, subcutaneous, spinal, or other parenteral routes of administration, for example by injection or infusion. For other embodiments, a PD-L1 targeting molecule or pharmaceutical composition may be administered by a non-parenteral route, such as a topical, epidermal or mucosal route of administration, for example, intranasally, orally, vaginally, rectally, sublingually, or topically.

Therapeutic PD-L1 targeting molecules or pharmaceutical compositions may be administered with one or more of a variety of medical devices known in the art. For example, in one embodiment, a pharmaceutical composition may be administered with a needleless hypodermic injection device. Examples of well-known implants and modules are in the art, including e.g., implantable micro-infusion pumps for controlled rate delivery; devices for administering through the skin; infusion pumps for delivery at a precise infusion rate; variable flow implantable infusion devices for continuous drug delivery; and osmotic drug delivery systems. These and other such implants, delivery systems, and modules are known to those skilled in the art.

The PD-L1 targeting molecule or pharmaceutical composition may be administered alone or in combination with one or more other therapeutic or diagnostic agents. A combination therapy may include a PD-L1 targeting molecule, or pharmaceutical composition thereof, combined with at least one other therapeutic agent selected based on the particular patient, disease or condition to be treated. Examples of other such agents include, inter alia, a cytotoxic, anti-cancer or chemotherapeutic agent, an anti-inflammatory or anti-proliferative agent, an antimicrobial or antiviral agent, growth factors, cytokines, an analgesic, a therapeutically active small molecule or polypeptide, a single chain antibody, a classical antibody or fragment thereof, or a nucleic acid molecule which modulates one or more signaling pathways, and similar modulating therapeutic molecules which may complement or otherwise be beneficial in a therapeutic or prophylactic treatment regimen.

Treatment of a patient with PD-L1 targeting molecule or pharmaceutical composition preferably leads to cell death of targeted cells and/or the inhibition of growth of targeted cells. As such, cytotoxic, PD-L1 targeting molecules, and pharmaceutical compositions comprising them, will be useful in methods for treating a variety of pathological disorders in which killing or depleting target cells may be beneficial, such as, inter alia, cancer, tumors, other growth abnormalities, immune disorders, and infected cells. In some embodiments, a method comprises suppressing cell proliferation, and treating cell disorders, including neoplasia, overactive B-cells, and overactive T-cells.

In some embodiments, the PD-L1 targeting molecules and pharmaceutical compositions can be used to treat or prevent cancers, tumors (malignant and non-malignant), growth abnormalities, immune disorders, and microbial infections. In a further aspect, the above ex vivo method can be combined with the above in vivo method to provide methods of treating or preventing rejection in bone marrow transplant recipients, and for achieving immunological tolerance.

In some embodiments, a method for treating malignancies or neoplasms and other blood cell associated cancers in a mammalian subject, such as a human, comprises the step of administering to a subject in need thereof a therapeutically effective amount of a cytotoxic PD-L1 targeting molecule or pharmaceutical composition.

The PD-L1 targeting molecules and pharmaceutical compositions have varied applications, including, e.g., uses in removing unwanted T-cells, uses in modulating immune responses to treat graft versus host, uses as antiviral agents, uses as antimicrobial agents, and uses in purging transplantation tissues of unwanted cell types. The PD-L1 targeting molecules and pharmaceutical compositions are commonly anti-neoplastic agents—meaning they are capable of treating and/or preventing the development, maturation, or spread of neoplastic or malignant cells by inhibiting the growth and/or causing the death of cancer or tumor cells.

In some embodiments, the PD-L1 targeting molecule or pharmaceutical composition is used to treat a B-cell-, plasma cell- or antibody-mediated disease or disorder, such as for example leukemia, lymphoma, myeloma, Human Immunodeficiency Virus (HIV) related diseases, amyloidosis, hemolytic uremic syndrome, polyarteritis, septic shock, Crohn's Disease, rheumatoid arthritis, ankylosing spondylitis, psoriatic arthritis, ulcerative colitis, psoriasis, asthma, Sjorgren's syndrome, graft-versus-host disease, graft rejection, diabetes, vasculitis, scleroderma, and systemic lupus erythematosus.

In another aspect, some embodiments of the PD-L1 targeting molecules and pharmaceutical compositions are antimicrobial agents—meaning they are capable of treating and/or preventing the acquisition, development, or consequences of microbiological pathogenic infections, such as caused by viruses, bacteria, fungi, prions, or protozoans.

In some embodiments, a prophylaxis or treatment is provided for diseases or conditions mediated by T-cells or B-cells by administering the PD-L1 targeting molecule, or a pharmaceutical composition thereof, to a patient for the purpose of killing T-cells or B-cells in the patient. This usage is compatible with preparing or conditioning a patient for bone marrow transplantation, stem cell transplantation, tissue transplantation, or organ transplantation, regardless of the source of the transplanted material, e.g. human or non-human sources.

In some embodiments, host-versus-graft disease is treated or prevented in a bone-marrow receipient via the targeted cell-killing of host T-cells using a cytotoxic PD-L1 targeting molecule or pharmaceutical composition described herein.

Some embodiments of the PD-L1 targeting molecules and pharmaceutical compositions can be utilized in a method of treating cancer comprising administering to a patient, in need thereof, a therapeutically effective amount of a PD-L1 targeting molecule and/or pharmaceutical composition. In some embodiments, the cancer being treated is selected from the group consisting of: bone cancer (such as multiple myeloma or Ewing's sarcoma), breast cancer, central/peripheral nervous system cancer (such as brain cancer, neurofibromatosis, or glioblastoma), gastrointestinal cancer (such as stomach cancer or colorectal cancer), germ cell cancer (such as ovarian cancers and testicular cancers, glandular cancer (such as pancreatic cancer, parathyroid cancer, pheochromocytoma, salivary gland cancer, or thyroid cancer), head-neck cancer (such as nasopharyngeal cancer, oral cancer, or pharyngeal cancer), hematological cancers (such as leukemia, lymphoma, or myeloma), kidney-urinary tract cancer (such as renal cancer and bladder cancer), liver cancer, lung/pleura cancer (such as mesothelioma, small cell lung carcinoma, or non-small cell lung carcinoma), prostate cancer, sarcoma (such as angiosarcoma, fibrosarcoma, Kaposi's sarcoma, or synovial sarcoma), skin cancer (such as basal cell carcinoma, squamous cell carcinoma, or melanoma), and uterine cancer.

Some embodiments of the PD-L1 targeting molecules and pharmaceutical compositions can be utilized in a method of treating an immune disorder comprising administering to a patient, in need thereof, a therapeutically effective amount of the PD-L1 targeting molecules and/or pharmaceutical composition. In some embodiments, the immune disorder is related to an inflammation associated with a disease selected from the group consisting of: amyloidosis, ankylosing spondylitis, asthma, Crohn's disease, diabetes, graft rejection, graft-vs.-host disease, Hashimoto's thyroiditis, hemolytic uremic syndrome, HIV-related diseases, lupus erythematosus, multiple sclerosis, polyarteritis, psoriasis, psoriatic arthritis, rheumatoid arthritis, scleroderma, septic shock, Sjörgren's syndrome, ulcerative colitis, and vasculitis.

In some embodiments, a Shiga toxin effector polypeptide or PD-L1 targeting molecule is used as a component of a pharmaceutical composition or medicament for the treatment or prevention of a cancer, tumor, other growth abnormality, immune disorder, and/or microbial infection. For example, immune disorders presenting on the skin of a patient may be treated with such a medicament in efforts to reduce inflammation. In another example, skin tumors may be treated with such a medicament in efforts to reduce tumor size or eliminate the tumor completely.

Certain cytotoxic PD-L1 targeting molecules, and compositions thereof, may be used in molecular neurosurgery applications such as immunolesioning and neuronal tracing (see, Wiley R, Lappi D, Adv Drug Deliv Rev 55: 1043-54 (2003), for review). For example, the targeting domain may be selected or derived from various ligands, such as neurotransmitters and neuropeptides, which target specific neuronal cell types by binding neuronal surface receptors, such as a neuronal circuit specific G-protein coupled receptor. Similarly, the targeting domain may be selected from or derived from antibodies that bind neuronal surface receptors. Because certain Shiga toxin effector polypeptides robustly direct their own retrograde axonal transport, certain PD-L1 targeting molecules may be used to kill a neuron(s) which expresses the extracellular target at a site of cytotoxic protein injection distant from the cell body (see Llewellyn-Smith I et al., J Neurosci Methods 103: 83-90 (2000)). These targeted cytotoxic molecules that specifically target neuronal cell types have uses in neuroscience research, such as for elucidating mechanisms of sensations (see e.g. Mishra S, Hoon M, Science 340: 968-71 (2013), and creating model systems of neurodegenerative diseases, such as Parkinson's and Alzheimer's (see e.g. Hamlin A et al., PLoS One e53472 (2013)).

In some embodiments, a Shiga toxin effector polypeptide, PD-L1 targeting molecule, pharmaceutical composition, and/or diagnostic composition is used to label or detect the interiors of neoplastic cells and/or immune cell types. This method may be based on the ability of certain PD-L1 targeting molecules to enter specific cell types and route within cells via retrograde intracellular transport, to the interior compartments of specific cell types are labeled for detection. This can be performed on cells in situ within a patient or on cells and tissues removed from an organism, e.g. biopsy material.

In some embodiments, a method to detect the presence of a cell type for the purpose of information gathering regarding diseases, conditions and/or disorders comprises using a Shiga toxin effector polypeptide, PD-L1 targeting molecule, pharmaceutical composition, and/or diagnostic composition. The method may comprise contacting a cell with a diagnostically sufficient amount of a PD-L1 targeting molecule in order to detect the molecule by an assay or diagnostic technique. The phrase “diagnostically sufficient amount” refers to an amount that provides adequate detection and accurate measurement for information gathering purposes by the particular assay or diagnostic technique utilized. Generally, the diagnostically sufficient amount for whole organism in vivo diagnostic use will be a non-cumulative dose of between 0.001 to 10 milligrams of the detection promoting agent linked PD-L1 targeting molecule per kg of subject per subject. Typically, the amount of Shiga toxin effector polypeptide or PD-L1 targeting molecule used in these information gathering methods will be as low as possible provided that it is still a diagnostically sufficient amount. For example, for in vivo detection in an organism, the amount of Shiga toxin effector polypeptide, PD-L1 targeting molecule, or pharmaceutical composition administered to a subject will be as low as feasibly possible.

The cell-type specific targeting of PD-L1 targeting molecules combined with detection promoting agents provides a way to detect and image cells physically coupled with an extracellular PD-L1 bound by the binding region of the molecule. Imaging of cells using the PD-L1 targeting molecules may be performed in vitro or in vivo by any suitable technique known in the art. Diagnostic information may be collected using various methods known in the art, including whole body imaging of an organism or using ex vivo samples taken from an organism. The term “sample” used herein refers to any number of things, but not limited to, fluids such as blood, urine, serum, lymph, saliva, anal secretions, vaginal secretions, and semen, and tissues obtained by biopsy procedures. For example, various detection promoting agents may be utilized for non-invasive in vivo tumor imaging by techniques such as magnetic resonance imaging (MRI), optical methods (such as direct, fluorescent, and bioluminescent imaging), positron emission tomography (PET), single-photon emission computed tomography (SPECT), ultrasound, x-ray computed tomography, and combinations of the aforementioned (see, Kaur S et al., Cancer Lett 315: 97-111 (2012), for review).

Also provided herein is a method of using a Shiga toxin effector polypeptide, PD-L1 targeting molecule, or pharmaceutical composition in a diagnostic composition to label or detect the interiors of a hematologic cell, cancer cell, tumor cell, infected cell, and/or immune cell (see e.g., Koyama Y et al., Clin Cancer Res 13: 2936-45 (2007); Ogawa M et al., Cancer Res 69: 1268-72 (2009); Yang L et al., Small 5: 235-43 (2009)). Based on the ability of certain PD-L1 targeting molecules to enter specific cell types and route within cells via retrograde intracellular transport, the interior compartments of specific cell types are labeled for detection. This can be performed on cells in situ within a patient or on cells and tissues removed from an organism, e.g. biopsy material.

Diagnostic compositions described herein may be used to characterize a disease, disorder, or condition as potentially treatable by a related pharmaceutical composition. In some embodiments, a composition may be used to determine whether a patient belongs to a group that responds to a therapeutic strategy which makes use of a compound, composition or related method as described herein or is well suited for using a delivery device.

Diagnostic compositions may be used after a disease, e.g. a cancer, is detected in order to better characterize it, such as to monitor distant metastases, heterogeneity, and stage of cancer progression. The phenotypic assessment of disease disorder or infection can help prognostic and prediction during therapeutic decision making. In disease reoccurrence, certain methods may be used to determine if local or systemic problem.

Diagnostic compositions may be used to assess responses to therapies regardless of the type of the type of therapy, e.g. small molecule drug, biological drug, or cell-based therapy. For example, some embodiments of the diagnostics may be used to measure changes in tumor size, changes in antigen positive cell populations including number and distribution, or monitoring a different marker than the antigen targeted by a therapy already being administered to a patient (see Smith-Jones P et al., Nat. Biotechnol 22: 701-6 (2004); Evans M et al., Proc. Natl. Acad. Sci. USA 108: 9578-82 (2011)).

In some embodiments of the method used to detect the presence of a cell type may be used to gather information regarding diseases, disorders, and conditions, such as, for example bone cancer (such as multiple myeloma or Ewing's sarcoma), breast cancer, central/peripheral nervous system cancer (such as brain cancer, neurofibromatosis, or glioblastoma), gastrointestinal cancer (such as stomach cancer or colorectal cancer), germ cell cancer (such as ovarian cancers and testicular cancers, glandular cancer (such as pancreatic cancer, parathyroid cancer, pheochromocytoma, salivary gland cancer, or thyroid cancer), head-neck cancer (such as nasopharyngeal cancer, oral cancer, or pharyngeal cancer), hematological cancers (such as leukemia, lymphoma, or myeloma), kidney-urinary tract cancer (such as renal cancer and bladder cancer), liver cancer, lung/pleura cancer (such as mesothelioma, small cell lung carcinoma, or non-small cell lung carcinoma), prostate cancer, sarcoma (such as angiosarcoma, fibrosarcoma, Kaposi's sarcoma, or synovial sarcoma), skin cancer (such as basal cell carcinoma, squamous cell carcinoma, or melanoma), uterine cancer, AIDS, amyloidosis, ankylosing spondylitis, asthma, autism, cardiogenesis, Crohn's disease, diabetes, erythematosus, gastritis, graft rejection, graft-versus-host disease, Grave's disease, Hashimoto's thyroiditis, hemolytic uremic syndrome, HIV-related diseases, lupus erythematosus, lymphoproliferative disorders (including post-transplant lymphoproliferative disorders), multiple sclerosis, myasthenia gravis, neuroinflammation, polyarteritis, psoriasis, psoriatic arthritis, rheumatoid arthritis, scleroderma, septic shock, Sjörgren's syndrome, systemic lupus erythematosus, ulcerative colitis, vasculitis, cell proliferation, inflammation, leukocyte activation, leukocyte adhesion, leukocyte chemotaxis, leukocyte maturation, leukocyte migration, neuronal differentiation, acute lymphoblastic leukemia (ALL), T acute lymphocytic leukemia/lymphoma (ALL), acute myelogenous leukemia, acute myeloid leukemia (AML), B-cell chronic lymphocytic leukemia (B-CLL), B-cell prolymphocytic lymphoma, Burkitt's lymphoma (BL), chronic lymphocytic leukemia (CLL), chronic myelogenous leukemia (CIVIL-BP), chronic myeloid leukemia (CML), diffuse large B-cell lymphoma, follicular lymphoma, hairy cell leukemia (HCL), Hodgkin's Lymphoma (HL), intravascular large B-cell lymphoma, lymphomatoid granulomatosis, lymphoplasmacytic lymphoma, MALT lymphoma, mantle cell lymphoma, multiple myeloma (MM), natural killer cell leukemia, nodal marginal B-cell lymphoma, Non-Hodgkin's lymphoma (NHL), plasma cell leukemia, plasmacytoma, primary effusion lymphoma, pro-lymphocytic leukemia, promyelocytic leukemia, small lymphocytic lymphoma, splenic marginal zone lymphoma, T-cell lymphoma (TCL), heavy chain disease, monoclonal gammopathy, monoclonal immunoglobulin deposition disease, myelodusplastic syndromes (MDS), smoldering multiple myeloma, and Waldenstrom macroglobulinemia.

In some embodiments, the Shiga toxin effector polypeptides and PD-L1 targeting molecules, or pharmaceutical compositions thereof, are used for both diagnosis and treatment, or for diagnosis alone. In some situations, it would be desirable to determine or verify the HLA variant(s) and/or HLA alleles expressed in the subject and/or diseased tissue from the subject, such as, e.g., a patient in need of treatment, before selecting a Shiga toxin effector polypeptide or PD-L1 targeting molecule for use in treatment(s).

Any embodiment of the PD-L1 targeting molecule may be used with each individual embodiment of the methods.

The present invention is further illustrated by the following non-limiting examples of PD-L1 targeting molecules capable of specifically targeting PD-L1.

### EXAMPLES

The following examples demonstrate some embodiments of the compositions and methods described herein. However, it is to be understood that these examples are for illustration purposes only and do not intend, nor should any be construed, to be wholly definitive as to conditions and scope of this invention. The experiments in the following examples were carried out using standard techniques, which are well known and routine to those of skill in the art, except where otherwise described.

The following examples describe several, illustrative, PD-L1 targeting molecules. Each illustrative PD-L1 targeting molecule comprises at least one Shiga toxin A Subunit effector polypeptide linked, either directly or indirectly, to a cell-targeting binding region capable of specifically binding an extracellular part of a PD-L1 target biomolecule physically associated with a cellular surface of a cell. The Shiga toxin effector polypeptides in the Examples are de-immunized while retaining catalytic and/or cytotoxic activities, unless indicated herein as “inactivated.” Some of the illustrative PD-L1 targeting molecules comprise at least one Shiga toxin effector polypeptide component which (1) is de-immunized; (2) is on or proximal to an amino-terminus of a single-chain polypeptide component of the PD-L1 targeting molecule; (3) is furin-cleavage resistant; and/or (4) comprises an embedded CD8+ T-cell epitope (“C1”) that is heterologous to Shiga toxins.

The illustrative, cytotoxic, PD-L1 targeting molecules described below bound to cell-surface PD-L1 molecules expressed by targeted, tumor cell-types and were internalized by the targeted cells. Some of the internalized, PD-L1 targeting molecules shown in the Examples effectively routed their Shiga toxin effector polypeptides to the cytosols of target cells where the Shiga toxin effector polypeptides inactivated ribosomes and subsequently caused the apoptotic death of the targeted cells. In addition, certain, illustrative PD-L1 targeting molecules were able to effectively deliver immunogenic, T-cell epitopes to the WIC class I pathway of target cells to induce intercellular T-cell engagement, resulting in cytokine secretion and/or target cell killing.

### Example 1. Illustrative PD-L1 Targeting Molecules can Directly Kill Target Cells and Induce Indirect, Immune-Cell Mediated, Target Cell Killing

### I. Construction of Illustrative PD-L1 Targeting Molecules

Illustrative PD-L1 targeting molecules were created and tested. The PD-L1 targeting molecules each comprised one or more fusion proteins comprising 1) a PD-L1 binding region comprising an immunoglobulin-type binding domain and 2) a de-immunized Shiga toxin effector A Subunit polypeptide component which is furin-cleavage resistant. All of the PD-L1 targeting molecules tested in the experiments of this Example were produced in a bacterial system and purified by column chromatography using techniques known to the skilled worker. Catalytically inactive Shiga toxin effector polypeptides were used in some of the PD-L1 targeting molecules to help differentiate enzymatic contributions to target cell killing from CD8+ T-cell epitope delivery contributions. The PD-L1 targeting molecules comprising a catalytically inactive Shiga toxin effector polypeptide are labeled in the Examples as “inactivated.” Each of these inactivated molecules comprises a single amino acid residue substitution which severly reduces Shiga toxin A subunit catalytic activity in vitro.

The illustrative PD-L1 targeting molecules that were produced and tested in this Example (see Table 4, below) include those comprising a binding region having the amino acid sequence of SEQ ID NO: 38 (scFva) or SEQ ID NO: 39 (scFvb). Some of the PD-L1 binding molecules also comprise one or more copies of a CD8+ T-cell epitope (C1, SEQ ID NO: 77), and some comprise an endoplasmic reticulum retention/signal motif (ER).

TABLE 4
PD-L1 Targeting Molecules

| | SEQ ID NO |
|---|---|
| PD-L1 binding molecule | (Molecule No) |
| DI-SLTA-1::scFva::C1 | 191 (115694) |
| DI-SLTA-1::scFva | 86 (115695) |
| DI-SLTA-1::scFva::C1::C1 | 87 (115765) |
| inactivated DI-SLTA-1::scFva::C1::C1 | 88 (115826) |
| DI-SLTA-1::scFvb::C1 | 85 |
| DI-SLTA-1::scFvb | 84 |
| ER-DI-SLTA-1::scFva::C1::C1 | 212 (115773) |

### II. PD-L1 Binding by Illustrative PD-L1 Targeting Molecules

The binding of the illustrative PD-L1 targeting molecule to a PD-L1 molecule can be analyzed using standard techniques. For example, purified recombinant PD-L1 can be used to characterize a PD-L1 targeting molecule's binding characterstics, such as, e.g., using an ELISA assay. To perform a PD-L1 target binding ELISA, plates are coated with human, cynomolgus monkey or mouse recombinant PD-L1 targeted by the scFv component of the PD-L1 targeting molecule. Preparations are made in PBS and incubated overnight at 4 degrees Celsius (° C.). The plates are blocked with 3% milk-PBS-T for 2 hours, then washed and incubated for 1 hour at room temperature with PD-L1 targeting molecule. The plates are washed again with PBS-T and incubated for 1 hour at room temperature with a mouse monoclonal antibody that binds to the Shiga toxin derived component of the PD-L1 targeting molecule. The plates are washed with PBS-T, then incubated with anti-mouse Fc-HRP antibody for 1 hour at room temperature. Following the incubation, the plates are washed again with PBS-T. Binding of PD-L1 targeting molecules is detected following incubation with TMB Ultra reagent (Thermo Fisher Scientific, Waltham Mass., U.S.) and the stopping of the reaction with HCl. Absorbance is then measured at 450 nanometer (nm).

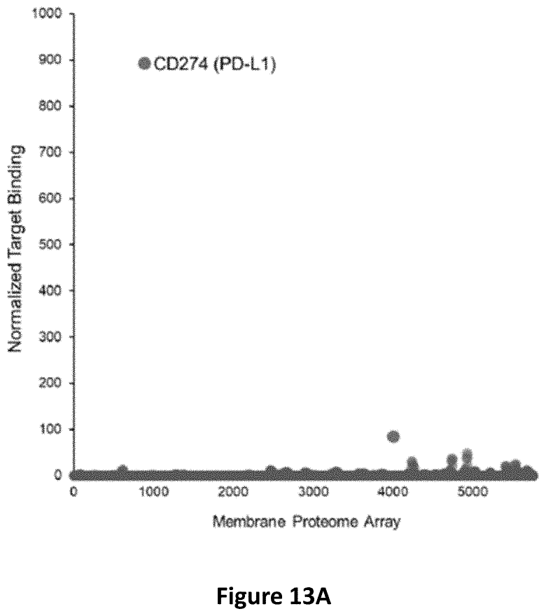

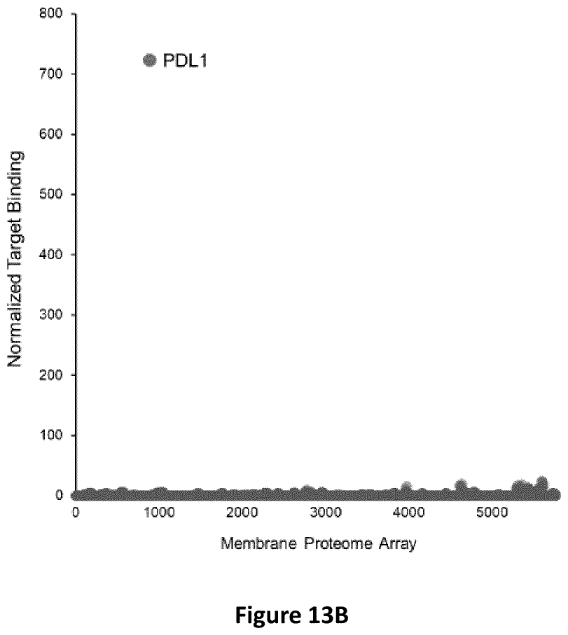

The binding specificity, affinity, and selectivity of an illustrative PD-L1 targeting molecule were tested by analyzing binding to a membrane proteome array, which comprised 5,300 different human proteins known to be expressed on the cell surface of HEK-293T cells (Integral Molecular, Inc., Philadelphia, Pa., U.S.). Only human PD-L1 (CD274) was identified and validated among the 5,300 proteins as a bound target for DI-SLTA-1::scFva::C1 (SEQ ID NO:191), DI-SLTA-1::scFva::C1::C1 (SEQ ID NO: 87), and DI-SLTA-1::scFva (SEQ ID NO:86). Both DI-SLTA-1::scFva::C1 and DI-SLTA-1::scFva exhibited robust and specific binding to human PD-L1 (FIG. 13A-13B).

A routine method involving a mutational analysis known to the skilled worker was used to evaluate critical contact points or “hotspots” in the PD-L1 binding regions (see e.g. Zhang et al., Oncotarget, 2017). Contact residues for DI-SLTA-1::scFva::C1, DI-SLTA-1::scFva::C1::C1, and DI-SLTA-1::scFva binding to human PD-L1 where mapped to residues F42, D122, and Y123. Critical contact residues for DI-SLTA-1::scFva::C1 binding to human PD-L1 where mapped to residues F42, D122, and Y123 within PD-L1.

### III. PD-L1 Expressing Target Cell Binding by Illustrative PD-L1 Targeting Molecules was Evaluated Using Target Positive Cell Lines

The binding of illustrative PD-L1 targeting molecules to a PD-L1 expressing cell can be analyzed using standard techniques, for example using indirect flow cytometry methods.

The binding characteristics of various illustrative PD-L1 targeting molecules to their biomolecular targets was determined using an indirect flow cytometry-based assay known to the skilled worker. For each reaction, about 0.3×10⁶ cells expressing PD-L1 were collected, washed in PBS+1% BSA and incubated for 1 hour at 4° C. with serial dilutions of a PD-L1 targeting molecule sample. After incubation, the cells were washed and incubated for one hour at 4° C. with a FITC-conjugated antibody, which bound to the SLT-1A component of the PD-L1 targeting molecules. Cells were then washed and resuspended in PBS and analyzed by flow-cytometry (Athena, Cytek Bio) using the blue laser (488 nm excitation). Target cells incubated with anti-SLT-1A-FITC only were used as the background control. Mean fluorescent intensity (MFI) values were normalized by subtracting the MFI of background controls and plotted as a function of concentration.

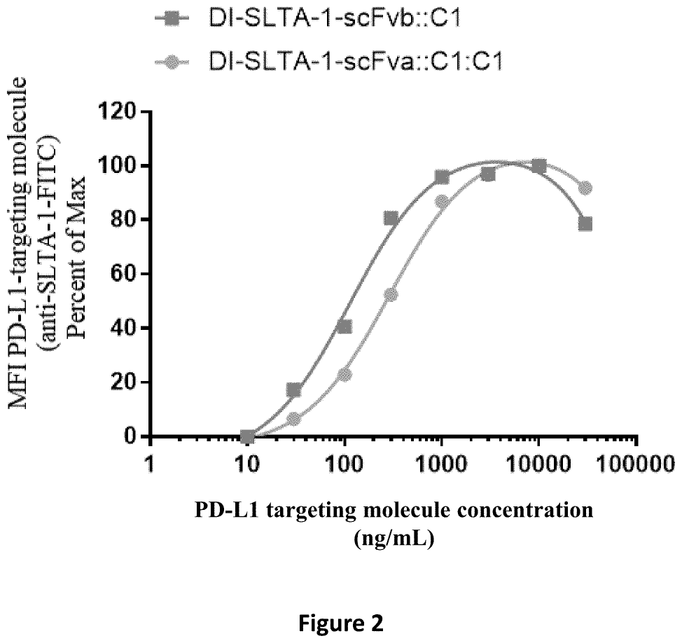

Target cell binding profiles of illustrative PD-L1 targeting molecules are shown in FIG. 2. Specifically, FIG. 2 shows the amount of binding of illustrative PD-L1 targeting molecules DI-SLTA-1::scFvb::C1 (SEQ ID NO:85) and DI-SLTA-1::scFva::C1::C1 (SEQ ID NO: 87) to PD-L1 positive HCC1954 cells at different concentrations of PD-L1 targeting molecule. On the Y-axis is plotted the percentage of maximum mean fluorescence intensity (MFI) for PD-L1 targeting molecule, and the X-axis shows PD-L1 targeting molecule concentration in ng/mL. These data demonstrate that illustrative PD-L1 targeting molecules bind human PD-L1 with nanomolar (nM) affinity.

### IV. Testing the Cytotoxic Activities of Illustrative PD-L1 Targeting Molecules

The cytotoxic activities of illustrative PD-L1 targeting molecules are measured using a tissue culture cell-based toxicity assay. The assays use either target biomolecule positive (PD-L1+) or target biomolecule negative (PD-L1−) cells.

In this example, the cell-kill assays were performed as follows. Human tumor cell line cells were plated (typically at 2×10³ cells per well for adherent cells, plated the day prior to protein addition, or 7.5×10³ cells per well for suspension cells, plated the same day as protein addition) in 20 μL cell culture medium in 384-well plates. A series of 10-fold dilutions of the proteins to be tested was prepared in an appropriate buffer, and 5 μL of the dilutions or only buffer as a negative control were added to the cells. Control wells containing only cell culture medium were used for baseline correction. The cell samples were incubated with the proteins or just buffer for 3 or 5 days at 37° C. and in an atmosphere of 5% carbon dioxide (CO₂). The total cell survival or percent viability was determined using a luminescent readout using the CellTiter-Glo® Luminescent Cell Viability Assay (G7573 Promega Madison, Wis., U.S.) according to the manufacturer's instructions as measured in relative light units (RLU). The Percent Viability of experimental wells was calculated using the following equation: (Test RLU−Average Media RLU)÷(Average Cells RLU−Average Media RLU)×100. Log protein concentration versus Percent Viability was plotted in Prism (GraphPad Prism, San Diego, Calif., U.S.) and log (inhibitor) versus response (3 parameter) analysis were used to determine the half-maximal cytotoxic concentration (CD₅₀) value (i.e., the concentration of exogenously administered PD-L1 targeting molecule which kills half the cells in a homogenous cell population) for the tested proteins. The CD₅₀ values for each illustrative PD-L1 targeting molecule tested was calculated when possible.

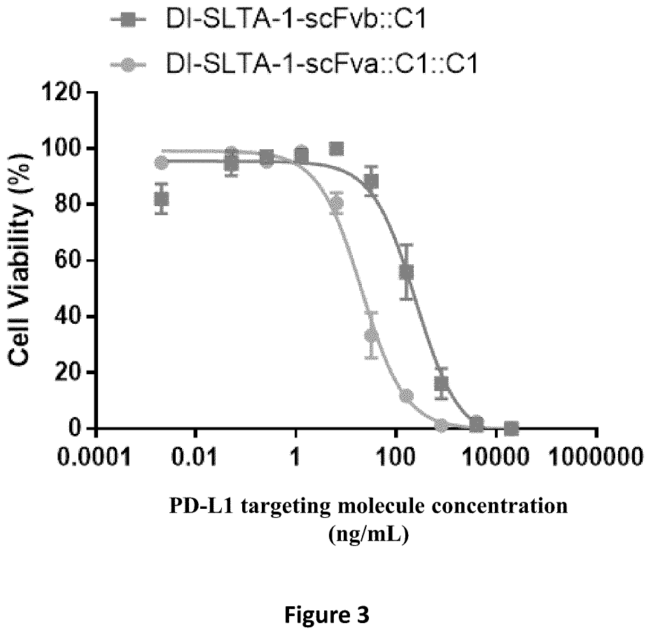

FIG. 3 shows the cytotoxicity of illustrative PD-L1 targeting molecules DI-SLTA-1::scFvb::C1 (SEQ ID NO:85) and DI-SLTA-1::scFva::C1::C1 (SEQ ID NO: 87) to PD-L1 positive HCC1954 cells at different concentrations of PD-L1 targeting molecule. On the Y-axis is plotted the percentage of cell viability, versus the PD-L1 targeting molecule concentration in ng/mL on the X-axis. These PD-L1 targeting molecules exhibited potent cytotoxicity to target positive cell lines. In a PD-L1 positive cell kill assay, the CD₅₀ values for DI-SLTA-1::scFva and DI-SLTA-1::scFva::C1::C1 were comparable (data not shown).

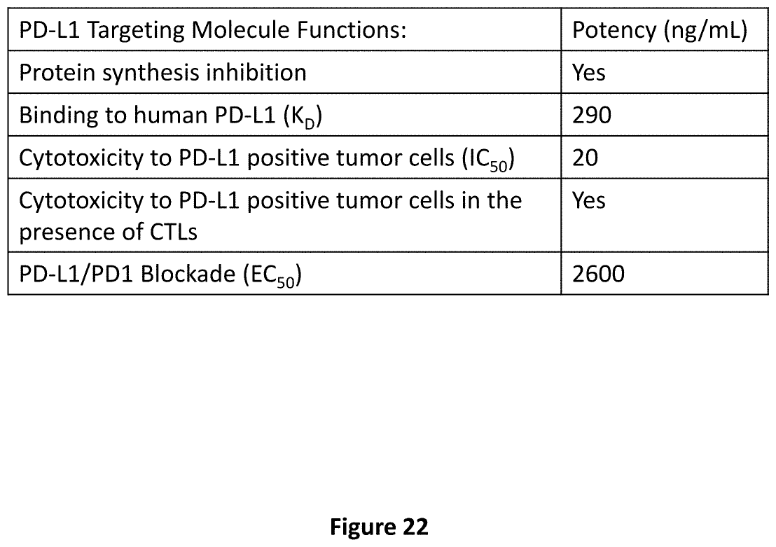

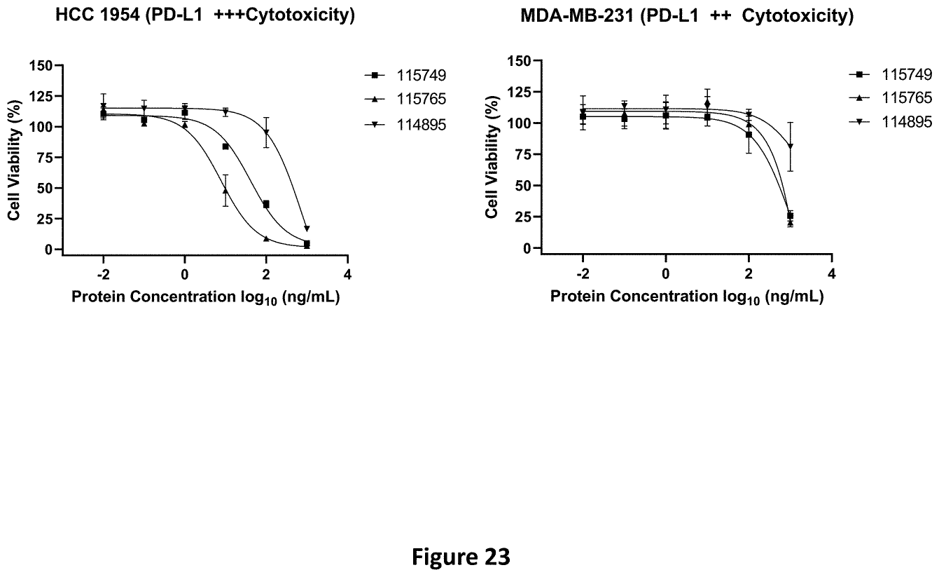

The concentration of DI-SLTA-1::scFva::C1::C1 for optimal binding and cytotoxic potency to PD-L1 expressing human cells may be determined from the empirical results shown in FIGS. 3 and 22. The cytotoxic potency of DI-SLTA-1::scFvb::C1 (SEQ ID NO:85) to PD-L1 expressing human cells may be favorable at concentrations below full receptor occupancy conditions (see FIGS. 2-3).

### V. Testing PD-L1/PD-1 Signaling Interference by PD-L1 Targeting Molecules

The PD-1/PD-L1 signaling interaction was investigated in the presence of illustrative PD-L1 targeting molecules using a PD-L1/PD-1 blockage reporter kit (Promega®, Madison, Wis., U.S.), which is based on a Jurkat NFAT-Luc reporter system activated by the release of PD-1 inhibition. Atezolizumab, a known inhibitor of the PD-L1/PD-1 interaction, was used a positive control and PBS was used as a negative control. Target cells expressing PD-L1 (provided with the kit) were incubated with proteins for 30 minutes and then co-cultured for six hours at 37° C. with Jurkat T-cells expressing PD-1 (provided with the kit) expressing an NFAT-Luc reporter under the control of a TCR. BioG10™ reagent (luciferin) was added to wells of plate and plates were read for luminescence using a luminometer according to standard procedure (Promega).

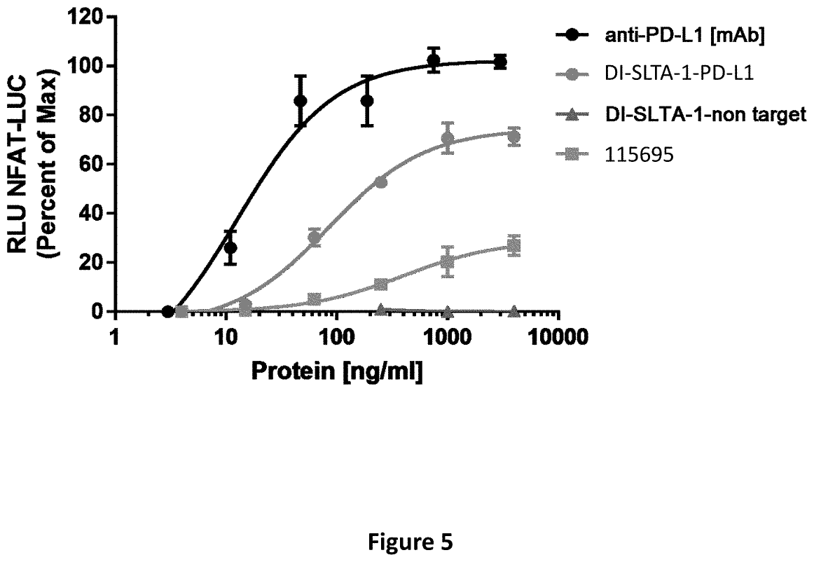

FIG. 5 shows blockade of PD-1/PD-L1 signaling caused by administration of an anti-PD-L1 monoclonal antibody (mAb), a PD-L1 targeting molecule (DI-SLTA-1-PD-L1), a DI-SLTA-1 molecule without a targeting domain (DI-DLTA-1-non target) and molecule 115695 (SEQ ID NO: 86) to PD-1 positive Jurkat T-cells expressing an NFAT-Luc reporter under the control of a T-cell receptor (TCR). The relative light units (RLU) measured from the luciferase reporter as a percentage of maximum is plotted on the Y-axis, and the PD-L1 targeting molecule concentration is plotted in ng/mL on the X-axis.

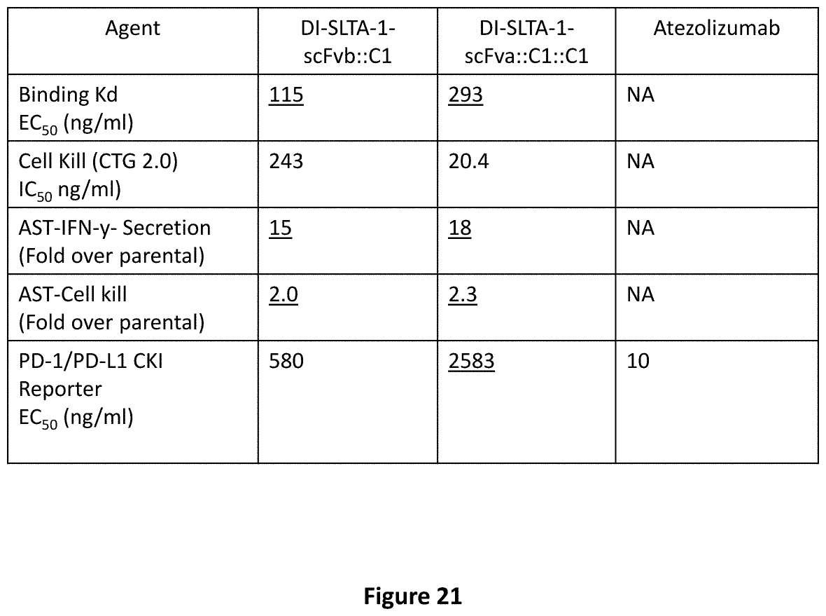

The PD-L1 signaling half-maximal effective concentration (EC₅₀) value (concentration at which blockade is 50% of positive control) or half-maximal PD-L1 signaling inhibitory concentration (IC₅₀) value of DI-SLTA-1::scFvb::C1 (SEQ ID NO:85) in this PD-1/PD-L1 Blockade assay was over 100-fold greater than the CD₅₀ value for HCC1954 cells (see FIG. 21), which suggests that potent killing of target cell killing may occur at PD-L1 targeting molecule concentrations that are not sufficient to cause significant PD-1/PD-L1 signaling blockade activity.

VI. Testing CD8+ T-Cell Epitope Delivery and MHC-Peptide Complex (pMHC is) Expression Induction Capabilities of Illustrative PD-L1 Targeting Molecules

The successful delivery of a fused CD8+ T-cell epitope from an extracellular molecule to the interior of a cell and into the MHC class I presentation pathway can be determined by detecting a specific cell surface, MHC class I molecule/epitope complex (pMHC I) on the cell surface after exogenous administration of the delivery molecule. To test whether a PD-L1 targeting molecule can deliver a fused, CD8+ T-cell epitope to the MHC class I presentation pathway of target cells, flow cytometry-based assays known to the skilled worker are employed which detect MHC Class I molecules complexed with specific epitopes.

### VII. Testing Cytotoxic T-Cell Mediated Cytolysis of Intoxicated Target Cells and Other Immune Responses Triggered by MHC Class I Presentation of T-Cell Epitopes Delivered by PD-L1 Targeting Molecules

In this Example, standard assays known in the art were used to investigate the functional consequences of target cells' MHC class I presentation of T-cell epitopes delivered by illustrative PD-L1 targeting molecules, such as, e.g., CTL-mediated target cell killing and cytokine release by CTLs.

A CTL-based cytotoxicity assay was used to assess the consequences of CD8+ T-cell epitope presentation. The assay involves tissue-cultured target cells and T-cells. PD-L1+ target cells were intoxicated with illustrative PD-L1 targeting molecules by incubating the cells (typically for 4 hours or 16 hours or more) in standard conditions, including at 37° C. and an atmosphere with 5% carbon dioxide, to allow for intoxication by the PD-L1 targeting. After incubation, the target cells were washed. Next, CTLs were added to the treated target cells and incubated to allow for the CTLs to recognize and bind any target cells displaying epitope-peptide/MHC class I complexes (pMHC Is). Then certain functional consequences of pMHC I recognition were investigated using standard methods known to the skilled worker, including epitope-presenting target cell killing by CTL-mediated cytolysis, and the release of cytokines, such as IFN-γ or interleukins by ELISA. For example, for detection of cytokine secretion, supernatant was harvested from co-culture experiments at 48 hours after treatment and human IFN-γ cytokine levels were detected using an ELISA Max kit (BioLegend, San Diego, Calif., U.S.A.) according to the manufacturer's protocol. Changes in absorbance at 450 nm were compared to a standard curve and analyzed via GraphPad Prism Software.

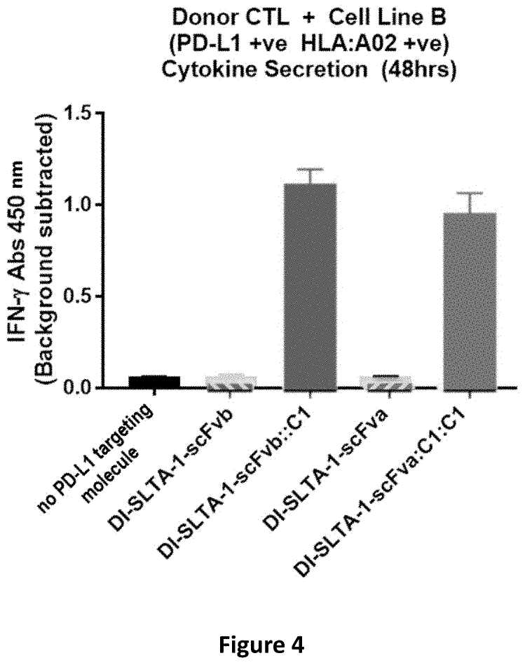

FIG. 4 shows the amount of human interferon gamma (IFN-γ) secretion induced 48 hours after exogenous administration of illustrative PD-L1 targeting molecules DI-SLTA-1::scFvb (SEQ ID NO:84), DI-SLTA-1::scFvb::C1 (SEQ ID NO:85), DI-SLTA-1::scFva (SEQ ID NO:86), and DI-SLTA-1::scFva::C1::C1 (SEQ ID NO: 87) to PD-L1 positive/HLA:A02 positive MDA-MB-231 cells in the presence of donor cytotoxic T-cells. The label “No PD-L1 targeting molecule” refers to a sample to which no PD-L1 targeting molecule was administered.

The data in FIG. 4 shows that the CD8+ T-cell epitope-peptide cargo must be present to elicit significant human interferon gamma secretion in this assay. DI-SLTA-1::scFvb::C1 (SEQ ID NO:85) and DI-SLTA-1::scFva::C1::C1 (SEQ ID NO: 87) showed similar abilities to deliver a CD8+ T-cell epitope delivery-dependent response (IFN-γ secretion) in co-culture models with CMV antigen specific-CTLs and HLA:A2 positive/PD-L1 positive target cells (MDA-MB-231 cells). Similar results were also observed for DI-SLTA-1::scFva::C1::C1 (SEQ ID NO: 87, 115765).

To test for T-cell enrichment, PBMCs from HLA-typed donors were isolated from Leukopaks (All Cells, Alameda, Calif., U.S.) by density centrifugation. Donor 1 (D1) was homozygous for the HLA:A2 allele, and donor 2 (D2) was heterozygous at the HLA-A locus, carrying only one copy of the HLA:A2 allele. For CMV-CTL enrichment, cells were treated with C1 peptide (SEQ ID NO:77) (CMV-pp65 for HLA:A02) in the presence of a cytokine cocktail. Cells were expanded in vitro for one to two weeks, and CMV-CTL expansion was measured by flow cytometry using fluorophore conjugated TCR-binding, MHC-I pentamers (ProImmune, Inc., Sarasota, Fla., U.S.).

Co-culture assays were also performed. Briefly, PD-L1 positive cells were plated at 10,000 to 20,000 cells per well in 96-well plates (˜40% confluence) and incubated with illustrative PD-L1 targeting molecules as described above. After 4 hours or 24 hours, the PD-L1 targeting molecule was washed away and then cells were co-cultured with T-cells or just cell-culture medium. PD-L1 positive/HLA:A2 positive MDA-MB-231 cells that express a fluorescent tag (Essen Bioscience, Inc. Ann Arbor, Mich., U.S.A.) or MCF-7 cells were used as targets in IncuCyte assays. Cell viability over time was measured using a IncuCyte-S3-Live cell imager (Essen Bioscience), and phase and fluorescent images were captured every four to six hours. Percent viability was measured by fluorescent cell counts via IncuCyte-S3 software package (Essen Bioscience). Viability data was plotted as total fluorescent cell counts and normalized to time zero. CD₅₀ values were calculated by non-linear regression and plotted using GraphPad® Prism® Software.

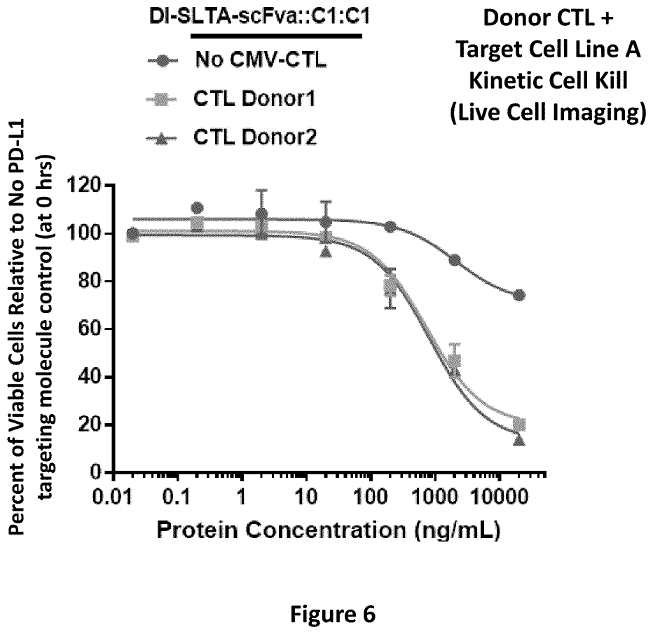

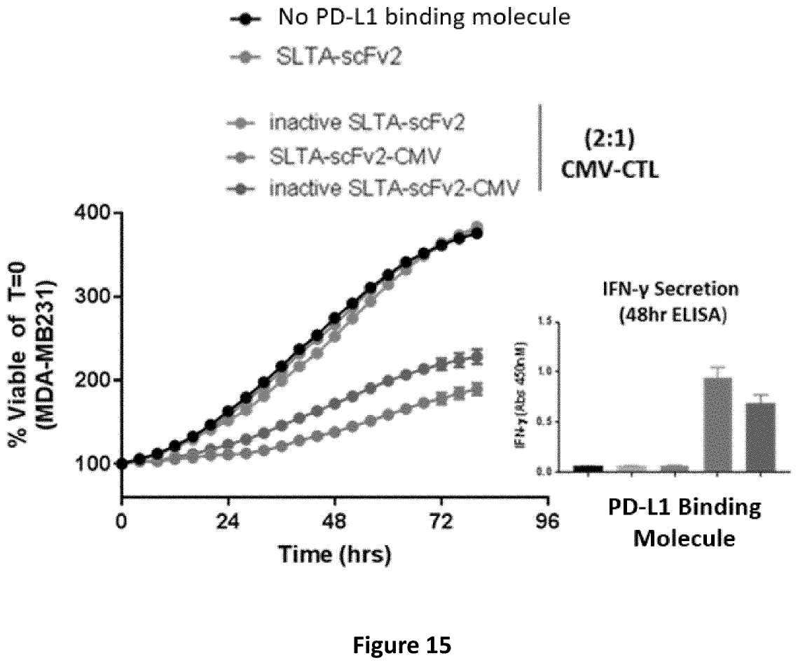

FIG. 6 and FIG. 15 show dose-dependent cytotoxicities of illustrative PD-L1 targeting molecules DI-SLTA-1::scFva (SEQ ID NO:86) (no CMV-CTL) and DI-SLTA-1::scFva::C1::C1 (SEQ ID NO: 87) to PD-L1 positive/HLA:A02 negative HCC1954 cells in the presence of donor cytotoxic T-cells after acute exposure (4 hours) to the PD-L1 targeting molecule. On the Y-axis is plotted the percentage of cell viability versus the PD-L1 targeting molecule concentration in ng/mL on the X-axis. Certain results from this experiment are reported in Table 5.

TABLE 5
CTL-dependent Cytotoxicity Induced by Illustrative
PD-L1 Targeting Molecules Occurs with HLA:A02
Homozygous or Heterozygous Donor CMV-CTLs

| PD-L1 targeting molecule - | cytokine secretion |
|---|---|
| CMV-CTL donor cell type | (fold over control) |
| DI-SLTA-1::scFvb::C1 + Donor 1 cells | 4.8 |
| (homozygous) | |
| DI-SLTA-1::scFva::C1::C1 + Donor 2 | 7.5 |
| cells (heterozygous) | |

The results in FIG. 6 and FIG. 15 show that CD8+ T-cell epitope delivery and induction of T-cell engagement occurs within 4 hours, which is consistent with PD-L1 targeting molecule delivering of antigen and mobilizaztion of surface peptide/complex to the surface of cells to be available as soon as CTLs are added in the assay. Thus, this mechanism of action results in the initiation of a mechanism of cell kill that may result in increased target cell cytoxicity when combined with the slower acting cell-kill mechanism of ribosomal inhibition. The cytotoxicity and CTL engagement results shown in FIG. 6, FIG. 15, and Table 5 showed no significant difference between donor 1 (D1) and donor 2 (D2). Thus, although the T-cell engagement mechanism of cell kill may be restricted to HLA:A2 matched donor CMV-CTLs, this cell-kill mechanism (MOA-2) can be achieved with either full or heterozygous haplotype matching to the CD8+ T-cell antigen payload.

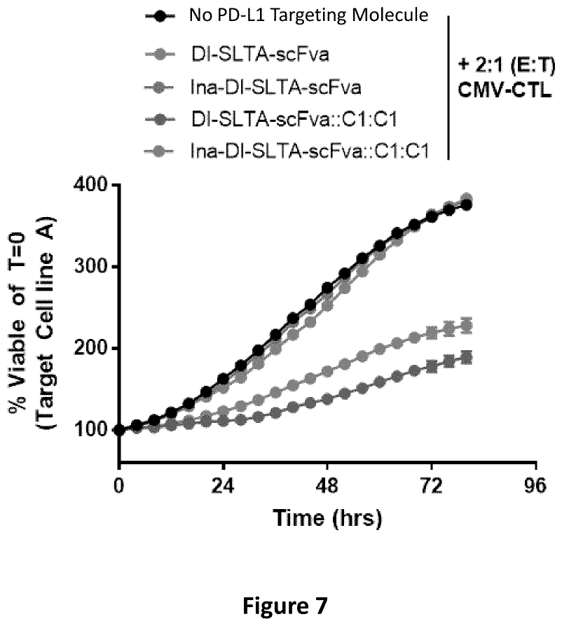

FIG. 7 shows the cytotoxicity of illustrative PD-L1 targeting molecules DI-SLTA-1::scFva (SEQ ID NO:86), inactivated DI-SLTA-1::scFva (SEQ ID NO:88), DI-SLTA-1::scFva::C1::C1 (SEQ ID NO: 87), and inactivated DI-SLTA-1::scFva::C1::C1 to PD-L1 positive/HLA:A02 positive MDA-MB-231 cells in the presence of donor cytotoxic T-cells (at a ratio of 2 target cells to 1 cytotoxic T-cell (2:1 (E:T))) in a kinetic analysis. On the Y-axis is plotted the percentage of cell viability versus the time in hours on the X-axis. In FIG. 7, the term “CMV-CTL” refers to CMV antigen specific-CTLs, such as, e.g., those CTLs having T-cell receptors (TCRs) recognizing the C1 peptide (SEQ ID NO:77).

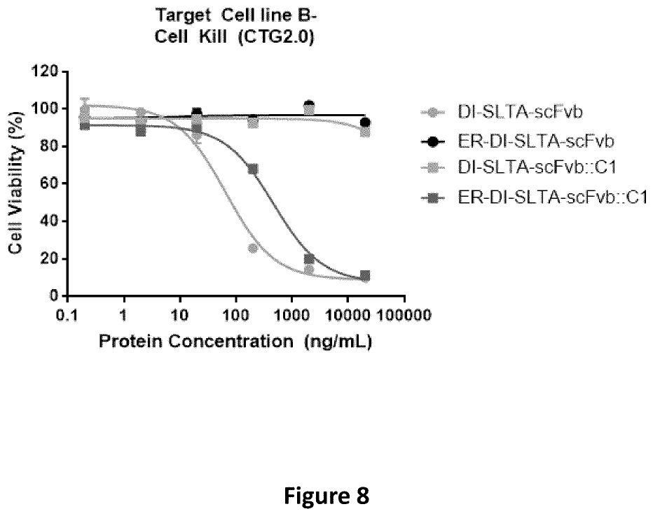

FIG. 8 shows dose-dependent cytotoxicity of DI-SLTA-1::scFvb (SEQ ID NO:84), ER-DI-SLTA-1::scFvb, DI-SLTA-1::scFvb::C1 (SEQ ID NO:85), and ER-DI-SLTA-1::scFvb::C1 to PD-L1 positive MDA-MB-231 cells. On the Y-axis is plotted the percentage of cell viability versus the PD-L1 targeting molecule concentration in ng/mL on the X-axis.

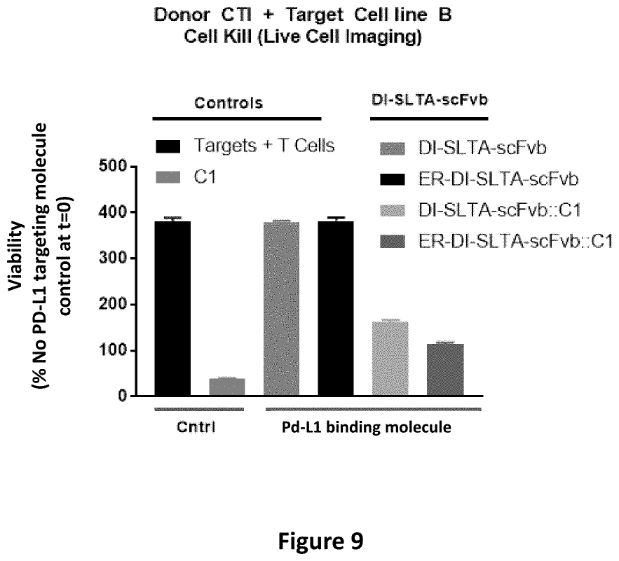

FIG. 9 shows the cytotoxicity of illustrative PD-L1 targeting molecules DI-SLTA-1::scFvb (SEQ ID NO:84), ER-DI-SLTA-1::scFvb, DI-SLTA-1::scFvb::C1 (SEQ ID NO:85), and ER-DI-SLTA-1::scFvb::C1 to PD-L1 positive/HLA:A02 positive MDA-MB-231 cells in the presence of donor cytotoxic T-cells. The percentage of cell viability is shown on the Y-axis with controls on the left. In FIG. 9, the term “cntrl” refers to control experiments performed in the absence of PD-L1 targeting molecule.

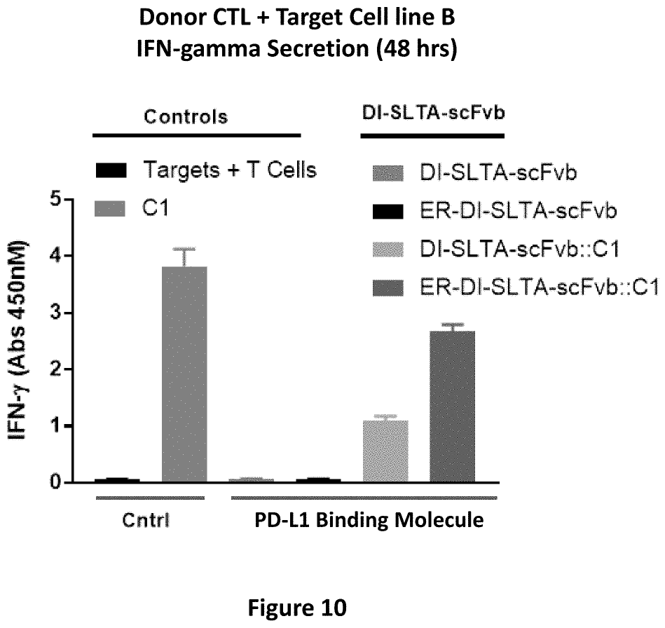

FIG. 10 shows the amount of human interferon gamma (IFN-γ) secretion induced 48 hours after exogenous administration of an illustrative PD-L1 targeting molecule to PD-L1 positive/HLA:A02 positive MDA-MB-231 cells in the presence of donor cytotoxic T-cells. The data shown is for the illustrative PD-L1 targeting molecules DI-SLTA-1::scFvb (SEQ ID NO:84), ER-DI-SLTA-1::scFvb, DI-SLTA-1::scFvb::C1 (SEQ ID NO:85), and ER-DI-SLTA-1::scFvb::C1.

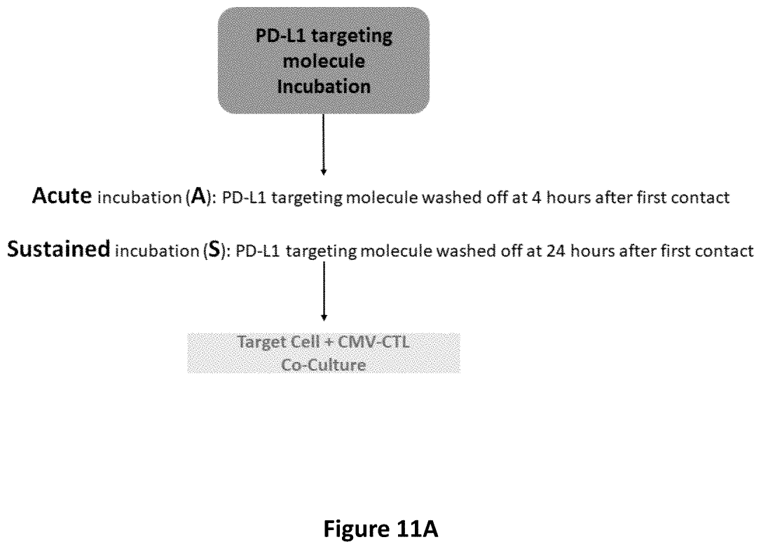

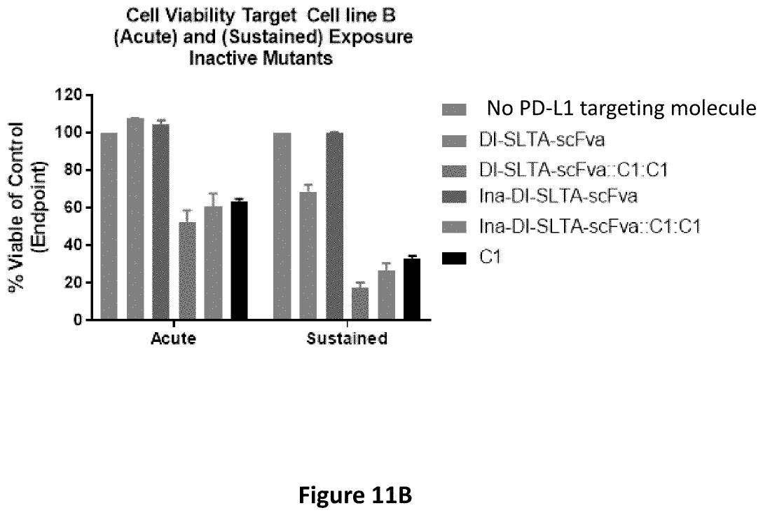

FIG. 11B shows the cytotoxicity of illustrative PD-L1 targeting molecules to PD-L1 positive/HLA:A02 positive MDA-MB-231 cells after acute (4 hour, followed by washout) or sustained (24 hour) exposure to the PD-L1 targeting molecule, according to the protocol shown in FIG. 11A). The percentage of cell viability is shown on the Y-axis. The data shown is for the illustrative PD-L1 targeting molecules DI-SLTA-1::scFva (SEQ ID NO:86), ER-DI-SLTA-1::scFva::C1::C1, inactivated DI-SLTA-1::scFva (SEQ ID NO:88), and inactivated DI-SLTA-1::scFva::C1::C1.

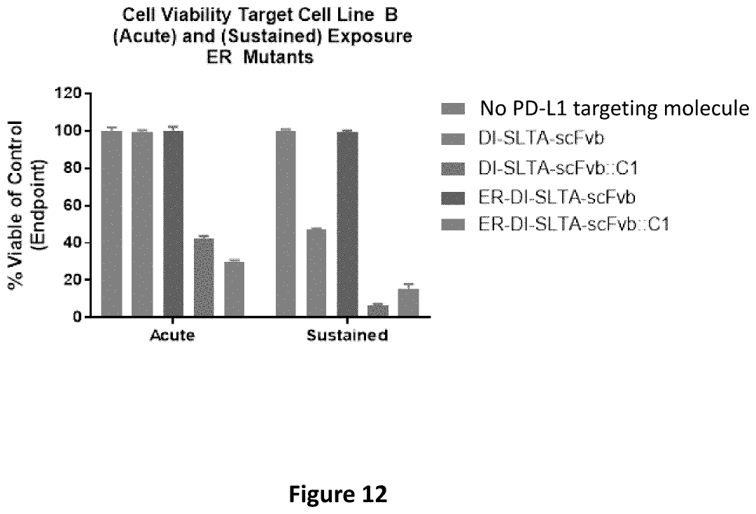

FIG. 12 shows the cytotoxicity of illustrative PD-L1 targeting molecules to PD-L1 positive/HLA:A02 positive MDA-MB-231 cells after acute or sustained exposure to the PD-L1 targeting molecule. The percentage of cell viability is shown on the Y-axis. The data shown is for the illustrative PD-L1 targeting molecules DI-SLTA-1::scFvb (SEQ ID NO:84), DI-SLTA-1::scFvb::C1 (SEQ ID NO:85), ER-DI-SLTA-1::scFvb, and ER-DI-SLTA-1::scFvb::C1.

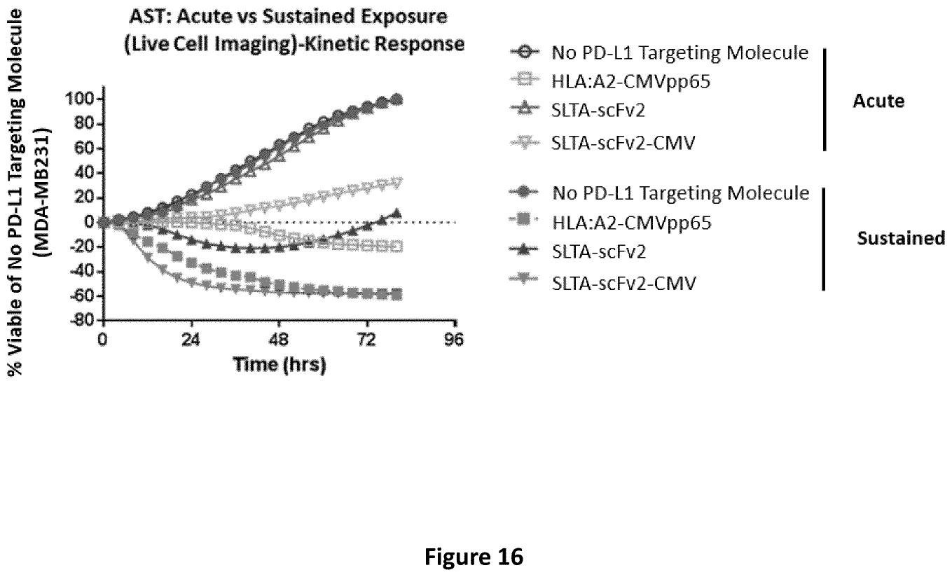

Taken together, the data in FIGS. 11B and 12 shows that delivery of the CD8+ T-cell epitope and presentation thereof on the surface of the cell occurs under acute and sustained exposure. The data shown in FIG. 16 shows that the tested PD-L1 targeting molecules act quickly to induce cell death and T cell activation under both short or long exposure, with longer exposure resulting in more activity (i.e., cell death and T-cell activation).

The cytotoxicity of illustrative PD-L1 targeting molecules to PD-L1 positive cells (target cells) in the presence of cytotoxic lymphocytes is affected by the MHC class I molecule expressed by the PD-L1 positive target cell and the TCR expressed by the cytotoxic lymphocyte in the co-culture. Target cells expressing HLA:A2 are killed more readily by C1 epitope carrying PD-L1 targeting molecules. Similarly, PD-L1 negative cells expressing HLA:A2 (MCF-7 cells) are not killed in any significant amounts.

The inactivated PD-L1 targeting molecules lacking Shiga toxin effector catalytic activity were still able to elicit potent CTL-mediated cytotoxicity via intercellular CTL engagement (indirect killing mechanism of action based on delivered epitope presentation) as a result of PD-L1 targeting molecules delivering the C1 epitope-peptide to the target cell's MHC class I presentation pathway. The ER-DI-SLTA-1 component was designed to prevent the scaffold from readily escaping the endoplasmic reticulum. Constructs comprising such a Shiga toxin effector polypeptide can still be cytotoxic due to the presence of the CD8+ T-cell epitope cargo despite their predicted intracellular localization patterns preventing contact with ribosomes. The ER-DI-SLTA-1 component exhibits wild-type levels of catalytic activity in an in vitro translation inhibition assay.

FIGS. 13A and 13B show that an illustrative PD-L1 targeting molecules exhibited binding to human PD-L1 that was robust and specific using a membrane proteome array, which comprised 5,300 different human proteins known to be expressed on the cell surface of HEK-293T. Only human PD-L1 (CD274) was identified as a high affinity binder among the 5,300 proteins as a bound target for two illustrative PD-L1 targeting molecules comprising the same scFv (FIG. 13). FIG. 13A shows that DI-SLTA-1::scFva::C1 (SEQ ID NO:87) binding to human PD-L1 was robust and specific. FIG. 13B shows that DI-SLTA-1::scFva (SEQ ID NO:86) binding to human PD-L1 was robust and specific.

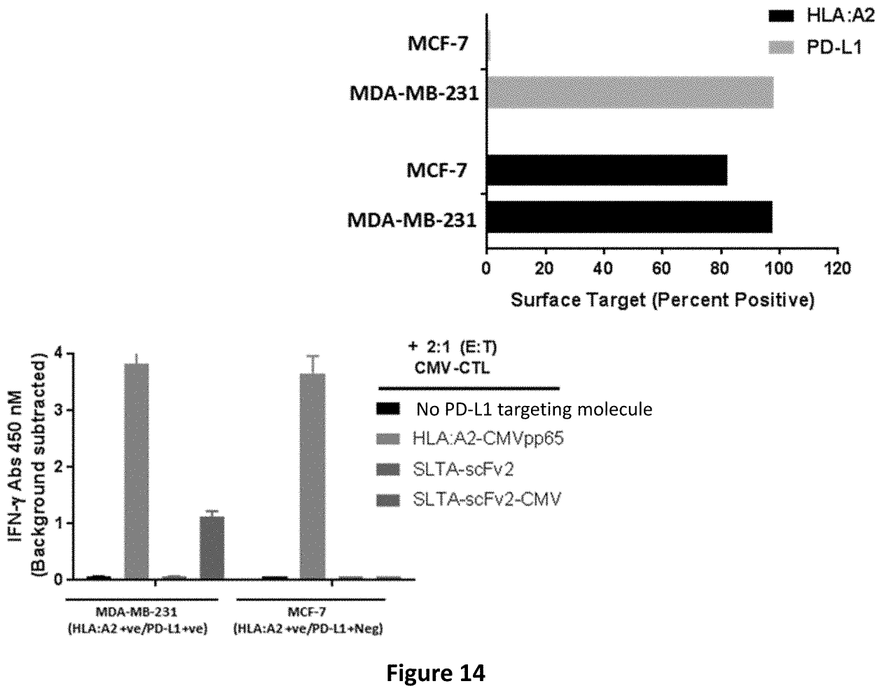

FIG. 14 shows the results of a T-cell engagement and stimulation assay experiment as described above and with other results from the experiment shown in FIG. 4. The top of FIG. 14 shows the relative cell-surface expression levels of PD-L1 and the MHC class I allele HLA:A2 for two tumor cell lines: MCF-7 and MDA-MB-231. The bottom of FIG. 14 shows the amount of human IFN-γ secretion induced after exogenous administration of illustrative PD-L1 targeting molecules to PD-L1 positive/HLA:A02 positive MDA-MB-231 cells or PD-L1 negative MCF-7 cells in the presence of donor cytotoxic T-cells which can recognize the epitope-peptide C1 (SEQ ID NO:77) complexed with MHC class I molecules. The lower graph shows DI-SLTA-1::scFva::C1::C1 administration to PD-L1 positive MDA-MB-231 cells in co-culture with CMV-antigen specific cytotoxic T lymphocytes (CTL) at a ratio of 2 target cells to 1 cytotoxic T-cell (“2:1 (E:T) CMV-CTL”) resulted in human interferon gamma (IFN-γ) secretion; however, administration of DI-SLTA-1::scFva::C1::C1 to PD-L1 negative MCF-7 cells did not. The label “No ETB” refers to no PD-L1 targeting molecule administration to that sample. The data in FIG. 14 shows that the CD8+ T-cell epitope-peptide cargo must be present to elicit significant human interferon gamma secretion in this assay as DI-SLTA-1::scFva (SEQ ID NO:86) failed to induce any significant amount of interferon gamma (IFN-γ) secretion. The data in FIG. 14 shows that the CD8+ T-cell epitope-peptide cargo must be present and target cells must express PD-L1 for administration of a PD-L1 targeting molecule to elicit significant human interferon gamma secretion in this assay. In FIG. 14, “SLTA-scFv2” refers to DI-SLTA-1::scFva (SEQ ID NO:86), and “SLTA-scFv2-CMV” refers to DI-SLTA-1::scFva::C1::C1. In FIG. 14, HLA:A2-CMVpp65 refers to T-cell epitope-peptide C1 (SEQ ID NO:77).

FIG. 15 shows PD-L1 targeting molecule treatment induces CTL-mediated cytotoxicity and human interferon gamma (IFN-γ) secretion in co-cultures of PD-L1 positive tumor cells (MDA-MB231) with antigen-specific CTLs at a ratio of 2 target cells to 1 CTL (“2:1 CMV-CTL”). In FIG. 15, “SLTA-scFv2” refers to DI-SLTA-1::scFva (SEQ ID NO:86), “SLTA-scFv2-CMV” refers to DI-SLTA-1::scFva::C1::C1, and “inactive SLTA-scFv2” refers to DI-SLTA-1::scFva (SEQ ID NO:86), and “inactive SLTA-scFv2-CMV” refers to “inactivated DI-SLTA-1::scFva::C1::C1”. The bottom right portion of FIG. 15 shows the induction of human interferon gamma (IFN-γ) secretion in co-cultures of PD-L1 positive tumor cells with antigen-specific CTLs at a ratio of 2 target cells to 1 CTL (“2:1 CMV-CTL”). Both DI-SLTA-1::scFva::C1::C1 and “inactivated DI-SLTA-1::scFva::C1::C1” were capable of inducing of human interferon gamma (IFN-γ) secretion, whereas samples lacking PD-L1 targeting molecule or PD-L1 targeting molecule comprising a C1 epitope did not induce any significant amount of IFN-γ secretion. Notably, even though the inactivated construct was not able to inactivate ribosomes, it was still able too deliver the CD8+ T-cell epitope. The data in FIG. 15 reveals the cooperative nature of these two mechanisms of action.

FIG. 16 shows the cytotoxicity of illustrative PD-L1 targeting molecules to PD-L1 positive/HLA:A02 positive MDA-MB-231 cells after acute (4 hours) or sustained (24 hours) exposure to the PD-L1 targeting molecule. The percentage of cell viability as compared to a negative control is shown on the Y-axis. The data shown is for the illustrative PD-L1 targeting molecules DI-SLTA-1::scFva (SEQ ID NO:86) and DI-SLTA-1::scFva::C1::C1 (SEQ ID NO: 87). In FIG. 16, the abbreviation “hrs” denotes “hours” and the abbreviation “Abs” denotes absorbance. In FIG. 16, “SLTA::scFv2” refers to DI-SLTA-1::scFva (SEQ ID NO:86), and “SLTA-scFv2-CMV” refers to DI-SLTA-1::scFva::C1::C1 (SEQ ID NO: 87). In FIG. 16, HLA:A2-CMVpp65 refers to T-cell epitope-peptide C1 (SEQ ID NO:77). Antigen delivery induced, T-cell mediated cytotoxicity occurs rapidly enough to observe during both acute and sustained exposure despite the presence of direct target cell kill via protein synthesis inhibition (compare DI-SLTA-1::scFva::C1::C1 to DI-SLTA-1::scFva for either condition). The results in FIG. 16 suggest antigen delivery induced, T-cell mediated cytotoxicity occurs at kinetic rates and potencies at both acute and sustained exposure conditions which adds to the direct target cell-kill potency, which appears to have slower kinetics. Thus, the two mechanisms of action may both contribute to producing cell kill under either acute or sustained exposure conditions (see e.g. FIGS. 11A, 11B, 12, 15-16, 18-19, and 21).

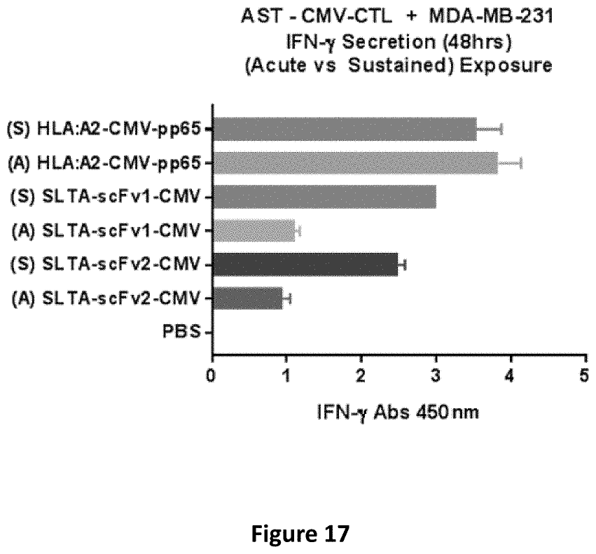

FIG. 17 shows the induction of human interferon gamma (IFN-γ) secretion in co-cultures of PD-L1 positive tumor cells (which are also HLA:A2 positive) with antigen-specific CTLs (having TCRs recognizing the C1 peptide (SEQ ID NO:77)) after 48 hours with samples subjected to either an acute (4 hours) or sustained (24 hours) exposure to a PD-L1 targeting molecule. Both DI-SLTA-1::scFvb::C1 (SEQ ID NO:85) and DI-SLTA-1::scFva::C1::C1 (SEQ ID NO: 87) were capable of inducing of human interferon gamma (IFN-γ) secretion by means of either an acute or sustained exposure. In FIG. 17, the term “SLTA-scFv1-CMV” refers to DI-SLTA-1::scFvb::C1, and the term “SLTA-scFv2-CMV” refers to DI-SLTA-1::scFva::C1::C1. In FIG. 17, HLA:A2-CMV-pp65 refers to samples treated only with the T-cell epitope-peptide C1 (SEQ ID NO:77).

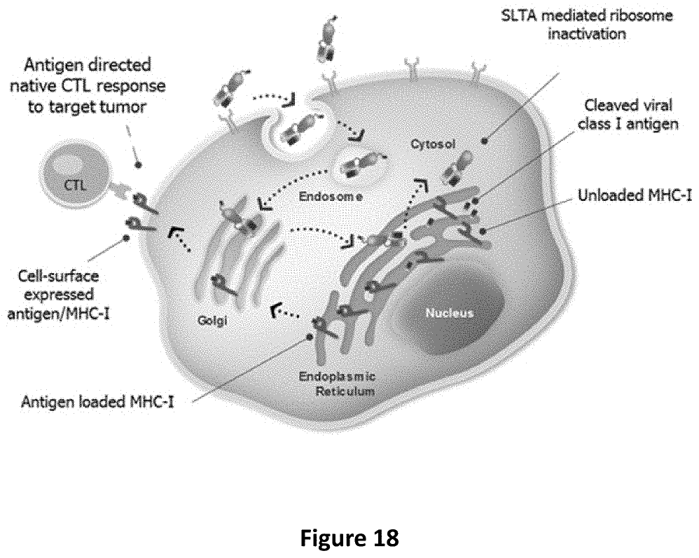

FIG. 18 visually depicts how two mechanisms of action for cell killing may be performed by a single PD-L1 targeting molecule. Mechanism of action #1 (MOA-1) is based on the catalytic damaging of PD-L1 expressing cell ribosomes and requires cytosolic localization of a Shiga toxin A Subunit A1 fragment component. Mechanism of action #2 (MOA-2) is based on the delivery of a CD8+ T-cell epitope-peptide cargo to the MHC class I presentation pathway. MOA-1 occurs with or without the presence of the epitope-peptide cargo. The presence of an epitope-peptide cargo required for MOA-2 does not have a significant impact on the function of MOA-1. MOA-2 involves the cleavage of the epitope-peptide cargo away from the PD-L1 targeting molecule and loading of an epitope-peptide (e.g. a viral antigen) onto a MHC class I molecule in the endoplasmic reticulum to form a complex. The MHC class I molecule-epitope-peptide complex then travels from the endoplasmic reticulum throught the Golgi and to the PD-L1 expressing cell surface. On the cell-surface, the antigen-MHC class I molecule complex induces cytotoxic T lymphocyte (CTL) responses to the antigen displaying cell. In this model, both MOA-1 and MOA-2 depend on the PD-L1 targeting molecule docking with PD-L1 on the surface of a target cell, cellular internalization of the PD-L1 targeting molecule, and retrograde transport of the Shiga toxin A Subunit A1 fragment component and the CD8+ T-cell epitope from the endosome to reach the endoplasmic reticulum.

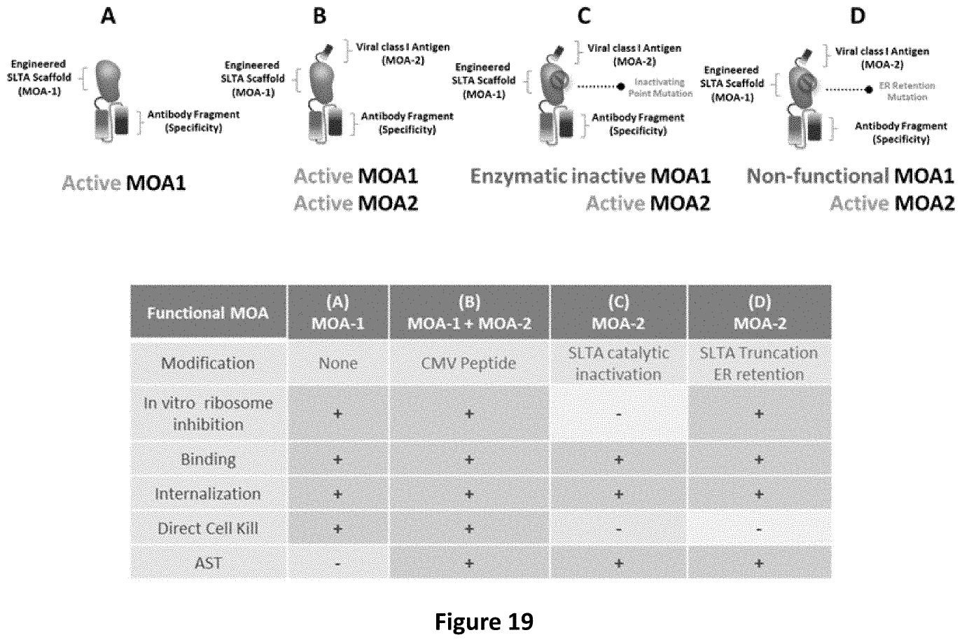

FIG. 19 summarizes the results described throughout Example 1. There are at least two different mechanisms of action that may be provided by a PD-L1 targeting molecule, described as MOA-1 and MOA-2. The empirical data in Example 1 demonstrates that a single PD-L1 targeting molecule can exhibit MOA2 activity concomitantly with MOA1 activity. The data also shows that MOA2 activity may be achieved in the absence of MOA1 activity, such as, e.g., by using PD-L1 targeting molecules having Shiga toxin A Subunit effector polypeptide components which are catalytically inactive and/or truncated at their carboxy-terminus, and are thereby predicted to result in retention in the endoplasmic reticulum instead of retranslocating to the cytosol (see e.g. La Pointe 2005). There are at least four different PD-L1 targeting molecule formats described: (A) active and capable of performing MOA-1 but not MOA-2, (B) active with both MOA-1 and MOA-2, (C) active with MOA-2 but not MOA-1 due to enzymatic inactivation via point mutation, and (D) active with MOA-2 but not MOA-1 due to endoplasmic reticulum localization (i.e. cytosolic localization is blocked) caused by a carboxy-terminal truncation of the A1 fragment of the Shiga toxin A Subunit effector polypeptide. For the PD-L1 targeting molecule format shown in (D), the Shiga toxin A Subunit effector polypeptide of the PD-L1 targeting molecule still retains catatlyic activity in vitro to depurinate ribosomes and inhibit protein synthesis but this catalytic activity does not occur in vivo because the molecule's subcellular localization (retention in the lumen of the endoplasmic reticulum) prevents contact with ribosomes. In FIG. 19, the abbreviation “AST” denotes “antigen seeding technology”, which refers to the ability to create immuno-stimulation in a local area of a target cell(s) within an organism via delivery of a CD8+ T-cell epitope for MHC class I presentation, such as, e.g., delivery of T-cell epitope-peptide C1 (SEQ ID NO:77) by a PD-L1 targeting molecule, resulting in cytokine secretion and/or cytotoxic T lymphocyte intercellular engagement and target cell-killing.

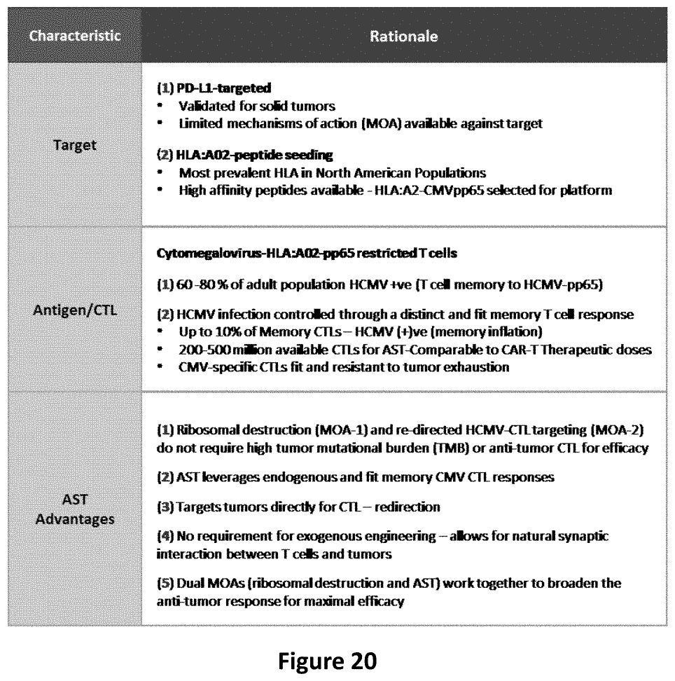

FIG. 20 describes the rationale behind the choice of CD8+ T-cell epitope cargos for delivery by a PD-L1 targeting molecule. The C1 peptide (SEQ ID NO:77) from the human cytomegalovirus (CMV) pp65 protein is recognized by HLA:A02 with high affinity. HLA:A02 is the most prevalent HLA allele in North American patient populations. About 60 to 80 percent of the adult population is CMV positive, meaning these people carry memory T-cells specific to pp65 antigens and these immune cells are chronically functioning to contain residual CMV in fashion that may be resistant to T-cell dysfunction, such as anergy. Antigen seeding via antigen cargo delivery to the MHC class I presentation pathway may function to redict endogenous T-cells specific to CMV antigen to tumor cells by altering the immunophenotype of the tumor site thereby mimicking an infected tissue state. The antigen seeding technology approach differs from CAR-T therapies and may offers specific advantages over CAR-T therapies, including no reqirement for exogenous engeineering, operating via natural synaptic interactions between the patient's endogenous T-cells and tumor cells, and no requirement for anti-tumor cytotoxic lymphocyte involvement.

FIG. 21 summarizes the results for DI-SLTA-1::scFva::C1 (SEQ ID NO:87) and DI-SLTA-1::scFva::C1::C1 descrived above in Example 1 and in comparison to the approved anti-PD-L1 antibody therapeutic atezolizumab. In FIG. 21, the term EC₅₀ means the half-maximal effective concentration, the term IC₅₀ means the half-maximal inhibitory concentration, and the abbreviation “NT” denotes “not tested.” In FIG. 21, the phrase “fold over parental” refers to the measured value divided by the measured value for the same PD-L1 targeting molecule scaffold lacking a carboxy-terminal viral antigen, e.g. C1 or C1::C1. As noted above, the abbreviation “AST” denotes “antigen seeding technology”, which refers to the ability to create immuno-stimulation in a local area of a target cell(s) within an organism via delivery of a CD8+ T-cell epitope for MEW class I presentation, such as, e.g., delivery of T-cell epitope-peptide C1 (SEQ ID NO:77) by a PD-L1 targeting molecule resulting in cytokine secretion and/or cytotoxic T lymphocyte intercellular engagement and target cell-killing.

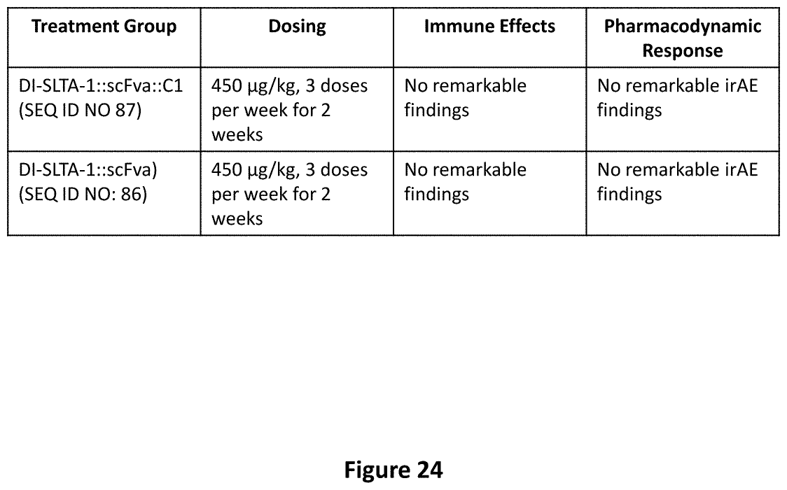

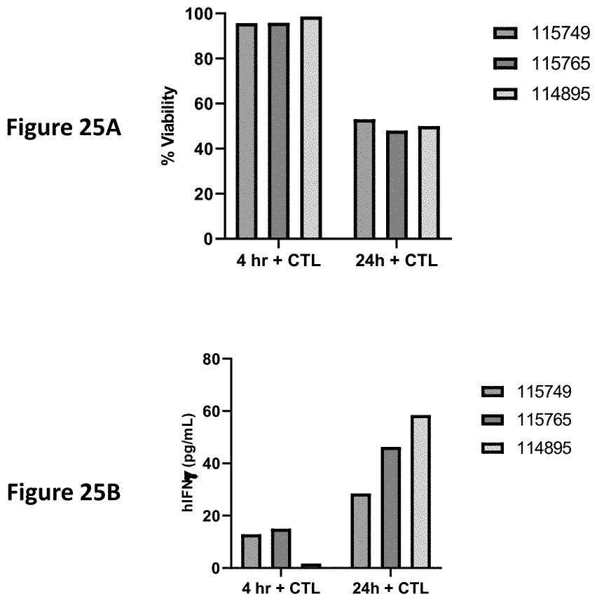

The PD-L1 binding molecules of SEQ ID NO: 86 (SI-SLTA::scFvca, 115695) and 87 (DI-SLTA-1::scFva::C1::C1, 115765) did not display signs of direct clearance of immune subsets, immune subset expansion, or tissue infiltration to promote irAE development in in vivo studies with non-human primates. Briefly, the PD-L1 binding molecules were administered to non-human primates every 3 days for a period of 2 weeks. The DI-SLTA-1::scFva::C1::C1 was administered at a dose of 25, 150, or 450 ug/kg, and the DI-SLT-A::scFva was administered at a dose of NHPs at 50, 150, or 450 μg/kg. The data are summarized in FIG. 24. In vivo depletion of monocytes either of these molecules was not observed. Additionally, none of the following were observed: (a) increases in lymphocyte populations, (ii) cytokine profiles associated with T-cell activation and/or immune checkpoint inhibitor treatment, (iii) infiltration of T-lymphocytes into cardiac tissue, (iv) myocarditis.

### VIII. Comparison of the Activities of PD-L1 Binding Molecules

An additional study was to compare the ability of various PD-L1 binding molecules to induce an antigen-specific T-lymphocyte (AST) response in target cells, as a function of time target cells are exposed to the molecules.

PD-L1 binding molecules comprising distinct binding domains were compared against each other in a co-culture cell viability assay. Briefly, PD-L1 binding molecules were incubated with PD-L1 positive and HLA:A*02 positive target cells at high density for either 4 hours or 24 hours (See Table 6). The binding molecules were then washed off. Each PD-L1 binding molecule tested comprised a peptide antigen (NLVPMVATV, SEQ ID NO: 77) for delivery to the target cells. Subsequently, cytotoxic T lymphoctes (CTLs) restricted to the peptide antigen delivered by the binding molecules were co-cultured with the pre-trated target cells, at an effector cell to target cell ratio of 1:1. After 48 hours of co-culture, supernatants were harvested and used for detection of IFN-γ as a readout for CTL activation by ELISA. Viability was also measured at 60-72 hours using an IncuCtye S3 (Sartorius) system, as determined by confluency of the monolayer. PD-L1 binding molecules were compared for their ability to promote direct cell kill or mediate T cell activation (i.e., IFN-γ secretion) after acute incubation and washout (4 h) or sustained incubation with target (24 h).

TABLE 6
PD-L1 and HLA:A*02 Positive Target Cells and HLA:A*02
NLVPMVATV (SEQ ID NO: 77) Restricted Effector Cells

| Donor ID or | | |
|---|---|---|
| Cell Line | Cell Type | Characteristics |
| MDA-MB-231 | Epithelial; | PD-L1 + and HLA:A*02 + |
| | adenocarcinoma | 10,000 cells/well in 40 μL media |
| | | in a 96-well plate |
| HCMV-CTL | Primary T Cell | HLA:A*02 NLVPMVATV restricted |
| | | Used at 1:1 Effector to target ratio |

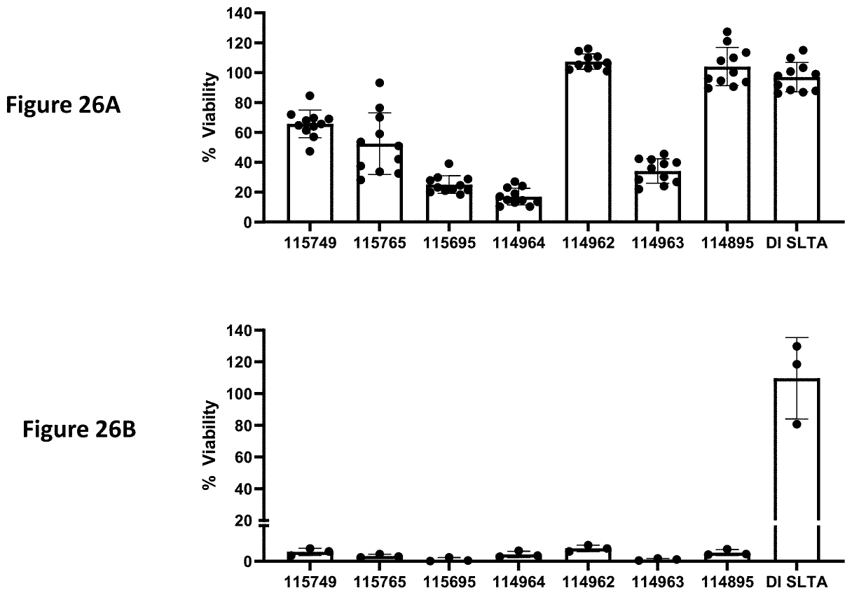

Results are shown in FIGS. 26A and 26B and summarized below in Table 7. The molecules 115749 and 114895 are PD-L1 binding molecules having different binding regions and are described in PCT/US2020/051589 which is incorporated by reference herein in its entirety. In FIGS. 26A and 26B, data for the molecules are listed in the following order from left to right: 115749, 115765, 114895. The PD-L1 binding molecules tested demonstrated T cell dependent activation and cell kill. The potency of T cell activation, as measured by IFN-γ secretion as well as the cell-kill response elicited in the presence of CTLs was increased overall for 24 hour ‘sustained’ incubation experiments as compared to 4 hour ‘acute’ PD-L1 binding molecule incubation experiments (Figure. 26A).

TABLE 7
Co-Culture Assay Results: MDA-MB-231 and HCMV-CTL Primary
Human T Cells Treated with PD-L1 Binding Molecule
PD-L1 positive human breast tumor cell line (MDA-MB-231)

| | T-cell activation | | T-cell cytotoxicity | |
|---|---|---|---|---|
| | (IFN-γ, pg/mL) | | (Max % kill) | |
| PD-L1 binding | Acute | Sustained | Acute | Sustained |
| molecule | (4 h) | (24 h) | (4 h) | (24 h) |
| 115765 | 15 | 46 | 96 | 48 |

PD-L1 negative cells (MCF7)

| | |
|---|---|
| T-cell activation | T-cell cytotoxicity |
| (IFN-γ, pg/mL) | (CD50 ng/mL) |

| PD-L1 binding | Acute | Sustained | Acute | Sustained |
|---|---|---|---|---|
| molecule | (4 h) | (24 h) | (4 h) | (24 h) |
| 115765 | >10,000 | >10,000 | >10,000 | 1338 |

The ability of the PD-L1 binding molecules to specifically deplete immune cells was also tested. The goal of these studies was to determine whether these molecules display activities that correlate to monocyte depletion and the development of immune-related adverse events (irAE) in non-human primates (NHPs) that were not observed with other PD-L1 binding molecules.

PD-L1 binding molecules were incubated with either (i) primary human monocytes (IC) treated with IFN-γ (100 IU/mL) to induce PD-L1 expression, (ii) HCC1954 tumor cells, which express high levels of PD-L1, or (iii) MCF7 tumor cells, which do not express PD-L1 (See Table 8). The PD-L1 binding molecules were added to cells using a dose series, with the final PD-L1 binding molecule concentration ranging from 0.631-20,000 ng/mL (6- or 8-fold dilutions) and left in culture throughout the duration of the study. On Day 5 of the assay, cells were evaluated for viability using a standard Cell Titer Glo assay, as described above.

TABLE 8
Target cells

| Donor ID or | | Cell Number for Cytotoxicity |
|---|---|---|
| Cell Line | Cell Type | Assay |
| Donor 1 | CD14 + Monocyte | 5,000 cells/well in 20 μL media |
| CC00152 | | in a 384-well plate |
| Donor 1 M6206 | CD14 + Monocyte | 5,000 cells/well in 20 μL media |
| | | in a 384-well plate |
| Donor 3 M6541 | CD14 + Monocyte | 5,000 cells/well in 20 μL media |
| | | in a 384-well plate |
| Donor 4 M7488 | CD14 + Monocyte | 5,000 cells/well in 20 μL media |
| | | in a 384-well plate |
| HCC1954 | Epithelial; ductal | 1,000 cells/well in 20 μL media |
| | carcinoma | in a 384-well plate |
| MCF7 | Epithelial; | 1,000 cells/well in 20 μL media |
| | adenocarcinoma | in a 384-well plate |

Results are shown in FIG. 26A, 26B and summarized in Table 9 below. Molecules 115765 and 114895 elicited a dose-dependent depletion of a representative IC population of primary monocytes across cells from four individual donors. Similar responses were observed with the PD-L1 binding molecules 114963, 114964, and 115695 which lack a C-terminal CD8+ T-cell epitope-peptide cargo. 114962 which lacks a C-terminal peptide-antigen cargo failed to target monocytes for cell depletion. A negative control, DI-SLTA alone did not show direct activity on monocytes tested in the assay.

In contrast to differential activity of different PD-L1 binding molecules toward monocytes, PD-L1 binding molecules showed similar potency toward the TC line, HCC1954, which expresses the PD-L1 target, and similar lack of activity towared the PD-L1 negative control line MCF7.

TABLE 9
Co-Culture Assay Results: HCC1954 (TC) and Primary Human T
Cells (IC) Treated with PD-L1 Binding Molecule
PD-L1 positive human breast tumor cell line (HCC1954)

| | | |
|---|---|---|
| | Cytotoxicity | Cytotoxicity |
| PD-L1 binding | (CD50 ng/mL) | (Max % kill) |

| molecule | TC | IC | TC | IC |
|---|---|---|---|---|
| 115765 | 4.7 | >20,000 | 99 | 27 |
| 115695 | 29 | 5969 | 99 | 34 |

All PD-L1 binding molecules tested elicited potent cell kill of the TC control line (Max response >90%) and displayed potencies at less than 10 ng/mL with the exception of 114962, 115749, and 114895 (>35 ng/mL).

### VIII. Quantifying Cytotoxic T-Cell Mediated Cytolysis of Intoxicated Target Cells and Other Immune Responses Triggered by MHC Class I Presentation of T-Cell Epitopes Delivered by PD-L1 Targeting Molecules

The activation of CTLs by target cells displaying epitope-peptide/MHC class I complexes (pMHC Is) is quantified using commercially available CTL response assays, e.g. CytoTox96® non-radioactive assays (Promega, Madison, Wis., U.S.), Granzyme B ELISpot assays (Mabtech, Inc., Cincinnati, Ohio, U.S.), caspase activity assays, and LAMP-1 translocation flow cytometric assays. To specifically monitor CTL-mediated killing of target cells, carboxyfluorescein succinimidyl ester (CFSE) is used to target-cells for in vitro and in vivo investigation as described in the art (see e.g. Durward M et al., J Vis Exp 45 pii 2250 (2010)).

In summary, the data presented in Example 1 demonstrates that Shiga toxin effector functions, particularly subcellular routing, can be retained at high levels despite the presence of a fused epitope-peptide on the carboxy-terminus and the presence of numerous mutations in the Shiga toxin derived component providing de-immunization and protease-cleavage resistance. Furthermore, several PD-L1 targeting molecules exhibit a level of epitope cargo delivery sufficient to produce a level of epitope-MHC class I presentation to stimulate intercellular, T-cell engagement with epitope-cargo-presenting cells.

### Example 2. PD-L1 Targeting Molecules that Bind to PD-L1 and Comprise a Shiga Toxin A Subunit Scaffold

In this example, a binding region which binds an extracellular part of a PD-L1 target biomolecule comprising a heavy chain variable region comprising three CDRs, each comprising or consisting essentially of an amino acid sequence show in any one of SEQ ID NO:22, SEQ ID NO:23, SEQ ID NO:24, SEQ ID NO:36, and SEQ ID NO:37 and a light chain variable region comprising three CDRs, having at least 95%, 96%, 97%, 98%, or 99% identity to SEQ ID NO:19, SEQ ID NO:20, and SEQ ID NO:21 is fused to a Shiga toxin effector polypeptide (such as, e.g., any one of SEQ ID NOs: 1-18, 41-69, and 261-284). The Shiga toxin effector region is derived from the A subunit of a Shiga toxin or Shiga-like toxin (e.g. any one of SEQ ID NOs: 1-18), optionally such that it comprises a combination of sub-regions described herein to provide two or more of the following: 1) de-immunization, 2) protease-cleavage resistance, and/or 3) an embedded or inserted, heterologous, T-cell epitope (such as e.g., a Shiga toxin effector polypeptide described in any one of WO 2014/164680, WO 2014/164693, WO 2015/138435, WO 2015/138452, WO 2015/113005, WO 2015/113007, WO 2015/191764, WO 2016/196344, WO 2017/019623, WO 2018/106895, and WO 2018/140427).

The resulting fusion protein is produced and purified as a single-chain polypeptide or multimer, which is optionally multivalent (i.e. has two or more PD-L1 binding regions). The illustrative proteins of this example are optionally created with a carboxy-terminal KDEL-type signal motif using techniques known in the art and optionally linked to an additional exogenous material, such as, a CD8+ T-cell epitope-peptide cargo and/or detection promoting agent(s). The illustrative proteins of this example are tested as described in the previous examples using cells expressing the appropriate PD-L1 molecule. The illustrative PD-L1 targeting fusion proteins of this example may be used, e.g., to kill PD-L1 expressing cells, to label subcellular compartments of target cells and to diagnose and treat diseases, conditions, and/or disorders, such as, e.g. various cancers and tumors.

### NUMBERED EMBODIMENTS

Notwithstanding the appended claims, the following numbered embodiments are set forth herein:

1. A PD-L1 binding molecule comprising: A) a Shiga toxin A subunit effector polypeptide; and B) a binding region capable of specifically binding an extracellular part of PD-L1, wherein the binding region comprises (a) a light chain variable region comprising: (i) a CDR1 comprising the amino acid sequence TGTSSDVGSYNRVS (SEQ ID NO:19), (ii) a CDR2 comprising the amino acid sequence EVSNRPS (SEQ ID NO:20), and (iii) a CDR3 comprising the amino acid sequence SSHTTSGTYV (SEQ ID NO:21); and (b) a heavy chain variable region comprising: (i) a CDR1 comprising the amino acid sequence SYAIS (SEQ ID NO:22), (ii) a CDR2 comprising the amino acid sequence GIIPIFGTANYAQKFQG (SEQ ID NO:23), and (iii) a CDR3 comprising the amino acid sequence DQGYAHAFDI (SEQ ID NO:24).
2. The PD-L1 binding molecule of embodiment 1, wherein the Shiga toxin A subunit effector polypeptide comprises a proteinaceous linker that links the light chain variable region and the heavy chain variable region.
3. The PD-L1 binding molecule of embodiment 2, wherein the linker is about 3 to about 50 amino acids in length.
4. The PD-L1 binding molecule of any one of embodiments 1-3, wherein the binding region is an scFv.
5. The PD-L1 binding molecule of any on of embodiments 1-4, wherein the light chain variable region comprises the sequence of any one of SEQ ID NO: 25 or 27, or a sequence at least 90% identical thereto.
6. The PD-L1 binding molecule of any one of embodiments 1-5, wherein the heavy chain variable region comprises the sequence of SEQ ID NO: 26, or a sequence at least 90% identical thereto.
7. The PD-L1 binding molecule of any one of embodiment 1, wherein the binding region comprises a sequence of any one of SEQ ID NO: 28-35 or 38-40, or a sequence at least 90% identical thereto.
8. The PD-L1 binding molecule of embodiment 1, wherein the binding region comprises SEQ ID NO: 38, or a sequence at least 90% identical thereto.
9. The PD-L1 binding molecule of any one of embodiments 1-8, wherein the molecule comprises at least one CD8+ T-cell epitope.
10. The PD-L1 binding molecule of embodiment 9, wherein the at least one CD8+ T-cell epitope is located C-terminal to the binding region.
11. The PD-L1 binding molecule of any one of embodiments 9-10, wherein the CD8+ T-cell epitope comprises the sequence of SEQ ID NO: 77.
12. The PD-L1 binding molecule of any one of embodiments 9-11, wherein the molecule comprises at least two CD8+ T-cell epitopes.
13. The PD-L1 binding molecule of embodiment 12, wherein the at least two CD8+ T-cell epitopes are different.
14. The PD-L1 binding molecule of embodiment 12, wherein the at least two CD8+ T-cell epitopes are the same.
15. The PD-L1 binding molecule of any one of embodiments 1-14, wherein the Shiga toxin A subunit effector polypeptide has at least 95% amino acid sequence identity to a wild-type Shiga toxin A Subunit amino acid sequence selected from: amino acid residues 75 to 251 of any one of SEQ ID NOs: 1-18, amino acid residues 1 to 241 of any one of SEQ ID NOs: 1-18, amino acid residues 1 to 251 of any one of SEQ ID NOs: 1-18, and amino acid residues 1 to 261 of any one of SEQ ID NOs: 1-18.
16. The PD-L1 binding molecule of any one of embodiments 1-14, wherein the Shiga toxin A subunit effector polypeptide comprises the sequence of any one of SEQ ID NO: 1-18, 41-69, 259, or 261-284.
17. The PD-L1 binding molecule of any one of embodiments 1-14, wherein the Shiga toxin A subunit effector polypeptide comprises the sequence of SEQ ID NO: 41.
18. The PD-L1 binding molecule of any one of embodiments 1-17, wherein the Shiga toxin A subunit effector polypeptide comprises a disrupted furin-cleavage site.
19. The PD-L1 binding molecule of any one of embodiments 1-18, wherein the Shiga toxin A subunit effector polypeptide and binding region are fused, forming a continuous polypeptide.
20. The PD-L1 binding molecule of embodiment 19, wherein the single continuous polypeptide comprises, from N-terminus to C-terminus, the Shiga toxin A subunit effector polypeptide and the binding region.
21. The PD-L1 binding molecule of embodiment ane of embodiments 9-11, wherein the Shiga toxin A subunit effector polypeptide and the binding reion are fused, forming a continuous polypeptide, and wherein the single continuous polypeptide comprises, from N-terminus to C-terminus, the Shiga toxin A subunit effector polypeptide, the binding region, and the heterologous CD8+ T-cell epitope.
22. The PD-L1 binding molecule of embodiment 1, wherein the molecule comprises the amino acid sequence of any one of SEQ ID NOs: 84-110, 166, or 188-256.
23. The PD-L1 binding molecule of embodiment 1, wherein the molecule comprises the amino acid sequence of SEQ ID NO: 86 or 87.
24. A pharmaceutical composition comprising the PD-L1 binding molecule of any one of embodiments 1-23, and at least one pharmaceutically acceptable excipient or carrier.
25. A polynucleotide encoding the PD-L1 binding molecule of any one of embodiments 1-23, or a complement thereof.
26. An expression vector comprising a polynucleotide according to embodiment 25.
27. A host cell comprising a polynucleotide according to embodiment 25 or an expression vector according to embodiment 26.
28. A method of killing a PD-L1 expressing cell, the method comprising the step of contacting the cell with a PD-L1 binding molecule according to any one of embodiments 1-23 or a pharmaceutical composition according to embodiment 24.
29. A method of treating a disease, disorder, or condition involving a PD-L1 expressing cell type, the method comprising the step administering to a patient in need thereof an effective amount of a PD-L1 binding molecule according to any one of embodiments 1-23 or a pharmaceutical composition according to embodiment 24.
30. The method of embodiment 29, wherein the disease, disorder, or condition selected from: cancer, tumor, immune disorder, and microbial infection.
31. The method of embodiments 30, wherein the cancer is selected from: bone cancer, breast cancer, central/peripheral nervous system cancer, gastrointestinal cancer, germ cell cancer, glandular cancer, head-neck cancer, hematological cancer, kidney-urinary tract cancer, liver cancer, lung/pleura cancer, prostate cancer, sarcoma, skin cancer, and uterine cancer.
32. The method of embodiment 30, wherein the immune disorder is associated with a disease selected from: amyloidosis, ankylosing spondylitis, asthma, Crohn's disease, diabetes, graft rejection, graft versus host disease, Hashimoto's thyroiditis, hemolytic uremic syndrome, HIV-related disease, lupus erythmatosus, multiple sclerosis, polyarteritis, psoriasis, psoriatic arthritis, rheumatoid arthritis, scleroderma, septic shock, Sjögren's syndrome, ulcerative colitis, and vasculitis.
32. A method for purifying the PD-L1 binding molecule of any one of embodiments 1-23 from a cellular lysate comprising the PD-L1 binding molecule, the method comprising (i) contacting the cellular lysate with a bacterial protein L to create a protein L-PD-L1 binding molecule complex, and (ii) separating the protein L-PD-L1 binding molecule complex from the cellular lysate.

While some embodiments of the invention have been described by way of illustration, it will be apparent that the invention may be put into practice with many modifications, variations and adaptations, and with the use of numerous equivalents or alternative solutions that are within the scope of persons skilled in the art, without departing from the spirit of the invention or exceeding the scope of the claims.

All publications, patents, and patent applications are herein incorporated by reference in their entirety to the same extent as if each individual publication, patent or patent application was specifically and individually indicated to be incorporated by reference in its entirety. The international patent application publications WO 2014/164680, WO 2014/164693, WO 2015/138435, WO 2015/138452, WO 2015/113005, WO 2015/113007, WO 2015/191764, WO 2016/196344, WO 2017/019623, WO 2018/106895, WO 2018/140427, WO 2019/183093, WO 2020/154475, and PCT/US2020/051589 are each incorporated herein by reference in its entirety. The disclosures of U.S. patent applications 62/902,243, 62/933,197, 62/970,610, 62/644,832, 63/041,288, 63/041,291, US2015/259428, US2016/17784, and US2017/143814 are each incorporated herein by reference in its entirety. The complete disclosures of all electronically available biological sequence information from GenBank (National Center for Biotechnology Information, U.S.) for amino acid and nucleotide sequences cited herein are each incorporated herein by reference in their entirety.

What is claimed is:

1. A PD-L1 binding molecule comprising:
A) a Shiga toxin A subunit effector polypeptide; and
B) a binding region capable of specifically binding an extracellular part of PD-L1, wherein the binding region comprises
(a) a light chain variable region comprising:
(i) a CDR1 comprising the amino acid sequence TGTSSDVGSYNRVS (SEQ ID NO:19),
(ii) a CDR2 comprising the amino acid sequence EVSNRPS (SEQ ID NO:20), and
(iii) a CDR3 comprising the amino acid sequence SSHTTSGTYV (SEQ ID NO:21);
and
(b) a heavy chain variable region comprising:
(i) a CDR1 comprising the amino acid sequence SYAIS (SEQ ID NO:22),
(ii) a CDR2 comprising the amino acid sequence GIIPIFGTANYAQKFQG (SEQ ID NO:23), and
(iii) a CDR3 comprising the amino acid sequence DQGYAHAFDI (SEQ ID NO:24).

2. The PD-L1 binding molecule of claim 1, wherein the Shiga toxin A subunit effector polypeptide comprises a proteinaceous linker that links the light chain variable region and the heavy chain variable region.

3. The PD-L1 binding molecule of claim 2, wherein the linker is about 3 to about 50 amino acids in length.

4. The PD-L1 binding molecule of claim 1, wherein the light chain variable region comprises the sequence of any one of SEQ ID NO: 25 or 27, or a sequence at least 90% identical thereto, and the heavy chain variable region comprises the sequence of SEQ ID NO: 26, or a sequence at least 90% identical thereto.

5. The PD-L1 binding molecule of claim 1, wherein the binding region comprises a sequence of any one of SEQ ID NO: 28-35 or 38-40, or a sequence at least 90% identical thereto.

6. The PD-L1 binding molecule of claim 1, wherein the binding region comprises SEQ ID NO: 38, or a sequence at least 90% identical thereto.

7. The PD-L1 binding molecule of claim 1, wherein the molecule comprises at least one CD8+ T-cell epitope.

8. The PD-L1 binding molecule of claim 7, wherein the at least one CD8+ T-cell epitope is located C-terminal to the binding region.

9. The PD-L1 binding molecule of claim 7, wherein the CD8+ T-cell epitope comprises the sequence of SEQ ID NO: 77.

10. The PD-L1 binding molecule claim 1, wherein the Shiga toxin A subunit effector polypeptide has at least 95% amino acid sequence identity to a wild-type Shiga toxin A Subunit amino acid sequence selected from:
amino acid residues 75 to 251 of any one of SEQ ID NOs: 1-18,
amino acid residues 1 to 241 of any one of SEQ ID NOs: 1-18,
amino acid residues 1 to 251 of any one of SEQ ID NOs: 1-18, and
amino acid residues 1 to 261 of any one of SEQ ID NOs: 1-18.

11. The PD-L1 binding molecule of claim 1, wherein the Shiga toxin A subunit effector polypeptide comprises the sequence of any one of SEQ ID NO: 1-18, 41-69, or 261-284.

12. The PD-L1 binding molecule of claim 1, wherein the Shiga toxin A subunit effector polypeptide comprises the sequence of SEQ ID NO: 41.

13. The PD-L1 binding molecule of claim 1, wherein the Shiga toxin A subunit effector polypeptide comprises a disrupted furin-cleavage site.

14. The PD-L1 binding molecule of claim 1, wherein the Shiga toxin A subunit effector polypeptide and binding region are fused, forming a continuous polypeptide that comprises, from N-terminus to C-terminus, the Shiga toxin A subunit effector polypeptide and the binding region.

15. The PD-L1 binding molecule of claim 1, wherein the molecule comprises the amino acid sequence of any one of SEQ ID NOs: 84-110, 166, or 188-256.

16. The PD-L1 binding molecule of claim 1, wherein the molecule comprises the amino acid sequence of SEQ ID NO: 86 or 87.

17. A pharmaceutical composition comprising the PD-L1 binding molecule of claim 1, and at least one pharmaceutically acceptable excipient or carrier.

18. A polynucleotide encoding the PD-L1 binding molecule of claim 1, or a complement thereof.

19. A method of treating a disease, disorder, or condition involving a PD-L1 expressing cell type, the method comprising the step administering to a patient in need thereof an effective amount of a PD-L1 binding molecule of claim 1.

20. The method of claim 19, wherein the disease, disorder, or condition is a cancer selected from: bone cancer, breast cancer, central/peripheral nervous system cancer, gastrointestinal cancer, germ cell cancer, glandular cancer, head-neck cancer, hematological cancer, kidney-urinary tract cancer, liver cancer, lung/pleura cancer, prostate cancer, sarcoma, skin cancer, and uterine cancer.
